SEQUENCE LISTING (1131 sequences)

SEQ ID NO: 1 - DNA, 1743 bp, Bos taurus
tcccaggtgcagctgcgggagtcgggccccagcctggtgaagccctcacagaccctctccctcacctgcacggtctctggattctcattaagcgattatcgtgtaggctgggtccgccaggctccaggaaaggcgctggagtggctcggtgtaatgtataatgacggaaccactgcgtataggccagccctgctatcccggctccgaatcaccagggacaactccaagaaccaagtcgccctgtcactaagcagcgtaacaactgaggacacggccacatactactgtacaagaataattcctgtgtgtacttggccttatggtgactatggctgttatgacatcttgggccaaggactcctggtcaccgtctcctcagaaggtgaatcacacccgagagtcttccccctggtgtcctgcgtgagctccccatccgatgagagcacggtggccctgggctgcctggcccgggacttcgtgcccaattcagtcagcttctcctggaagttcaacaacagcacagtcagcagcgagagattctggaccttccccgaagtcctgagggacggcttgtggtcggcctcctctcaggtggtcctgccctcctcaagcgcctttcaagggccggatgactacctggtgtgcgaagtccagcaccccaagggaggaaagaccgtcggcaccgtgagggtggtcgctacaaaggcggaagtgctgtccccagtcgtgagtgtctttgtcccgcctcgcaacagcctctctggtgacggcaatagcaagtccagcctcatctgccaggccacggacttcagccccaaacagatctccttgtcctggtttcgtgatggaaagcggatagtgtctggcatttctgaaggccaggtggagactgtgcagtcctcacccgtaactttcagggcctacagcatgctgaccatcacggagagagactggctcagccagaacgtgtacacctgccaggtagaacacaacaaggaaaccttccagaagaacgtgtcctcctcatgtgatgttgcaccaccatctcccatcggggtcttcaccatccccccatccttcgccgacatcttcctcacgaagtcagccaagctgtcctgtctggtcacaaacctggcctcctatgatggcctgaacatcagctggtcccgtcagaacggcaaggccctggagacccacacgtattttgagcgacacctcaacgacaccttcagcgcccggggtgaggcctcggtctgctcggaggactgggagtccggagaggagttcacgtgcacagtggcccactcggacctgcccttcccagaaaagaacaccgtctccaagcccaaagatgtcgccatgaaaccgccgtccgtgtacctgctgcctccaacgcgggaacagctgagcctgcgggagtcggcctccgtcacctgcctggtgaagggcttcgcgcccgcggacgtgttcgtgcagtggctgcagaggggggagcccgtgaccaagagcaagtacgtgaccagcagcccggcgcccgagccccaggaccccagcgtgtactttgtgcacagcatcctgacggtggccgaggaggactggagcaaaggggagacctacacctgcgtcgtgggccacgaggccctgccccacatggtcaccgagcggaccgtggacaagtccaccggtaaacccaccctgtacaacgtgtccctggtcctgtctgacacagccagcacctgctac

SEQ ID NO: 2 - protein, 581 aa, Bos taurus
SQVQLRESGPSLVKPSQTLSLTCTVSGFSLSDYRVGWVRQAPGKALEWLGVMYNDGTTAYRPALLSRLRITRDNSKNQVALSLSSVTTEDTATYYCTRIIPVCTWPYGDYGCYDILGQGLLVTVSSEGESHPRVFPLVSCVSSPSDESTVALGCLARDFVPNSVSFSWKFNNSTVSSERFWTFPEVLRDGLWSASSQVVLPSSSAFQGPDDYLVCEVQHPKGGKTVGTVRVVATKAEVLSPVVSVFVPPRNSLSGDGNSKSSLICQATDFSPKQISLSWFRDGKRIVSGISEGQVETVQSSPVTFRAYSMLTITERDWLSQNVYTCQVEHNKETFQKNVSSSCDVAPPSPIGVFTIPPSFADIFLTKSAKLSCLVTNLASYDGLNISWSRQNGKALETHTYFERHLNDTFSARGEASVCSEDWESGEEFTCTVAHSDLPFPEKNTVSKPKDVAMKPPSVYLLPPTREQLSLRESASVTCLVKGFAPADVFVQWLQRGEPVTKSKYVTSSPAPEPQDPSVYFVHSILTVAEEDWSKGETYTCVVGHEALPHMVTERTVDKSTGKPTLYNVSLVLSDTASTCY

SEQ ID NO: 3 - DNA, 2055 bp, Bos taurus
gaccctgagagaactgtgagatggtgcaccatttcaactcatgaggccaataagtgtgccagttttcgtgaaaatgtgcttcgtatcttggagagtggtccttttgtctcctgtgtgaagaaaacctcacacatggattgcatcaaggctatctcgaataacgaagcagatgctgtgacgttggatggaggtttggtgtatgaggcaggcctgaagcccaacaacctgaagcctgtggtggcagagttccatgggacaaaagacaacccacaaactcactattatgctgtggcggtggtgaagaaggacactgacttcaagctgaatgagctcagaggcaagaagtcctgccacacaggcctcggcaggtccgctgggtggaacatccccatgggcaaactttataaggaattgcctgatccacaggaatcaattcagagagctgcggccaacttcttctctgccagctgtgtcccctgtgcggatcaatcatcatttcccaaactctgtcaactgtgtgcggggaaagggacagacaagtgtgcctgctccaaccacgaaccatacttcggctactcaggggcctttaaatgtctgatggagggcgctggggatgtggcctttgtcaagcactcaacggtattcgacaacctgcctaacccagaagacaggaaaaactatgagctgctctgcggggacaacacccggaagtctgtagataattaccaagaatgctacttggcaatggttccttcccatgcggttgtggctcgaactgtgggcggcaaggaggatgtgatctgggaacttctcaatcacgcccaggaacattttggcaaagataaaccagacaatttccagcttttccaatcccctcatgggaaggacctgctgtttaaggactctgctgatgggtttttaaagattccttctaagatggattttgagctgtacttgggatatgaatatgtcactgctcttcagaatctaagagaaagtaaacccccggactcctcaaaggatgagtgcatggtgaagtggtgtgcaattggtcaccaggagaggacaaagtgtgatcggtggagtgggttcagcggcggggcaatagagtgtgaaacagcagagaacactgaagagtgcatcgccaagatcatgaaaggagaagctgatgccatgagcttggacggaggctacctctacatagcaggcaaatgtggcctggtgcctgtcctggcagagaactacaaaactgagggtgaaagttgcaagaacacaccagaaaaagggtatcttgctgtagctgtggttaaaacatcagatgctaatatcaactggaacaatctgaaagacaagaagtcctgccacactgcagtagacagaaccgctggctggaacatccccatgggtttgctctacagcaagattaataactgtaaatttgatgaatttttcagcgcaggttgtgcacctggatctccgcgaaattccagtctctgtgctctgtgcattggctcagagaagggtacaggaaaggagtgtgttcccaacagcaatgaaagatactatggctatacaggggctttcaggtgtctggtggagaagggagacgtggcctttgtgaaggaccagactgtcatacagaacactgacggaaataataatgaagcatgggcaaaaaatctgaagaaggaaaattttgaagtactatgcaaagatggcaccaggaaacctgtgacagatgctgagaactgccacctggcccgaggcccgaatcatgctgtggtctcacggaaagataaggcaacttgtgtggagaaaatattaaacaaacagcaggatgattttggaaaatctgtaaccgactgcacgagcaatttttgtttattccaatcaaattccaaggaccttctgttcagggatgacactaaatgtttggcttcaattgcgaaaaaaacatatgactcctacttaggggatgactacgtcagagctatgaccaacctgagacaatgctcaacctcaaaactcctggaagcatgcactttccacaaacct

SEQ ID NO: 4 - protein, 685 aa, Bos taurus
DPERTVRWCTISTHEANKCASFRENVLRILESGPFVSCVKKTSHMDCIKAISNNEADAVTLDGGLVYEAGLKPNNLKPVVAEFHGTKDNPQTHYYAVAVVKKDTDFKLNELRGKKSCHTGLGRSAGWNIPMGKLYKELPDPQESIQRAAANFFSASCVPCADQSSFPKLCQLCAGKGTDKCACSNHEPYFGYSGAFKCLMEGAGDVAFVKHSTVFDNLPNPEDRKNYELLCGDNTRKSVDNYQECYLAMVPSHAVVARTVGGKEDVIWELLNHAQEHFGKDKPDNFQLFQSPHGKDLLFKDSADGFLKIPSKMDFELYLGYEYVTALQNLRESKPPDSSKDECMVKWCAIGHQERTKCDRWSGFSGGAIECETAENTEECIAKIMKGEADAMSLDGGYLYIAGKCGLVPVLAENYKTEGESCKNTPEKGYLAVAVVKTSDANINWNNLKDKKSCHTAVDRTAGWNIPMGLLYSKINNCKFDEFFSAGCAPGSPRNSSLCALCIGSEKGTGKECVPNSNERYYGYTGAFRCLVEKGDVAFVKDQTVIQNTDGNNNEAWAKNLKKENFEVLCKDGTRKPVTDAENCHLARGPNHAVVSRKDKATCVEKILNKQQDDFGKSVTDCTSNFCLFQSNSKDLLFRDDTKCLASIAKKTYDSYLGDDYVRAMTNLRQCSTSKLLEACTFHKP

SEQ ID NO: 5 - DNA, 2070 bp, Bos taurus
gacacaattgcacaggccgccagcaccaccaccatctctgatgctgtgagtaaggtcaagatccaggtcaacaaggccttcctggattcccggaccaggctgaagacgaccttgagctctgaggcacccaccacccaacagctctcagagtacttcaagcacgcaaagggccggacccgcacggccattcgcaacgggcaggtgtgggaggagtccttaaagaggctgaggcgggacacaaccctgaccaacgtcacagaccctagcctggacttgactgcactctcctgggaggtgggctgcggtgccccggttcctctggtgaaatgtgatgaaaacagcccttaccgcaccatcacgggagactgtaataacaggaggagccccgcactgggcgccgccaacagggcgctggcgcgctggctgccggcggagtacgaggacgggctcgccctgcccttcggctggacgcagaggaagacgcgcaacggcttccgcgtcccgctggcccgggaggtatccaacaaaattgtaggctacctggacgaagagggtgttctggaccaaaacaggtccctgctcttcatgcagtggggtcagattgtggaccacgacctggacttcgccccggaaacggaactggggagcaacgagcactctaaaacccagtgtgaggagtactgtatccagggagacaactgcttccccatcatgttcccgaaaaatgatcccaagttgaagactcaagggaaatgcatgcctttcttccgagccgggtttgtctgccccactccaccttaccagtcgttggcccgagaacagatcaatgctgtgacctccttcctggacgccagcttagtgtacggctctgagcccagtctggccagccgtctccggaacctcagcagcccgctgggcctcatggctgtcaaccaagaagcctgggaccacgggctggcctacctgccctttaacaacaagaagccgagcccctgtgagttcatcaacaccaccgcccgcgtgccctgtttcctggcgggagattttcgagcctcagagcagattctgctggccactgcccacaccctccttctccgggagcacaaccggctggccagagaactaaagaaactcaaccctcactggaatggagagaagctctaccaggaagcccggaaaatcctgggagccttcatacagatcatcacctttagggactacctacccattgtgctaggtagtgagatgcagaagtggatcccgccctaccaaggctataataactctgtggatccccgaatttccaatgtcttcacctttgccttccgctttggccacatggaggttccctccactgtgtcccgcctggatgagaattaccagccatggggtccggaagcagagctccccctacacaccctcttcttcaacacctggaggataatcaaagacggtggaattgaccctctggtgcggggtctgctggccaagaagtccaaactgatgaatcaggataaaatggtgacgagtgagctgcgcaacaagcttttccagcccactcacaagatccacggctttgacctggctgctatcaacttacagcgttgccgagaccatgggatgcctgggtacaactcctggaggggcttctgtggcctctcacagcccaagacactgaaggggctgcagactgtgctgaagaacaagatactggctaagaagttaatggatctctataagacccccgacaacattgacatctggattggaggcaacgctgagcccatggtagaaaggggccgggtggggccactcctggcctgcctcctagggaggcaattccagcagatacgtgatggggacaggttctggtgggagaaccctggggtcttcactgagaagcagcgggactctctacagaaagtgtccttctcacgcctcatctgtgacaacacccacatcacgaaggtcccgctgcatgccttccaggccaacaactacccacatgactttgtggattgctcaaccgttgataagctggatctctcaccctgggcctccagggagaat

SEQ ID NO: 6 - protein, 690 aa, Bos taurus
DTIAQAASTTTISDAVSKVKIQVNKAFLDSRTRLKTTLSSEAPTTQQLSEYFKHAKGRTRTAIRNGQVWEESLKRLRRDTTLTNVTDPSLDLTALSWEVGCGAPVPLVKCDENSPYRTITGDCNNRRSPALGAANRALARWLPAEYEDGLALPFGWTQRKTRNGFRVPLAREVSNKIVGYLDEEGVLDQNRSLLFMQWGQIVDHDLDFAPETELGSNEHSKTQCEEYCIQGDNCFPIMFPKNDPKLKTQGKCMPFFRAGFVCPTPPYQSLAREQINAVTSFLDASLVYGSEPSLASRLRNLSSPLGLMAVNQEAWDHGLAYLPFNNKKPSPCEFINTTARVPCFLAGDFRASEQILLATAHTLLLREHNRLARELKKLNPHWNGEKLYQEARKILGAFIQIITFRDYLPIVLGSEMQKWIPPYQGYNNSVDPRISNVFTFAFRFGHMEVPSTVSRLDENYQPWGPEAELPLHTLFFNTWRIIKDGGIDPLVRGLLAKKSKLMNQDKMVTSELRNKLFQPTHKIHGFDLAAINLQRCRDHGMPGYNSWRGFCGLSQPKTLKGLQTVLKNKILAKKLMDLYKTPDNIDIWIGGNAEPMVERGRVGPLLACLLGRQFQQIRDGDRFWWENPGVFTEKQRDSLQKVSFSRLICDNTHITKVPLHAFQANNYPHDFVDCSTVDKLDLSPWASREN

SEQ ID NO: 7 - DNA, 1356 bp, Bos taurus
tcccaggtgcagctgcgggagtcgggccccagcctggtgaagccctcacagaccctctccctcacgtgcacggtgtctggattctcattgagcagctatgctctaacctgggtccgccaggctccagggaaggcgctggagtgggttggtggtataaccagtggtggaaccacatactataatccagccctgaaatcccggctcagcatcaccaaggagaactccaagagccaagtctctctgtcagtgagcagcgtgacacctgaggacacagccacatactactgtgcaagaagtacttatggtgaggttggtgatggtgccatcgccgatgcctggggccaaggactcctggtcaccgtctcctcagcctccaccacagccccgaaagtctaccctctgagttcttgctgcggggacaagtccagctccaccgtgaccctgggctgcctggtctccagctacatgcccgagccggtgaccgtgacctggaactcgggtgccctgaagagcggcgtgcacaccttcccggctgtccttcagtcctccgggctgtactctctcagcagcatggtgaccgtgcccggcagcacctcaggacagaccttcacctgcaacgtagcccacccggccagcagcaccaaggtggacaaggctgttgatcccacatgcaaaccatcaccctgtgactgttgcccaccccctgagctccccggaggaccctctgtcttcatcttcccaccgaaacccaaggacaccctcacaatctcgggaacgcccgaggtcacgtgtgtggtggtggacgtgggccacgatgaccccgaggtgaagttctcctggttcgtggacgacgtggaggtaaacacagccacgacgaagccgagagaggagcagttcaacagcacctaccgcgtggtcagcgccctgcgcatccagcaccaggactggactggaggaaaggagttcaagtgcaaggtccacaacgaaggcctcccggcccccatcgtgaggaccatctccaggaccaaagggccggcccgggagccgcaggtgtatgtcctggccccaccccaggaagagctcagcaaaagcacggtcagcctcacctgcatggtcaccagcttctacccagactacatcgccgtggagtggcagagaaacgggcagcctgagtcggaggacaagtacggcacgaccccgccccagctggacgccgacagctcctacttcctgtacagcaagctcagggtggacaggaacagctggcaggaaggagacacctacacgtgtgtggtgatgcacgaggccctgcacaatcactacacgcagaagtccacctctaagtctgcgggtaaa

SEQ ID NO: 8 - protein, 452 aa, Bos taurus
SQVQLRESGPSLVKPSQTLSLTCTVSGFSLSSYALTWVRQAPGKALEWVGGITSGGTTYYNPALKSRLSITKENSKSQVSLSVSSVTPEDTATYYCARSTYGEVGDGAIADAWGQGLLVTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 9 - DNA, 1350 bp, Bos taurus
atggcatctgttgcagctgaaccacaactgagtgtggtgaccagggtggccaacctccccttggtgagctccacgtatgatctggtctcctcggcttacatcagtagaaaggatcagtatccttacttgaagtctctgtgtgagatggcagagaagggcatgaagaccatcacttctgtggctgtgaccagcgcgctgccaatcatccaaaagttagagccacaaattgctgttgccaatacctatgcgtgtaaggggctagacaggattgaggagaagctgcctattctgaatcagccaacaaaccaggttgtggccaatgccaaaggggctatgactggggcaaaagatgctgtgacgactactgtgaccggggccaaggattctgtggccagcacaatcacgggggtggtggacaggaccaagggagctgtgactggtagtgtggagaagaccaagtccgtggtcagtggcagcattaacacagtcctacgaagtcgggtgatgcagctgatgagcagtggagtagaaaatgcactcaccaaatcagagctgctggtagaccagtacctccctcttaccaaggatgaactagaaaaagaagccaaaaaagtggaaggattcgatatggttcagaagccgagttactatgttagactgggatctctgtccaccaagctgcgctcacgggcctaccagcaggccctttgtagggttgaagaagctaagcgaaaaggccaggagaccatttctcagctccattccgccttcaacctgagtgaacttgccaggaagaatgtgcataatgccaaccagaaaattcaggatgctcaggataagctctatctgtcctggctggagtggaagagaagcatcggctacgatgatacagatgaatcccactgtgctgagcacattgagtcacgtactcttgctattgcccggaacctgactcagcagctccagaccatgtgccacaccctcctgtccaacatccaggggttaccacagaacattcaagatcgggccaaccacttgggggtgatggctggtgacatctactctgtgtttcgcaatgctgcctcctttaaagaagtgtctgatggcctcctcgcttccagcaaggggcagctgcagaaaatgaaggagtctttagatgatgtgatggattatcttgttaacaacacacccctcaactggctggtaggtcccttttatccccaggtgaccgagtctgagagtgctcaggccccaggtacaaccaggaggcctggcaggtggagtagaaaacaccctaaacccgtccctgtcagcaatgcagagggcagccagccagatgacagctcctct

SEQ ID NO: 10 - protein, 450 aa, Bos taurus
MASVAAEPQLSVVTRVANLPLVSSTYDLVSSAYISRKDQYPYLKSLCEMAEKGMKTITSVAVTSALPIIQKLEPQIAVANTYACKGLDRIEEKLPILNQPTNQVVANAKGAMTGAKDAVTTTVTGAKDSVASTITGVVDRTKGAVTGSVEKTKSVVSGSINTVLRSRVMQLMSSGVENALTKSELLVDQYLPLTKDELEKEAKKVEGFDMVQKPSYYVRLGSLSTKLRSRAYQQALCRVEEAKRKGQETISQLHSAFNLSELARKNVHNANQKIQDAQDKLYLSWLEWKRSIGYDDTDESHCAEHIESRTLAIARNLTQQLQTMCHTLLSNIQGLPQNIQDRANHLGVMAGDIYSVFRNAASFKEVSDGLLASSKGQLQKMKESLDDVMDYLVNNTPLNWLVGPFYPQVTESESAQAPGTTRRPGRWSRKHPKPVPVSNAEGSQPDDSSS

SEQ ID NO: 11 - DNA, 2217 bp, Bos taurus
aagagtcccatcttcggtcccgaggaggtgaccagcgtggaaggccgctcagtgtccatcaagtgctactacccgcccacctccgtcaaccggcacacgcgcaagtactggtgccggcagggagcccagggccgctgcacgaccctcatctcctcggagggctacgtctccgacgactacgtgggcagagccaacctcaccaacttcccggagagcggcacgtttgtggtggacatcagccatctcacccataaagactcagggcgctacaagtgtggcctgggcattagcagccgtggccttaacttcgatgtgagcctggaggtcagccaagatcctgcacaggcaagtcatgcccacgtctacactgtagacctgggcaggactgtgaccatcaactgccctttcacgcgtgcgaattctgagaagagaaaatccttgtgcaagaagacaatccaggactgtttccaagttgtcgactccaccgggtatgtgagcaacagctataaagacagagcacatatcagtatcctaggtaccaacacattagtgttcagcgttgtcatcaaccgagtcaagctcagtgatgctgggatgtatgtctgccaggctggggacgatgccaaagccgataaaatcaacattgacctccaggtgctggagcctgagcctgagctggtttatggagacttgaggagctcggtgacctttgactgttccctgggccccgaggtggcaaatgtgcccaaatttctgtgccagaagaagaatgggggagcttgcaatgtggtcatcaacacgttggggaagaaggctcaggacttccagggcaggatcgtgtccgtgcccaaggacaatggtgtcttcagtgtgcacattaccagcctgaggaaagaggacgcagggcgctacgtgtgcggggcccagcctgagggtgagccccaggacggctggcctgtgcaggcctggcaactcttcgtcaatgaagagacggcaatccccgcaagcccctccgtggtgaaaggtgtgaggggaggctctgtgaccgtatcttgcccctacaacccgaaggatgccaacagcgcgaagtactggtgtcactgggaagaggctcaaaacggccgctgcccgcggctggtggagagccgggggctgataaaggagcagtacgagggcaggctggcgctgcttaccgagccgggcaacggcacctacaccgtcatcctcaaccagctcaccgaccaggacaccggcttctactggtgcgtgaccgacggcgacacgcgctggatctccacagtggagctcaaggttgtccaaggagaaccaagcctcaaggtacccaagaacgtcacggcttggctgggagagcccttaaagctctcctgccacttcccctgcaaattctactcctttgagaagtactggtgtaagtggagcaacagaggctgcagcgccctgcccacccagaacgacggccccagccaggcctttgtgagctgtgaccagaacagccaggtcgtctccctgaacctggacacagtcaccaaggaggatgaaggctggtactggtgtggagtgaaggagggcccccgatatggggagacggcggctgtctacgtggcagtggagagcagggtgaaggggtcccaaggcgccaagcaagtgaaagctgcccctgcgggggcggcaatacagtcgagggccggggagatccagaacaaagcccttctggaccccagctttttcgcaaaggaaagtgtgaaggacgctgctggtggacccggagcacctgcagatcctggccgccctacaggatacagcgggagctccaaagcactggtctccaccctggtgcccctggccctggtcctggtcgcaggggtcgtggcgatcggggtggtccgagcccggcacaggaagaacgtcgaccggatttcaatcaggagctaccggacagatatcagcatgtcagactttgagaactccagggattttgaaggacgtgacaacatgggagcctctccagaggcccaagagacgtctctcggagggaaggacgagtttgccaccactaccgaggacaccgtggagagcaaagaacccaagaaggcaaagaggtcgtccaaggaggaagccgacgaggccttcaccaccttcctcctccaggccaaaaacctggcctccgccgcaacccagaacggcccgacagaagcc

SEQ ID NO: 12 - protein, 739 aa, Bos taurus
KSPIFGPEEVTSVEGRSVSIKCYYPPTSVNRHTRKYWCRQGAQGRCTTLISSEGYVSDDYVGRANLTNFPESGTFVVDISHLTHKDSGRYKCGLGISSRGLNFDVSLEVSQDPAQASHAHVYTVDLGRTVTINCPFTRANSEKRKSLCKKTIQDCFQVVDSTGYVSNSYKDRAHISILGTNTLVFSVVINRVKLSDAGMYVCQAGDDAKADKINIDLQVLEPEPELVYGDLRSSVTFDCSLGPEVANVPKFLCQKKNGGACNVVINTLGKKAQDFQGRIVSVPKDNGVFSVHITSLRKEDAGRYVCGAQPEGEPQDGWPVQAWQLFVNEETAIPASPSVVKGVRGGSVTVSCPYNPKDANSAKYWCHWEEAQNGRCPRLVESRGLIKEQYEGRLALLTEPGNGTYTVILNQLTDQDTGFYWCVTDGDTRWISTVELKVVQGEPSLKVPKNVTAWLGEPLKLSCHFPCKFYSFEKYWCKWSNRGCSALPTQNDGPSQAFVSCDQNSQVVSLNLDTVTKEDEGWYWCGVKEGPRYGETAAVYVAVESRVKGSQGAKQVKAAPAGAAIQSRAGEIQNKALLDPSFFAKESVKDAAGGPGAPADPGRPTGYSGSSKALVSTLVPLALVLVAGVVAIGVVRARHRKNVDRISIRSYRTDISMSDFENSRDFEGRDNMGASPEAQETSLGGKDEFATTTEDTVESKEPKKAKRSSKEEADEAFTTFLLQAKNLASAATQNGPTEA

SEQ ID NO: 13 - DNA, 1938 bp, Bos taurus
aaccccatgtattccatgatcacccccaacatcctgcgtgtggagagcgaggagactgtggtgctggaggcccacggaggacaagggactattcaggtctcggtcaccgtccacgacttcccggccaagaaacaagtgctgtcaaacgaaaacacccagctgaacagcaacaatggctacctgagcaccgtcaccatcaagatcccggccagcaaagagttaaaatccgacaaggggcacaagttcgtgactgtcgtagcgaccttcgggaatgtccaggtggagaaggtggtgctgatcagccttcagagcgggtacctcttcatccagacggacaagaccatctacaccccaggctccacggtcctctatcgggtcttcactgttgaccacaagctgctgcccgtgggccagacggttttcatcaccatcgagacccctgatggcatccctgtcaagcgggactccaagtcttctcagaaccagtttggcattttgaccttgtcctggaacattccagagctagtgaacatgggggtgtggaagatcaaagcctactatgaagattctccgcagcaggtcttctccgctgagtttgaggtgaaggaatatgtgcttcccagttttgaggtccaactagagcctgaagagaaattctactacattgatgacccagatggcctgaaggtcaacatcattgccaggttcttgtacggggaacaggtggacggaacagctttcgtcatctttggggtccaggatggcgaccggagaatttcattgactcactccctcacccgtgttccgatcaacgatggtaatggggaggccatactgaaacgtcaggttttgctgaatggagtacagccctcccgagctgatgccctggtgggcaagtccatatatgtgtccgccaccgtcatcttgcaatcaggcagtgacatggtggaggcagagcgcaccgggatccccattgtgacttccccctaccagatccatttcaccaagacccccaagttcttcaaacctgccatgcccttcgacctcatggtgtacgtgacaaaccccgacggctcccccgctcgccacatcccggtggtgacccagggcagcaatgtgcagtccttgacccaggatgatggcgtggccaagctaagcatcaacacacagaacaaacgggatcccctgaccatcaccgtacgcacaaaaaaggacaatatccccgaggggcgacaggccaccaggaccatgcaggccctaccctacaacacccaggggaactccaataactacctgcacctctctgtgccccgcgtggagctcaagcctggggagactctcaacgtcaacttccacctgcgcacggaccccggcgagcaagccaagatccgctactacacctacatgatcatgaacaaggggaagctgttgaaggtgggacgccagtaccgagagcccggccaggacctagtggtgttgcccttgaccatcacgtcagacttcatcccttccttccgcctggtggcctattacacactgattaatgccaaaggccagagggaggtggtggccgactccgtgtgggtggatgtcaaggactcgtgtatgggcacgctggtggtgaaaaatggtgggaaggaagagaaacaccatcgacccgggcaacagataaccctgaagatcgaggctgaccagggggcccgagtggggctcgtggccgtggacaagggcgtgtttgtgctgaataagaagaacaaattgacccagagaaagatctgggacgtggtggagaaagcggacattggctgcaccccaggcagtggaagaaactatgctggcgtcttcacggatgcaggactcactctcaagaccagccagggcctggagacccagcagagggcagatccgcagtgcccgcaaccagccacccgccgacgc

SEQ ID NO: 14 - protein, 646 aa, Bos taurus
NPMYSMITPNILRVESEETVVLEAHGGQGTIQVSVTVHDFPAKKQVLSNENTQLNSNNGYLSTVTIKIPASKELKSDKGHKFVTVVATFGNVQVEKVVLISLQSGYLFIQTDKTIYTPGSTVLYRVFTVDHKLLPVGQTVFITIETPDGIPVKRDSKSSQNQFGILTLSWNIPELVNMGVWKIKAYYEDSPQQVFSAEFEVKEYVLPSFEVQLEPEEKFYYIDDPDGLKVNIIARFLYGEQVDGTAFVIFGVQDGDRRISLTHSLTRVPINDGNGEAILKRQVLLNGVQPSRADALVGKSIYVSATVILQSGSDMVEAERTGIPIVTSPYQIHFTKTPKFFKPAMPFDLMVYVTNPDGSPARHIPVVTQGSNVQSLTQDDGVAKLSINTQNKRDPLTITVRTKKDNIPEGRQATRTMQALPYNTQGNSNNYLHLSVPRVELKPGETLNVNFHLRTDPGEQAKIRYYTYMIMNKGKLLKVGRQYREPGQDLVVLPLTITSDFIPSFRLVAYYTLINAKGQREVVADSVWVDVKDSCMGTLVVKNGGKEEKHHRPGQQITLKIEADQGARVGLVAVDKGVFVLNKKNKLTQRKIWDVVEKADIGCTPGSGRNYAGVFTDAGLTLKTSQGLETQQRADPQCPQPATRRR

SEQ ID NO: 15 - DNA, 645 bp, Bos taurus
caggctgtgctgactcagccgtcctccgtgtccggctccctgggccagagggtctccatcacctgctctggaagcagcagcaacatcggtagagataatgtgggctggtaccaacaggtcccaggatcgggcctcagaaccatcatctatagtagtagcagtcgaccctcgggggtccccgaccgattctccggctccaagtctggcaacacagccaccctgaccatcagctccctccaggctgaggacgaggcggattatttctgtgtagcttatgacagtagtagtagtactgttttcggcagcgggaccacactgaccgtcctgggtcagcccaagtccccaccctcggtcaccctgttcccgccctccacggaggagctcaacggcaacaaggccaccctggtgtgtctcatcagcgacttctacccgggtagcgtgaccgtggtctggaaggcagacggcagcaccatcacccgcaacgtggagaccacccgggcctccaaacagagcaacagcaagtacgcggccagcagctacctgagcctgacgagcagcgactggaaatcgaaaggcagttacagctgcgaggtcacgcacgaggggagcaccgtgacgaagacagtgaagccctcagagtgttct

SEQ ID NO: 16 - protein, 215 aa, Bos taurus
QAVLTQPSSVSGSLGQRVSITCSGSSSNIGRDNVGWYQQVPGSGLRTIIYSSSSRPSGVPDRFSGSKSGNTATLTISSLQAEDEADYFCVAYDSSSSTVFGSGTTLTVLGQPKSPPSVTLFPPSTEELNGNKATLVCLISDFYPGSVTVVWKADGSTITRNVETTRASKQSNSKYAASSYLSLTSSDWKSKGSYSCEVTHEGSTVTKTVKPSECS

SEQ ID NO: 17 - DNA, 1524 bp, Bos taurus
gaacagcaaggttacagaaactccaccaacactggttcctatgaaaaggacttggattgcggagcccctggcaccccagaggcccagctctgttttgacccctgccagaattacaccctcctgaatgaaccctttcgaagcacagagaatacagaagacatccaaggctgtgacagtgacaaacacggctggtaccgatttgtgggggacggaggagtgaggatgccagaggactgtgtccccacgttccgatgccagacgtctgcacccctgtggctgaatggcacccatcctggccttggggagggcatcgtcaaccgcactgcctgtgcccactggagtggcaactgctgcctctggaagacagaggtgctggtgaaagcctgcccgggcccatactacgtgtaccggttggagggcacccctcaatgtagtctgagatactgcacagaccctgccaccgcggaggacaagtgtgacaggacctgccgccccgaggaggagtgtcgcctcgtcagtggcacctggggctgcttctgcagacaggacctcaacgtctccgatgtgcacagtttgcagcctcagctggactgtggggacacagagatcaaagtgtcactggacaagtgtctgctgggaagtctgggtttcggagatgaggtccatgcctacctgcgggacgggaactggaactgcagcagcctcaggcagagtgaagaggagaactggatatctgtgaccaatcccacccaggctggtgcctgcggaaacattctggagagaaaccagacccatgccatctacataaacacgctctccttagtcaacgatttcatcatcagagacaccatcctcagcatcaacttccagtgtgcctacccactggacatgaaagtgagccttcaaatggccctgcagcccatcgtaagttccctgaacatcactgtggatggggagggagagttcactgtcaggatggccctctttcaagaccaggactacacgtctccttatgaagggactgcagtcatgctgtctgttgaatccatgctctatgtgggcaccatcttggagagaggggacacgtcccgatttaacctggtgttgaagaactgctacgccacgcccactgaagacaagactgaccctgtgaagtacttcatcatcaggaacagctgcccaaatcaacgtgattccaccatctccgtggaagagaatggggtgtcagcagaaagccggttctcagttcagatgttcaagtttgctggaaattatgacctagttttcctgcattgtgaggtttctctctgtgatttcattaaagaagagtgccaaccgtcttgctcaagaagtcaactccgcagtgaaggggtggctattgatccagcccgagttctagatctgggacccatcactcgcaaaggtgcccagtctcttggtgtcatgagtggaacacccaacactgcagggttcctggtggcctggcccctggttctcctgcctgtcctcctggctgggctgttc

SEQ ID NO: 18 - protein, 508 aa, Bos taurus
EQQGYRNSTNTGSYEKDLDCGAPGTPEAQLCFDPCQNYTLLNEPFRSTENTEDIQGCDSDKHGWYRFVGDGGVRMPEDCVPTFRCQTSAPLWLNGTHPGLGEGIVNRTACAHWSGNCCLWKTEVLVKACPGPYYVYRLEGTPQCSLRYCTDPATAEDKCDRTCRPEEECRLVSGTWGCFCRQDLNVSDVHSLQPQLDCGDTEIKVSLDKCLLGSLGFGDEVHAYLRDGNWNCSSLRQSEEENWISVTNPTQAGACGNILERNQTHAIYINTLSLVNDFIIRDTILSINFQCAYPLDMKVSLQMALQPIVSSLNITVDGEGEFTVRMALFQDQDYTSPYEGTAVMLSVESMLYVGTILERGDTSRFNLVLKNCYATPTEDKTDPVKYFIIRNSCPNQRDSTISVEENGVSAESRFSVQMFKFAGNYDLVFLHCEVSLCDFIKEECQPSCSRSQLRSEGVAIDPARVLDLGPITRKGAQSLGVMSGTPNTAGFLVAWPLVLLPVLLAGLF

SEQ ID NO: 19 - DNA, 1227 bp, Bos taurus
gcctccggtgacttctgtgactccagcttgtgcctacatggtgggacctgcttgttgaacgaggacagaaccccccccttctactgcctctgccccgaaggcttcacgggcctcctatgcaacgagacagagcacggtccctgtttcccaaacccctgccacaatgatgctgagtgccaggtaactgacgactcccaccgaggggatgtcttcatccagtacatctgcaagtgccctcttggatacgtgggcatccactgtgagaccacctgcacctcaccgctgggcatgcagacgggtgccattgccgactcccagatctctgcctcgtccatgcatttgggcttcatgggtttgcagcgctgggccccggagctagcccgcctgcaccagacgggcatcgtcaacgcttggacatccggcaactatgacaaaaacccctggatccaggtgaacctgatgcggaagatgtgggtgacgggtgtggtgacccagggggccagccgcgcagggagtgctgagtacctgaagacttttaaagtggcctacagcaccgatgggcgccaattccagttcatccaggttgcagggcggtcaggagataagatatttataggtaatgtgaacaacagcggcctgaagattaacctgtttgatacccccttggagacgcagtatgtgagactggtacccatcatctgccaccggggctgcaccctccgctttgaactccttggctgtgagttgaatggatgcactgaacccctaggcctgaaggataataccatccccaacaagcagatcacagcctccagctactacaaaacctggggcctgagtgcctttagctggtttccctactacgcacgactggataatcagggcaagttcaacgcctggaccgcccagaccaacagtgcctctgagtggctgcagattgacctgggctcccagaagcgagtcacgggcatcatcacccagggtgcccgagactttggccacattcaatatgtggctgcctacagggtggcctatggtgatgatggtgtgacctggactgagtacaaggacccgggggcctcagaaagcaagattttccctggtaacatggacaataattcccacaagaagaacatatttgagacgccgttccaggctcgcttcgtgcggatccagcccgtggcctggcacaaccgtatcaccctgcgagtggagctgctgggctgt

SEQ ID NO: 20 - protein, 409 aa, Bos taurus
ASGDFCDSSLCLHGGTCLLNEDRTPPFYCLCPEGFTGLLCNETEHGPCFPNPCHNDAECQVTDDSHRGDVFIQYICKCPLGYVGIHCETTCTSPLGMQTGAIADSQISASSMHLGFMGLQRWAPELARLHQTGIVNAWTSGNYDKNPWIQVNLMRKMWVTGVVTQGASRAGSAEYLKTFKVAYSTDGRQFQFIQVAGRSGDKIFIGNVNNSGLKINLFDTPLETQYVRLVPIICHRGCTLRFELLGCELNGCTEPLGLKDNTIPNKQITASSYYKTWGLSAFSWFPYYARLDNQGKFNAWTAQTNSASEWLQIDLGSQKRVTGIITQGARDFGHIQYVAAYRVAYGDDGVTWTEYKDPGASESKIFPGNMDNNSHKKNIFETPFQARFVRIQPVAWHNRITLRVELLGC

SEQ ID NO: 21 - DNA, 846 bp, Bos taurus
gaggagacccaggctgggaattactctctaagcttcctctacaccgggctgtccaagccccgtgagggcttccccagcttccaggccgttgcctacctcaatgaccagcccttcttccactacaatagtgaaggcaggagggccgagcccctggcgccatggagtcaggtggaaggaatggaggactgggagaaggagagtgcccttcagagggccagagaggacatcttcatggagaccctgagcgatatcatggactactacaaggacagagaagggtctcacacctttcaaggagcattcggctgtgagctccggaataacgaaagtagtggagcattctgggggtacgcctacgatggccaggacttcatcaagttcgacaaagaaatcccagcctgggtccctctggacccagcagctcagaacaccaagcggaagtgggaagcagaggcagtctacgtgcagcgggccaaggcctacctggaagaggagtgcccagggatgctgcggaggtacctgccctacagcaggacccacctggaccgacaagagtctccttctgtgtcggtcaccgggcacgcggctccagggcacaagaggacactcaagtgcctggcctacgacttctacccacgaagcatcgggctgcactggactcgggctggtgacgcccaggaggctgagtcagggggcgacgtgctccccagcggcaacggcacttaccagtcctgggtggtggtgggggtcccccctgaggaccaagccccgtactcctgccacgtggagcacagaagcctgacccggcccctcaccgtcccgtgggacccgcggcaacaagccgag

SEQ ID NO: 22 - protein, 282 aa, Bos taurus
EETQAGNYSLSFLYTGLSKPREGFPSFQAVAYLNDQPFFHYNSEGRRAEPLAPWSQVEGMEDWEKESALQRAREDIFMETLSDIMDYYKDREGSHTFQGAFGCELRNNESSGAFWGYAYDGQDFIKFDKEIPAWVPLDPAAQNTKRKWEAEAVYVQRAKAYLEEECPGMLRRYLPYSRTHLDRQESPSVSVTGHAAPGHKRTLKCLAYDFYPRSIGLHWTRAGDAQEAESGGDVLPSGNGTYQSWVVVGVPPEDQAPYSCHVEHRSLTRPLTVPWDPRQQAE

SEQ ID NO: 23 - DNA, 390 bp, Bos taurus
gagccggtgaaattcaaggactgcggttcttgggtcggagttataaaggaagtgaatgtgagcccatgtcccacccagccttgcaaactgcacagaggacagtcttacagtgttaatgtcacgttcaccagtaatactcagtctcaaagtagcaaggctgtggtacatggcattgtgatgggcatcccagttccctttcccattcctgagtctgatggttgtaagtctggaatcagatgccccatcgaaaaagataagacctataactacgtgaacaaactacccgtgaagaatgaatacccctccataaaagtggtggtggagtgggaactcacggatgacaaaaaccaacgtttcttctgctggcagatcccaatagaggttgaagcc

SEQ ID NO: 24 - protein, 130 aa, Bos taurus
EPVKFKDCGSWVGVIKEVNVSPCPTQPCKLHRGQSYSVNVTFTSNTQSQSSKAVVHGIVMGIPVPFPIPESDGCKSGIRCPIEKDKTYNYVNKLPVKNEYPSIKVVVEWELTDDKNQRFFCWQIPIEVEA

SEQ ID NO: 25 - DNA, 1080 bp, Bos taurus
gaggtcaatgccgaccaggtggccaccgtgatttgggactacttcagccagctgggcaacaatgccaagaaggcggtggaacatatccagaagtctgagctcacccagcagctcaacaccctcttccaagataaacttggggaagtgagcacctacacggacgacctgcagaagaagctggtgccctttgccacggaactgcatgaacggctgaccaaggactccgagaagctgaaggaggagattcggaaggagctggaggacctgcgggcccggctgctgccccacgccacggaggtgagccagaagatcggggacaacgtgcgcgagctacagcagcgcctgggaccatacgcagaggagctgcgcacccaggtcgacacccaggcgcagcagctgcggcgccagctgacgccctatgtggagcgcatggagaaggtgatgcggcagaacctggaccagctgcaggcctcgctggcgccctacgccgaggagctccaggccacggtcaaccagcgcgtggaagagctcaaggggcgcctgacgccctacgcggaccagctccagaccaagatcgaggagaacgtggaggaactgcgccgcagcctggccccttatgctcaggacgtccaggggaagctcaaccaccagctggagggcctggcctttcagatgaagaagcatgccgaggagctaaaggccaagatctcagccaaagccgaggagctgcggcaggggctggtgcccctggtcaacagcgtgcatggaagtcagctgggcaatgccgaggacttgcagaagtcactggccgagctgagcagccgcctggaccagcaggtggaggacttccgccgcaccgtggggccctacggcgagaccttcaacaaagccatggtgcagcagctggacacgctcaggcagaagctgggccccctggcgggggacgtggaagatcacctgagtttcctggagaaggatctgagggacaaggtcagcagcttcttcaacaccctcaaggagaaagagagccaggcccccgccctccccgcgcaggaggagatgcccgtcccattggggggc

SEQ ID NO: 26 - protein, 360 aa, Bos taurus
EVNADQVATVIWDYFSQLGNNAKKAVEHIQKSELTQQLNTLFQDKLGEVSTYTDDLQKKLVPFATELHERLTKDSEKLKEEIRKELEDLRARLLPHATEVSQKIGDNVRELQQRLGPYAEELRTQVDTQAQQLRRQLTPYVERMEKVMRQNLDQLQASLAPYAEELQATVNQRVEELKGRLTPYADQLQTKIEENVEELRRSLAPYAQDVQGKLNHQLEGLAFQMKKHAEELKAKISAKAEELRQGLVPLVNSVHGSQLGNAEDLQKSLAELSSRLDQQVEDFRRTVGPYGETFNKAMVQQLDTLRQKLGPLAGDVEDHLSFLEKDLRDKVSSFFNTLKEKESQAPALPAQEEMPVPLGG

SEQ ID NO: 27 - protein, 15 aa, Artificial Sequence
  epitope
QNVYTCQVEHNKETF

SEQ ID NO: 28 - protein, 15 aa, Artificial Sequence
  epitope
QNVYTCQVEHNKETF

SEQ ID NO: 29 - protein, 7 aa, Artificial Sequence
  epitope
SQNVYTC

SEQ ID NO: 30 - protein, 7 aa, Artificial Sequence
  epitope
TCQVEHN

SEQ ID NO: 31 - protein, 7 aa, Artificial Sequence
  epitope
HNKETFQ

SEQ ID NO: 32 - protein, 15 aa, Artificial Sequence
  epitope
QNVYTCQVEHNKETF

SEQ ID NO: 33 - protein, 8 aa, Artificial Sequence
  epitope
LSQNVYTC

SEQ ID NO: 34 - protein, 4 aa, Artificial Sequence
  epitope
HNKE

SEQ ID NO: 35 - protein, 15 aa, Artificial Sequence
  epitope
QNVYTCQVEHNKETF

SEQ ID NO: 36 - protein, 4 aa, Artificial Sequence
  epitope
HNKE

SEQ ID NO: 37 - protein, 15 aa, Artificial Sequence
  epitope
QNVYTCQVEHNKETF

SEQ ID NO: 38 - protein, 15 aa, Artificial Sequence
  epitope
  misc_feature (1)..(3): Xaa can be any naturally occurring amino acid
  misc_feature (5)..(5): Xaa can be any naturally occurring amino acid
  misc_feature (8)..(10): Xaa can be any naturally occurring amino acid
  misc_feature (13)..(15): Xaa can be any naturally occurring amino acid
XXXYXCQXXXNKXXX

SEQ ID NO: 39 - protein, 4 aa, Artificial Sequence
  epitope
HNKE

SEQ ID NO: 40 - protein, 15 aa, Artificial Sequence
  epitope
QNVYTCQVEHNKETF

SEQ ID NO: 41 - protein, 10 aa, Artificial Sequence
  epitope
DWLSQNVYTC

SEQ ID NO: 42 - protein, 15 aa, Artificial Sequence
  epitope
QNVYTCQVEHNKETF

SEQ ID NO: 43 - protein, 8 aa, Artificial Sequence
  epitope
LSQNVYTC

SEQ ID NO: 44 - protein, 10 aa, Artificial Sequence
  epitope
NVYTCQVEHN

SEQ ID NO: 45 - protein, 10 aa, Artificial Sequence
  epitope
YTCQVEHNKE

SEQ ID NO: 46 - protein, 6 aa, Artificial Sequence
  epitope
HNKETF

SEQ ID NO: 47 - protein, 15 aa, Artificial Sequence
  epitope
QNVYTCQVEHNKETF

SEQ ID NO: 48 - protein, 15 aa, Artificial Sequence
  epitope
QNVYTCQVEHNKETF

SEQ ID NO: 49 - protein, 15 aa, Artificial Sequence
  epitope
  misc_feature (1)..(3): Xaa can be any naturally occurring amino acid
  misc_feature (5)..(6): Xaa can be any naturally occurring amino acid
  misc_feature (8)..(10): Xaa can be any naturally occurring amino acid
XXXYXXQXXXNKETF

SEQ ID NO: 50 - protein, 15 aa, Artificial Sequence
  epitope
  misc_feature (1)..(10): Xaa can be any naturally occurring amino acid
  misc_feature (14)..(14): Xaa can be any naturally occurring amino acid
XXXXXXXXXXNKEXF

SEQ ID NO: 51 - protein, 10 aa, Artificial Sequence
  epitope
YTCQVEHNKE

SEQ ID NO: 52 - protein, 10 aa, Artificial Sequence
  epitope
  misc_feature (2)..(3): Xaa can be any naturally occurring amino acid
  misc_feature (5)..(7): Xaa can be any naturally occurring amino acid
YXXQXXXNKE

SEQ ID NO: 53 - protein, 6 aa, Artificial Sequence
  epitope
HNKETF

SEQ ID NO: 54 - protein, 6 aa, Artificial Sequence
  epitope
  misc_feature (1)..(1): Xaa can be any naturally occurring amino acid
XNKETF

SEQ ID NO: 55 - protein, 6 aa, Artificial Sequence
  epitope
  misc_feature (1)..(1): Xaa can be any naturally occurring amino acid
  misc_feature (5)..(5): Xaa can be any naturally occurring amino acid
XNKEXF

SEQ ID NO: 56 - protein, 6 aa, Artificial Sequence
  epitope
HNKETF

SEQ ID NO: 57 - protein, 10 aa, Artificial Sequence
  epitope
YTCQVEHNKE

SEQ ID NO: 58 - protein, 6 aa, Artificial Sequence
  epitope
HNKETF

SEQ ID NO: 59 - protein, 15 aa, Artificial Sequence
  epitope
TKSKYVTSSPAPEPQ

SEQ ID NO: 60 - protein, 15 aa, Artificial Sequence
  epitope
TKSKYVTSSPAPEPQ

SEQ ID NO: 61 - protein, 15 aa, Artificial Sequence
  epitope
  misc_feature (1)..(1): Xaa can be any naturally occurring amino acid
  misc_feature (5)..(7): Xaa can be any naturally occurring amino acid
XKSKXXXSSPAPEPQ

SEQ ID NO: 62 - protein, 15 aa, Artificial Sequence
  epitope
  misc_feature (1)..(1): Xaa can be any naturally occurring amino acid
  misc_feature (3)..(3): Xaa can be any naturally occurring amino acid
  misc_feature (5)..(13): Xaa can be any naturally occurring amino acid
XKXKXXXXXXXXXPQ

SEQ ID NO: 63 - protein, 9 aa, Artificial Sequence
  epitope
KSKYVTSSP

SEQ ID NO: 64 - protein, 9 aa, Artificial Sequence
  epitope
  misc_feature (4)..(6): Xaa can be any naturally occurring amino acid
KSKXXXSSP

SEQ ID NO: 65 - protein, 8 aa, Artificial Sequence
  epitope
KSKYVTSS

SEQ ID NO: 66 - protein, 8 aa, Artificial Sequence
  epitope
  misc_feature (4)..(6): Xaa can be any naturally occurring amino acid
KSKXXXSS

SEQ ID NO: 67 - protein, 4 aa, Artificial Sequence
  epitope
YVTS

SEQ ID NO: 68 - protein, 15 aa, Artificial Sequence
  epitope
TKSKYVTSSPAPEPQ

SEQ ID NO: 69 - protein, 15 aa, Artificial Sequence
  epitope
  misc_feature (1)..(1): Xaa can be any naturally occurring amino acid
  misc_feature (3)..(3): Xaa can be any naturally occurring amino acid
  misc_feature (5)..(8): Xaa can be any naturally occurring amino acid
  misc_feature (12)..(12): Xaa can be any naturally occurring amino acid
XKXKXXXXSPAXEPQ

SEQ ID NO: 70 - protein, 6 aa, Artificial Sequence
  epitope
SKYVTS

SEQ ID NO: 71 - protein, 5 aa, Artificial Sequence
  epitope
KYVTS

SEQ ID NO: 72 - protein, 10 aa, Artificial Sequence
  epitope
PAPEPQDPSV

SEQ ID NO: 73 - protein, 10 aa, Artificial Sequence
  epitope
  misc_feature (3)..(3): Xaa can be any naturally occurring amino acid
PAXEPQDPSV

SEQ ID NO: 74 - protein, 15 aa, Artificial Sequence
  epitope
TKSKYVTSSPAPEPQ

SEQ ID NO: 75 - protein, 7 aa, Artificial Sequence
  epitope
SKYVTSS

SEQ ID NO: 76 - protein, 15 aa, Artificial Sequence
  epitope
TKSKYVTSSPAPEPQ

SEQ ID NO: 77 - protein, 9 aa, Artificial Sequence
  epitope
KSKYVTSSP

SEQ ID NO: 78 - protein, 8 aa, Artificial Sequence
  epitope
KSKYVTSS

SEQ ID NO: 79 - protein, 6 aa, Artificial Sequence
  epitope
SKYVTS

SEQ ID NO: 80 - protein, 10 aa, Artificial Sequence
  epitope
YVTSSPAPEP

SEQ ID NO: 81 - protein, 15 aa, Artificial Sequence
  epitope
TKSKYVTSSPAPEPQ

SEQ ID NO: 82 - protein, 15 aa, Artificial Sequence
  epitope
  misc_feature (1)..(1): Xaa can be any naturally occurring amino acid
  misc_feature (5)..(6): Xaa can be any naturally occurring amino acid
  misc_feature (12)..(12): Xaa can be any naturally occurring amino acid
XKSKXXTSSPAXEPQ

SEQ ID NO: 83 - protein, 8 aa, Artificial Sequence
  epitope
KSKYVTSS

SEQ ID NO: 84 - protein, 6 aa, Artificial Sequence
  epitope
SKYVTS

SEQ ID NO: 85 - protein, 6 aa, Artificial Sequence
  epitope
  misc_feature (3)..(4): Xaa can be any naturally occurring amino acid
SKXXTS

SEQ ID NO: 86 - protein, 5 aa, Artificial Sequence
  epitope
KYVTS

SEQ ID NO: 87 - protein, 6 aa, Artificial Sequence
  epitope
KYVTSS

SEQ ID NO: 88 - protein, 4 aa, Artificial Sequence
  epitope
YVTS

SEQ ID NO: 89 - protein, 6 aa, Artificial Sequence
  epitope
SKYVTS

SEQ ID NO: 90 - protein, 6 aa, Artificial Sequence
  epitope
KYVTSS

SEQ ID NO: 91 - protein, 4 aa, Artificial Sequence
  epitope
YVTS

SEQ ID NO: 92 - protein, 15 aa, Artificial Sequence
  epitope
  misc_feature (1)..(1): Xaa can be any naturally occurring amino acid
  misc_feature (3)..(3): Xaa can be any naturally occurring amino acid
  misc_feature (6)..(6): Xaa can be any naturally occurring amino acid
  misc_feature (9)..(9): Xaa can be any naturally occurring amino acid
XKXKYXTSXPAPEPQ

SEQ ID NO: 93 - protein, 15 aa, Artificial Sequence
  epitope
  misc_feature (1)..(3): Xaa can be any naturally occurring amino acid
  misc_feature (6)..(6): Xaa can be any naturally occurring amino acid
  misc_feature (8)..(9): Xaa can be any naturally occurring amino acid
  misc_feature (12)..(15): Xaa can be any naturally occurring amino acid
XXXKYXTXXPAXXXX

SEQ ID NO: 94 - protein, 9 aa, Artificial Sequence
  epitope
KSKYVTSSP

SEQ ID NO: 95 - protein, 10 aa, Artificial Sequence
  epitope
KYVTSSPAPE

SEQ ID NO: 96 - protein, 5 aa, Artificial Sequence
  epitope
KYVTS

SEQ ID NO: 97 - protein, 5 aa, Artificial Sequence
  epitope
  misc_feature (3)..(3): Xaa can be any naturally occurring amino acid
KYXTS

SEQ ID NO: 98 - protein, 15 aa, Artificial Sequence
  epitope
CGDNTRKSVDNYQEC

SEQ ID NO: 99 - protein, 15 aa, Artificial Sequence
  epitope
CGDNTRKSVDNYQEC

SEQ ID NO: 100 - protein, 15 aa, Artificial Sequence
  epitope
  misc_feature (13)..(15): Xaa can be any naturally occurring amino acid
CGDNTRKSVDNYXXX

SEQ ID NO: 101 - protein, 15 aa, Artificial Sequence
  epitope
  misc_feature (1)..(1): Xaa can be any naturally occurring amino acid
  misc_feature (9)..(9): Xaa can be any naturally occurring amino acid
  misc_feature (12)..(15): Xaa can be any naturally occurring amino acid
XGDNTRKSXDNXXXX

SEQ ID NO: 102 - protein, 10 aa, Artificial Sequence
  epitope
KSVDNYQECY

SEQ ID NO: 103 - protein, 10 aa, Artificial Sequence
  epitope
  misc_feature (7)..(9): Xaa can be any naturally occurring amino acid
KSVDNYXXXY

SEQ ID NO: 104 - protein, 10 aa, Artificial Sequence
  epitope
  misc_feature (3)..(3): Xaa can be any naturally occurring amino acid
  misc_feature (6)..(9): Xaa can be any naturally occurring amino acid
KSXDNXXXXY

SEQ ID NO: 105 - protein, 9 aa, Artificial Sequence
  epitope
DNYQECYLA

SEQ ID NO: 106 - protein, 9 aa, Artificial Sequence
  epitope
  misc_feature (4)..(6): Xaa can be any naturally occurring amino acid
DNYXXXYLA

SEQ ID NO: 107 - protein, 9 aa, Artificial Sequence
  epitope
  misc_feature (3)..(6): Xaa can be any naturally occurring amino acid
DNXXXXYLA

SEQ ID NO: 108 - protein, 15 aa, Artificial Sequence
  epitope
CGDNTRKSVDNYQEC

SEQ ID NO: 109 - protein, 15 aa, Artificial Sequence
  epitope
  misc_feature (1)..(1): Xaa can be any naturally occurring amino acid
  misc_feature (5)..(5): Xaa can be any naturally occurring amino acid
  misc_feature (9)..(9): Xaa can be any naturally occurring amino acid
  misc_feature (13)..(15): Xaa can be any naturally occurring amino acid
XGDNXRKSXDNYXXX

SEQ ID NO: 110 - protein, 15 aa, Artificial Sequence
  epitope
  misc_feature (1)..(1): Xaa can be any naturally occurring amino acid
  misc_feature (3)..(6): Xaa can be any naturally occurring amino acid
  misc_feature (9)..(9): Xaa can be any naturally occurring amino acid
  misc_feature (13)..(15): Xaa can be any naturally occurring amino acid
XGXXXXKSXDNYXXX

SEQ ID NO: 111 - protein, 10 aa, Artificial Sequence
  epitope
KSVDNYQECY

SEQ ID NO: 112 - protein, 10 aa, Artificial Sequence
  epitope
  misc_feature (3)..(3): Xaa can be any naturally occurring amino acid
  misc_feature (7)..(9): Xaa can be any naturally occurring amino acid
KSXDNYXXXY

SEQ ID NO: 113 - protein, 9 aa, Artificial Sequence
  epitope
DNYQECYLA

SEQ ID NO: 114 - protein, 15 aa, Artificial Sequence
  epitope
CGDNTRKSVDNYQEC

SEQ ID NO: 115 - protein, 15 aa, Artificial Sequence
  epitope
  misc_feature (1)..(2): Xaa can be any naturally occurring amino acid
  misc_feature (4)..(5): Xaa can be any naturally occurring amino acid
  misc_feature (9)..(9): Xaa can be any naturally occurring amino acid
  misc_feature (12)..(15): Xaa can be any naturally occurring amino acid
XXDXXRKSXDNXXXX

SEQ ID NO: 116 - protein, 4 aa, Artificial Sequence
  epitope
DNTR

SEQ ID NO: 117 - protein, 10 aa, Artificial Sequence
  epitope
TRKSVDNYQE

SEQ ID NO: 118 - protein, 10 aa, Artificial Sequence
  epitope
  misc_feature (1)..(1): Xaa can be any naturally occurring amino acid
  misc_feature (5)..(5): Xaa can be any naturally occurring amino acid
  misc_feature (8)..(10): Xaa can be any naturally occurring amino acid
XRKSXDNXXX

SEQ ID NO: 119 - protein, 10 aa, Artificial Sequence
  epitope
KSVDNYQECY

SEQ ID NO: 120 - protein, 10 aa, Artificial Sequence
  epitope
  misc_feature (3)..(3): Xaa can be any naturally occurring amino acid
  misc_feature (6)..(9): Xaa can be any naturally occurring amino acid
KSXDNXXXXY

SEQ ID NO: 121 - protein, 15 aa, Artificial Sequence
  epitope
CGDNTRKSVDNYQEC

SEQ ID NO: 122 - protein, 15 aa, Artificial Sequence
  epitope
CGDNTRKSVDNYQEC

SEQ ID NO: 123 - protein, 15 aa, Artificial Sequence
  epitope
  misc_feature (1)..(1): Xaa can be any naturally occurring amino acid
  misc_feature (8)..(9): Xaa can be any naturally occurring amino acid
  misc_feature (13)..(15): Xaa can be any naturally occurring amino acid
XGDNTRKXXDNYXXX

SEQ ID NO: 124 - protein, 15 aa, Artificial Sequence
  epitope
  misc_feature (1)..(1): Xaa can be any naturally occurring amino acid
  misc_feature (7)..(10): Xaa can be any naturally occurring amino acid
  misc_feature (12)..(15): Xaa can be any naturally occurring amino acid
XGDNTRXXXXNXXXX

SEQ ID NO: 125 - protein, 4 aa, Artificial Sequence
  epitope
DNTR

SEQ ID NO: 126 - protein, 10 aa, Artificial Sequence
  epitope
KSVDNYQECY

SEQ ID NO: 127 - protein, 10 aa, Artificial Sequence
  epitope
  misc_feature (2)..(3): Xaa can be any naturally occurring amino acid
  misc_feature (7)..(9): Xaa can be any naturally occurring amino acid
KXXDNYXXXY

SEQ ID NO: 128 - protein, 15 aa, Artificial Sequence
  epitope
  misc_feature (1)..(2): Xaa can be any naturally occurring amino acid
  misc_feature (4)..(5): Xaa can be any naturally occurring amino acid
  misc_feature (9)..(9): Xaa can be any naturally occurring amino acid
  misc_feature (13)..(15): Xaa can be any naturally occurring amino acid
XXDXXRKSXDNYXXX

SEQ ID NO: 129 - protein, 15 aa, Artificial Sequence
  epitope
  misc_feature (1)..(1): Xaa can be any naturally occurring amino acid
  misc_feature (3)..(5): Xaa can be any naturally occurring amino acid
  misc_feature (13)..(15): Xaa can be any naturally occurring amino acid
XGXXXRKSVDNYXXX

SEQ ID NO: 130 - protein, 15 aa, Artificial Sequence
  epitope
  misc_feature (1)..(6): Xaa can be any naturally occurring amino acid
  misc_feature (8)..(9): Xaa can be any naturally occurring amino acid
  misc_feature (13)..(15): Xaa can be any naturally occurring amino acid
XXXXXXKXXDNYXXX

SEQ ID NO: 131 - protein, 15 aa, Artificial Sequence
  epitope
  misc_feature (1)..(1): Xaa can be any naturally occurring amino acid
  misc_feature (14)..(15): Xaa can be any naturally occurring amino acid
XGDNTRKSVDNYQXX

SEQ ID NO: 132 - protein, 15 aa, Artificial Sequence
  epitope
  misc_feature (1)..(5): Xaa can be any naturally occurring amino acid
  misc_feature (8)..(9): Xaa can be any naturally occurring amino acid
  misc_feature (13)..(15): Xaa can be any naturally occurring amino acid
XXXXXRKXXDNYXXX

SEQ ID NO: 133 - protein, 15 aa, Artificial Sequence
  epitope
  misc_feature (1)..(5): Xaa can be any naturally occurring amino acid
  misc_feature (14)..(15): Xaa can be any naturally occurring amino acid
XXXXXRKSVDNYQXX

SEQ ID NO: 134 - protein, 15 aa, Artificial Sequence
  epitope
  misc_feature (1)..(6): Xaa can be any naturally occurring amino acid
  misc_feature (8)..(8): Xaa can be any naturally occurring amino acid
  misc_feature (13)..(15): Xaa can be any naturally occurring amino acid
XXXXXXKXVDNYXXX

SEQ ID NO: 135 - protein, 7 aa, Artificial Sequence
  epitope
DNTRKSV

SEQ ID NO: 136 - protein, 7 aa, Artificial Sequence
  epitope
  misc_feature (1)..(3): Xaa can be any naturally occurring amino acid
XXXRKSV

SEQ ID NO: 137 - protein, 10 aa, Artificial Sequence
  epitope
TRKSVDNYQE

SEQ ID NO: 138 - protein, 10 aa, Artificial Sequence
  epitope
  misc_feature (1)..(1): Xaa can be any naturally occurring amino acid
  misc_feature (10)..(10): Xaa can be any naturally occurring amino acid
XRKSVDNYQX

SEQ ID NO: 139 - protein, 10 aa, Artificial Sequence
  epitope
  misc_feature (1)..(2): Xaa can be any naturally occurring amino acid
  misc_feature (4)..(4): Xaa can be any naturally occurring amino acid
  misc_feature (9)..(10): Xaa can be any naturally occurring amino acid
XXKXVDNYXX

SEQ ID NO: 140 - protein, 15 aa, Artificial Sequence
  epitope
  misc_feature (1)..(1): Xaa can be any naturally occurring amino acid
  misc_feature (8)..(9): Xaa can be any naturally occurring amino acid
  misc_feature (12)..(15): Xaa can be any naturally occurring amino acid
XGDNTRKXXDNXXXX

SEQ ID NO: 141 - protein, 15 aa, Artificial Sequence
  epitope
  misc_feature (1)..(2): Xaa can be any naturally occurring amino acid
  misc_feature (5)..(5): Xaa can be any naturally occurring amino acid
  misc_feature (7)..(10): Xaa can be any naturally occurring amino acid
  misc_feature (12)..(15): Xaa can be any naturally occurring amino acid
XXDNXRXXXXNXXXX

SEQ ID NO: 142 - protein, 15 aa, Artificial Sequence
  epitope
  misc_feature (1)..(1): Xaa can be any naturally occurring amino acid
  misc_feature (3)..(3): Xaa can be any naturally occurring amino acid
  misc_feature (9)..(9): Xaa can be any naturally occurring amino acid
  misc_feature (12)..(12): Xaa can be any naturally occurring amino acid
  misc_feature (14)..(15): Xaa can be any naturally occurring amino acid
XGXNTRKSXDNXQXX

SEQ ID NO: 143 - protein, 15 aa, Artificial Sequence
  epitope
  misc_feature (1)..(5): Xaa can be any naturally occurring amino acid
  misc_feature (9)..(9): Xaa can be any naturally occurring amino acid
  misc_feature (12)..(15): Xaa can be any naturally occurring amino acid
XXXXXRKSXDNXXXX

SEQ ID NO: 144 - protein, 4 aa, Artificial Sequence
  epitope
DNTR

SEQ ID NO: 145 - protein, 15 aa, Artificial Sequence
  epitope
SPHGKDLLFKDSADG

SEQ ID NO: 146 - protein, 15 aa, Artificial Sequence
  epitope
SPHGKDLLFKDSADG

SEQ ID NO: 147 - protein, 6 aa, Artificial Sequence
  epitope
SPHGKD

SEQ ID NO: 148 - protein, 8 aa, Artificial Sequence
  epitope
KDLLFKDS

SEQ ID NO: 149 - protein, 15 aa, Artificial Sequence
  epitope
SPHGKDLLFKDSADG

SEQ ID NO: 150 - protein, 10 aa, Artificial Sequence
  epitope
QLFQSPHGKD

SEQ ID NO: 151 - protein, 15 aa, Artificial Sequence
  epitope
SPHGKDLLFKDSADG

SEQ ID NO: 152 - protein, 15 aa, Artificial Sequence
  epitope
  misc_feature (1)..(2): Xaa can be any naturally occurring amino acid
  misc_feature (7)..(9): Xaa can be any naturally occurring amino acid
  misc_feature (11)..(15): Xaa can be any naturally occurring amino acid
XXHGKDXXXKXXXXX

SEQ ID NO: 153 - protein, 9 aa, Artificial Sequence
  epitope
KDSADGFLK

SEQ ID NO: 154 - protein, 15 aa, Artificial Sequence
  epitope
SPHGKDLLFKDSADG

SEQ ID NO: 155 - protein, 15 aa, Artificial Sequence
  epitope
  misc_feature (1)..(2): Xaa can be any naturally occurring amino acid
  misc_feature (6)..(9): Xaa can be any naturally occurring amino acid
  misc_feature (12)..(12): Xaa can be any naturally occurring amino acid
  misc_feature (14)..(15): Xaa can be any naturally occurring amino acid
XXHGKXXXXKDXAXX

SEQ ID NO: 156 - protein, 9 aa, Artificial Sequence
  epitope
QSPHGKDLL

SEQ ID NO: 157 - protein, 9 aa, Artificial Sequence
  epitope
  misc_feature (2)..(3): Xaa can be any naturally occurring amino acid
  misc_feature (7)..(9): Xaa can be any naturally occurring amino acid
QXXHGKXXX

SEQ ID NO: 158 - protein, 6 aa, Artificial Sequence
  epitope
KDSADG

SEQ ID NO: 159 - protein, 15 aa, Artificial Sequence
  epitope
SPHGKDLLFKDSADG

SEQ ID NO: 160 - protein, 15 aa, Artificial Sequence
  epitope
  misc_feature (1)..(1): Xaa can be any naturally occurring amino acid
  misc_feature (6)..(15): Xaa can be any naturally occurring amino acid
XPHGKXXXXXXXXXX

SEQ ID NO: 161 - protein, 10 aa, Artificial Sequence
  epitope
QLFQSPHGKD

SEQ ID NO: 162 - protein, 10 aa, Artificial Sequence
  epitope
  misc_feature (5)..(5): Xaa can be any naturally occurring amino acid
  misc_feature (10)..(10): Xaa can be any naturally occurring amino acid
QLFQXPHGKX

SEQ ID NO: 163 - protein, 5 aa, Artificial Sequence
  epitope
GKDLL

SEQ ID NO: 164 - protein, 8 aa, Artificial Sequence
  epitope
KDSADGFL

SEQ ID NO: 165 - protein, 15 aa, Artificial Sequence
  epitope
SPHGKDLLFKDSADG

SEQ ID NO: 166 - protein, 8 aa, Artificial Sequence
  epitope
KDSADGFL

SEQ ID NO: 167 - protein, 15 aa, Artificial Sequence
  epitope
SPHGKDLLFKDSADG

SEQ ID NO: 168 - protein, 10 aa, Artificial Sequence
  epitope
QLFQSPHGKD

SEQ ID NO: 169 - protein, 15 aa, Artificial Sequence
  epitope
SPHGKDLLFKDSADG

SEQ ID NO: 170 - protein, 15 aa, Artificial Sequence
  epitope
SPHGKDLLFKDSADG

SEQ ID NO: 171 - protein, 15 aa, Artificial Sequence
  epitope
  misc_feature (2)..(2): Xaa can be any naturally occurring amino acid
  misc_feature (4)..(4): Xaa can be any naturally occurring amino acid
  misc_feature (7)..(10): Xaa can be any naturally occurring amino acid
  misc_feature (12)..(13): Xaa can be any naturally occurring amino acid
SXHXKDXXXXDXXDG

SEQ ID NO: 172 - protein, 6 aa, Artificial Sequence
  epitope
KDLLFK

SEQ ID NO: 173 - protein, 5 aa, Artificial Sequence
  epitope
DLLFK

SEQ ID NO: 174 - protein, 10 aa, Artificial Sequence
  epitope
FKDSADGFLK

SEQ ID NO: 175 - protein, 10 aa, Artificial Sequence
  epitope
  misc_feature (1)..(2): Xaa can be any naturally occurring amino acid
  misc_feature (4)..(5): Xaa can be any naturally occurring amino acid
XXDXXDGFLK

SEQ ID NO: 176 - protein, 15 aa, Artificial Sequence
  epitope
SPHGKDLLFKDSADG

SEQ ID NO: 177 - protein, 9 aa, Artificial Sequence
  epitope
PHGKDLLFK

SEQ ID NO: 178 - protein, 10 aa, Artificial Sequence
  epitope
GKDLLFKDSA

SEQ ID NO: 179 - protein, 6 aa, Artificial Sequence
  epitope
KDSADG

SEQ ID NO: 180 - protein, 15 aa, Artificial Sequence
  epitope
  misc_feature (1)..(2): Xaa can be any naturally occurring amino acid
  misc_feature (4)..(4): Xaa can be any naturally occurring amino acid
  misc_feature (6)..(9): Xaa can be any naturally occurring amino acid
  misc_feature (13)..(15): Xaa can be any naturally occurring amino acid
XXHXKXXXXKDSXXX

SEQ ID NO: 181 - protein, 7 aa, Artificial Sequence
  epitope
DLLFKDS

SEQ ID NO: 182 - protein, 5 aa, Artificial Sequence
  epitope
GKDLL

SEQ ID NO: 183 - protein, 15 aa, Artificial Sequence
  epitope
  misc_feature (1)..(2): Xaa can be any naturally occurring amino acid
  misc_feature (4)..(4): Xaa can be any naturally occurring amino acid
  misc_feature (6)..(7): Xaa can be any naturally occurring amino acid
  misc_feature (11)..(15): Xaa can be any naturally occurring amino acid
XXHXKXXLFKXXXXX

SEQ ID NO: 184 - protein, 6 aa, Artificial Sequence
  epitope
KDLLFK

SEQ ID NO: 185 - protein, 5 aa, Artificial Sequence
  epitope
DLLFK

SEQ ID NO: 186 - protein, 6 aa, Artificial Sequence
  epitope
KDSADG

SEQ ID NO: 187 - protein, 15 aa, Artificial Sequence
  epitope
  misc_feature (1)..(2): Xaa can be any naturally occurring amino acid
  misc_feature (4)..(4): Xaa can be any naturally occurring amino acid
  misc_feature (7)..(7): Xaa can be any naturally occurring amino acid
  misc_feature (13)..(15): Xaa can be any naturally occurring amino acid
XXHXKDXLFKDSXXX

SEQ ID NO: 188 - protein, 15 aa, Artificial Sequence
  epitope
  misc_feature (1)..(2): Xaa can be any naturally occurring amino acid
  misc_feature (4)..(4): Xaa can be any naturally occurring amino acid
  misc_feature (6)..(9): Xaa can be any naturally occurring amino acid
  misc_feature (12)..(15): Xaa can be any naturally occurring amino acid
XXHXKXXXXKDXXXX

SEQ ID NO: 189 - protein, 6 aa, Artificial Sequence
  epitope
KDLLFK

SEQ ID NO: 190 - protein, 6 aa, Artificial Sequence
  epitope
  misc_feature (3)..(3): Xaa can be any naturally occurring amino acid
KDXLFK

SEQ ID NO: 191 - protein, 15 aa, Artificial Sequence
  epitope
  misc_feature (1)..(4): Xaa can be any naturally occurring amino acid
  misc_feature (6)..(6): Xaa can be any naturally occurring amino acid
  misc_feature (11)..(15): Xaa can be any naturally occurring amino acid
XXXXKXLLFKXXXXX

SEQ ID NO: 192 - protein, 4 aa, Artificial Sequence
  epitope
LLFK

SEQ ID NO: 193 - protein, 6 aa, Artificial Sequence
  epitope
SPHGKD

SEQ ID NO: 194 - protein, 7 aa, Artificial Sequence
  epitope
FKDSADG

SEQ ID NO: 195 - protein, 4 aa, Artificial Sequence
  epitope
KDSA

SEQ ID NO: 196 - protein, 15 aa, Artificial Sequence
  epitope
HSTVFDNLPNPEDRK

SEQ ID NO: 197 - protein, 15 aa, Artificial Sequence
  epitope
HSTVFDNLPNPEDRK

SEQ ID NO: 198 - protein, 15 aa, Artificial Sequence
  epitope
  misc_feature (1)..(9): Xaa can be any naturally occurring amino acid
XXXXXXXXXNPEDRK

SEQ ID NO: 199 - protein, 10 aa, Artificial Sequence
  epitope
FVKHSTVFDN

SEQ ID NO: 200 - protein, 8 aa, Artificial Sequence
  epitope
VKHSTVFD

SEQ ID NO: 201 - protein, 7 aa, Artificial Sequence
  epitope
KHSTVFD

SEQ ID NO: 202 - protein, 6 aa, Artificial Sequence
  epitope
NPEDRK

SEQ ID NO: 203 - protein, 5 aa, Artificial Sequence
  epitope
NPEDR

SEQ ID NO: 204 - protein, 15 aa, Artificial Sequence
  epitope
HSTVFDNLPNPEDRK

SEQ ID NO: 205 - protein, 7 aa, Artificial Sequence
  epitope
KHSTVFD

SEQ ID NO: 206 - protein, 6 aa, Artificial Sequence
  epitope
HSTVFD

SEQ ID NO: 207 - protein, 6 aa, Artificial Sequence
  epitope
NPEDRK

SEQ ID NO: 208 - protein, 5 aa, Artificial Sequence
  epitope
NPEDR

SEQ ID NO: 209 - protein, 15 aa, Artificial Sequence
  epitope
HSTVFDNLPNPEDRK

SEQ ID NO: 210 - protein, 9 aa, Artificial Sequence
  epitope
FVKHSTVFD

SEQ ID NO: 211 - protein, 7 aa, Artificial Sequence
  epitope
KHSTVFD

SEQ ID NO: 212 - protein, 6 aa, Artificial Sequence
  epitope
HSTVFD

SEQ ID NO: 213 - protein, 5 aa, Artificial Sequence
  epitope
NPEDR

SEQ ID NO: 214 - protein, 15 aa, Artificial Sequence
  epitope
HSTVFDNLPNPEDRK

SEQ ID NO: 215 - protein, 7 aa, Artificial Sequence
  epitope
KHSTVFD

SEQ ID NO: 216 - protein, 6 aa, Artificial Sequence
  epitope
HSTVFD

SEQ ID NO: 217 - protein, 5 aa, Artificial Sequence
  epitope
NPEDR

SEQ ID NO: 218 - protein, 15 aa, Artificial Sequence
  epitope
HSTVFDNLPNPEDRK

SEQ ID NO: 219 - protein, 6 aa, Artificial Sequence
  epitope
HSTVFD

SEQ ID NO: 220 - protein, 5 aa, Artificial Sequence
  epitope
NPEDR

SEQ ID NO: 221 - protein, 15 aa, Artificial Sequence
  epitope
HSTVFDNLPNPEDRK

SEQ ID NO: 222 - protein, 15 aa, Artificial Sequence
  epitope
HSTVFDNLPNPEDRK

SEQ ID NO: 223 - protein, 15 aa, Artificial Sequence
  epitope
  misc_feature (1)..(10): Xaa can be any naturally occurring amino acid
XXXXXXXXXXPEDRK

SEQ ID NO: 224 - protein, 7 aa, Artificial Sequence
  epitope
KHSTVFD

SEQ ID NO: 225 - protein, 6 aa, Artificial Sequence
  epitope
NPEDRK

SEQ ID NO: 226 - protein, 6 aa, Artificial Sequence
  epitope
  misc_feature (1)..(1): Xaa can be any naturally occurring amino acid
XPEDRK

SEQ ID NO: 227 - protein, 15 aa, Artificial Sequence
  epitope
HSTVFDNLPNPEDRK

SEQ ID NO: 228 - protein, 15 aa, Artificial Sequence
  epitope
HSTVFDNLPNPEDRK

SEQ ID NO: 229 - protein, 4 aa, Artificial Sequence
  epitope
TVFD

SEQ ID NO: 230 - protein, 5 aa, Artificial Sequence
  epitope
NPEDR

SEQ ID NO: 231 - protein, 5 aa, Artificial Sequence
  epitope
STVFD

SEQ ID NO: 232 - protein, 6 aa, Artificial Sequence
  epitope
NPEDRK

SEQ ID NO: 233 - protein, 5 aa, Artificial Sequence
  epitope
NPEDR

SEQ ID NO: 234 - protein, 5 aa, Artificial Sequence
  epitope
STVFD

SEQ ID NO: 235 - protein, 5 aa, Artificial Sequence
  epitope
NPEDR

SEQ ID NO: 236 - protein, 6 aa, Artificial Sequence
  epitope
HSTVFD

SEQ ID NO: 237 - protein, 4 aa, Artificial Sequence
  epitope
TVFD

SEQ ID NO: 238 - protein, 5 aa, Artificial Sequence
  epitope
NPEDR

SEQ ID NO: 239 - protein, 15 aa, Artificial Sequence
  epitope
  misc_feature (1)..(1): Xaa can be any naturally occurring amino acid
  misc_feature (3)..(10): Xaa can be any naturally occurring amino acid
  misc_feature (15)..(15): Xaa can be any naturally occurring amino acid
XSXXXXXXXXPEDRX

SEQ ID NO: 240 - protein, 10 aa, Artificial Sequence
  epitope
DNLPNPEDRK

SEQ ID NO: 241 - protein, 5 aa, Artificial Sequence
  epitope
NPEDR

SEQ ID NO: 242 - protein, 5 aa, Artificial Sequence
  epitope
  misc_feature (1)..(1): Xaa can be any naturally occurring amino acid
XPEDR

SEQ ID NO: 243 - protein, 6 aa, Artificial Sequence
  epitope
NPEDRK

SEQ ID NO: 244 - protein, 6 aa, Artificial Sequence
  epitope
HSTVFD

SEQ ID NO: 245 - protein, 6 aa, Artificial Sequence
  epitope
NPEDRK

SEQ ID NO: 246 - protein, 5 aa, Artificial Sequence
  epitope
STVFD

SEQ ID NO: 247 - protein, 4 aa, Artificial Sequence
  epitope
TVFD

SEQ ID NO: 248 - protein, 6 aa, Artificial Sequence
  epitope
NPEDRK

SEQ ID NO: 249 - protein, 15 aa, Artificial Sequence
  epitope
  misc_feature (1)..(9): Xaa can be any naturally occurring amino acid
XXXXXXXXXNPEDRK

SEQ ID NO: 250 - protein, 15 aa, Artificial Sequence
  epitope
  misc_feature (1)..(10): Xaa can be any naturally occurring amino acid
XXXXXXXXXXPEDRK

SEQ ID NO: 251 - protein, 15 aa, Artificial Sequence
  epitope
  misc_feature (1)..(10): Xaa can be any naturally occurring amino acid
  misc_feature (15)..(15): Xaa can be any naturally occurring amino acid
XXXXXXXXXXPEDRX

SEQ ID NO: 252 - protein, 6 aa, Artificial Sequence
  epitope
NPEDRK

SEQ ID NO: 253 - protein, 5 aa, Artificial Sequence
  epitope
NPEDR

SEQ ID NO: 254 - protein, 5 aa, Artificial Sequence
  epitope
  misc_feature (1)..(1): Xaa can be any naturally occurring amino acid
XPEDR

SEQ ID NO: 255 - protein, 15 aa, Artificial Sequence
  epitope
DCTSNFCLFQSNSKD

SEQ ID NO: 256 - protein, 15 aa, Artificial Sequence
  epitope
DCTSNFCLFQSNSKD

SEQ ID NO: 257 - protein, 5 aa, Artificial Sequence
  epitope
QSNSK

SEQ ID NO: 258 - protein, 15 aa, Artificial Sequence
  epitope
DCTSNFCLFQSNSKD

SEQ ID NO: 259 - protein, 15 aa, Artificial Sequence
  epitope
  misc_feature (1)..(8): Xaa can be any naturally occurring amino acid
XXXXXXXXFQSNSKD

SEQ ID NO: 260 - protein, 8 aa, Artificial Sequence
  epitope
CLFQSNSK

SEQ ID NO: 261 - protein, 5 aa, Artificial Sequence
  epitope
QSNSK

SEQ ID NO: 262 - protein, 15 aa, Artificial Sequence
  epitope
DCTSNFCLFQSNSKD

SEQ ID NO: 263 - protein, 15 aa, Artificial Sequence
  epitope
  misc_feature (1)..(8): Xaa can be any naturally occurring amino acid
XXXXXXXXFQSNSKD

SEQ ID NO: 264 - protein, 5 aa, Artificial Sequence
  epitope
QSNSK

SEQ ID NO: 265 - protein, 15 aa, Artificial Sequence
  epitope
DCTSNFCLFQSNSKD

SEQ ID NO: 266 - protein, 15 aa, Artificial Sequence
  epitope
  misc_feature (1)..(5): Xaa can be any naturally occurring amino acid
  misc_feature (12)..(12): Xaa can be any naturally occurring amino acid
XXXXXFCLFQSXSKD

SEQ ID NO: 267 - protein, 8 aa, Artificial Sequence
  epitope
LFQSNSKD

SEQ ID NO: 268 - protein, 6 aa, Artificial Sequence
  epitope
FQSNSK

SEQ ID NO: 269 - protein, 6 aa, Artificial Sequence
  epitope
  misc_feature (4)..(4): Xaa can be any naturally occurring amino acid
FQSXSK

SEQ ID NO: 270 - protein, 4 aa, Artificial Sequence
  epitope
QSNS

SEQ ID NO: 271 - protein, 15 aa, Artificial Sequence
  epitope
DCTSNFCLFQSNSKD

SEQ ID NO: 272 - protein, 15 aa, Artificial Sequence
  epitope
  misc_feature (1)..(6): Xaa can be any naturally occurring amino acid
  misc_feature (10)..(13): Xaa can be any naturally occurring amino acid
  misc_feature (15)..(15): Xaa can be any naturally occurring amino acid
XXXXXXCLFXXXXKX

SEQ ID NO: 273 - protein, 6 aa, Artificial Sequence
  epitope
FQSNSK

SEQ ID NO: 274 - protein, 5 aa, Artificial Sequence
  epitope
QSNSK

SEQ ID NO: 275 - protein, 4 aa, Artificial Sequence
  epitope
QSNS

SEQ ID NO: 276 - protein, 5 aa, Artificial Sequence
  epitope
QSNSK

SEQ ID NO: 277 - protein, 15 aa, Artificial Sequence
  epitope
  misc_feature (1)..(8): Xaa can be any naturally occurring amino acid
  misc_feature (10)..(11): Xaa can be any naturally occurring amino acid
XXXXXXXXFXXNSKD

SEQ ID NO: 278 - protein, 5 aa, Artificial Sequence
  epitope
QSNSK

SEQ ID NO: 279 - protein, 8 aa, Artificial Sequence
  epitope
CLFQSNSK

SEQ ID NO: 280 - protein, 5 aa, Artificial Sequence
  epitope
QSNSK

SEQ ID NO: 281 - protein, 5 aa, Artificial Sequence
  epitope
QSNSK

SEQ ID NO: 282 - protein, 15 aa, Artificial Sequence
  epitope
  misc_feature (1)..(6): Xaa can be any naturally occurring amino acid
  misc_feature (10)..(12): Xaa can be any naturally occurring amino acid
XXXXXXCLFXXXSKD

SEQ ID NO: 283 - protein, 8 aa, Artificial Sequence
  epitope
CLFQSNSK

SEQ ID NO: 284 - protein, 5 aa, Artificial Sequence
  epitope
QSNSK

SEQ ID NO: 285 - protein, 5 aa, Artificial Sequence
  epitope
QSNSK

SEQ ID NO: 286 - protein, 4 aa, Artificial Sequence
  epitope
QSNS

SEQ ID NO: 287 - protein, 15 aa, Artificial Sequence
  epitope
  misc_feature (1)..(8): Xaa can be any naturally occurring amino acid
  misc_feature (10)..(11): Xaa can be any naturally occurring amino acid
XXXXXXXXFXXNSKD

SEQ ID NO: 288 - protein, 5 aa, Artificial Sequence
  epitope
QSNSK

SEQ ID NO: 289 - protein, 15 aa, Artificial Sequence
  epitope
LAENYKTEGESCKNT

SEQ ID NO: 290 - protein, 15 aa, Artificial Sequence
  epitope
LAENYKTEGESCKNT

SEQ ID NO: 291 - protein, 15 aa, Artificial Sequence
  epitope
  misc_feature (1)..(5): Xaa can be any naturally occurring amino acid
  misc_feature (7)..(9): Xaa can be any naturally occurring amino acid
  misc_feature (12)..(12): Xaa can be any naturally occurring amino acid
  misc_feature (14)..(14): Xaa can be any naturally occurring amino acid
XXXXXKXXXESXKXT

SEQ ID NO: 292 - protein, 8 aa, Artificial Sequence
  epitope
AENYKTEG

SEQ ID NO: 293 - protein, 9 aa, Artificial Sequence
  epitope
YKTEGESCK

SEQ ID NO: 294 - protein, 9 aa, Artificial Sequence
  epitope
  misc_feature (1)..(1): Xaa can be any naturally occurring amino acid
  misc_feature (3)..(5): Xaa can be any naturally occurring amino acid
  misc_feature (8)..(8): Xaa can be any naturally occurring amino acid
XKXXXESXK

SEQ ID NO: 295 - protein, 9 aa, Artificial Sequence
  epitope
ESCKNTPEK

SEQ ID NO: 296 - protein, 8 aa, Artificial Sequence
  epitope
ESCKNTPE

SEQ ID NO: 297 - protein, 8 aa, Artificial Sequence
  epitope
  misc_feature (3)..(3): Xaa can be any naturally occurring amino acid
  misc_feature (5)..(5): Xaa can be any naturally occurring amino acid
ESXKXTPE

SEQ ID NO: 298 - protein, 15 aa, Artificial Sequence
  epitope
LAENYKTEGESCKNT

SEQ ID NO: 299 - protein, 15 aa, Artificial Sequence
  epitope
  misc_feature (1)..(4): Xaa can be any naturally occurring amino acid
  misc_feature (7)..(8): Xaa can be any naturally occurring amino acid
XXXXYKXXGESCKNT

SEQ ID NO: 300 - protein, 9 aa, Artificial Sequence
  epitope
YKTEGESCK

SEQ ID NO: 301 - protein, 5 aa, Artificial Sequence
  epitope
KTEGE

SEQ ID NO: 302 - protein, 9 aa, Artificial Sequence
  epitope
  misc_feature (3)..(4): Xaa can be any naturally occurring amino acid
YKXXGESCK

SEQ ID NO: 303 - protein, 8 aa, Artificial Sequence
  epitope
ESCKNTPE

SEQ ID NO: 304 - protein, 15 aa, Artificial Sequence
  epitope
LAENYKTEGESCKNT

SEQ ID NO: 305 - protein, 15 aa, Artificial Sequence
  epitope
  misc_feature (1)..(2): Xaa can be any naturally occurring amino acid
  misc_feature (4)..(5): Xaa can be any naturally occurring amino acid
  misc_feature (7)..(7): Xaa can be any naturally occurring amino acid
  misc_feature (11)..(11): Xaa can be any naturally occurring amino acid
  misc_feature (14)..(15): Xaa can be any naturally occurring amino acid
XXEXXKXEGEXCKXX

SEQ ID NO: 306 - protein, 9 aa, Artificial Sequence
  epitope
GESCKNTPE

SEQ ID NO: 307 - protein, 9 aa, Artificial Sequence
  epitope
  misc_feature (3)..(3): Xaa can be any naturally occurring amino acid
  misc_feature (6)..(7): Xaa can be any naturally occurring amino acid
GEXCKXXPE

SEQ ID NO: 308 - protein, 15 aa, Artificial Sequence
  epitope
LAENYKTEGESCKNT

SEQ ID NO: 309 - protein, 15 aa, Artificial Sequence
  epitope
  misc_feature (1)..(9): Xaa can be any naturally occurring amino acid
  misc_feature (12)..(12): Xaa can be any naturally occurring amino acid
  misc_feature (15)..(15): Xaa can be any naturally occurring amino acid
XXXXXXXXXESXKNX

SEQ ID NO: 310 - protein, 8 aa, Artificial Sequence
  epitope
KTEGESCK

SEQ ID NO: 311 - protein, 9 aa, Artificial Sequence
  epitope
ESCKNTPEK

SEQ ID NO: 312 - protein, 9 aa, Artificial Sequence
  epitope
  misc_feature (3)..(3): Xaa can be any naturally occurring amino acid
  misc_feature (6)..(6): Xaa can be any naturally occurring amino acid
ESXKNXPEK

SEQ ID NO: 313 - protein, 15 aa, Artificial Sequence
  epitope
LAENYKTEGESCKNT

SEQ ID NO: 314 - protein, 15 aa, Artificial Sequence
  epitope
  misc_feature (1)..(7): Xaa can be any naturally occurring amino acid
  misc_feature (12)..(12): Xaa can be any naturally occurring amino acid
  misc_feature (14)..(15): Xaa can be any naturally occurring amino acid
XXXXXXXEGESXKXX

SEQ ID NO: 315 - protein, 10 aa, Artificial Sequence
  epitope
NYKTEGESCK

SEQ ID NO: 316 - protein, 10 aa, Artificial Sequence
  epitope
  misc_feature (1)..(4): Xaa can be any naturally occurring amino acid
  misc_feature (9)..(9): Xaa can be any naturally occurring amino acid
XXXXEGESXK

SEQ ID NO: 317 - protein, 9 aa, Artificial Sequence
  epitope
ESCKNTPEK

SEQ ID NO: 318 - protein, 9 aa, Artificial Sequence
  epitope
  misc_feature (3)..(3): Xaa can be any naturally occurring amino acid
  misc_feature (5)..(6): Xaa can be any naturally occurring amino acid
ESXKXXPEK

SEQ ID NO: 319 - protein, 15 aa, Artificial Sequence
  epitope
  misc_feature (1)..(4): Xaa can be any naturally occurring amino acid
  misc_feature (7)..(7): Xaa can be any naturally occurring amino acid
  misc_feature (11)..(11): Xaa can be any naturally occurring amino acid
XXXXYKXEGEXCKNT

SEQ ID NO: 320 - protein, 15 aa, Artificial Sequence
  epitope
  misc_feature (1)..(8): Xaa can be any naturally occurring amino acid
  misc_feature (10)..(12): Xaa can be any naturally occurring amino acid
XXXXXXXXGXXXKNT

SEQ ID NO: 321 - protein, 9 aa, Artificial Sequence
  epitope
YKTEGESCK

SEQ ID NO: 322 - protein, 9 aa, Artificial Sequence
  epitope
  misc_feature (3)..(3): Xaa can be any naturally occurring amino acid
  misc_feature (7)..(7): Xaa can be any naturally occurring amino acid
YKXEGEXCK

SEQ ID NO: 323 - protein, 15 aa, Artificial Sequence
  epitope
  misc_feature (1)..(2): Xaa can be any naturally occurring amino acid
  misc_feature (4)..(4): Xaa can be any naturally occurring amino acid
  misc_feature (7)..(8): Xaa can be any naturally occurring amino acid
  misc_feature (11)..(11): Xaa can be any naturally occurring amino acid
XXEXYKXXGEXCKNT

SEQ ID NO: 324 - protein, 9 aa, Artificial Sequence
  epitope
YKTEGESCK

SEQ ID NO: 325 - protein, 7 aa, Artificial Sequence
  epitope
KTEGESC

SEQ ID NO: 326 - protein, 7 aa, Artificial Sequence
  epitope
  misc_feature (2)..(3): Xaa can be any naturally occurring amino acid
  misc_feature (6)..(6): Xaa can be any naturally occurring amino acid
KXXGEXC

SEQ ID NO: 327 - protein, 15 aa, Artificial Sequence
  epitope
  misc_feature (1)..(2): Xaa can be any naturally occurring amino acid
  misc_feature (7)..(11): Xaa can be any naturally occurring amino acid
XXENYKXXXXXCKNT

SEQ ID NO: 328 - protein, 9 aa, Artificial Sequence
  epitope
YKTEGESCK

SEQ ID NO: 329 - protein, 6 aa, Artificial Sequence
  epitope
KTEGES

SEQ ID NO: 330 - protein, 15 aa, Artificial Sequence
  epitope
  misc_feature (1)..(4): Xaa can be any naturally occurring amino acid
  misc_feature (6)..(9): Xaa can be any naturally occurring amino acid
XXXXYXXXXESCKNT

SEQ ID NO: 331 - protein, 15 aa, Artificial Sequence
  epitope
  misc_feature (1)..(10): Xaa can be any naturally occurring amino acid
XXXXXXXXXXSCKNT

SEQ ID NO: 332 - protein, 6 aa, Artificial Sequence
  epitope
KTEGES

SEQ ID NO: 333 - protein, 15 aa, Artificial Sequence
  epitope
  misc_feature (1)..(5): Xaa can be any naturally occurring amino acid
  misc_feature (7)..(8): Xaa can be any naturally occurring amino acid
  misc_feature (12)..(12): Xaa can be any naturally occurring amino acid
  misc_feature (14)..(15): Xaa can be any naturally occurring amino acid
XXXXXKXXGESXKXX

SEQ ID NO: 334 - protein, 15 aa, Artificial Sequence
  epitope
NHAVVSRKDKATCVE

SEQ ID NO: 335 - protein, 15 aa, Artificial Sequence
  epitope
NHAVVSRKDKATCVE

SEQ ID NO: 336 - protein, 15 aa, Artificial Sequence
  epitope
NHAVVSRKDKATCVE

SEQ ID NO: 337 - protein, 15 aa, Artificial Sequence
  epitope
NHAVVSRKDKATCVE

SEQ ID NO: 338 - protein, 9 aa, Artificial Sequence
  epitope
RGPNHAVVS

SEQ ID NO: 339 - protein, 15 aa, Artificial Sequence
  epitope
NHAVVSRKDKATCVE

SEQ ID NO: 340 - protein, 15 aa, Artificial Sequence
  epitope
  misc_feature (1)..(4): Xaa can be any naturally occurring amino acid
  misc_feature (6)..(7): Xaa can be any naturally occurring amino acid
  misc_feature (10)..(10): Xaa can be any naturally occurring amino acid
  misc_feature (12)..(15): Xaa can be any naturally occurring amino acid
XXXXVXXKDXAXXXX

SEQ ID NO: 341 - protein, 6 aa, Artificial Sequence
  epitope
RKDKAT

SEQ ID NO: 342 - protein, 15 aa, Artificial Sequence
  epitope
NHAVVSRKDKATCVE

SEQ ID NO: 343 - protein, 15 aa, Artificial Sequence
  epitope
NHAVVSRKDKATCVE

SEQ ID NO: 344 - protein, 6 aa, Artificial Sequence
  epitope
RKDKAT

SEQ ID NO: 345 - protein, 15 aa, Artificial Sequence
  epitope
  misc_feature (1)..(3): Xaa can be any naturally occurring amino acid
  misc_feature (12)..(15): Xaa can be any naturally occurring amino acid
XXXVVSRKDKAXXXX

SEQ ID NO: 346 - protein, 4 aa, Artificial Sequence
  epitope
RKDK

SEQ ID NO: 347 - protein, 15 aa, Artificial Sequence
  epitope
  misc_feature (1)..(4): Xaa can be any naturally occurring amino acid
  misc_feature (6)..(6): Xaa can be any naturally occurring amino acid
  misc_feature (9)..(9): Xaa can be any naturally occurring amino acid
  misc_feature (13)..(15): Xaa can be any naturally occurring amino acid
XXXXVXRKXKATXXX

SEQ ID NO: 348 - protein, 6 aa, Artificial Sequence
  epitope
RKDKAT

SEQ ID NO: 349 - protein, 6 aa, Artificial Sequence
  epitope
  misc_feature (3)..(3): Xaa can be any naturally occurring amino acid
RKXKAT

SEQ ID NO: 350 - protein, 15 aa, Artificial Sequence
  epitope
  misc_feature (1)..(4): Xaa can be any naturally occurring amino acid
  misc_feature (7)..(7): Xaa can be any naturally occurring amino acid
  misc_feature (9)..(9): Xaa can be any naturally occurring amino acid
  misc_feature (12)..(15): Xaa can be any naturally occurring amino acid
XXXXVSXKXKAXXXX

SEQ ID NO: 351 - protein, 15 aa, Artificial Sequence
  epitope
  misc_feature (1)..(3): Xaa can be any naturally occurring amino acid
  misc_feature (13)..(15): Xaa can be any naturally occurring amino acid
XXXVVSRKDKATXXX

SEQ ID NO: 352 - protein, 15 aa, Artificial Sequence
  epitope
  misc_feature (1)..(4): Xaa can be any naturally occurring amino acid
  misc_feature (11)..(15): Xaa can be any naturally occurring amino acid
XXXXVSRKDKXXXXX

SEQ ID NO: 353 - protein, 15 aa, Artificial Sequence
  epitope
ETELGSNEHSKTQCE

SEQ ID NO: 354 - protein, 15 aa, Artificial Sequence
  epitope
ETELGSNEHSKTQCE

SEQ ID NO: 355 - protein, 5 aa, Artificial Sequence
  epitope
SKTQC

SEQ ID NO: 356 - protein, 4 aa, Artificial Sequence
  epitope
SKTQ

SEQ ID NO: 357 - protein, 15 aa, Artificial Sequence
  epitope
ETELGSNEHSKTQCE

SEQ ID NO: 358 - protein, 5 aa, Artificial Sequence
  epitope
SKTQC

SEQ ID NO: 359 - protein, 15 aa, Artificial Sequence
  epitope
ETELGSNEHSKTQCE

SEQ ID NO: 360 - protein, 6 aa, Artificial Sequence
  epitope
HSKTQC

SEQ ID NO: 361 - protein, 5 aa, Artificial Sequence
  epitope
SKTQC

SEQ ID NO: 362 - protein, 15 aa, Artificial Sequence
  epitope
ETELGSNEHSKTQCE

SEQ ID NO: 363 - protein, 9 aa, Artificial Sequence
  epitope
FAPETELGS

SEQ ID NO: 364 - protein, 5 aa, Artificial Sequence
  epitope
SKTQC

SEQ ID NO: 365 - protein, 4 aa, Artificial Sequence
  epitope
SKTQ

SEQ ID NO: 366 - protein, 15 aa, Artificial Sequence
  epitope
ETELGSNEHSKTQCE

SEQ ID NO: 367 - protein, 15 aa, Artificial Sequence
  epitope
ETELGSNEHSKTQCE

SEQ ID NO: 368 - protein, 15 aa, Artificial Sequence
  epitope
ETELGSNEHSKTQCE

SEQ ID NO: 369 - protein, 7 aa, Artificial Sequence
  epitope
EHSKTQC

SEQ ID NO: 370 - protein, 4 aa, Artificial Sequence
  epitope
SKTQ

SEQ ID NO: 371 - protein, 4 aa, Artificial Sequence
  epitope
SKTQ

SEQ ID NO: 372 - protein, 15 aa, Artificial Sequence
  epitope
  misc_feature (1)..(7): Xaa can be any naturally occurring amino acid
  misc_feature (9)..(10): Xaa can be any naturally occurring amino acid
  misc_feature (13)..(14): Xaa can be any naturally occurring amino acid
XXXXXXXEXXKTXXE

SEQ ID NO: 373 - protein, 4 aa, Artificial Sequence
  epitope
SKTQ

SEQ ID NO: 374 - protein, 5 aa, Artificial Sequence
  epitope
SKTQC

SEQ ID NO: 375 - protein, 4 aa, Artificial Sequence
  epitope
SKTQ

SEQ ID NO: 376 - protein, 4 aa, Artificial Sequence
  epitope
SKTQ

SEQ ID NO: 377 - protein, 15 aa, Artificial Sequence
  epitope
LHTLFFNTWRIIKDG

SEQ ID NO: 378 - protein, 15 aa, Artificial Sequence
  epitope
LHTLFFNTWRIIKDG

SEQ ID NO: 379 - protein, 15 aa, Artificial Sequence
  epitope
  misc_feature (1)..(11): Xaa can be any naturally occurring amino acid
XXXXXXXXXXXIKDG

SEQ ID NO: 380 - protein, 10 aa, Artificial Sequence
  epitope
LPLHTLFFNT

SEQ ID NO: 381 - protein, 15 aa, Artificial Sequence
  epitope
LHTLFFNTWRIIKDG

SEQ ID NO: 382 - protein, 15 aa, Artificial Sequence
  epitope
LHTLFFNTWRIIKDG

SEQ ID NO: 383 - protein, 15 aa, Artificial Sequence
  epitope
LHTLFFNTWRIIKDG

SEQ ID NO: 384 - protein, 4 aa, Artificial Sequence
  epitope
RIIK

SEQ ID NO: 385 - protein, 15 aa, Artificial Sequence
  epitope
SFEKYWCKWSNRGCS

SEQ ID NO: 386 - protein, 15 aa, Artificial Sequence
  epitope
SFEKYWCKWSNRGCS

SEQ ID NO: 387 - protein, 15 aa, Artificial Sequence
  epitope
  misc_feature (1)..(1): Xaa can be any naturally occurring amino acid
XFEKYWCKWSNRGCS

SEQ ID NO: 388 - protein, 15 aa, Artificial Sequence
  epitope
  misc_feature (1)..(2): Xaa can be any naturally occurring amino acid
  misc_feature (12)..(15): Xaa can be any naturally occurring amino acid
XXEKYWCKWSNXXXX

SEQ ID NO: 389 - protein, 5 aa, Artificial Sequence
  epitope
FEKYW

SEQ ID NO: 390 - protein, 5 aa, Artificial Sequence
  epitope
  misc_feature (1)..(1): Xaa can be any naturally occurring amino acid
XEKYW

SEQ ID NO: 391 - protein, 10 aa, Artificial Sequence
  epitope
YWCKWSNRGC

SEQ ID NO: 392 - protein, 10 aa, Artificial Sequence
  epitope
  misc_feature (8)..(10): Xaa can be any naturally occurring amino acid
YWCKWSNXXX

SEQ ID NO: 393 - protein, 15 aa, Artificial Sequence
  epitope
SFEKYWCKWSNRGCS

SEQ ID NO: 394 - protein, 15 aa, Artificial Sequence
  epitope
  misc_feature (1)..(1): Xaa can be any naturally occurring amino acid
XFEKYWCKWSNRGCS

SEQ ID NO: 395 - protein, 15 aa, Artificial Sequence
  epitope
  misc_feature (1)..(2): Xaa can be any naturally occurring amino acid
  misc_feature (15)..(15): Xaa can be any naturally occurring amino acid
XXEKYWCKWSNRGCX

SEQ ID NO: 396 - protein, 15 aa, Artificial Sequence
  epitope
SFEKYWCKWSNRGCS

SEQ ID NO: 397 - protein, 15 aa, Artificial Sequence
  epitope
  misc_feature (1)..(1): Xaa can be any naturally occurring amino acid
  misc_feature (6)..(6): Xaa can be any naturally occurring amino acid
  misc_feature (12)..(15): Xaa can be any naturally occurring amino acid
XFEKYXCKWSNXXXX

SEQ ID NO: 398 - protein, 15 aa, Artificial Sequence
  epitope
  misc_feature (1)..(2): Xaa can be any naturally occurring amino acid
  misc_feature (6)..(6): Xaa can be any naturally occurring amino acid
  misc_feature (12)..(15): Xaa can be any naturally occurring amino acid
XXEKYXCKWSNXXXX

SEQ ID NO: 399 - protein, 15 aa, Artificial Sequence
  epitope
  misc_feature (1)..(2): Xaa can be any naturally occurring amino acid
  misc_feature (4)..(8): Xaa can be any naturally occurring amino acid
  misc_feature (12)..(15): Xaa can be any naturally occurring amino acid
XXEXXXXXWSNXXXX

SEQ ID NO: 400 - protein, 15 aa, Artificial Sequence
  epitope
SFEKYWCKWSNRGCS

SEQ ID NO: 401 - protein, 15 aa, Artificial Sequence
  epitope
SFEKYWCKWSNRGCS

SEQ ID NO: 402 - protein, 15 aa, Artificial Sequence
  epitope
  misc_feature (1)..(1): Xaa can be any naturally occurring amino acid
XFEKYWCKWSNRGCS

SEQ ID NO: 403 - protein, 15 aa, Artificial Sequence
  epitope
  misc_feature (1)..(2): Xaa can be any naturally occurring amino acid
  misc_feature (6)..(6): Xaa can be any naturally occurring amino acid
  misc_feature (13)..(15): Xaa can be any naturally occurring amino acid
XXEKYXCKWSNRXXX

SEQ ID NO: 404 - protein, 15 aa, Artificial Sequence
  epitope
SFEKYWCKWSNRGCS

SEQ ID NO: 405 - protein, 15 aa, Artificial Sequence
  epitope
  misc_feature (1)..(3): Xaa can be any naturally occurring amino acid
  misc_feature (8)..(9): Xaa can be any naturally occurring amino acid
  misc_feature (11)..(11): Xaa can be any naturally occurring amino acid
  misc_feature (14)..(15): Xaa can be any naturally occurring amino acid
XXXKYWCXXSXRGXX

SEQ ID NO: 406 - protein, 15 aa, Artificial Sequence
  epitope
SFEKYWCKWSNRGCS

SEQ ID NO: 407 - protein, 15 aa, Artificial Sequence
  epitope
  misc_feature (1)..(3): Xaa can be any naturally occurring amino acid
  misc_feature (13)..(15): Xaa can be any naturally occurring amino acid
XXXKYWCKWSNRXXX

SEQ ID NO: 408 - protein, 15 aa, Artificial Sequence
  epitope
  misc_feature (1)..(1): Xaa can be any naturally occurring amino acid
  misc_feature (3)..(3): Xaa can be any naturally occurring amino acid
  misc_feature (13)..(15): Xaa can be any naturally occurring amino acid
XFXKYWCKWSNRXXX

SEQ ID NO: 409 - protein, 15 aa, Artificial Sequence
  epitope
  misc_feature (1)..(3): Xaa can be any naturally occurring amino acid
  misc_feature (6)..(7): Xaa can be any naturally occurring amino acid
  misc_feature (12)..(15): Xaa can be any naturally occurring amino acid
XXXKYXXKWSNXXXX

SEQ ID NO: 410 - protein, 15 aa, Artificial Sequence
  epitope
  misc_feature (1)..(1): Xaa can be any naturally occurring amino acid
  misc_feature (3)..(3): Xaa can be any naturally occurring amino acid
  misc_feature (14)..(15): Xaa can be any naturally occurring amino acid
XFXKYWCKWSNRGXX

SEQ ID NO: 411 - protein, 15 aa, Artificial Sequence
  epitope
  misc_feature (1)..(10): Xaa can be any naturally occurring amino acid
XXXXXXXXXXNRGCS

SEQ ID NO: 412 - protein, 15 aa, Artificial Sequence
  epitope
NLDTVTKEDEGWYWC

SEQ ID NO: 413 - protein, 15 aa, Artificial Sequence
  epitope
NLDTVTKEDEGWYWC

SEQ ID NO: 414 - protein, 8 aa, Artificial Sequence
  epitope
SLNLDTVT

SEQ ID NO: 415 - protein, 7 aa, Artificial Sequence
  epitope
LNLDTVT

SEQ ID NO: 416 - protein, 5 aa, Artificial Sequence
  epitope
LDTVT

SEQ ID NO: 417 - protein, 10 aa, Artificial Sequence
  epitope
TKEDEGWYWC

SEQ ID NO: 418 - protein, 4 aa, Artificial Sequence
  epitope
EGWY

SEQ ID NO: 419 - protein, 15 aa, Artificial Sequence
  epitope
NLDTVTKEDEGWYWC

SEQ ID NO: 420 - protein, 10 aa, Artificial Sequence
  epitope
VVSLNLDTVT

SEQ ID NO: 421 - protein, 8 aa, Artificial Sequence
  epitope
SLNLDTVT

SEQ ID NO: 422 - protein, 10 aa, Artificial Sequence
  epitope
VTKEDEGWYW

SEQ ID NO: 423 - protein, 7 aa, Artificial Sequence
  epitope
KEDEGWY

SEQ ID NO: 424 - protein, 10 aa, Artificial Sequence
  epitope
EGWYWCGVKE

SEQ ID NO: 425 - protein, 15 aa, Artificial Sequence
  epitope
NLDTVTKEDEGWYWC

SEQ ID NO: 426 - protein, 10 aa, Artificial Sequence
  epitope
VVSLNLDTVT

SEQ ID NO: 427 - protein, 9 aa, Artificial Sequence
  epitope
VSLNLDTVT

SEQ ID NO: 428 - protein, 10 aa, Artificial Sequence
  epitope
TKEDEGWYWC

SEQ ID NO: 429 - protein, 9 aa, Artificial Sequence
  epitope
KEDEGWYWC

SEQ ID NO: 430 - protein, 15 aa, Artificial Sequence
  epitope
NLDTVTKEDEGWYWC

SEQ ID NO: 431 - protein, 15 aa, Artificial Sequence
  epitope
  misc_feature (1)..(3): Xaa can be any naturally occurring amino acid
  misc_feature (6)..(6): Xaa can be any naturally occurring amino acid
  misc_feature (9)..(9): Xaa can be any naturally occurring amino acid
  misc_feature (11)..(15): Xaa can be any naturally occurring amino acid
XXXTVXKEXEXXXXX

SEQ ID NO: 432 - protein, 9 aa, Artificial Sequence
  epitope
VSLNLDTVT

SEQ ID NO: 433 - protein, 9 aa, Artificial Sequence
  epitope
  misc_feature (4)..(6): Xaa can be any naturally occurring amino acid
  misc_feature (9)..(9): Xaa can be any naturally occurring amino acid
VSLXXXTVX

SEQ ID NO: 434 - protein, 10 aa, Artificial Sequence
  epitope
TKEDEGWYWC

SEQ ID NO: 435 - protein, 9 aa, Artificial Sequence
  epitope
TKEDEGWYW

SEQ ID NO: 436 - protein, 8 aa, Artificial Sequence
  epitope
KEDEGWYW

SEQ ID NO: 437 - protein, 10 aa, Artificial Sequence
  epitope
LDTVTKEDEG

SEQ ID NO: 438 - protein, 7 aa, Artificial Sequence
  epitope
KEDEGWY

SEQ ID NO: 439 - protein, 15 aa, Artificial Sequence
  epitope
  misc_feature (1)..(4): Xaa can be any naturally occurring amino acid
  misc_feature (6)..(6): Xaa can be any naturally occurring amino acid
  misc_feature (11)..(15): Xaa can be any naturally occurring amino acid
XXXXVXKEDEXXXXX

SEQ ID NO: 440 - protein, 15 aa, Artificial Sequence
  epitope
  misc_feature (1)..(6): Xaa can be any naturally occurring amino acid
  misc_feature (11)..(15): Xaa can be any naturally occurring amino acid
XXXXXXKEDEXXXXX

SEQ ID NO: 441 - protein, 10 aa, Artificial Sequence
  epitope
VTKEDEGWYW

SEQ ID NO: 442 - protein, 9 aa, Artificial Sequence
  epitope
TKEDEGWYW

SEQ ID NO: 443 - protein, 9 aa, Artificial Sequence
  epitope
  misc_feature (1)..(1): Xaa can be any naturally occurring amino acid
  misc_feature (6)..(9): Xaa can be any naturally occurring amino acid
XKEDEXXXX

SEQ ID NO: 444 - protein, 6 aa, Artificial Sequence
  epitope
NLDTVT

SEQ ID NO: 445 - protein, 9 aa, Artificial Sequence
  epitope
TKEDEGWYW

SEQ ID NO: 446 - protein, 7 aa, Artificial Sequence
  epitope
KEDEGWY

SEQ ID NO: 447 - protein, 15 aa, Artificial Sequence
  epitope
  misc_feature (1)..(3): Xaa can be any naturally occurring amino acid
  misc_feature (11)..(15): Xaa can be any naturally occurring amino acid
XXXTVTKEDEXXXXX

SEQ ID NO: 448 - protein, 9 aa, Artificial Sequence
  epitope
KEDEGWYWC

SEQ ID NO: 449 - protein, 5 aa, Artificial Sequence
  epitope
KEDEG

SEQ ID NO: 450 - protein, 5 aa, Artificial Sequence
  epitope
  misc_feature (5)..(5): Xaa can be any naturally occurring amino acid
KEDEX

SEQ ID NO: 451 - protein, 15 aa, Artificial Sequence
  epitope
VKNGGKEEKHHRPGQ

SEQ ID NO: 452 - protein, 15 aa, Artificial Sequence
  epitope
VKNGGKEEKHHRPGQ

SEQ ID NO: 453 - protein, 7 aa, Artificial Sequence
  epitope
KNGGKEE

SEQ ID NO: 454 - protein, 4 aa, Artificial Sequence
  epitope
GGKE

SEQ ID NO: 455 - protein, 15 aa, Artificial Sequence
  epitope
VKNGGKEEKHHRPGQ

SEQ ID NO: 456 - protein, 15 aa, Artificial Sequence
  epitope
  misc_feature (1)..(2): Xaa can be any naturally occurring amino acid
  misc_feature (4)..(6): Xaa can be any naturally occurring amino acid
  misc_feature (9)..(12): Xaa can be any naturally occurring amino acid
XXNXXXEEXXXXPGQ

SEQ ID NO: 457 - protein, 8 aa, Artificial Sequence
  epitope
KNGGKEEK

SEQ ID NO: 458 - protein, 6 aa, Artificial Sequence
  epitope
KNGGKE

SEQ ID NO: 459 - protein, 10 aa, Artificial Sequence
  epitope
EKHHRPGQQI

SEQ ID NO: 460 - protein, 10 aa, Artificial Sequence
  epitope
  misc_feature (2)..(5): Xaa can be any naturally occurring amino acid
EXXXXPGQQI

SEQ ID NO: 461 - protein, 15 aa, Artificial Sequence
  epitope
VKNGGKEEKHHRPGQ

SEQ ID NO: 462 - protein, 10 aa, Artificial Sequence
  epitope
TLVVKNGGKE

SEQ ID NO: 463 - protein, 7 aa, Artificial Sequence
  epitope
NGGKEEK

SEQ ID NO: 464 - protein, 6 aa, Artificial Sequence
  epitope
GGKEEK

SEQ ID NO: 465 - protein, 10 aa, Artificial Sequence
  epitope
EKHHRPGQQI

SEQ ID NO: 466 - protein, 15 aa, Artificial Sequence
  epitope
VKNGGKEEKHHRPGQ

SEQ ID NO: 467 - protein, 15 aa, Artificial Sequence
  epitope
  misc_feature (1)..(3): Xaa can be any naturally occurring amino acid
  misc_feature (5)..(5): Xaa can be any naturally occurring amino acid
  misc_feature (9)..(13): Xaa can be any naturally occurring amino acid
XXXGXKEEXXXXXGQ

SEQ ID NO: 468 - protein, 10 aa, Artificial Sequence
  epitope
KNGGKEEKHH

SEQ ID NO: 469 - protein, 9 aa, Artificial Sequence
  epitope
KNGGKEEKH

SEQ ID NO: 470 - protein, 9 aa, Artificial Sequence
  epitope
KNGGKEEKH

SEQ ID NO: 471 - protein, 7 aa, Artificial Sequence
  epitope
NGGKEEK

SEQ ID NO: 472 - protein, 7 aa, Artificial Sequence
  epitope
  misc_feature (1)..(1): Xaa can be any naturally occurring amino acid
  misc_feature (3)..(3): Xaa can be any naturally occurring amino acid
  misc_feature (7)..(7): Xaa can be any naturally occurring amino acid
XGXKEEX

SEQ ID NO: 473 - protein, 10 aa, Artificial Sequence
  epitope
KEEKHHRPGQ

SEQ ID NO: 474 - protein, 10 aa, Artificial Sequence
  epitope
  misc_feature (4)..(8): Xaa can be any naturally occurring amino acid
KEEXXXXXGQ

SEQ ID NO: 475 - protein, 10 aa, Artificial Sequence
  epitope
EKHHRPGQQI

SEQ ID NO: 476 - protein, 10 aa, Artificial Sequence
  epitope
  misc_feature (2)..(6): Xaa can be any naturally occurring amino acid
EXXXXXGQQI

SEQ ID NO: 477 - protein, 15 aa, Artificial Sequence
  epitope
VKNGGKEEKHHRPGQ

SEQ ID NO: 478 - protein, 10 aa, Artificial Sequence
  epitope
VKNGGKEEKH

SEQ ID NO: 479 - protein, 8 aa, Artificial Sequence
  epitope
KNGGKEEK

SEQ ID NO: 480 - protein, 8 aa, Artificial Sequence
  epitope
KEEKHHRP

SEQ ID NO: 481 - protein, 10 aa, Artificial Sequence
  epitope
EKHHRPGQQI

SEQ ID NO: 482 - protein, 15 aa, Artificial Sequence
  epitope
VKNGGKEEKHHRPGQ

SEQ ID NO: 483 - protein, 9 aa, Artificial Sequence
  epitope
TLVVKNGGK

SEQ ID NO: 484 - protein, 4 aa, Artificial Sequence
  epitope
KEEK

SEQ ID NO: 485 - protein, 9 aa, Artificial Sequence
  epitope
KHHRPGQQI

SEQ ID NO: 486 - protein, 15 aa, Artificial Sequence
  epitope
VKNGGKEEKHHRPGQ

SEQ ID NO: 487 - protein, 10 aa, Artificial Sequence
  epitope
TLVVKNGGKE

SEQ ID NO: 488 - protein, 9 aa, Artificial Sequence
  epitope
TLVVKNGGK

SEQ ID NO: 489 - protein, 15 aa, Artificial Sequence
  epitope
  misc_feature (1)..(6): Xaa can be any naturally occurring amino acid
  misc_feature (9)..(9): Xaa can be any naturally occurring amino acid
  misc_feature (12)..(12): Xaa can be any naturally occurring amino acid
XXXXXXEEXHHXPGQ

SEQ ID NO: 490 - protein, 8 aa, Artificial Sequence
  epitope
KNGGKEEK

SEQ ID NO: 491 - protein, 9 aa, Artificial Sequence
  epitope
GKEEKHHRP

SEQ ID NO: 492 - protein, 4 aa, Artificial Sequence
  epitope
KEEK

SEQ ID NO: 493 - protein, 8 aa, Artificial Sequence
  epitope
KNGGKEEK

SEQ ID NO: 494 - protein, 6 aa, Artificial Sequence
  epitope
GGKEEK

SEQ ID NO: 495 - protein, 5 aa, Artificial Sequence
  epitope
GKEEK

SEQ ID NO: 496 - protein, 8 aa, Artificial Sequence
  epitope
KNGGKEEK

SEQ ID NO: 497 - protein, 10 aa, Artificial Sequence
  epitope
GGKEEKHHRP

SEQ ID NO: 498 - protein, 4 aa, Artificial Sequence
  epitope
KEEK

SEQ ID NO: 499 - protein, 6 aa, Artificial Sequence
  epitope
GGKEEK

SEQ ID NO: 500 - protein, 15 aa, Artificial Sequence
  epitope
  misc_feature (1)..(3): Xaa can be any naturally occurring amino acid
  misc_feature (5)..(6): Xaa can be any naturally occurring amino acid
  misc_feature (10)..(14): Xaa can be any naturally occurring amino acid
XXXGXXEEKXXXXXQ

SEQ ID NO: 501 - protein, 15 aa, Artificial Sequence
  epitope
ESEDKYGTTPPQLDA

SEQ ID NO: 502 - protein, 15 aa, Artificial Sequence
  epitope
ESEDKYGTTPPQLDA

SEQ ID NO: 503 - protein, 15 aa, Artificial Sequence
  epitope
  misc_feature (1)..(2): Xaa can be any naturally occurring amino acid
  misc_feature (7)..(9): Xaa can be any naturally occurring amino acid
  misc_feature (11)..(15): Xaa can be any naturally occurring amino acid
XXEDKYXXXPXXXXX

SEQ ID NO: 504 - protein, 15 aa, Artificial Sequence
  epitope
  misc_feature (1)..(2): Xaa can be any naturally occurring amino acid
  misc_feature (7)..(15): Xaa can be any naturally occurring amino acid
XXEDKYXXXXXXXXX

SEQ ID NO: 505 - protein, 7 aa, Artificial Sequence
  epitope
DKYGTTP

SEQ ID NO: 506 - protein, 7 aa, Artificial Sequence
  epitope
  misc_feature (4)..(6): Xaa can be any naturally occurring amino acid
DKYXXXP

SEQ ID NO: 507 - protein, 10 aa, Artificial Sequence
  epitope
YGTTPPQLDA

SEQ ID NO: 508 - protein, 15 aa, Artificial Sequence
  epitope
ESEDKYGTTPPQLDA

SEQ ID NO: 509 - protein, 15 aa, Artificial Sequence
  epitope
  misc_feature (1)..(1): Xaa can be any naturally occurring amino acid
  misc_feature (3)..(3): Xaa can be any naturally occurring amino acid
  misc_feature (7)..(15): Xaa can be any naturally occurring amino acid
XSXDKYXXXXXXXXX

SEQ ID NO: 510 - protein, 7 aa, Artificial Sequence
  epitope
EDKYGTT

SEQ ID NO: 511 - protein, 6 aa, Artificial Sequence
  epitope
EDKYGT

SEQ ID NO: 512 - protein, 10 aa, Artificial Sequence
  epitope
YGTTPPQLDA

SEQ ID NO: 513 - protein, 9 aa, Artificial Sequence
  epitope
YGTTPPQLD

SEQ ID NO: 514 - protein, 15 aa, Artificial Sequence
  epitope
ESEDKYGTTPPQLDA

SEQ ID NO: 515 - protein, 15 aa, Artificial Sequence
  epitope
  misc_feature (1)..(1): Xaa can be any naturally occurring amino acid
  misc_feature (7)..(15): Xaa can be any naturally occurring amino acid
XSEDKYXXXXXXXXX

SEQ ID NO: 516 - protein, 4 aa, Artificial Sequence
  epitope
EDKY

SEQ ID NO: 517 - protein, 10 aa, Artificial Sequence
  epitope
YGTTPPQLDA

SEQ ID NO: 518 - protein, 15 aa, Artificial Sequence
  epitope
ESEDKYGTTPPQLDA

SEQ ID NO: 519 - protein, 15 aa, Artificial Sequence
  epitope
  misc_feature (1)..(1): Xaa can be any naturally occurring amino acid
  misc_feature (8)..(8): Xaa can be any naturally occurring amino acid
  misc_feature (14)..(15): Xaa can be any naturally occurring amino acid
XSEDKYGXTPPQLXX

SEQ ID NO: 520 - protein, 15 aa, Artificial Sequence
  epitope
  misc_feature (1)..(2): Xaa can be any naturally occurring amino acid
  misc_feature (8)..(10): Xaa can be any naturally occurring amino acid
  misc_feature (13)..(15): Xaa can be any naturally occurring amino acid
XXEDKYGXXXPQXXX

SEQ ID NO: 521 - protein, 15 aa, Artificial Sequence
  epitope
  misc_feature (1)..(3): Xaa can be any naturally occurring amino acid
  misc_feature (8)..(10): Xaa can be any naturally occurring amino acid
  misc_feature (13)..(15): Xaa can be any naturally occurring amino acid
XXXDKYGXXXPQXXX

SEQ ID NO: 522 - protein, 5 aa, Artificial Sequence
  epitope
DKYGT

SEQ ID NO: 523 - protein, 5 aa, Artificial Sequence
  epitope
  misc_feature (5)..(5): Xaa can be any naturally occurring amino acid
DKYGX

SEQ ID NO: 524 - protein, 10 aa, Artificial Sequence
  epitope
YGTTPPQLDA

SEQ ID NO: 525 - protein, 9 aa, Artificial Sequence
  epitope
GTTPPQLDA

SEQ ID NO: 526 - protein, 9 aa, Artificial Sequence
  epitope
  misc_feature (2)..(2): Xaa can be any naturally occurring amino acid
  misc_feature (8)..(9): Xaa can be any naturally occurring amino acid
GXTPPQLXX

SEQ ID NO: 527 - protein, 15 aa, Artificial Sequence
  epitope
ESEDKYGTTPPQLDA

SEQ ID NO: 528 - protein, 15 aa, Artificial Sequence
  epitope
  misc_feature (1)..(1): Xaa can be any naturally occurring amino acid
  misc_feature (3)..(3): Xaa can be any naturally occurring amino acid
  misc_feature (7)..(8): Xaa can be any naturally occurring amino acid
  misc_feature (10)..(10): Xaa can be any naturally occurring amino acid
  misc_feature (12)..(15): Xaa can be any naturally occurring amino acid
XSXDKYXXTXPXXXX

SEQ ID NO: 529 - protein, 10 aa, Artificial Sequence
  epitope
SEDKYGTTPP

SEQ ID NO: 530 - protein, 7 aa, Artificial Sequence
  epitope
EDKYGTT

SEQ ID NO: 531 - protein, 7 aa, Artificial Sequence
  epitope
  misc_feature (1)..(1): Xaa can be any naturally occurring amino acid
  misc_feature (5)..(6): Xaa can be any naturally occurring amino acid
XDKYXXT

SEQ ID NO: 532 - protein, 10 aa, Artificial Sequence
  epitope
YGTTPPQLDA

SEQ ID NO: 533 - protein, 9 aa, Artificial Sequence
  epitope
YGTTPPQLD

SEQ ID NO: 534 - protein, 15 aa, Artificial Sequence
  epitope
ESEDKYGTTPPQLDA

SEQ ID NO: 535 - protein, 15 aa, Artificial Sequence
  epitope
  misc_feature (1)..(1): Xaa can be any naturally occurring amino acid
  misc_feature (8)..(8): Xaa can be any naturally occurring amino acid
  misc_feature (10)..(15): Xaa can be any naturally occurring amino acid
XSEDKYGXTXXXXXX

SEQ ID NO: 536 - protein, 9 aa, Artificial Sequence
  epitope
SEDKYGTTP

SEQ ID NO: 537 - protein, 8 aa, Artificial Sequence
  epitope
EDKYGTTP

SEQ ID NO: 538 - protein, 7 aa, Artificial Sequence
  epitope
EDKYGTT

SEQ ID NO: 539 - protein, 7 aa, Artificial Sequence
  epitope
  misc_feature (6)..(6): Xaa can be any naturally occurring amino acid
EDKYGXT

SEQ ID NO: 540 - protein, 15 aa, Artificial Sequence
  epitope
ESEDKYGTTPPQLDA

SEQ ID NO: 541 - protein, 6 aa, Artificial Sequence
  epitope
EDKYGT

SEQ ID NO: 542 - protein, 4 aa, Artificial Sequence
  epitope
EDKY

SEQ ID NO: 543 - protein, 10 aa, Artificial Sequence
  epitope
YGTTPPQLDA

SEQ ID NO: 544 - protein, 15 aa, Artificial Sequence
  epitope
  misc_feature (1)..(1): Xaa can be any naturally occurring amino acid
  misc_feature (7)..(8): Xaa can be any naturally occurring amino acid
  misc_feature (10)..(10): Xaa can be any naturally occurring amino acid
  misc_feature (12)..(15): Xaa can be any naturally occurring amino acid
XSEDKYXXTXPXXXX

SEQ ID NO: 545 - protein, 15 aa, Artificial Sequence
  epitope
  misc_feature (1)..(1): Xaa can be any naturally occurring amino acid
  misc_feature (3)..(3): Xaa can be any naturally occurring amino acid
  misc_feature (7)..(8): Xaa can be any naturally occurring amino acid
  misc_feature (10)..(10): Xaa can be any naturally occurring amino acid
  misc_feature (12)..(15): Xaa can be any naturally occurring amino acid
XSXDKYXXTXPXXXX

SEQ ID NO: 546 - protein, 15 aa, Artificial Sequence
  epitope
  misc_feature (1)..(1): Xaa can be any naturally occurring amino acid
  misc_feature (3)..(3): Xaa can be any naturally occurring amino acid
  misc_feature (7)..(15): Xaa can be any naturally occurring amino acid
XSXDKYXXXXXXXXX

SEQ ID NO: 547 - protein, 5 aa, Artificial Sequence
  epitope
EDKYG

SEQ ID NO: 548 - protein, 4 aa, Artificial Sequence
  epitope
EDKY

SEQ ID NO: 549 - protein, 10 aa, Artificial Sequence
  epitope
YGTTPPQLDA

SEQ ID NO: 550 - protein, 9 aa, Artificial Sequence
  epitope
YGTTPPQLD

SEQ ID NO: 551 - protein, 15 aa, Artificial Sequence
  epitope
  misc_feature (1)..(3): Xaa can be any naturally occurring amino acid
  misc_feature (7)..(8): Xaa can be any naturally occurring amino acid
  misc_feature (12)..(12): Xaa can be any naturally occurring amino acid
XXXDKYXXTPPXLDA

SEQ ID NO: 552 - protein, 15 aa, Artificial Sequence
  epitope
  misc_feature (1)..(3): Xaa can be any naturally occurring amino acid
  misc_feature (7)..(8): Xaa can be any naturally occurring amino acid
  misc_feature (10)..(15): Xaa can be any naturally occurring amino acid
XXXDKYXXTXXXXXX

SEQ ID NO: 553 - protein, 10 aa, Artificial Sequence
  epitope
SEDKYGTTPP

SEQ ID NO: 554 - protein, 8 aa, Artificial Sequence
  epitope
EDKYGTTP

SEQ ID NO: 555 - protein, 8 aa, Artificial Sequence
  epitope
  misc_feature (1)..(1): Xaa can be any naturally occurring amino acid
  misc_feature (5)..(6): Xaa can be any naturally occurring amino acid
XDKYXXTP

SEQ ID NO: 556 - protein, 8 aa, Artificial Sequence
  epitope
  misc_feature (1)..(1): Xaa can be any naturally occurring amino acid
  misc_feature (5)..(6): Xaa can be any naturally occurring amino acid
  misc_feature (8)..(8): Xaa can be any naturally occurring amino acid
XDKYXXTX

SEQ ID NO: 557 - protein, 10 aa, Artificial Sequence
  epitope
YGTTPPQLDA

SEQ ID NO: 558 - protein, 8 aa, Artificial Sequence
  epitope
GTTPPQLD

SEQ ID NO: 559 - protein, 8 aa, Artificial Sequence
  epitope
  misc_feature (1)..(2): Xaa can be any naturally occurring amino acid
  misc_feature (6)..(6): Xaa can be any naturally occurring amino acid
XXTPPXLD

SEQ ID NO: 560 - protein, 15 aa, Artificial Sequence
  epitope
  misc_feature (1)..(1): Xaa can be any naturally occurring amino acid
  misc_feature (3)..(3): Xaa can be any naturally occurring amino acid
  misc_feature (7)..(8): Xaa can be any naturally occurring amino acid
  misc_feature (13)..(15): Xaa can be any naturally occurring amino acid
XSXDKYXXTPPQXXX

SEQ ID NO: 561 - protein, 15 aa, Artificial Sequence
  epitope
  misc_feature (1)..(3): Xaa can be any naturally occurring amino acid
  misc_feature (7)..(11): Xaa can be any naturally occurring amino acid
  misc_feature (13)..(15): Xaa can be any naturally occurring amino acid
XXXDKYXXXXXQXXX

SEQ ID NO: 562 - protein, 9 aa, Artificial Sequence
  epitope
SEDKYGTTP

SEQ ID NO: 563 - protein, 4 aa, Artificial Sequence
  epitope
EDKY

SEQ ID NO: 564 - protein, 10 aa, Artificial Sequence
  epitope
YGTTPPQLDA

SEQ ID NO: 565 - protein, 7 aa, Artificial Sequence
  epitope
TTPPQLD

SEQ ID NO: 566 - protein, 7 aa, Artificial Sequence
  epitope
  misc_feature (1)..(1): Xaa can be any naturally occurring amino acid
  misc_feature (6)..(7): Xaa can be any naturally occurring amino acid
XTPPQXX

SEQ ID NO: 567 - protein, 15 aa, Artificial Sequence
  epitope
  misc_feature (1)..(1): Xaa can be any naturally occurring amino acid
  misc_feature (3)..(3): Xaa can be any naturally occurring amino acid
  misc_feature (7)..(8): Xaa can be any naturally occurring amino acid
  misc_feature (13)..(15): Xaa can be any naturally occurring amino acid
XSXDKYXXTPPQXXX

SEQ ID NO: 568 - protein, 15 aa, Artificial Sequence
  epitope
  misc_feature (1)..(3): Xaa can be any naturally occurring amino acid
  misc_feature (7)..(10): Xaa can be any naturally occurring amino acid
  misc_feature (12)..(15): Xaa can be any naturally occurring amino acid
XXXDKYXXXXPXXXX

SEQ ID NO: 569 - protein, 8 aa, Artificial Sequence
  epitope
EDKYGTTP

SEQ ID NO: 570 - protein, 10 aa, Artificial Sequence
  epitope
YGTTPPQLDA

SEQ ID NO: 571 - protein, 5 aa, Artificial Sequence
  epitope
YGTTP

SEQ ID NO: 572 - protein, 15 aa, Artificial Sequence
  epitope
  misc_feature (1)..(2): Xaa can be any naturally occurring amino acid
  misc_feature (7)..(8): Xaa can be any naturally occurring amino acid
  misc_feature (13)..(13): Xaa can be any naturally occurring amino acid
XXEDKYXXTPPQXDA

SEQ ID NO: 573 - protein, 15 aa, Artificial Sequence
  epitope
  misc_feature (1)..(2): Xaa can be any naturally occurring amino acid
  misc_feature (7)..(14): Xaa can be any naturally occurring amino acid
XXEDKYXXXXXXXXA

SEQ ID NO: 574 - protein, 9 aa, Artificial Sequence
  epitope
EDKYGTTPP

SEQ ID NO: 575 - protein, 9 aa, Artificial Sequence
  epitope
  misc_feature (5)..(6): Xaa can be any naturally occurring amino acid
EDKYXXTPP

SEQ ID NO: 576 - protein, 9 aa, Artificial Sequence
  epitope
  misc_feature (5)..(9): Xaa can be any naturally occurring amino acid
EDKYXXXXX

SEQ ID NO: 577 - protein, 10 aa, Artificial Sequence
  epitope
YGTTPPQLDA

SEQ ID NO: 578 - protein, 9 aa, Artificial Sequence
  epitope
YGTTPPQLD

SEQ ID NO: 579 - protein, 9 aa, Artificial Sequence
  epitope
  misc_feature (2)..(3): Xaa can be any naturally occurring amino acid
  misc_feature (8)..(8): Xaa can be any naturally occurring amino acid
YXXTPPQXD

SEQ ID NO: 580 - protein, 15 aa, Artificial Sequence
  epitope
  misc_feature (1)..(1): Xaa can be any naturally occurring amino acid
  misc_feature (3)..(3): Xaa can be any naturally occurring amino acid
  misc_feature (7)..(8): Xaa can be any naturally occurring amino acid
XSXDKYXXTPPQLDA

SEQ ID NO: 581 - protein, 15 aa, Artificial Sequence
  epitope
  misc_feature (1)..(3): Xaa can be any naturally occurring amino acid
  misc_feature (7)..(8): Xaa can be any naturally occurring amino acid
  misc_feature (10)..(10): Xaa can be any naturally occurring amino acid
  misc_feature (14)..(14): Xaa can be any naturally occurring amino acid
XXXDKYXXTXPQLXA

SEQ ID NO: 582 - protein, 15 aa, Artificial Sequence
  epitope
  misc_feature (1)..(3): Xaa can be any naturally occurring amino acid
  misc_feature (7)..(8): Xaa can be any naturally occurring amino acid
  misc_feature (10)..(10): Xaa can be any naturally occurring amino acid
  misc_feature (13)..(15): Xaa can be any naturally occurring amino acid
XXXDKYXXTXPQXXX

SEQ ID NO: 583 - protein, 9 aa, Artificial Sequence
  epitope
SEDKYGTTP

SEQ ID NO: 584 - protein, 5 aa, Artificial Sequence
  epitope
SEDKY

SEQ ID NO: 585 - protein, 5 aa, Artificial Sequence
  epitope
  misc_feature (2)..(2): Xaa can be any naturally occurring amino acid
SXDKY

SEQ ID NO: 586 - protein, 9 aa, Artificial Sequence
  epitope
YGTTPPQLD

SEQ ID NO: 587 - protein, 8 aa, Artificial Sequence
  epitope
YGTTPPQL

SEQ ID NO: 588 - protein, 8 aa, Artificial Sequence
  epitope
  misc_feature (2)..(3): Xaa can be any naturally occurring amino acid
YXXTPPQL

SEQ ID NO: 589 - protein, 8 aa, Artificial Sequence
  epitope
  misc_feature (2)..(3): Xaa can be any naturally occurring amino acid
  misc_feature (5)..(5): Xaa can be any naturally occurring amino acid
YXXTXPQL

SEQ ID NO: 590 - protein, 8 aa, Artificial Sequence
  epitope
  misc_feature (2)..(3): Xaa can be any naturally occurring amino acid
  misc_feature (5)..(5): Xaa can be any naturally occurring amino acid
  misc_feature (8)..(8): Xaa can be any naturally occurring amino acid
YXXTXPQX

SEQ ID NO: 591 - protein, 15 aa, Artificial Sequence
  epitope
  misc_feature (1)..(1): Xaa can be any naturally occurring amino acid
  misc_feature (3)..(4): Xaa can be any naturally occurring amino acid
  misc_feature (7)..(7): Xaa can be any naturally occurring amino acid
  misc_feature (10)..(10): Xaa can be any naturally occurring amino acid
  misc_feature (15)..(15): Xaa can be any naturally occurring amino acid
XSXXKYXTTXPQLDX

SEQ ID NO: 592 - protein, 15 aa, Artificial Sequence
  epitope
  misc_feature (1)..(4): Xaa can be any naturally occurring amino acid
  misc_feature (7)..(7): Xaa can be any naturally occurring amino acid
  misc_feature (10)..(10): Xaa can be any naturally occurring amino acid
  misc_feature (14)..(15): Xaa can be any naturally occurring amino acid
XXXXKYXTTXPQLXX

SEQ ID NO: 593 - protein, 15 aa, Artificial Sequence
  epitope
  misc_feature (1)..(4): Xaa can be any naturally occurring amino acid
  misc_feature (7)..(8): Xaa can be any naturally occurring amino acid
  misc_feature (10)..(10): Xaa can be any naturally occurring amino acid
  misc_feature (13)..(15): Xaa can be any naturally occurring amino acid
XXXXKYXXTXPQXXX

SEQ ID NO: 594 - protein, 9 aa, Artificial Sequence
  epitope
YGTTPPQLD

SEQ ID NO: 595 - protein, 8 aa, Artificial Sequence
  epitope
YGTTPPQL

SEQ ID NO: 596 - protein, 8 aa, Artificial Sequence
  epitope
  misc_feature (2)..(2): Xaa can be any naturally occurring amino acid
  misc_feature (5)..(5): Xaa can be any naturally occurring amino acid
YXTTXPQL

SEQ ID NO: 597 - protein, 8 aa, Artificial Sequence
  epitope
  misc_feature (2)..(3): Xaa can be any naturally occurring amino acid
  misc_feature (5)..(5): Xaa can be any naturally occurring amino acid
  misc_feature (8)..(8): Xaa can be any naturally occurring amino acid
YXXTXPQX

SEQ ID NO: 598 - protein, 15 aa, Artificial Sequence
  epitope
  misc_feature (1)..(2): Xaa can be any naturally occurring amino acid
  misc_feature (7)..(10): Xaa can be any naturally occurring amino acid
  misc_feature (12)..(15): Xaa can be any naturally occurring amino acid
XXEDKYXXXXPXXXX

SEQ ID NO: 599 - protein, 15 aa, Artificial Sequence
  epitope
  misc_feature (1)..(2): Xaa can be any naturally occurring amino acid
  misc_feature (7)..(15): Xaa can be any naturally occurring amino acid
XXEDKYXXXXXXXXX

SEQ ID NO: 600 - protein, 8 aa, Artificial Sequence
  epitope
EDKYGTTP

SEQ ID NO: 601 - protein, 7 aa, Artificial Sequence
  epitope
EDKYGTT

SEQ ID NO: 602 - protein, 7 aa, Artificial Sequence
  epitope
  misc_feature (5)..(7): Xaa can be any naturally occurring amino acid
EDKYXXX

SEQ ID NO: 603 - protein, 10 aa, Artificial Sequence
  epitope
YGTTPPQLDA

SEQ ID NO: 604 - protein, 8 aa, Artificial Sequence
  epitope
GTTPPQLD

SEQ ID NO: 605 - protein, 15 aa, Artificial Sequence
  epitope
TKENSKSQVSLSVSS

SEQ ID NO: 606 - protein, 15 aa, Artificial Sequence
  epitope
TKENSKSQVSLSVSS

SEQ ID NO: 607 - protein, 15 aa, Artificial Sequence
  epitope
  misc_feature (1)..(1): Xaa can be any naturally occurring amino acid
  misc_feature (3)..(3): Xaa can be any naturally occurring amino acid
  misc_feature (15)..(15): Xaa can be any naturally occurring amino acid
XKXNSKSQVSLSVSX

SEQ ID NO: 608 - protein, 15 aa, Artificial Sequence
  epitope
  misc_feature (1)..(1): Xaa can be any naturally occurring amino acid
  misc_feature (3)..(4): Xaa can be any naturally occurring amino acid
  misc_feature (9)..(9): Xaa can be any naturally occurring amino acid
  misc_feature (13)..(13): Xaa can be any naturally occurring amino acid
  misc_feature (15)..(15): Xaa can be any naturally occurring amino acid
XKXXSKSQXSLSXSX

SEQ ID NO: 609 - protein, 15 aa, Artificial Sequence
  epitope
  misc_feature (1)..(1): Xaa can be any naturally occurring amino acid
  misc_feature (3)..(4): Xaa can be any naturally occurring amino acid
  misc_feature (9)..(9): Xaa can be any naturally occurring amino acid
  misc_feature (11)..(11): Xaa can be any naturally occurring amino acid
  misc_feature (13)..(15): Xaa can be any naturally occurring amino acid
XKXXSKSQXSXSXXX

SEQ ID NO: 610 - protein, 5 aa, Artificial Sequence
  epitope
KENSK

SEQ ID NO: 611 - protein, 5 aa, Artificial Sequence
  epitope
  misc_feature (2)..(2): Xaa can be any naturally occurring amino acid
KXNSK

SEQ ID NO: 612 - protein, 4 aa, Artificial Sequence
  epitope
ENSK

SEQ ID NO: 613 - protein, 15 aa, Artificial Sequence
  epitope
TKENSKSQVSLSVSS

SEQ ID NO: 614 - protein, 15 aa, Artificial Sequence
  epitope
TKENSKSQVSLSVSS

SEQ ID NO: 615 - protein, 10 aa, Artificial Sequence
  epitope
VSLSVSSVTP

SEQ ID NO: 616 - protein, 15 aa, Artificial Sequence
  epitope
TKENSKSQVSLSVSS

SEQ ID NO: 617 - protein, 15 aa, Artificial Sequence
  epitope
  misc_feature (1)..(2): Xaa can be any naturally occurring amino acid
  misc_feature (9)..(15): Xaa can be any naturally occurring amino acid
XXENSKSQXXXXXXX

SEQ ID NO: 618 - protein, 15 aa, Artificial Sequence
  epitope
  misc_feature (1)..(3): Xaa can be any naturally occurring amino acid
  misc_feature (8)..(15): Xaa can be any naturally occurring amino acid
XXXNSKSXXXXXXXX

SEQ ID NO: 619 - protein, 15 aa, Artificial Sequence
  epitope
TKENSKSQVSLSVSS

SEQ ID NO: 620 - protein, 15 aa, Artificial Sequence
  epitope
  misc_feature (1)..(1): Xaa can be any naturally occurring amino acid
  misc_feature (9)..(15): Xaa can be any naturally occurring amino acid
XKENSKSQXXXXXXX

SEQ ID NO: 621 - protein, 15 aa, Artificial Sequence
  epitope
TKENSKSQVSLSVSS

SEQ ID NO: 622 - protein, 9 aa, Artificial Sequence
  epitope
SLSVSSVTP

SEQ ID NO: 623 - protein, 15 aa, Artificial Sequence
  epitope
TKENSKSQVSLSVSS

SEQ ID NO: 624 - protein, 15 aa, Artificial Sequence
  epitope
TKENSKSQVSLSVSS

SEQ ID NO: 625 - protein, 15 aa, Artificial Sequence
  epitope
  misc_feature (1)..(1): Xaa can be any naturally occurring amino acid
  misc_feature (3)..(4): Xaa can be any naturally occurring amino acid
  misc_feature (7)..(13): Xaa can be any naturally occurring amino acid
XKXXSKXXXXXXXSS

SEQ ID NO: 626 - protein, 4 aa, Artificial Sequence
  epitope
NSKS

SEQ ID NO: 627 - protein, 15 aa, Artificial Sequence
  epitope
  misc_feature (1)..(2): Xaa can be any naturally occurring amino acid
  misc_feature (7)..(15): Xaa can be any naturally occurring amino acid
XXENSKXXXXXXXXX

SEQ ID NO: 628 - protein, 15 aa, Artificial Sequence
  epitope
  misc_feature (1)..(3): Xaa can be any naturally occurring amino acid
  misc_feature (8)..(15): Xaa can be any naturally occurring amino acid
XXXNSKSXXXXXXXX

SEQ ID NO: 629 - protein, 15 aa, Artificial Sequence
  epitope
  misc_feature (1)..(2): Xaa can be any naturally occurring amino acid
  misc_feature (4)..(4): Xaa can be any naturally occurring amino acid
  misc_feature (7)..(7): Xaa can be any naturally occurring amino acid
  misc_feature (9)..(10): Xaa can be any naturally occurring amino acid
XXEXSKXQXXLSVSS

SEQ ID NO: 630 - protein, 15 aa, Artificial Sequence
  epitope
  misc_feature (1)..(2): Xaa can be any naturally occurring amino acid
  misc_feature (4)..(4): Xaa can be any naturally occurring amino acid
  misc_feature (7)..(10): Xaa can be any naturally occurring amino acid
  misc_feature (12)..(12): Xaa can be any naturally occurring amino acid
XXEXSKXXXXLXVSS

SEQ ID NO: 631 - protein, 15 aa, Artificial Sequence
  epitope
  misc_feature (2)..(2): Xaa can be any naturally occurring amino acid
  misc_feature (7)..(15): Xaa can be any naturally occurring amino acid
TXENSKXXXXXXXXX

SEQ ID NO: 632 - protein, 4 aa, Artificial Sequence
  epitope
ENSK

SEQ ID NO: 633 - protein, 15 aa, Artificial Sequence
  epitope
TTVTGAKDSVASTIT

SEQ ID NO: 634 - protein, 15 aa, Artificial Sequence
  epitope
TTVTGAKDSVASTIT

SEQ ID NO: 635 - protein, 15 aa, Artificial Sequence
  epitope
  misc_feature (1)..(6): Xaa can be any naturally occurring amino acid
  misc_feature (10)..(11): Xaa can be any naturally occurring amino acid
  misc_feature (13)..(15): Xaa can be any naturally occurring amino acid
XXXXXXKDSXXSXXX

SEQ ID NO: 636 - protein, 10 aa, Artificial Sequence
  epitope
AVTTTVTGAK

SEQ ID NO: 637 - protein, 9 aa, Artificial Sequence
  epitope
AVTTTVTGA

SEQ ID NO: 638 - protein, 4 aa, Artificial Sequence
  epitope
KDSV

SEQ ID NO: 639 - protein, 15 aa, Artificial Sequence
  epitope
TTVTGAKDSVASTIT

SEQ ID NO: 640 - protein, 15 aa, Artificial Sequence
  epitope
  misc_feature (6)..(6): Xaa can be any naturally occurring amino acid
  misc_feature (9)..(9): Xaa can be any naturally occurring amino acid
  misc_feature (12)..(15): Xaa can be any naturally occurring amino acid
TTVTGXKDXVAXXXX

SEQ ID NO: 641 - protein, 15 aa, Artificial Sequence
  epitope
  misc_feature (2)..(2): Xaa can be any naturally occurring amino acid
  misc_feature (4)..(4): Xaa can be any naturally occurring amino acid
  misc_feature (6)..(6): Xaa can be any naturally occurring amino acid
  misc_feature (8)..(9): Xaa can be any naturally occurring amino acid
  misc_feature (11)..(15): Xaa can be any naturally occurring amino acid
TXVXGXKXXVXXXXX

SEQ ID NO: 642 - protein, 10 aa, Artificial Sequence
  epitope
DAVTTTVTGA

SEQ ID NO: 643 - protein, 9 aa, Artificial Sequence
  epitope
AVTTTVTGA

SEQ ID NO: 644 - protein, 7 aa, Artificial Sequence
  epitope
TTTVTGA

SEQ ID NO: 645 - protein, 7 aa, Artificial Sequence
  epitope
  misc_feature (7)..(7): Xaa can be any naturally occurring amino acid
TTTVTGX

SEQ ID NO: 646 - protein, 7 aa, Artificial Sequence
  epitope
  misc_feature (3)..(3): Xaa can be any naturally occurring amino acid
  misc_feature (5)..(5): Xaa can be any naturally occurring amino acid
  misc_feature (7)..(7): Xaa can be any naturally occurring amino acid
TTXVXGX

SEQ ID NO: 647 - protein, 9 aa, Artificial Sequence
  epitope
TGAKDSVAS

SEQ ID NO: 648 - protein, 5 aa, Artificial Sequence
  epitope
DSVAS

SEQ ID NO: 649 - protein, 4 aa, Artificial Sequence
  epitope
DSVA

SEQ ID NO: 650 - protein, 15 aa, Artificial Sequence
  epitope
TTVTGAKDSVASTIT

SEQ ID NO: 651 - protein, 9 aa, Artificial Sequence
  epitope
AVTTTVTGA

SEQ ID NO: 652 - protein, 6 aa, Artificial Sequence
  epitope
DSVAST

SEQ ID NO: 653 - protein, 15 aa, Artificial Sequence
  epitope
TTVTGAKDSVASTIT

SEQ ID NO: 654 - protein, 15 aa, Artificial Sequence
  epitope
TTVTGAKDSVASTIT

SEQ ID NO: 655 - protein, 10 aa, Artificial Sequence
  epitope
AVTTTVTGAK

SEQ ID NO: 656 - protein, 7 aa, Artificial Sequence
  epitope
TTTVTGA

SEQ ID NO: 657 - protein, 10 aa, Artificial Sequence
  epitope
TVTGAKDSVA

SEQ ID NO: 658 - protein, 8 aa, Artificial Sequence
  epitope
KDSVASTI

SEQ ID NO: 659 - protein, 6 aa, Artificial Sequence
  epitope
DSVAST

SEQ ID NO: 660 - protein, 15 aa, Artificial Sequence
  epitope
TTVTGAKDSVASTIT

SEQ ID NO: 661 - protein, 15 aa, Artificial Sequence
  epitope
  misc_feature (1)..(6): Xaa can be any naturally occurring amino acid
  misc_feature (8)..(9): Xaa can be any naturally occurring amino acid
  misc_feature (13)..(13): Xaa can be any naturally occurring amino acid
  misc_feature (15)..(15): Xaa can be any naturally occurring amino acid
XXXXXXKXXVASXIX

SEQ ID NO: 662 - protein, 9 aa, Artificial Sequence
  epitope
AVTTTVTGA

SEQ ID NO: 663 - protein, 8 aa, Artificial Sequence
  epitope
KDSVASTI

SEQ ID NO: 664 - protein, 8 aa, Artificial Sequence
  epitope
  misc_feature (2)..(3): Xaa can be any naturally occurring amino acid
  misc_feature (7)..(7): Xaa can be any naturally occurring amino acid
KXXVASXI

SEQ ID NO: 665 - protein, 15 aa, Artificial Sequence
  epitope
  misc_feature (1)..(4): Xaa can be any naturally occurring amino acid
  misc_feature (6)..(6): Xaa can be any naturally occurring amino acid
  misc_feature (12)..(15): Xaa can be any naturally occurring amino acid
XXXXGXKDSVAXXXX

SEQ ID NO: 666 - protein, 15 aa, Artificial Sequence
  epitope
  misc_feature (1)..(6): Xaa can be any naturally occurring amino acid
  misc_feature (12)..(15): Xaa can be any naturally occurring amino acid
XXXXXXKDSVAXXXX

SEQ ID NO: 667 - protein, 15 aa, Artificial Sequence
  epitope
  misc_feature (1)..(6): Xaa can be any naturally occurring amino acid
  misc_feature (10)..(10): Xaa can be any naturally occurring amino acid
  misc_feature (12)..(15): Xaa can be any naturally occurring amino acid
XXXXXXKDSXAXXXX

SEQ ID NO: 668 - protein, 5 aa, Artificial Sequence
  epitope
KDSVA

SEQ ID NO: 669 - protein, 4 aa, Artificial Sequence
  epitope
KDSV

SEQ ID NO: 670 - protein, 15 aa, Artificial Sequence
  epitope
  misc_feature (2)..(3): Xaa can be any naturally occurring amino acid
  misc_feature (8)..(8): Xaa can be any naturally occurring amino acid
  misc_feature (10)..(15): Xaa can be any naturally occurring amino acid
TXXTGAKXSXXXXXX

SEQ ID NO: 671 - protein, 15 aa, Artificial Sequence
  epitope
  misc_feature (2)..(3): Xaa can be any naturally occurring amino acid
  misc_feature (6)..(6): Xaa can be any naturally occurring amino acid
  misc_feature (8)..(8): Xaa can be any naturally occurring amino acid
  misc_feature (10)..(15): Xaa can be any naturally occurring amino acid
TXXTGXKXSXXXXXX

SEQ ID NO: 672 - protein, 7 aa, Artificial Sequence
  epitope
GAKDSVA

SEQ ID NO: 673 - protein, 7 aa, Artificial Sequence
  epitope
  misc_feature (4)..(4): Xaa can be any naturally occurring amino acid
  misc_feature (6)..(7): Xaa can be any naturally occurring amino acid
GAKXSXX

SEQ ID NO: 674 - protein, 15 aa, Artificial Sequence
  epitope
NQKIQDAQDKLYLSW

SEQ ID NO: 675 - protein, 15 aa, Artificial Sequence
  epitope
NQKIQDAQDKLYLSW

SEQ ID NO: 676 - protein, 10 aa, Artificial Sequence
  epitope
AQDKLYLSWL

SEQ ID NO: 677 - protein, 15 aa, Artificial Sequence
  epitope
NQKIQDAQDKLYLSW

SEQ ID NO: 678 - protein, 15 aa, Artificial Sequence
  epitope
NQKIQDAQDKLYLSW

SEQ ID NO: 679 - protein, 15 aa, Artificial Sequence
  epitope
  misc_feature (1)..(1): Xaa can be any naturally occurring amino acid
  misc_feature (4)..(9): Xaa can be any naturally occurring amino acid
XQKXXXXXXKLYLSW

SEQ ID NO: 680 - protein, 15 aa, Artificial Sequence
  epitope
  misc_feature (1)..(1): Xaa can be any naturally occurring amino acid
  misc_feature (4)..(10): Xaa can be any naturally occurring amino acid
XQKXXXXXXXLYLSW

SEQ ID NO: 681 - protein, 10 aa, Artificial Sequence
  epitope
AQDKLYLSWL

SEQ ID NO: 682 - protein, 10 aa, Artificial Sequence
  epitope
  misc_feature (1)..(3): Xaa can be any naturally occurring amino acid
XXXKLYLSWL

SEQ ID NO: 683 - protein, 10 aa, Artificial Sequence
  epitope
  misc_feature (1)..(4): Xaa can be any naturally occurring amino acid
XXXXLYLSWL

SEQ ID NO: 684 - protein, 15 aa, Artificial Sequence
  epitope
NQKIQDAQDKLYLSW

SEQ ID NO: 685 - protein, 15 aa, Artificial Sequence
  epitope
NQKIQDAQDKLYLSW

SEQ ID NO: 686 - protein, 10 aa, Artificial Sequence
  epitope
DKLYLSWLEW

SEQ ID NO: 687 - protein, 15 aa, Artificial Sequence
  epitope
  misc_feature (6)..(15): Xaa can be any naturally occurring amino acid
NQKIQXXXXXXXXXX

SEQ ID NO: 688 - protein, 15 aa, Artificial Sequence
  epitope
  misc_feature (6)..(7): Xaa can be any naturally occurring amino acid
  misc_feature (9)..(9): Xaa can be any naturally occurring amino acid
  misc_feature (15)..(15): Xaa can be any naturally occurring amino acid
NQKIQXXQXKLYLSX

SEQ ID NO: 689 - protein, 15 aa, Artificial Sequence
  epitope
  misc_feature (1)..(1): Xaa can be any naturally occurring amino acid
  misc_feature (6)..(9): Xaa can be any naturally occurring amino acid
  misc_feature (15)..(15): Xaa can be any naturally occurring amino acid
XQKIQXXXXKLYLSX

SEQ ID NO: 690 - protein, 15 aa, Artificial Sequence
  epitope
  misc_feature (1)..(1): Xaa can be any naturally occurring amino acid
  misc_feature (6)..(11): Xaa can be any naturally occurring amino acid
  misc_feature (14)..(15): Xaa can be any naturally occurring amino acid
XQKIQXXXXXXYLXX

SEQ ID NO: 691 - protein, 8 aa, Artificial Sequence
  epitope
QDKLYLSW

SEQ ID NO: 692 - protein, 8 aa, Artificial Sequence
  epitope
  misc_feature (2)..(2): Xaa can be any naturally occurring amino acid
  misc_feature (8)..(8): Xaa can be any naturally occurring amino acid
QXKLYLSX

SEQ ID NO: 693 - protein, 8 aa, Artificial Sequence
  epitope
  misc_feature (1)..(2): Xaa can be any naturally occurring amino acid
  misc_feature (8)..(8): Xaa can be any naturally occurring amino acid
XXKLYLSX

SEQ ID NO: 694 - protein, 15 aa, Artificial Sequence
  epitope
EDWEKESALQRARED

SEQ ID NO: 695 - protein, 15 aa, Artificial Sequence
  epitope
EDWEKESALQRARED

SEQ ID NO: 696 - protein, 15 aa, Artificial Sequence
  epitope
  misc_feature (1)..(2): Xaa can be any naturally occurring amino acid
  misc_feature (7)..(8): Xaa can be any naturally occurring amino acid
  misc_feature (11)..(13): Xaa can be any naturally occurring amino acid
XXWEKEXXLQXXXED

SEQ ID NO: 697 - protein, 15 aa, Artificial Sequence
  epitope
  misc_feature (1)..(2): Xaa can be any naturally occurring amino acid
  misc_feature (7)..(15): Xaa can be any naturally occurring amino acid
XXWEKEXXXXXXXXX

SEQ ID NO: 698 - protein, 7 aa, Artificial Sequence
  epitope
WEKESAL

SEQ ID NO: 699 - protein, 7 aa, Artificial Sequence
  epitope
  misc_feature (5)..(6): Xaa can be any naturally occurring amino acid
WEKEXXL

SEQ ID NO: 700 - protein, 7 aa, Artificial Sequence
  epitope
  misc_feature (5)..(7): Xaa can be any naturally occurring amino acid
WEKEXXX

SEQ ID NO: 701 - protein, 6 aa, Artificial Sequence
  epitope
EKESAL

SEQ ID NO: 702 - protein, 6 aa, Artificial Sequence
  epitope
  misc_feature (4)..(5): Xaa can be any naturally occurring amino acid
EKEXXL

SEQ ID NO: 703 - protein, 15 aa, Artificial Sequence
  epitope
EDWEKESALQRARED

SEQ ID NO: 704 - protein, 15 aa, Artificial Sequence
  epitope
  misc_feature (1)..(2): Xaa can be any naturally occurring amino acid
  misc_feature (7)..(15): Xaa can be any naturally occurring amino acid
XXWEKEXXXXXXXXX

SEQ ID NO: 705 - protein, 10 aa, Artificial Sequence
  epitope
MEDWEKESAL

SEQ ID NO: 706 - protein, 10 aa, Artificial Sequence
  epitope
DWEKESALQR

SEQ ID NO: 707 - protein, 8 aa, Artificial Sequence
  epitope
DWEKESAL

SEQ ID NO: 708 - protein, 7 aa, Artificial Sequence
  epitope
DWEKESA

SEQ ID NO: 709 - protein, 7 aa, Artificial Sequence
  epitope
  misc_feature (1)..(1): Xaa can be any naturally occurring amino acid
  misc_feature (6)..(7): Xaa can be any naturally occurring amino acid
XWEKEXX

SEQ ID NO: 710 - protein, 15 aa, Artificial Sequence
  epitope
EDWEKESALQRARED

SEQ ID NO: 711 - protein, 4 aa, Artificial Sequence
  epitope
WEKE

SEQ ID NO: 712 - protein, 15 aa, Artificial Sequence
  epitope
EDWEKESALQRARED

SEQ ID NO: 713 - protein, 8 aa, Artificial Sequence
  epitope
DWEKESAL

SEQ ID NO: 714 - protein, 8 aa, Artificial Sequence
  epitope
DWEKESAL

SEQ ID NO: 715 - protein, 5 aa, Artificial Sequence
  epitope
WEKES

SEQ ID NO: 716 - protein, 4 aa, Artificial Sequence
  epitope
WEKE

SEQ ID NO: 717 - protein, 15 aa, Artificial Sequence
  epitope
EDWEKESALQRARED

SEQ ID NO: 718 - protein, 15 aa, Artificial Sequence
  epitope
  misc_feature (1)..(4): Xaa can be any naturally occurring amino acid
  misc_feature (7)..(7): Xaa can be any naturally occurring amino acid
  misc_feature (10)..(10): Xaa can be any naturally occurring amino acid
  misc_feature (12)..(12): Xaa can be any naturally occurring amino acid
  misc_feature (14)..(14): Xaa can be any naturally occurring amino acid
XXXXKEXALXRXRXD

SEQ ID NO: 719 - protein, 7 aa, Artificial Sequence
  epitope
WEKESAL

SEQ ID NO: 720 - protein, 5 aa, Artificial Sequence
  epitope
WEKES

SEQ ID NO: 721 - protein, 10 aa, Artificial Sequence
  epitope
KESALQRARE

SEQ ID NO: 722 - protein, 10 aa, Artificial Sequence
  epitope
  misc_feature (3)..(3): Xaa can be any naturally occurring amino acid
  misc_feature (6)..(6): Xaa can be any naturally occurring amino acid
  misc_feature (8)..(8): Xaa can be any naturally occurring amino acid
  misc_feature (10)..(10): Xaa can be any naturally occurring amino acid
KEXALXRXRX

SEQ ID NO: 723 - protein, 15 aa, Artificial Sequence
  epitope
EDWEKESALQRARED

SEQ ID NO: 724 - protein, 6 aa, Artificial Sequence
  epitope
WEKESA

SEQ ID NO: 725 - protein, 4 aa, Artificial Sequence
  epitope
WEKE

SEQ ID NO: 726 - protein, 10 aa, Artificial Sequence
  epitope
KESALQRARE

SEQ ID NO: 727 - protein, 15 aa, Artificial Sequence
  epitope
EDWEKESALQRARED

SEQ ID NO: 728 - protein, 7 aa, Artificial Sequence
  epitope
MEDWEKE

SEQ ID NO: 729 - protein, 7 aa, Artificial Sequence
  epitope
KESALQR

SEQ ID NO: 730 - protein, 10 aa, Artificial Sequence
  epitope
LQRAREDIFM

SEQ ID NO: 731 - protein, 15 aa, Artificial Sequence
  epitope
EDWEKESALQRARED

SEQ ID NO: 732 - protein, 4 aa, Artificial Sequence
  epitope
WEKE

SEQ ID NO: 733 - protein, 15 aa, Artificial Sequence
  epitope
  misc_feature (1)..(4): Xaa can be any naturally occurring amino acid
  misc_feature (7)..(7): Xaa can be any naturally occurring amino acid
  misc_feature (9)..(10): Xaa can be any naturally occurring amino acid
  misc_feature (12)..(12): Xaa can be any naturally occurring amino acid
  misc_feature (14)..(14): Xaa can be any naturally occurring amino acid
XXXXKEXAXXRXRXD

SEQ ID NO: 734 - protein, 10 aa, Artificial Sequence
  epitope
DWEKESALQR

SEQ ID NO: 735 - protein, 9 aa, Artificial Sequence
  epitope
WEKESALQR

SEQ ID NO: 736 - protein, 7 aa, Artificial Sequence
  epitope
WEKESAL

SEQ ID NO: 737 - protein, 10 aa, Artificial Sequence
  epitope
KESALQRARE

SEQ ID NO: 738 - protein, 10 aa, Artificial Sequence
  epitope
  misc_feature (3)..(3): Xaa can be any naturally occurring amino acid
  misc_feature (5)..(6): Xaa can be any naturally occurring amino acid
  misc_feature (8)..(8): Xaa can be any naturally occurring amino acid
  misc_feature (10)..(10): Xaa can be any naturally occurring amino acid
KEXAXXRXRX

SEQ ID NO: 739 - protein, 7 aa, Artificial Sequence
  epitope
WEKESAL

SEQ ID NO: 740 - protein, 6 aa, Artificial Sequence
  epitope
EKESAL

SEQ ID NO: 741 - protein, 15 aa, Artificial Sequence
  epitope
  misc_feature (1)..(2): Xaa can be any naturally occurring amino acid
  misc_feature (7)..(12): Xaa can be any naturally occurring amino acid
XXWEKEXXXXXXRED

SEQ ID NO: 742 - protein, 15 aa, Artificial Sequence
  epitope
  misc_feature (1)..(4): Xaa can be any naturally occurring amino acid
  misc_feature (6)..(12): Xaa can be any naturally occurring amino acid
XXXXKXXXXXXXRED

SEQ ID NO: 743 - protein, 9 aa, Artificial Sequence
  epitope
WEKESALQR

SEQ ID NO: 744 - protein, 5 aa, Artificial Sequence
  epitope
KESAL

SEQ ID NO: 745 - protein, 15 aa, Artificial Sequence
  epitope
  misc_feature (1)..(2): Xaa can be any naturally occurring amino acid
  misc_feature (7)..(12): Xaa can be any naturally occurring amino acid
  misc_feature (14)..(15): Xaa can be any naturally occurring amino acid
XXWEKEXXXXXXRXX

SEQ ID NO: 746 - protein, 10 aa, Artificial Sequence
  epitope
DWEKESALQR

SEQ ID NO: 747 - protein, 10 aa, Artificial Sequence
  epitope
  misc_feature (1)..(1): Xaa can be any naturally occurring amino acid
  misc_feature (6)..(10): Xaa can be any naturally occurring amino acid
XWEKEXXXXX

SEQ ID NO: 748 - protein, 10 aa, Artificial Sequence
  epitope
DWEKESALQR

SEQ ID NO: 749 - protein, 8 aa, Artificial Sequence
  epitope
ESALQRAR

SEQ ID NO: 750 - protein, 5 aa, Artificial Sequence
  epitope
WEKES

SEQ ID NO: 751 - protein, 4 aa, Artificial Sequence
  epitope
WEKE

SEQ ID NO: 752 - protein, 15 aa, Artificial Sequence
  epitope
  misc_feature (1)..(2): Xaa can be any naturally occurring amino acid
  misc_feature (7)..(7): Xaa can be any naturally occurring amino acid
  misc_feature (9)..(12): Xaa can be any naturally occurring amino acid
  misc_feature (14)..(15): Xaa can be any naturally occurring amino acid
XXWEKEXAXXXXRXX

SEQ ID NO: 753 - protein, 15 aa, Artificial Sequence
  epitope
  misc_feature (1)..(2): Xaa can be any naturally occurring amino acid
  misc_feature (7)..(15): Xaa can be any naturally occurring amino acid
XXWEKEXXXXXXXXX

SEQ ID NO: 754 - protein, 9 aa, Artificial Sequence
  epitope
WEKESALQR

SEQ ID NO: 755 - protein, 7 aa, Artificial Sequence
  epitope
WEKESAL

SEQ ID NO: 756 - protein, 7 aa, Artificial Sequence
  epitope
  misc_feature (5)..(5): Xaa can be any naturally occurring amino acid
  misc_feature (7)..(7): Xaa can be any naturally occurring amino acid
WEKEXAX

SEQ ID NO: 757 - protein, 7 aa, Artificial Sequence
  epitope
  misc_feature (5)..(7): Xaa can be any naturally occurring amino acid
WEKEXXX

SEQ ID NO: 758 - protein, 15 aa, Artificial Sequence
  epitope
LSDIMDYYKDREGSH

SEQ ID NO: 759 - protein, 15 aa, Artificial Sequence
  epitope
LSDIMDYYKDREGSH

SEQ ID NO: 760 - protein, 6 aa, Artificial Sequence
  epitope
KDREGS

SEQ ID NO: 761 - protein, 15 aa, Artificial Sequence
  epitope
LSDIMDYYKDREGSH

SEQ ID NO: 762 - protein, 15 aa, Artificial Sequence
  epitope
  misc_feature (1)..(8): Xaa can be any naturally occurring amino acid
  misc_feature (10)..(10): Xaa can be any naturally occurring amino acid
XXXXXXXXKXREGSH

SEQ ID NO: 763 - protein, 15 aa, Artificial Sequence
  epitope
  misc_feature (1)..(8): Xaa can be any naturally occurring amino acid
  misc_feature (10)..(11): Xaa can be any naturally occurring amino acid
XXXXXXXXKXXEGSH

SEQ ID NO: 764 - protein, 6 aa, Artificial Sequence
  epitope
KDREGS

SEQ ID NO: 765 - protein, 5 aa, Artificial Sequence
  epitope
KDREG

SEQ ID NO: 766 - protein, 5 aa, Artificial Sequence
  epitope
  misc_feature (2)..(2): Xaa can be any naturally occurring amino acid
KXREG

SEQ ID NO: 767 - protein, 4 aa, Artificial Sequence
  epitope
DREG

SEQ ID NO: 768 - protein, 15 aa, Artificial Sequence
  epitope
LSDIMDYYKDREGSH

SEQ ID NO: 769 - protein, 15 aa, Artificial Sequence
  epitope
  misc_feature (1)..(8): Xaa can be any naturally occurring amino acid
  misc_feature (10)..(10): Xaa can be any naturally occurring amino acid
  misc_feature (14)..(15): Xaa can be any naturally occurring amino acid
XXXXXXXXKXREGXX

SEQ ID NO: 770 - protein, 5 aa, Artificial Sequence
  epitope
KDREG

SEQ ID NO: 771 - protein, 5 aa, Artificial Sequence
  epitope
  misc_feature (2)..(2): Xaa can be any naturally occurring amino acid
KXREG

SEQ ID NO: 772 - protein, 4 aa, Artificial Sequence
  epitope
DREG

SEQ ID NO: 773 - protein, 15 aa, Artificial Sequence
  epitope
LSDIMDYYKDREGSH

SEQ ID NO: 774 - protein, 6 aa, Artificial Sequence
  epitope
KDREGS

SEQ ID NO: 775 - protein, 15 aa, Artificial Sequence
  epitope
LSDIMDYYKDREGSH

SEQ ID NO: 776 - protein, 4 aa, Artificial Sequence
  epitope
DREG

SEQ ID NO: 777 - protein, 6 aa, Artificial Sequence
  epitope
KDREGS

SEQ ID NO: 778 - protein, 5 aa, Artificial Sequence
  epitope
KDREG

SEQ ID NO: 779 - protein, 4 aa, Artificial Sequence
  epitope
DREG

SEQ ID NO: 780 - protein, 6 aa, Artificial Sequence
  epitope
KDREGS

SEQ ID NO: 781 - protein, 4 aa, Artificial Sequence
  epitope
KDRE

SEQ ID NO: 782 - protein, 5 aa, Artificial Sequence
  epitope
KDREG

SEQ ID NO: 783 - protein, 15 aa, Artificial Sequence
  epitope
  misc_feature (1)..(6): Xaa can be any naturally occurring amino acid
  misc_feature (11)..(15): Xaa can be any naturally occurring amino acid
XXXXXXYYKDXXXXX

SEQ ID NO: 784 - protein, 5 aa, Artificial Sequence
  epitope
KDREG

SEQ ID NO: 785 - protein, 15 aa, Artificial Sequence
  epitope
  misc_feature (1)..(8): Xaa can be any naturally occurring amino acid
XXXXXXXXKDREGSH

SEQ ID NO: 786 - protein, 15 aa, Artificial Sequence
  epitope
  misc_feature (1)..(8): Xaa can be any naturally occurring amino acid
  misc_feature (10)..(11): Xaa can be any naturally occurring amino acid
XXXXXXXXKXXEGSH

SEQ ID NO: 787 - protein, 6 aa, Artificial Sequence
  epitope
KDREGS

SEQ ID NO: 788 - protein, 5 aa, Artificial Sequence
  epitope
KDREG

SEQ ID NO: 789 - protein, 4 aa, Artificial Sequence
  epitope
DREG

SEQ ID NO: 790 - protein, 15 aa, Artificial Sequence
  epitope
  misc_feature (1)..(8): Xaa can be any naturally occurring amino acid
  misc_feature (11)..(11): Xaa can be any naturally occurring amino acid
  misc_feature (14)..(15): Xaa can be any naturally occurring amino acid
XXXXXXXXKDXEGXX

SEQ ID NO: 791 - protein, 4 aa, Artificial Sequence
  epitope
DREG

SEQ ID NO: 792 - protein, 15 aa, Artificial Sequence
  epitope
DPATAEDKCDRTCRP

SEQ ID NO: 793 - protein, 15 aa, Artificial Sequence
  epitope
DPATAEDKCDRTCRP

SEQ ID NO: 794 - protein, 15 aa, Artificial Sequence
  epitope
  misc_feature (1)..(2): Xaa can be any naturally occurring amino acid
XXATAEDKCDRTCRP

SEQ ID NO: 795 - protein, 10 aa, Artificial Sequence
  epitope
ATAEDKCDRT

SEQ ID NO: 796 - protein, 10 aa, Artificial Sequence
  epitope
AEDKCDRTCR

SEQ ID NO: 797 - protein, 15 aa, Artificial Sequence
  epitope
DPATAEDKCDRTCRP

SEQ ID NO: 798 - protein, 15 aa, Artificial Sequence
  epitope
  misc_feature (13)..(13): Xaa can be any naturally occurring amino acid
DPATAEDKCDRTXRP

SEQ ID NO: 799 - protein, 15 aa, Artificial Sequence
  epitope
  misc_feature (1)..(2): Xaa can be any naturally occurring amino acid
  misc_feature (12)..(14): Xaa can be any naturally occurring amino acid
XXATAEDKCDRXXXP

SEQ ID NO: 800 - protein, 15 aa, Artificial Sequence
  epitope
  misc_feature (1)..(2): Xaa can be any naturally occurring amino acid
  misc_feature (12)..(15): Xaa can be any naturally occurring amino acid
XXATAEDKCDRXXXX

SEQ ID NO: 801 - protein, 4 aa, Artificial Sequence
  epitope
EDKC

SEQ ID NO: 802 - protein, 15 aa, Artificial Sequence
  epitope
DPATAEDKCDRTCRP

SEQ ID NO: 803 - protein, 15 aa, Artificial Sequence
  epitope
  misc_feature (1)..(2): Xaa can be any naturally occurring amino acid
  misc_feature (5)..(5): Xaa can be any naturally occurring amino acid
  misc_feature (13)..(15): Xaa can be any naturally occurring amino acid
XXATXEDKCDRTXXX

SEQ ID NO: 804 - protein, 15 aa, Artificial Sequence
  epitope
  misc_feature (1)..(6): Xaa can be any naturally occurring amino acid
  misc_feature (9)..(9): Xaa can be any naturally occurring amino acid
  misc_feature (12)..(15): Xaa can be any naturally occurring amino acid
XXXXXXDKXDRXXXX

SEQ ID NO: 805 - protein, 15 aa, Artificial Sequence
  epitope
DPATAEDKCDRTCRP

SEQ ID NO: 806 - protein, 15 aa, Artificial Sequence
  epitope
  misc_feature (5)..(6): Xaa can be any naturally occurring amino acid
DPATXXDKCDRTCRP

SEQ ID NO: 807 - protein, 15 aa, Artificial Sequence
  epitope
  misc_feature (1)..(1): Xaa can be any naturally occurring amino acid
  misc_feature (5)..(6): Xaa can be any naturally occurring amino acid
XPATXXDKCDRTCRP

SEQ ID NO: 808 - protein, 8 aa, Artificial Sequence
  epitope
AEDKCDRT

SEQ ID NO: 809 - protein, 8 aa, Artificial Sequence
  epitope
AEDKCDRT

SEQ ID NO: 810 - protein, 6 aa, Artificial Sequence
  epitope
EDKCDR

SEQ ID NO: 811 - protein, 15 aa, Artificial Sequence
  epitope
DPATAEDKCDRTCRP

SEQ ID NO: 812 - protein, 15 aa, Artificial Sequence
  epitope
  misc_feature (13)..(13): Xaa can be any naturally occurring amino acid
DPATAEDKCDRTXRP

SEQ ID NO: 813 - protein, 15 aa, Artificial Sequence
  epitope
  misc_feature (1)..(1): Xaa can be any naturally occurring amino acid
  misc_feature (13)..(15): Xaa can be any naturally occurring amino acid
XPATAEDKCDRTXXX

SEQ ID NO: 814 - protein, 15 aa, Artificial Sequence
  epitope
  misc_feature (1)..(1): Xaa can be any naturally occurring amino acid
  misc_feature (5)..(6): Xaa can be any naturally occurring amino acid
  misc_feature (12)..(15): Xaa can be any naturally occurring amino acid
XPATXXDKCDRXXXX

SEQ ID NO: 815 - protein, 10 aa, Artificial Sequence
  epitope
RYCTDPATAE

SEQ ID NO: 816 - protein, 10 aa, Artificial Sequence
  epitope
  misc_feature (5)..(5): Xaa can be any naturally occurring amino acid
RYCTXPATAE

SEQ ID NO: 817 - protein, 10 aa, Artificial Sequence
  epitope
  misc_feature (5)..(5): Xaa can be any naturally occurring amino acid
  misc_feature (9)..(10): Xaa can be any naturally occurring amino acid
RYCTXPATXX

SEQ ID NO: 818 - protein, 10 aa, Artificial Sequence
  epitope
AEDKCDRTCR

SEQ ID NO: 819 - protein, 10 aa, Artificial Sequence
  epitope
  misc_feature (1)..(2): Xaa can be any naturally occurring amino acid
  misc_feature (8)..(10): Xaa can be any naturally occurring amino acid
XXDKCDRXXX

SEQ ID NO: 820 - protein, 15 aa, Artificial Sequence
  epitope
DPATAEDKCDRTCRP

SEQ ID NO: 821 - protein, 15 aa, Artificial Sequence
  epitope
  misc_feature (1)..(3): Xaa can be any naturally occurring amino acid
  misc_feature (5)..(5): Xaa can be any naturally occurring amino acid
  misc_feature (9)..(9): Xaa can be any naturally occurring amino acid
  misc_feature (13)..(15): Xaa can be any naturally occurring amino acid
XXXTXEDKXDRTXXX

SEQ ID NO: 822 - protein, 15 aa, Artificial Sequence
  epitope
  misc_feature (1)..(6): Xaa can be any naturally occurring amino acid
  misc_feature (9)..(9): Xaa can be any naturally occurring amino acid
  misc_feature (12)..(15): Xaa can be any naturally occurring amino acid
XXXXXXDKXDRXXXX

SEQ ID NO: 823 - protein, 5 aa, Artificial Sequence
  epitope
EDKCD

SEQ ID NO: 824 - protein, 15 aa, Artificial Sequence
  epitope
DPATAEDKCDRTCRP

SEQ ID NO: 825 - protein, 9 aa, Artificial Sequence
  epitope
YCTDPATAE

SEQ ID NO: 826 - protein, 10 aa, Artificial Sequence
  epitope
TDPATAEDKC

SEQ ID NO: 827 - protein, 9 aa, Artificial Sequence
  epitope
ATAEDKCDR

SEQ ID NO: 828 - protein, 15 aa, Artificial Sequence
  epitope
DPATAEDKCDRTCRP

SEQ ID NO: 829 - protein, 15 aa, Artificial Sequence
  epitope
  misc_feature (1)..(6): Xaa can be any naturally occurring amino acid
  misc_feature (9)..(9): Xaa can be any naturally occurring amino acid
  misc_feature (11)..(11): Xaa can be any naturally occurring amino acid
  misc_feature (14)..(15): Xaa can be any naturally occurring amino acid
XXXXXXDKXDXTCXX

SEQ ID NO: 830 - protein, 5 aa, Artificial Sequence
  epitope
EDKCD

SEQ ID NO: 831 - protein, 15 aa, Artificial Sequence
  epitope
  misc_feature (1)..(3): Xaa can be any naturally occurring amino acid
  misc_feature (13)..(15): Xaa can be any naturally occurring amino acid
XXXTAEDKCDRTXXX

SEQ ID NO: 832 - protein, 15 aa, Artificial Sequence
  epitope
  misc_feature (1)..(6): Xaa can be any naturally occurring amino acid
  misc_feature (12)..(15): Xaa can be any naturally occurring amino acid
XXXXXXDKCDRXXXX

SEQ ID NO: 833 - protein, 5 aa, Artificial Sequence
  epitope
AEDKC

SEQ ID NO: 834 - protein, 15 aa, Artificial Sequence
  epitope
  misc_feature (2)..(3): Xaa can be any naturally occurring amino acid
  misc_feature (6)..(6): Xaa can be any naturally occurring amino acid
  misc_feature (12)..(15): Xaa can be any naturally occurring amino acid
DXXTAXDKCDRXXXX

SEQ ID NO: 835 - protein, 15 aa, Artificial Sequence
  epitope
  misc_feature (1)..(3): Xaa can be any naturally occurring amino acid
  misc_feature (5)..(6): Xaa can be any naturally occurring amino acid
  misc_feature (9)..(10): Xaa can be any naturally occurring amino acid
  misc_feature (12)..(15): Xaa can be any naturally occurring amino acid
XXXTXXDKXXRXXXX

SEQ ID NO: 836 - protein, 10 aa, Artificial Sequence
  epitope
DPATAEDKCD

SEQ ID NO: 837 - protein, 10 aa, Artificial Sequence
  epitope
  misc_feature (2)..(3): Xaa can be any naturally occurring amino acid
  misc_feature (6)..(6): Xaa can be any naturally occurring amino acid
DXXTAXDKCD

SEQ ID NO: 838 - protein, 9 aa, Artificial Sequence
  epitope
AEDKCDRTC

SEQ ID NO: 839 - protein, 8 aa, Artificial Sequence
  epitope
AEDKCDRT

SEQ ID NO: 840 - protein, 8 aa, Artificial Sequence
  epitope
  misc_feature (2)..(2): Xaa can be any naturally occurring amino acid
  misc_feature (8)..(8): Xaa can be any naturally occurring amino acid
AXDKCDRX

SEQ ID NO: 841 - protein, 15 aa, Artificial Sequence
  epitope
  misc_feature (1)..(6): Xaa can be any naturally occurring amino acid
  misc_feature (13)..(13): Xaa can be any naturally occurring amino acid
XXXXXXDKCDRTXRP

SEQ ID NO: 842 - protein, 15 aa, Artificial Sequence
  epitope
  misc_feature (1)..(6): Xaa can be any naturally occurring amino acid
  misc_feature (13)..(15): Xaa can be any naturally occurring amino acid
XXXXXXDKCDRTXXX

SEQ ID NO: 843 - protein, 9 aa, Artificial Sequence
  epitope
ATAEDKCDR

SEQ ID NO: 844 - protein, 9 aa, Artificial Sequence
  epitope
EDKCDRTCR

SEQ ID NO: 845 - protein, 6 aa, Artificial Sequence
  epitope
EDKCDR

SEQ ID NO: 846 - protein, 6 aa, Artificial Sequence
  epitope
  misc_feature (1)..(1): Xaa can be any naturally occurring amino acid
XDKCDR

SEQ ID NO: 847 - protein, 15 aa, Artificial Sequence
  epitope
  misc_feature (1)..(1): Xaa can be any naturally occurring amino acid
  misc_feature (9)..(15): Xaa can be any naturally occurring amino acid
XPATAEDKXXXXXXX

SEQ ID NO: 848 - protein, 15 aa, Artificial Sequence
  epitope
  misc_feature (1)..(1): Xaa can be any naturally occurring amino acid
  misc_feature (4)..(6): Xaa can be any naturally occurring amino acid
  misc_feature (9)..(15): Xaa can be any naturally occurring amino acid
XPAXXXDKXXXXXXX

SEQ ID NO: 849 - protein, 15 aa, Artificial Sequence
  epitope
  misc_feature (1)..(3): Xaa can be any naturally occurring amino acid
  misc_feature (6)..(8): Xaa can be any naturally occurring amino acid
  misc_feature (12)..(15): Xaa can be any naturally occurring amino acid
XXXTAXXXCDRXXXX

SEQ ID NO: 850 - protein, 15 aa, Artificial Sequence
  epitope
  misc_feature (1)..(3): Xaa can be any naturally occurring amino acid
  misc_feature (6)..(9): Xaa can be any naturally occurring amino acid
  misc_feature (12)..(15): Xaa can be any naturally occurring amino acid
XXXTAXXXXDRXXXX

SEQ ID NO: 851 - protein, 9 aa, Artificial Sequence
  epitope
AEDKCDRTC

SEQ ID NO: 852 - protein, 6 aa, Artificial Sequence
  epitope
AEDKCD

SEQ ID NO: 853 - protein, 15 aa, Artificial Sequence
  epitope
  misc_feature (1)..(4): Xaa can be any naturally occurring amino acid
  misc_feature (6)..(6): Xaa can be any naturally occurring amino acid
  misc_feature (12)..(15): Xaa can be any naturally occurring amino acid
XXXXAXDKCDRXXXX

SEQ ID NO: 854 - protein, 15 aa, Artificial Sequence
  epitope
  misc_feature (1)..(6): Xaa can be any naturally occurring amino acid
  misc_feature (12)..(15): Xaa can be any naturally occurring amino acid
XXXXXXDKCDRXXXX

SEQ ID NO: 855 - protein, 15 aa, Artificial Sequence
  epitope
  misc_feature (1)..(6): Xaa can be any naturally occurring amino acid
  misc_feature (9)..(9): Xaa can be any naturally occurring amino acid
  misc_feature (12)..(15): Xaa can be any naturally occurring amino acid
XXXXXXDKXDRXXXX

SEQ ID NO: 856 - protein, 7 aa, Artificial Sequence
  epitope
AEDKCDR

SEQ ID NO: 857 - protein, 6 aa, Artificial Sequence
  epitope
AEDKCD

SEQ ID NO: 858 - protein, 6 aa, Artificial Sequence
  epitope
  misc_feature (2)..(2): Xaa can be any naturally occurring amino acid
AXDKCD

SEQ ID NO: 859 - protein, 6 aa, Artificial Sequence
  epitope
  misc_feature (1)..(2): Xaa can be any naturally occurring amino acid
XXDKCD

SEQ ID NO: 860 - protein, 15 aa, Artificial Sequence
  epitope
  misc_feature (2)..(3): Xaa can be any naturally occurring amino acid
DXXTAEDKCDRTCRP

SEQ ID NO: 861 - protein, 15 aa, Artificial Sequence
  epitope
  misc_feature (1)..(3): Xaa can be any naturally occurring amino acid
  misc_feature (5)..(5): Xaa can be any naturally occurring amino acid
  misc_feature (13)..(13): Xaa can be any naturally occurring amino acid
XXXTXEDKCDRTXRP

SEQ ID NO: 862 - protein, 15 aa, Artificial Sequence
  epitope
  misc_feature (1)..(3): Xaa can be any naturally occurring amino acid
  misc_feature (5)..(6): Xaa can be any naturally occurring amino acid
  misc_feature (13)..(13): Xaa can be any naturally occurring amino acid
XXXTXXDKCDRTXRP

SEQ ID NO: 863 - protein, 10 aa, Artificial Sequence
  epitope
PATAEDKCDR

SEQ ID NO: 864 - protein, 9 aa, Artificial Sequence
  epitope
AEDKCDRTC

SEQ ID NO: 865 - protein, 7 aa, Artificial Sequence
  epitope
AEDKCDR

SEQ ID NO: 866 - protein, 6 aa, Artificial Sequence
  epitope
AEDKCD

SEQ ID NO: 867 - protein, 6 aa, Artificial Sequence
  epitope
  misc_feature (1)..(1): Xaa can be any naturally occurring amino acid
XEDKCD

SEQ ID NO: 868 - protein, 6 aa, Artificial Sequence
  epitope
  misc_feature (1)..(2): Xaa can be any naturally occurring amino acid
XXDKCD

SEQ ID NO: 869 - protein, 15 aa, Artificial Sequence
  epitope
TEDIQGCDSDKHGWY

SEQ ID NO: 870 - protein, 15 aa, Artificial Sequence
  epitope
TEDIQGCDSDKHGWY

SEQ ID NO: 871 - protein, 15 aa, Artificial Sequence
  epitope
  misc_feature (2)..(2): Xaa can be any naturally occurring amino acid
  misc_feature (6)..(7): Xaa can be any naturally occurring amino acid
  misc_feature (9)..(9): Xaa can be any naturally occurring amino acid
  misc_feature (13)..(14): Xaa can be any naturally occurring amino acid
TXDIQXXDXDKHXXY

SEQ ID NO: 872 - protein, 15 aa, Artificial Sequence
  epitope
  misc_feature (2)..(2): Xaa can be any naturally occurring amino acid
  misc_feature (6)..(7): Xaa can be any naturally occurring amino acid
  misc_feature (9)..(9): Xaa can be any naturally occurring amino acid
  misc_feature (12)..(14): Xaa can be any naturally occurring amino acid
TXDIQXXDXDKXXXY

SEQ ID NO: 873 - protein, 10 aa, Artificial Sequence
  epitope
IQGCDSDKHG

SEQ ID NO: 874 - protein, 10 aa, Artificial Sequence
  epitope
  misc_feature (3)..(4): Xaa can be any naturally occurring amino acid
  misc_feature (6)..(6): Xaa can be any naturally occurring amino acid
  misc_feature (10)..(10): Xaa can be any naturally occurring amino acid
IQXXDXDKHX

SEQ ID NO: 875 - protein, 10 aa, Artificial Sequence
  epitope
  misc_feature (3)..(4): Xaa can be any naturally occurring amino acid
  misc_feature (6)..(6): Xaa can be any naturally occurring amino acid
  misc_feature (9)..(10): Xaa can be any naturally occurring amino acid
IQXXDXDKXX

SEQ ID NO: 876 - protein, 9 aa, Artificial Sequence
  epitope
DSDKHGWYR

SEQ ID NO: 877 - protein, 9 aa, Artificial Sequence
  epitope
  misc_feature (2)..(2): Xaa can be any naturally occurring amino acid
  misc_feature (6)..(7): Xaa can be any naturally occurring amino acid
DXDKHXXYR

SEQ ID NO: 878 - protein, 9 aa, Artificial Sequence
  epitope
  misc_feature (2)..(2): Xaa can be any naturally occurring amino acid
  misc_feature (5)..(7): Xaa can be any naturally occurring amino acid
DXDKXXXYR

SEQ ID NO: 879 - protein, 10 aa, Artificial Sequence
  epitope
DKHGWYRFVG

SEQ ID NO: 880 - protein, 10 aa, Artificial Sequence
  epitope
  misc_feature (4)..(5): Xaa can be any naturally occurring amino acid
DKHXXYRFVG

SEQ ID NO: 881 - protein, 10 aa, Artificial Sequence
  epitope
  misc_feature (3)..(5): Xaa can be any naturally occurring amino acid
DKXXXYRFVG

SEQ ID NO: 882 - protein, 15 aa, Artificial Sequence
  epitope
TEDIQGCDSDKHGWY

SEQ ID NO: 883 - protein, 15 aa, Artificial Sequence
  epitope
  misc_feature (2)..(2): Xaa can be any naturally occurring amino acid
  misc_feature (6)..(15): Xaa can be any naturally occurring amino acid
TXDIQXXXXXXXXXX

SEQ ID NO: 884 - protein, 8 aa, Artificial Sequence
  epitope
ENTEDIQG

SEQ ID NO: 885 - protein, 8 aa, Artificial Sequence
  epitope
  misc_feature (4)..(4): Xaa can be any naturally occurring amino acid
  misc_feature (8)..(8): Xaa can be any naturally occurring amino acid
ENTXDIQX

SEQ ID NO: 886 - protein, 10 aa, Artificial Sequence
  epitope
IQGCDSDKHG

SEQ ID NO: 887 - protein, 6 aa, Artificial Sequence
  epitope
DKHGWY

SEQ ID NO: 888 - protein, 15 aa, Artificial Sequence
  epitope
TEDIQGCDSDKHGWY

SEQ ID NO: 889 - protein, 10 aa, Artificial Sequence
  epitope
ENTEDIQGCD

SEQ ID NO: 890 - protein, 15 aa, Artificial Sequence
  epitope
TEDIQGCDSDKHGWY

SEQ ID NO: 891 - protein, 4 aa, Artificial Sequence
  epitope
DSDK

SEQ ID NO: 892 - protein, 10 aa, Artificial Sequence
  epitope
DKHGWYRFVG

SEQ ID NO: 893 - protein, 15 aa, Artificial Sequence
  epitope
TEDIQGCDSDKHGWY

SEQ ID NO: 894 - protein, 7 aa, Artificial Sequence
  epitope
NTEDIQG

SEQ ID NO: 895 - protein, 15 aa, Artificial Sequence
  epitope
TEDIQGCDSDKHGWY

SEQ ID NO: 896 - protein, 10 aa, Artificial Sequence
  epitope
STENTEDIQG

SEQ ID NO: 897 - protein, 10 aa, Artificial Sequence
  epitope
EDIQGCDSDK

SEQ ID NO: 898 - protein, 10 aa, Artificial Sequence
  epitope
IQGCDSDKHG

SEQ ID NO: 899 - protein, 8 aa, Artificial Sequence
  epitope
IQGCDSDK

SEQ ID NO: 900 - protein, 7 aa, Artificial Sequence
  epitope
DKHGWYR

SEQ ID NO: 901 - protein, 6 aa, Artificial Sequence
  epitope
DKHGWY

SEQ ID NO: 902 - protein, 15 aa, Artificial Sequence
  epitope
TEDIQGCDSDKHGWY

SEQ ID NO: 903 - protein, 10 aa, Artificial Sequence
  epitope
IQGCDSDKHG

SEQ ID NO: 904 - protein, 6 aa, Artificial Sequence
  epitope
DKHGWY

SEQ ID NO: 905 - protein, 15 aa, Artificial Sequence
  epitope
TEDIQGCDSDKHGWY

SEQ ID NO: 906 - protein, 15 aa, Artificial Sequence
  epitope
TEDIQGCDSDKHGWY

SEQ ID NO: 907 - protein, 15 aa, Artificial Sequence
  epitope
  misc_feature (1)..(2): Xaa can be any naturally occurring amino acid
  misc_feature (4)..(4): Xaa can be any naturally occurring amino acid
  misc_feature (6)..(10): Xaa can be any naturally occurring amino acid
  misc_feature (12)..(12): Xaa can be any naturally occurring amino acid
XXDXQXXXXXKXGWY

SEQ ID NO: 908 - protein, 15 aa, Artificial Sequence
  epitope
  misc_feature (1)..(4): Xaa can be any naturally occurring amino acid
  misc_feature (6)..(12): Xaa can be any naturally occurring amino acid
XXXXQXXXXXXXGWY

SEQ ID NO: 909 - protein, 6 aa, Artificial Sequence
  epitope
DKHGWY

SEQ ID NO: 910 - protein, 6 aa, Artificial Sequence
  epitope
  misc_feature (1)..(1): Xaa can be any naturally occurring amino acid
  misc_feature (3)..(3): Xaa can be any naturally occurring amino acid
XKXGWY

SEQ ID NO: 911 - protein, 15 aa, Artificial Sequence
  epitope
  misc_feature (1)..(2): Xaa can be any naturally occurring amino acid
  misc_feature (6)..(10): Xaa can be any naturally occurring amino acid
  misc_feature (12)..(15): Xaa can be any naturally occurring amino acid
XXDIQXXXXXKXXXX

SEQ ID NO: 912 - protein, 10 aa, Artificial Sequence
  epitope
IQGCDSDKHG

SEQ ID NO: 913 - protein, 9 aa, Artificial Sequence
  epitope
IQGCDSDKH

SEQ ID NO: 914 - protein, 6 aa, Artificial Sequence
  epitope
DKHGWY

SEQ ID NO: 915 - protein, 6 aa, Artificial Sequence
  epitope
DKHGWY

SEQ ID NO: 916 - protein, 4 aa, Artificial Sequence
  epitope
DKHG

SEQ ID NO: 917 - protein, 10 aa, Artificial Sequence
  epitope
EDIQGCDSDK

SEQ ID NO: 918 - protein, 10 aa, Artificial Sequence
  epitope
IQGCDSDKHG

SEQ ID NO: 919 - protein, 8 aa, Artificial Sequence
  epitope
IQGCDSDK

SEQ ID NO: 920 - protein, 6 aa, Artificial Sequence
  epitope
DKHGWY

SEQ ID NO: 921 - protein, 10 aa, Artificial Sequence
  epitope
IQGCDSDKHG

SEQ ID NO: 922 - protein, 7 aa, Artificial Sequence
  epitope
SDKHGWY

SEQ ID NO: 923 - protein, 4 aa, Artificial Sequence
  epitope
SDKH

SEQ ID NO: 924 - protein, 15 aa, Artificial Sequence
  epitope
  misc_feature (1)..(2): Xaa can be any naturally occurring amino acid
  misc_feature (6)..(6): Xaa can be any naturally occurring amino acid
  misc_feature (9)..(9): Xaa can be any naturally occurring amino acid
XXDIQXCDXDKHGWY

SEQ ID NO: 925 - protein, 15 aa, Artificial Sequence
  epitope
  misc_feature (1)..(2): Xaa can be any naturally occurring amino acid
  misc_feature (4)..(4): Xaa can be any naturally occurring amino acid
  misc_feature (6)..(9): Xaa can be any naturally occurring amino acid
  misc_feature (12)..(15): Xaa can be any naturally occurring amino acid
XXDXQXXXXDKXXXX

SEQ ID NO: 926 - protein, 10 aa, Artificial Sequence
  epitope
IQGCDSDKHG

SEQ ID NO: 927 - protein, 6 aa, Artificial Sequence
  epitope
DKHGWY

SEQ ID NO: 928 - protein, 15 aa, Artificial Sequence
  epitope
SESKIFPGNMDNNSH

SEQ ID NO: 929 - protein, 15 aa, Artificial Sequence
  epitope
SESKIFPGNMDNNSH

SEQ ID NO: 930 - protein, 5 aa, Artificial Sequence
  epitope
ESKIF

SEQ ID NO: 931 - protein, 10 aa, Artificial Sequence
  epitope
NMDNNSHKKN

SEQ ID NO: 932 - protein, 8 aa, Artificial Sequence
  epitope
MDNNSHKK

SEQ ID NO: 933 - protein, 15 aa, Artificial Sequence
  epitope
SESKIFPGNMDNNSH

SEQ ID NO: 934 - protein, 9 aa, Artificial Sequence
  epitope
ESKIFPGNM

SEQ ID NO: 935 - protein, 8 aa, Artificial Sequence
  epitope
ESKIFPGN

SEQ ID NO: 936 - protein, 9 aa, Artificial Sequence
  epitope
NMDNNSHKK

SEQ ID NO: 937 - protein, 8 aa, Artificial Sequence
  epitope
MDNNSHKK

SEQ ID NO: 938 - protein, 5 aa, Artificial Sequence
  epitope
MDNNS

SEQ ID NO: 939 - protein, 15 aa, Artificial Sequence
  epitope
SESKIFPGNMDNNSH

SEQ ID NO: 940 - protein, 4 aa, Artificial Sequence
  epitope
SKIF

SEQ ID NO: 941 - protein, 10 aa, Artificial Sequence
  epitope
NMDNNSHKKN

SEQ ID NO: 942 - protein, 15 aa, Artificial Sequence
  epitope
SESKIFPGNMDNNSH

SEQ ID NO: 943 - protein, 15 aa, Artificial Sequence
  epitope
  misc_feature (5)..(12): Xaa can be any naturally occurring amino acid
  misc_feature (14)..(15): Xaa can be any naturally occurring amino acid
SESKXXXXXXXXNXX

SEQ ID NO: 944 - protein, 6 aa, Artificial Sequence
  epitope
SESKIF

SEQ ID NO: 945 - protein, 10 aa, Artificial Sequence
  epitope
SKIFPGNMDN

SEQ ID NO: 946 - protein, 7 aa, Artificial Sequence
  epitope
MDNNSHK

SEQ ID NO: 947 - protein, 6 aa, Artificial Sequence
  epitope
MDNNSH

SEQ ID NO: 948 - protein, 5 aa, Artificial Sequence
  epitope
MDNNS

SEQ ID NO: 949 - protein, 15 aa, Artificial Sequence
  epitope
SESKIFPGNMDNNSH

SEQ ID NO: 950 - protein, 15 aa, Artificial Sequence
  epitope
  misc_feature (1)..(5): Xaa can be any naturally occurring amino acid
XXXXXFPGNMDNNSH

SEQ ID NO: 951 - protein, 9 aa, Artificial Sequence
  epitope
ESKIFPGNM

SEQ ID NO: 952 - protein, 9 aa, Artificial Sequence
  epitope
  misc_feature (1)..(4): Xaa can be any naturally occurring amino acid
XXXXFPGNM

SEQ ID NO: 953 - protein, 10 aa, Artificial Sequence
  epitope
FPGNMDNNSH

SEQ ID NO: 954 - protein, 9 aa, Artificial Sequence
  epitope
NMDNNSHKK

SEQ ID NO: 955 - protein, 6 aa, Artificial Sequence
  epitope
MDNNSH

SEQ ID NO: 956 - protein, 15 aa, Artificial Sequence
  epitope
SESKIFPGNMDNNSH

SEQ ID NO: 957 - protein, 5 aa, Artificial Sequence
  epitope
ESKIF

SEQ ID NO: 958 - protein, 4 aa, Artificial Sequence
  epitope
SKIF

SEQ ID NO: 959 - protein, 15 aa, Artificial Sequence
  epitope
SESKIFPGNMDNNSH

SEQ ID NO: 960 - protein, 15 aa, Artificial Sequence
  epitope
SESKIFPGNMDNNSH

SEQ ID NO: 961 - protein, 7 aa, Artificial Sequence
  epitope
ASESKIF

SEQ ID NO: 962 - protein, 5 aa, Artificial Sequence
  epitope
MDNNS

SEQ ID NO: 963 - protein, 15 aa, Artificial Sequence
  epitope
SESKIFPGNMDNNSH

SEQ ID NO: 964 - protein, 5 aa, Artificial Sequence
  epitope
ESKIF

SEQ ID NO: 965 - protein, 15 aa, Artificial Sequence
  epitope
  misc_feature (8)..(15): Xaa can be any naturally occurring amino acid
SESKIFPXXXXXXXX

SEQ ID NO: 966 - protein, 5 aa, Artificial Sequence
  epitope
ESKIF

SEQ ID NO: 967 - protein, 8 aa, Artificial Sequence
  epitope
ESKIFPGN

SEQ ID NO: 968 - protein, 5 aa, Artificial Sequence
  epitope
ESKIF

SEQ ID NO: 969 - protein, 15 aa, Artificial Sequence
  epitope
  misc_feature (1)..(1): Xaa can be any naturally occurring amino acid
  misc_feature (3)..(3): Xaa can be any naturally occurring amino acid
  misc_feature (5)..(13): Xaa can be any naturally occurring amino acid
XEXKXXXXXXXXXSH

SEQ ID NO: 970 - protein, 7 aa, Artificial Sequence
  epitope
ESKIFPG

SEQ ID NO: 971 - protein, 5 aa, Artificial Sequence
  epitope
ESKIF

SEQ ID NO: 972 - protein, 6 aa, Artificial Sequence
  epitope
MDNNSH

SEQ ID NO: 973 - protein, 5 aa, Artificial Sequence
  epitope
MDNNS

SEQ ID NO: 974 - protein, 15 aa, Artificial Sequence
  epitope
  misc_feature (1)..(5): Xaa can be any naturally occurring amino acid
  misc_feature (7)..(7): Xaa can be any naturally occurring amino acid
XXXXXFXGNMDNNSH

SEQ ID NO: 975 - protein, 5 aa, Artificial Sequence
  epitope
ESKIF

SEQ ID NO: 976 - protein, 6 aa, Artificial Sequence
  epitope
MDNNSH

SEQ ID NO: 977 - protein, 15 aa, Artificial Sequence
  epitope
  misc_feature (1)..(8): Xaa can be any naturally occurring amino acid
XXXXXXXXNMDNNSH

SEQ ID NO: 978 - protein, 15 aa, Artificial Sequence
  epitope
  misc_feature (1)..(8): Xaa can be any naturally occurring amino acid
  misc_feature (12)..(12): Xaa can be any naturally occurring amino acid
XXXXXXXXNMDXNSH

SEQ ID NO: 979 - protein, 10 aa, Artificial Sequence
  epitope
SESKIFPGNM

SEQ ID NO: 980 - protein, 10 aa, Artificial Sequence
  epitope
SKIFPGNMDN

SEQ ID NO: 981 - protein, 10 aa, Artificial Sequence
  epitope
  misc_feature (1)..(6): Xaa can be any naturally occurring amino acid
XXXXXXNMDN

SEQ ID NO: 982 - protein, 5 aa, Artificial Sequence
  epitope
MDNNS

SEQ ID NO: 983 - protein, 5 aa, Artificial Sequence
  epitope
  misc_feature (3)..(3): Xaa can be any naturally occurring amino acid
MDXNS

SEQ ID NO: 984 - protein, 9 aa, Artificial Sequence
  epitope
ESKIFPGNM

SEQ ID NO: 985 - protein, 8 aa, Artificial Sequence
  epitope
SKIFPGNM

SEQ ID NO: 986 - protein, 4 aa, Artificial Sequence
  epitope
SKIF

SEQ ID NO: 987 - protein, 5 aa, Artificial Sequence
  epitope
MDNNS

SEQ ID NO: 988 - protein, 7 aa, Artificial Sequence
  epitope
ESKIFPG

SEQ ID NO: 989 - protein, 5 aa, Artificial Sequence
  epitope
ESKIF

SEQ ID NO: 990 - protein, 15 aa, Artificial Sequence
  epitope
  misc_feature (8)..(13): Xaa can be any naturally occurring amino acid
  misc_feature (15)..(15): Xaa can be any naturally occurring amino acid
SESKIFPXXXXXXSX

SEQ ID NO: 991 - protein, 15 aa, Artificial Sequence
  epitope
  misc_feature (2)..(3): Xaa can be any naturally occurring amino acid
  misc_feature (7)..(15): Xaa can be any naturally occurring amino acid
SXXKIFXXXXXXXXX

SEQ ID NO: 992 - protein, 10 aa, Artificial Sequence
  epitope
SESKIFPGNM

SEQ ID NO: 993 - protein, 10 aa, Artificial Sequence
  epitope
  misc_feature (8)..(10): Xaa can be any naturally occurring amino acid
SESKIFPXXX

SEQ ID NO: 994 - protein, 10 aa, Artificial Sequence
  epitope
  misc_feature (2)..(3): Xaa can be any naturally occurring amino acid
  misc_feature (7)..(10): Xaa can be any naturally occurring amino acid
SXXKIFXXXX

SEQ ID NO: 995 - protein, 10 aa, Artificial Sequence
  epitope
SKIFPGNMDN

SEQ ID NO: 996 - protein, 10 aa, Artificial Sequence
  epitope
  misc_feature (6)..(10): Xaa can be any naturally occurring amino acid
SKIFPXXXXX

SEQ ID NO: 997 - protein, 15 aa, Artificial Sequence
  epitope
QPKSPPSVTLFPPST

SEQ ID NO: 998 - protein, 15 aa, Artificial Sequence
  epitope
QPKSPPSVTLFPPST

SEQ ID NO: 999 - protein, 15 aa, Artificial Sequence
  epitope
  misc_feature (1)..(2): Xaa can be any naturally occurring amino acid
  misc_feature (5)..(6): Xaa can be any naturally occurring amino acid
  misc_feature (8)..(8): Xaa can be any naturally occurring amino acid
  misc_feature (12)..(12): Xaa can be any naturally occurring amino acid
XXKSXXSXTLFXPST

SEQ ID NO: 1000 - protein, 15 aa, Artificial Sequence
  epitope
  misc_feature (1)..(2): Xaa can be any naturally occurring amino acid
  misc_feature (4)..(6): Xaa can be any naturally occurring amino acid
  misc_feature (8)..(8): Xaa can be any naturally occurring amino acid
  misc_feature (12)..(12): Xaa can be any naturally occurring amino acid
  misc_feature (15)..(15): Xaa can be any naturally occurring amino acid
XXKXXXSXTLFXPSX

SEQ ID NO: 1001 - protein, 15 aa, Artificial Sequence
  epitope
  misc_feature (1)..(2): Xaa can be any naturally occurring amino acid
  misc_feature (4)..(9): Xaa can be any naturally occurring amino acid
  misc_feature (12)..(12): Xaa can be any naturally occurring amino acid
  misc_feature (15)..(15): Xaa can be any naturally occurring amino acid
XXKXXXXXXLFXPSX

SEQ ID NO: 1002 - protein, 6 aa, Artificial Sequence
  epitope
KSPPSV

SEQ ID NO: 1003 - protein, 5 aa, Artificial Sequence
  epitope
SPPSV

SEQ ID NO: 1004 - protein, 10 aa, Artificial Sequence
  epitope
SVTLFPPSTE

SEQ ID NO: 1005 - protein, 10 aa, Artificial Sequence
  epitope
  misc_feature (2)..(2): Xaa can be any naturally occurring amino acid
  misc_feature (6)..(6): Xaa can be any naturally occurring amino acid
SXTLFXPSTE

SEQ ID NO: 1006 - protein, 10 aa, Artificial Sequence
  epitope
  misc_feature (2)..(2): Xaa can be any naturally occurring amino acid
  misc_feature (6)..(6): Xaa can be any naturally occurring amino acid
  misc_feature (9)..(9): Xaa can be any naturally occurring amino acid
SXTLFXPSXE

SEQ ID NO: 1007 - protein, 10 aa, Artificial Sequence
  epitope
  misc_feature (1)..(3): Xaa can be any naturally occurring amino acid
  misc_feature (6)..(6): Xaa can be any naturally occurring amino acid
  misc_feature (9)..(9): Xaa can be any naturally occurring amino acid
XXXLFXPSXE

SEQ ID NO: 1008 - protein, 15 aa, Artificial Sequence
  epitope
QPKSPPSVTLFPPST

SEQ ID NO: 1009 - protein, 10 aa, Artificial Sequence
  epitope
VLGQPKSPPS

SEQ ID NO: 1010 - protein, 10 aa, Artificial Sequence
  epitope
GQPKSPPSVT

SEQ ID NO: 1011 - protein, 9 aa, Artificial Sequence
  epitope
KSPPSVTLF

SEQ ID NO: 1012 - protein, 4 aa, Artificial Sequence
  epitope
SPPS

SEQ ID NO: 1013 - protein, 7 aa, Artificial Sequence
  epitope
LFPPSTE

SEQ ID NO: 1014 - protein, 15 aa, Artificial Sequence
  epitope
QPKSPPSVTLFPPST

SEQ ID NO: 1015 - protein, 15 aa, Artificial Sequence
  epitope
  misc_feature (2)..(2): Xaa can be any naturally occurring amino acid
  misc_feature (5)..(5): Xaa can be any naturally occurring amino acid
  misc_feature (8)..(15): Xaa can be any naturally occurring amino acid
QXKSXPSXXXXXXXX

SEQ ID NO: 1016 - protein, 5 aa, Artificial Sequence
  epitope
KSPPS

SEQ ID NO: 1017 - protein, 5 aa, Artificial Sequence
  epitope
  misc_feature (3)..(3): Xaa can be any naturally occurring amino acid
KSXPS

SEQ ID NO: 1018 - protein, 15 aa, Artificial Sequence
  epitope
QPKSPPSVTLFPPST

SEQ ID NO: 1019 - protein, 15 aa, Artificial Sequence
  epitope
  misc_feature (1)..(2): Xaa can be any naturally occurring amino acid
  misc_feature (5)..(6): Xaa can be any naturally occurring amino acid
  misc_feature (9)..(9): Xaa can be any naturally occurring amino acid
  misc_feature (12)..(15): Xaa can be any naturally occurring amino acid
XXKSXXSVXLFXXXX

SEQ ID NO: 1020 - protein, 15 aa, Artificial Sequence
  epitope
  misc_feature (1)..(2): Xaa can be any naturally occurring amino acid
  misc_feature (5)..(7): Xaa can be any naturally occurring amino acid
  misc_feature (9)..(9): Xaa can be any naturally occurring amino acid
  misc_feature (12)..(15): Xaa can be any naturally occurring amino acid
XXKSXXXVXLFXXXX

SEQ ID NO: 1021 - protein, 10 aa, Artificial Sequence
  epitope
TVLGQPKSPP

SEQ ID NO: 1022 - protein, 5 aa, Artificial Sequence
  epitope
SPPSV

SEQ ID NO: 1023 - protein, 10 aa, Artificial Sequence
  epitope
SVTLFPPSTE

SEQ ID NO: 1024 - protein, 10 aa, Artificial Sequence
  epitope
LFPPSTEELN

SEQ ID NO: 1025 - protein, 15 aa, Artificial Sequence
  epitope
QPKSPPSVTLFPPST

SEQ ID NO: 1026 - protein, 9 aa, Artificial Sequence
  epitope
VLGQPKSPP

SEQ ID NO: 1027 - protein, 8 aa, Artificial Sequence
  epitope
KSPPSVTL

SEQ ID NO: 1028 - protein, 7 aa, Artificial Sequence
  epitope
KSPPSVT

SEQ ID NO: 1029 - protein, 4 aa, Artificial Sequence
  epitope
KSPP

SEQ ID NO: 1030 - protein, 10 aa, Artificial Sequence
  epitope
LFPPSTEELN

SEQ ID NO: 1031 - protein, 8 aa, Artificial Sequence
  epitope
LFPPSTEE

SEQ ID NO: 1032 - protein, 15 aa, Artificial Sequence
  epitope
  misc_feature (5)..(10): Xaa can be any naturally occurring amino acid
QPKSXXXXXXFPPST

SEQ ID NO: 1033 - protein, 15 aa, Artificial Sequence
  epitope
  misc_feature (1)..(2): Xaa can be any naturally occurring amino acid
  misc_feature (5)..(12): Xaa can be any naturally occurring amino acid
XXKSXXXXXXXXPST

SEQ ID NO: 1034 - protein, 4 aa, Artificial Sequence
  epitope
SPPS

SEQ ID NO: 1035 - protein, 15 aa, Artificial Sequence
  epitope
  misc_feature (1)..(2): Xaa can be any naturally occurring amino acid
  misc_feature (5)..(6): Xaa can be any naturally occurring amino acid
  misc_feature (8)..(10): Xaa can be any naturally occurring amino acid
  misc_feature (12)..(15): Xaa can be any naturally occurring amino acid
XXKSXXSXXXFXXXX

SEQ ID NO: 1036 - protein, 4 aa, Artificial Sequence
  epitope
SPPS

SEQ ID NO: 1037 - protein, 9 aa, Artificial Sequence
  epitope
SVTLFPPST

SEQ ID NO: 1038 - protein, 15 aa, Artificial Sequence
  epitope
GIPVPFPIPESDGCK

SEQ ID NO: 1039 - protein, 15 aa, Artificial Sequence
  epitope
GIPVPFPIPESDGCK

SEQ ID NO: 1040 - protein, 15 aa, Artificial Sequence
  epitope
  misc_feature (1)..(2): Xaa can be any naturally occurring amino acid
  misc_feature (9)..(9): Xaa can be any naturally occurring amino acid
  misc_feature (14)..(14): Xaa can be any naturally occurring amino acid
XXPVPFPIXESDGXK

SEQ ID NO: 1041 - protein, 15 aa, Artificial Sequence
  epitope
  misc_feature (1)..(3): Xaa can be any naturally occurring amino acid
  misc_feature (8)..(9): Xaa can be any naturally occurring amino acid
  misc_feature (13)..(15): Xaa can be any naturally occurring amino acid
XXXVPFPXXESDXXX

SEQ ID NO: 1042 - protein, 6 aa, Artificial Sequence
  epitope
ESDGCK

SEQ ID NO: 1043 - protein, 6 aa, Artificial Sequence
  epitope
  misc_feature (5)..(5): Xaa can be any naturally occurring amino acid
ESDGXK

SEQ ID NO: 1044 - protein, 15 aa, Artificial Sequence
  epitope
GIPVPFPIPESDGCK

SEQ ID NO: 1045 - protein, 6 aa, Artificial Sequence
  epitope
ESDGCK

SEQ ID NO: 1046 - protein, 15 aa, Artificial Sequence
  epitope
GIPVPFPIPESDGCK

SEQ ID NO: 1047 - protein, 9 aa, Artificial Sequence
  epitope
IVMGIPVPF

SEQ ID NO: 1048 - protein, 5 aa, Artificial Sequence
  epitope
IPVPF

SEQ ID NO: 1049 - protein, 6 aa, Artificial Sequence
  epitope
ESDGCK

SEQ ID NO: 1050 - protein, 4 aa, Artificial Sequence
  epitope
ESDG

SEQ ID NO: 1051 - protein, 15 aa, Artificial Sequence
  epitope
GIPVPFPIPESDGCK

SEQ ID NO: 1052 - protein, 6 aa, Artificial Sequence
  epitope
ESDGCK

SEQ ID NO: 1053 - protein, 15 aa, Artificial Sequence
  epitope
GIPVPFPIPESDGCK

SEQ ID NO: 1054 - protein, 15 aa, Artificial Sequence
  epitope
  misc_feature (1)..(9): Xaa can be any naturally occurring amino acid
  misc_feature (14)..(14): Xaa can be any naturally occurring amino acid
XXXXXXXXXESDGXK

SEQ ID NO: 1055 - protein, 6 aa, Artificial Sequence
  epitope
ESDGCK

SEQ ID NO: 1056 - protein, 6 aa, Artificial Sequence
  epitope
  misc_feature (5)..(5): Xaa can be any naturally occurring amino acid
ESDGXK

SEQ ID NO: 1057 - protein, 15 aa, Artificial Sequence
  epitope
  misc_feature (2)..(5): Xaa can be any naturally occurring amino acid
  misc_feature (12)..(14): Xaa can be any naturally occurring amino acid
GXXXXFPIPESXXXK

SEQ ID NO: 1058 - protein, 15 aa, Artificial Sequence
  epitope
  misc_feature (1)..(5): Xaa can be any naturally occurring amino acid
  misc_feature (7)..(8): Xaa can be any naturally occurring amino acid
  misc_feature (12)..(14): Xaa can be any naturally occurring amino acid
XXXXXFXXPESXXXK

SEQ ID NO: 1059 - protein, 10 aa, Artificial Sequence
  epitope
IPVPFPIPES

SEQ ID NO: 1060 - protein, 10 aa, Artificial Sequence
  epitope
  misc_feature (1)..(4): Xaa can be any naturally occurring amino acid
XXXXFPIPES

SEQ ID NO: 1061 - protein, 10 aa, Artificial Sequence
  epitope
  misc_feature (1)..(4): Xaa can be any naturally occurring amino acid
  misc_feature (6)..(7): Xaa can be any naturally occurring amino acid
XXXXFXXPES

SEQ ID NO: 1062 - protein, 6 aa, Artificial Sequence
  epitope
ESDGCK

SEQ ID NO: 1063 - protein, 4 aa, Artificial Sequence
  epitope
ESDG

SEQ ID NO: 1064 - protein, 9 aa, Artificial Sequence
  epitope
GIPVPFPIP

SEQ ID NO: 1065 - protein, 8 aa, Artificial Sequence
  epitope
IPVPFPIP

SEQ ID NO: 1066 - protein, 7 aa, Artificial Sequence
  epitope
IPVPFPI

SEQ ID NO: 1067 - protein, 10 aa, Artificial Sequence
  epitope
FPIPESDGCK

SEQ ID NO: 1068 - protein, 15 aa, Artificial Sequence
  epitope
  misc_feature (1)..(10): Xaa can be any naturally occurring amino acid
XXXXXXXXXXSDGCK

SEQ ID NO: 1069 - protein, 15 aa, Artificial Sequence
  epitope
  misc_feature (1)..(10): Xaa can be any naturally occurring amino acid
  misc_feature (14)..(14): Xaa can be any naturally occurring amino acid
XXXXXXXXXXSDGXK

SEQ ID NO: 1070 - protein, 6 aa, Artificial Sequence
  epitope
ESDGCK

SEQ ID NO: 1071 - protein, 6 aa, Artificial Sequence
  epitope
  misc_feature (1)..(1): Xaa can be any naturally occurring amino acid
XSDGCK

SEQ ID NO: 1072 - protein, 6 aa, Artificial Sequence
  epitope
  misc_feature (1)..(1): Xaa can be any naturally occurring amino acid
  misc_feature (5)..(5): Xaa can be any naturally occurring amino acid
XSDGXK

SEQ ID NO: 1073 - protein, 15 aa, Artificial Sequence
  epitope
  misc_feature (1)..(9): Xaa can be any naturally occurring amino acid
  misc_feature (13)..(13): Xaa can be any naturally occurring amino acid
XXXXXXXXXESDXCK

SEQ ID NO: 1074 - protein, 6 aa, Artificial Sequence
  epitope
ESDGCK

SEQ ID NO: 1075 - protein, 6 aa, Artificial Sequence
  epitope
  misc_feature (4)..(4): Xaa can be any naturally occurring amino acid
ESDXCK

SEQ ID NO: 1076 - protein, 15 aa, Artificial Sequence
  epitope
LTKDSEKLKEEIRKE

SEQ ID NO: 1077 - protein, 15 aa, Artificial Sequence
  epitope
LTKDSEKLKEEIRKE

SEQ ID NO: 1078 - protein, 15 aa, Artificial Sequence
  epitope
  misc_feature (1)..(6): Xaa can be any naturally occurring amino acid
  misc_feature (12)..(12): Xaa can be any naturally occurring amino acid
  misc_feature (14)..(14): Xaa can be any naturally occurring amino acid
XXXXXXKLKEEXRXE

SEQ ID NO: 1079 - protein, 4 aa, Artificial Sequence
  epitope
KEEI

SEQ ID NO: 1080 - protein, 15 aa, Artificial Sequence
  epitope
LTKDSEKLKEEIRKE

SEQ ID NO: 1081 - protein, 4 aa, Artificial Sequence
  epitope
KDSE

SEQ ID NO: 1082 - protein, 9 aa, Artificial Sequence
  epitope
KLKEEIRKE

SEQ ID NO: 1083 - protein, 10 aa, Artificial Sequence
  epitope
KEEIRKELED

SEQ ID NO: 1084 - protein, 15 aa, Artificial Sequence
  epitope
LTKDSEKLKEEIRKE

SEQ ID NO: 1085 - protein, 15 aa, Artificial Sequence
  epitope
  misc_feature (1)..(3): Xaa can be any naturally occurring amino acid
  misc_feature (5)..(5): Xaa can be any naturally occurring amino acid
  misc_feature (7)..(8): Xaa can be any naturally occurring amino acid
  misc_feature (10)..(12): Xaa can be any naturally occurring amino acid
  misc_feature (14)..(14): Xaa can be any naturally occurring amino acid
XXXDXEXXKXXXRXE

SEQ ID NO: 1086 - protein, 15 aa, Artificial Sequence
  epitope
LTKDSEKLKEEIRKE

SEQ ID NO: 1087 - protein, 15 aa, Artificial Sequence
  epitope
  misc_feature (1)..(5): Xaa can be any naturally occurring amino acid
  misc_feature (10)..(12): Xaa can be any naturally occurring amino acid
XXXXXEKLKXXXRKE

SEQ ID NO: 1088 - protein, 8 aa, Artificial Sequence
  epitope
ERLTKDSE

SEQ ID NO: 1089 - protein, 10 aa, Artificial Sequence
  epitope
LTKDSEKLKE

SEQ ID NO: 1090 - protein, 10 aa, Artificial Sequence
  epitope
  misc_feature (1)..(5): Xaa can be any naturally occurring amino acid
  misc_feature (10)..(10): Xaa can be any naturally occurring amino acid
XXXXXEKLKX

SEQ ID NO: 1091 - protein, 7 aa, Artificial Sequence
  epitope
LKEEIRK

SEQ ID NO: 1092 - protein, 7 aa, Artificial Sequence
  epitope
  misc_feature (3)..(5): Xaa can be any naturally occurring amino acid
LKXXXRK

SEQ ID NO: 1093 - protein, 15 aa, Artificial Sequence
  epitope
LTKDSEKLKEEIRKE

SEQ ID NO: 1094 - protein, 10 aa, Artificial Sequence
  epitope
RLTKDSEKLK

SEQ ID NO: 1095 - protein, 6 aa, Artificial Sequence
  epitope
KLKEEI

SEQ ID NO: 1096 - protein, 15 aa, Artificial Sequence
  epitope
  misc_feature (1)..(2): Xaa can be any naturally occurring amino acid
  misc_feature (12)..(15): Xaa can be any naturally occurring amino acid
XXKDSEKLKEEXXXX

SEQ ID NO: 1097 - protein, 15 aa, Artificial Sequence
  epitope
  misc_feature (1)..(2): Xaa can be any naturally occurring amino acid
  misc_feature (5)..(7): Xaa can be any naturally occurring amino acid
  misc_feature (12)..(15): Xaa can be any naturally occurring amino acid
XXKDXXXLKEEXXXX

SEQ ID NO: 1098 - protein, 4 aa, Artificial Sequence
  epitope
KDSE

SEQ ID NO: 1099 - protein, 9 aa, Artificial Sequence
  epitope
KLKEEIRKE

SEQ ID NO: 1100 - protein, 9 aa, Artificial Sequence
  epitope
  misc_feature (6)..(9): Xaa can be any naturally occurring amino acid
KLKEEXXXX

SEQ ID NO: 1101 - protein, 9 aa, Artificial Sequence
  epitope
  misc_feature (1)..(1): Xaa can be any naturally occurring amino acid
  misc_feature (6)..(9): Xaa can be any naturally occurring amino acid
XLKEEXXXX

SEQ ID NO: 1102 - protein, 6 aa, Artificial Sequence
  epitope
LTKDSE

SEQ ID NO: 1103 - protein, 10 aa, Artificial Sequence
  epitope
KDSEKLKEEI

SEQ ID NO: 1104 - protein, 9 aa, Artificial Sequence
  epitope
KLKEEIRKE

SEQ ID NO: 1105 - protein, 6 aa, Artificial Sequence
  epitope
KLKEEI

SEQ ID NO: 1106 - protein, 15 aa, Artificial Sequence
  epitope
  misc_feature (1)..(1): Xaa can be any naturally occurring amino acid
  misc_feature (3)..(3): Xaa can be any naturally occurring amino acid
  misc_feature (5)..(5): Xaa can be any naturally occurring amino acid
  misc_feature (12)..(12): Xaa can be any naturally occurring amino acid
XTXDXEKLKEEXRKE

SEQ ID NO: 1107 - protein, 15 aa, Artificial Sequence
  epitope
  misc_feature (1)..(3): Xaa can be any naturally occurring amino acid
  misc_feature (5)..(6): Xaa can be any naturally occurring amino acid
  misc_feature (11)..(12): Xaa can be any naturally occurring amino acid
  misc_feature (15)..(15): Xaa can be any naturally occurring amino acid
XXXDXXKLKEXXRKX

SEQ ID NO: 1108 - protein, 8 aa, Artificial Sequence
  epitope
EKLKEEIR

SEQ ID NO: 1109 - protein, 6 aa, Artificial Sequence
  epitope
EKLKEE

SEQ ID NO: 1110 - protein, 5 aa, Artificial Sequence
  epitope
KLKEE

SEQ ID NO: 1111 - protein, 5 aa, Artificial Sequence
  epitope
  misc_feature (5)..(5): Xaa can be any naturally occurring amino acid
KLKEX

SEQ ID NO: 1112 - protein, 15 aa, Artificial Sequence
  epitope
  misc_feature (3)..(4): Xaa can be any naturally occurring amino acid
  misc_feature (6)..(8): Xaa can be any naturally occurring amino acid
  misc_feature (13)..(14): Xaa can be any naturally occurring amino acid
LTXXSXXXKEEIXXE

SEQ ID NO: 1113 - protein, 5 aa, Artificial Sequence
  epitope
KEEIR

SEQ ID NO: 1114 - protein, 4 aa, Artificial Sequence
  epitope
KEEI

SEQ ID NO: 1115 - protein, 10 aa, Artificial Sequence
  epitope
YLSQTEENKE

SEQ ID NO: 1116 - protein, 6 aa, Artificial Sequence
  epitope
HPEDRK

SEQ ID NO: 1117 - protein, 5 aa, Artificial Sequence
  epitope
KPEDR

SEQ ID NO: 1118 - protein, 5 aa, Artificial Sequence
  epitope
VPEDR

SEQ ID NO: 1119 - protein, 7 aa, Artificial Sequence
  epitope
KRRGESC

SEQ ID NO: 1120 - protein, 7 aa, Artificial Sequence
  epitope
KADGESC

SEQ ID NO: 1121 - protein, 15 aa, Artificial Sequence
  epitope
EWQNRLEEIKKTMFE

SEQ ID NO: 1122 - protein, 15 aa, Artificial Sequence
  epitope
EKTFECGECGKTFWE

SEQ ID NO: 1123 - protein, 7 aa, Artificial Sequence
  epitope
TGGKEEK

SEQ ID NO: 1124 - protein, 10 aa, Artificial Sequence
  epitope
KEESVPLVGQ

SEQ ID NO: 1125 - protein, 7 aa, Artificial Sequence
  epitope
DKYELPP

SEQ ID NO: 1126 - protein, 9 aa, Artificial Sequence
  epitope
EDKYRKINE

SEQ ID NO: 1127 - protein, 9 aa, Artificial Sequence
  epitope
EDKYGTTTS

SEQ ID NO: 1128 - protein, 5 aa, Artificial Sequence
  epitope
KINSK

SEQ ID NO: 1129 - protein, 7 aa, Artificial Sequence
  epitope
WEKEMKL

SEQ ID NO: 1130 - protein, 10 aa, Artificial Sequence
  epitope
LNDKCDRLRK

SEQ ID NO: 1131 - protein, 5 aa, Artificial Sequence
  epitope
KLKER

(19) United States
(12) Patent Application Publication
(10) Pub. No.: US 2022/0323575 A1
(43) Pub. Date: Oct. 13, 2022

(54) NOVEL MILK ALLERGY ANTIGEN

(71) Applicants: FUJITA ACADEMY, Aichi (JP); HOYU CO., LTD., Aichi (JP)

(72) Inventors: Kayoko Matsunaga, Aichi (JP); Akiko Yagami, Aichi (JP); Yasuto Kondo, Aichi (JP); Yuji Aoki, Aichi (JP); Erika Hasegawa, Aichi (JP); Tomomi Sakai, Aichi (JP)

(21) Appl. No.: 17/708,653

(22) Filed: Mar. 30, 2022

(30) Foreign Application Priority Data
Mar. 31, 2021 (JP) 2021-061611

(51) Int. Cl. A61K 39/35; A61K 49/00; A61K 35/20

(52) U.S. Cl. CPC A61K 39/35; A61K 49/0004; A61K 35/20

(57) ABSTRACT

The present invention provides a novel milk allergy antigen, a milk allergy diagnosis method or diagnosis kit, a composition comprising the antigen, a milk or processed milk product with the antigen removed, and a tester composition for determining the presence or absence of the milk antigen in an object of interest. The present invention also pertains to an antigen epitope-comprising polypeptide, a kit for diagnosing an allergy comprising the polypeptide, diagnostic composition, a diagnosis method, a composition comprising the polypeptide, and a raw material or processed product in which the polypeptide-comprising antigen is eliminated or reduced. The present invention further pertains to a tester composition for determining the presence or absence of an antigen in an object of interest.

### CROSS-REFERENCE TO RELATED APPLICATIONS

This application claims foreign priority under 35 U.S.C. § 119(a) to Japanese Patent Application No. 2021-061611, filed on Mar. 31, 2021, the entire contents of which are incorporated herein by reference.

### REFERENCE TO SEQUENCE LISTING

The instant application contains a Sequence Listing which has been filed electronically in ASCII format and is hereby incorporated by reference in its entirety. Said ASCII copy, created on Mar. 29, 2022 and having a size of 381,791 bytes, is named 129249_01202_Seqlisting.txt.

### TECHNICAL FIELD

The present invention relates to a novel milk allergy antigen. The present invention also relates to a kit, a composition and a method for diagnosing milk allergy. The present invention further relates to a composition comprising the antigen and a raw material or processed product in which the antigen is eliminated or reduced. The present invention furthermore relates to a tester composition for determining the presence or absence of a milk antigen in an object of interest.

In addition, the present invention relates to an epitope-comprising polypeptide antigen. In addition, the present invention relates to a kit for diagnosing an allergy, a diagnostic composition, and a diagnosis method comprising the polypeptide. In addition, the present invention relates to a method for providing an indicator for diagnosing an allergy in a subject. In addition, the present invention relates to a composition comprising the polypeptide and a raw material or processed product in which the polypeptide is eliminated or reduced. Further, the present invention relates to a method for producing a raw material or processed product in which the polypeptide is eliminated or reduced. Furthermore, the present invention relates to a tester composition for determining the presence or absence of the polypeptide-comprising antigen in an object of interest.

### BACKGROUND OF THE INVENTION

An IgE antibody specific to a particular antigen (hereinafter, also referred to as an allergen) is produced in serum or tissues of a patient with allergy. This Ig E antibody interacts with the particular antigen and causes physiological consequences, which trigger an allergic reaction. Antigens refer to a food/foodstuff, etc., which causes an allergy symptom in a broad sense, and refer to proteins (hereinafter, also referred to as an allergen component) included in foods/foodstuffs, etc., to which specific IgE antibodies bind in a narrow sense.

In conventional allergy test agents, antigen reagents have often been prepared by simply grinding candidate food/foodstuff, etc. (PTL 1). Consequently, it is possible to detect the positive reaction in the allergy test only when, in a conventional antigen reagent comprising many types of allergen components, the content of an allergen component exceeds a threshold that allows determination of a positive reaction for binding to an IgE antibody. As a result, the diagnosis efficiency cannot be said to be sufficiently high.

In the allergen candidate foods/foodstuffs, some allergen components are pointed out and have been commercialized as test kits. To enhance the reliability of allergy test, the allergen components have to be identified exhaustively: however, the rate of detecting a patient by measuring the above allergen components is still insufficient. It is critical to identify a novel milk allergen(s) not only increasing the accuracy of a diagnostic agent but also providing a target for a low-allergen food, a low-allergen foodstuff, or a therapeutic agent.

Meanwhile, in the field of protein separation and purification, a method for separating and purifying various proteins from a small amount of a sample has recently been used, wherein a 2D electrophoresis consisting of isoelectric focusing as a first dimension, and SDS-PAGE (sodium dodecyl sulfate-polyacrylamide gel electrophoresis) as a second dimension is performed. The present applicants have hitherto developed a 2D electrophoresis with increased separation capacity (PTLs 2 to 5).

Allergen-specific IgE antibodies recognize and bind to epitopes which are particular amino acid sequences in allergen components. Although the allergen components have been analyzed at an epitope level in several cases (NPL 1), the number is so far very low. In addition, no allergy diagnosis kits using an epitope-comprising polypeptide are currently available in the market.
PTL1: Japanese Patent Laid-Open No. 2002-286716
PTL2: Japanese Patent Laid-Open No. 2011-33544
PTL3: Japanese Patent Laid-Open No. 2011-33546
PTL4: Japanese Patent Laid-Open No. 2011-33547
PTL5: Japanese Patent Laid-Open No. 2011-33548
NPL1: Matsuo, H., et al., J. Biol. Chem., (2004), Vol. 279, No. 13, pp. 12135-12140

### SUMMARY OF THE INVENTION

The invention provides a novel allergy protein antigen. In addition, the invention provides an allergy diagnosis method and diagnosis kit comprising the antigen. In addition, the invention provides a composition comprising the antigen and a raw material or processed product in which the antigen is eliminated or reduced. Further, the invention provides a tester composition for determining the presence or absence of the antigen in an object of interest.

In addition, the present invention provides an epitope-comprising polypeptide antigen. In addition, the present invention provides a kit, a composition and a method for diagnosing allergy comprising the polypeptide. In addition, the invention provides a method for providing an indicator for diagnosing an allergy in a subject. In addition, the invention provides a composition comprising the polypeptide and a raw material or processed product in which the polypeptide-comprising antigen is eliminated or reduced. Further, the invention relates to a method for producing a raw material or processed product in which the antigen is eliminated or reduced. Furthermore, the invention provides a tester composition for determining the presence or absence of the polypeptide-comprising antigen in an object of interest.

The present inventors have conducted intensive studies to solve the above problems and have identified an antigen(s) that causes a milk allergy. As a result, the inventors succeeded in identification of a novel protein antigen(s) to which an IgE antibody in the serum of a patient with milk allergy specifically binds.

Meanwhile, epitopes have relatively short amino acid sequences. Thus, if the same amino acid sequence is present in different allergen components, the IgE antibody can bind to a plurality of allergen components. The different allergen components share an epitope, so that the IgE antibody from the allergic patient can bind to the epitope in such components. Thus, the antigen has cross-reactivity. Accordingly, an epitope identified herein enables, for example, diagnosis and treatment of an allergy with cross-reactivity and detection of a plurality of allergen components comprising the epitope.

The term “antigen” used herein means and encompasses both a narrowly defined protein antigen and a protein-derived “epitope” unless otherwise indicated. When explicitly indicated, the “antigen” is used to mean either a protein or a protein-derived epitope.

Based on the above findings, the invention has been completed. Although not limited, the invention includes the following items.

[Item 1] A kit for diagnosing an allergy comprising at least one of the following polypeptides (E1) to (E25):

(E1) a polypeptide comprising at least one amino acid sequence selected from amino acid sequences set forth in SEQ ID NOs: 27 to 58 and 1115;

(E2) a polypeptide comprising at least one amino acid sequence selected from amino acid sequences set forth in SEQ ID NOs: 59 to 97;

(E3) a polypeptide comprising at least one amino acid sequence selected from amino acid sequences set forth in SEQ ID NOs: 98 to 144;

(E4) a polypeptide comprising at least one amino acid sequence selected from amino acid sequences set forth in SEQ ID NOs: 145 to 195;

(E5) a polypeptide comprising at least one amino acid sequence selected from amino acid sequences set forth in SEQ ID NOs: 196 to 254 and 1116 to 1118;

(E6) a polypeptide comprising at least one amino acid sequence selected from amino acid sequences set forth in SEQ ID NOs: 255 to 288;

(E7) a polypeptide comprising at least one amino acid sequence selected from amino acid sequences set forth in SEQ ID NOs: 289 to 333 and 1119 to 1120;

(E8) a polypeptide comprising at least one amino acid sequence selected from amino acid sequences set forth in SEQ ID NOs: 334 to 352;

(E9) a polypeptide comprising at least one amino acid sequence selected from amino acid sequences set forth in SEQ ID NOs: 353 to 376 and 1121 to 1122;

(E10) a polypeptide comprising at least one amino acid sequence selected from amino acid sequences set forth in SEQ ID NOs: 377 to 384;

(E11) a polypeptide comprising at least one amino acid sequence selected from amino acid sequences set forth in SEQ ID NOs:385 to 411;

(E12) a polypeptide comprising at least one amino acid sequence selected from amino acid sequences set forth in SEQ ID NOs: 412 to 450;

(E13) a polypeptide comprising at least one amino acid sequence selected from amino acid sequences set forth in SEQ ID NOs: 451 to 500 and 1123 to 1124;

(E14) a polypeptide comprising at least one amino acid sequence selected from amino acid sequences set forth in SEQ ID NOs: 501 to 604 and 1125 to 1127;

(E15) a polypeptide comprising at least one amino acid sequence selected from amino acid sequences set forth in SEQ ID NOs: 605 to 632 and 1128;

(E16) a polypeptide comprising at least one amino acid sequence selected from amino acid sequences set forth in SEQ ID NOs: 633 to 673;

(E17) a polypeptide comprising at least one amino acid sequence selected from amino acid sequences set forth in SEQ ID NOs: 674 to 693;

(E18) a polypeptide comprising at least one amino acid sequence selected from amino acid sequences set forth in SEQ ID NOs: 694 to 757 and 1129;

(E19) a polypeptide comprising at least one amino acid sequence selected from amino acid sequences set forth in SEQ ID NOs: 758 to 791;

(E20) a polypeptide comprising at least one amino acid sequence selected from amino acid sequences set forth in SEQ ID NOs: 792 to 868 and 1130;

(E21) a polypeptide comprising at least one amino acid sequence selected from amino acid sequences set forth in SEQ ID NOs: 869 to 927;

(E22) a polypeptide comprising at least one amino acid sequence selected from amino acid sequences set forth in SEQ ID NOs: 928 to 996;

(E23) a polypeptide comprising at least one amino acid sequence selected from amino acid sequences set forth in SEQ ID NOs: 997 to 1037;

(E24) a polypeptide comprising at least one amino acid sequence selected from amino acid sequences set forth in SEQ ID NOs: 1038 to 1075; or (E25) a polypeptide comprising at least one amino acid sequence selected from amino acid sequences set forth in SEQ ID NOs:1076 to 1114 and 1131.

[Item 2] A composition for diagnosing an allergy comprising at least one polypeptide as defined in any of (E1) to (E25) in item 1.
[Item 3] A method for providing an indicator for diagnosing an allergy in a subject, comprising the steps of:

(i) contacting a sample obtained from the subject with an antigen, wherein the sample is a solution comprising an IgE antibody;

(ii) detecting binding between the IgE antibody present in the sample obtained from the subject and the antigen; and

(iii) when the binding between the IgE antibody in the subject and the antigen is detected, an indicator of the fact that the subject is allergic is provided,

wherein the antigen is at least one polypeptide as defined in any of (E1) to (E25) in item 1.

[Item 4] An antigen, which is at least one polypeptide as defined in any of (E1) to (E25) in item 1 and is causative of an allergy.
[Item 5] A composition comprising at least one antigen according to item 4.
[Item 6] The composition according to item 5, wherein the composition is intended for the treatment of an allergy.
[Item 7] A tester composition for determining the presence or absence of an antigen in an object of interest, comprising an antibody that binds to at least one polypeptide as defined in any of (E1) to (E25) in item 1.
[Item 8] A tester composition for determining the presence or absence of an antigen in an object of interest, comprising at least one primer comprising a portion of a nucleotide sequence, and/or a portion of a complementary strand thereof, of a nucleic acid encoding a polypeptide as defined in any of (E1) to (E25) in item 1.
[Item 9] A tester composition for determining the presence or absence of an IgE antibody in a subject, comprising a polypeptide as defined in any of (E1) to (E25) in item 1.
[Item 10] A method for determining the presence or absence of a polypeptide as defined in any of (E1) to (E25) in item 1 in a raw material or processed product, comprising detecting the polypeptide as defined in any of (E1) to (E25) in item 1 in the raw material or processed product.
[Item 11] A raw material or processed product in which an antigen is eliminated or reduced, wherein the antigen is at least one polypeptide as defined in any of (E1) to (E25) in item 1.
[Item 12] A method for producing a raw material or processed product in which an antigen is eliminated or reduced, the method comprising the step of confirming that the antigen has been eliminated or reduced in a production process of the raw material or the processed product, wherein the antigen is at least one polypeptide as defined in any of (E1) to (E25) in item 1.

The invention can provide a novel allergy (e.g., milk allergy) antigen. Since a novel allergen component which is causative of an allergy has been identified in the present invention, it has become possible to provide a highly sensitive allergy diagnosis method and diagnosis kit, a composition comprising the antigen, a raw material or processed product in which the antigen is eliminated or reduced, and a tester composition for determining the presence or absence of the antigen in an object of interest.

In addition, the invention can provide a novel polypeptide antigen comprising an epitope of a protein antigen. Use of a polypeptide in the invention makes it possible to provide a highly sensitive allergy (e.g., milk allergy) diagnosis kit, diagnostic composition, and diagnosis method, a composition comprising the polypeptide, a tester composition for determining the presence or absence of the polypeptide-comprising antigen in an object of interest, and a raw material or processed product in which the polypeptide is eliminated or reduced and a method for producing the raw material or processed product.

### BRIEF DESCRIPTIONS OF THE DRAWINGS

FIG. 1 is a photograph of a gel showing a protein electrophoresis pattern obtained by 2D-electrophoresis of proteins contained in milk. The left-side bands in the photograph are bands of molecular weight markers, and the numerical values on the left side in the photograph indicate respective molecular weights (KDa) of molecular weight markers. The numerals at the top of the photograph represent isoelectric points.

FIG. 2 is a photograph of an immunoblot of a 2D electrophoresis of proteins contained in milk, obtained with serum from a patient with milk allergy. Spots 1 to 14 where IgE antibodies in serum of the patient with milk allergy specifically reacted in comparison with a healthy individual are each enclosed with a white line.

FIG. 3 shows the results of examining cross-reactivity to peptides having the amino acid sequence of each epitope by ELISA using serum from a patient (P1) with milk allergy to.

FIG. 4 shows the results of checking cross-reactivity to peptides having the amino acid sequence of each epitope by ELISA using serum from a patient (P5) with milk allergy.

FIG. 5 shows the results of checking cross-reactivity to peptides having the amino acid sequence of each epitope by ELISA using serum from a patient (P8) with milk allergy.

FIG. 6 shows the results of examining cross-reactivity to peptides having the amino acid sequence of each epitope by ELISA using serum from a patient (P20) with milk allergy.

### DETAILED DESCRIPTIONS OF THE INVENTION

Hereinbelow, the invention will be described specifically. However, the invention is not limited to them.

Unless otherwise defined herein, scientific terms and technical terms used with respect to the invention have meanings commonly understood by those skilled in the art.

As used herein, the allergy refers to a state in which an in vivo unwanted hypersensitivity reaction occurs when a certain antigen re-enters into the body of an individual which has been sensitized with the certain antigen. Upon contact with an antigen or intake of the antigen, an allergic reaction may occur. Here, the contact refers to touch to an object, and refers to, in the case of a human body, in particular, attachment to the skin, mucosa (e.g., an eye, a lip), etc. In addition, the intake refers to incorporation into the body, and refers to incorporation by inhalation or through an oral route, etc. Generally, the allergic reaction caused by food ingestion refers to, in particular, a food allergy. In a preferred embodiment, the allergy may be food allergy. In many food allergy diseases, an IgE antibody specific to an antigen is produced in blood and tissues. The IgE antibody binds to a mast cell or basophil. When an antigen specific to such an IgE antibody re-enters into the body of a patient with allergic disease, the antigen binds to the IgE antibody bound to a mast cell or basophil to exert a physiological effect of the IgE antibody-antigen interaction. Examples of such a physiological effect include release of histamine, serotonin, heparin, eosinophil chemotactic factors, various leukotrienes or the like. These released substances can trigger an allergic reaction caused by the combination of an IgE antibody with a specific antigen. Specifically, the IgE antibody recognizes and binds to an epitope, which is a specific amino acid sequence in the specific antigen. As a result, the antigen-mediated allergic reaction appears via the above pathway.

The allergy of interest in the invention is not particularly limited as long as the allergy is against an allergen (antigen) comprising an epitope to be used. In an embodiment, examples of the allergen include milk, a dairy product, a processed milk product, meat, a processed meat product, grains, seafood, a fruit, a vegetable, nuts (seeds), edible grass, an egg, a processed egg, etc., that are ingested by living organisms or parasites that live in living organisms (especially humans).

The origin of the milk is not particularly limited. In an embodiment, milk derived from a cow, goat, sheep, or the like may be included. In an embodiment, the milk is cow's milk. Examples of the milk include, but are not limited to, raw milk, pasteurized milk, ingredient-adjusted milk, low-fat milk, or fat-free milk. The dairy product is a product produced by processing milk as a main raw material. Examples of the dairy product include cream, butter, butter oil, cheese, whey, whey concentrate, ice cream, milk concentrate, skimmed milk concentrate, unsweetened condensed milk, unsweetened skimmed condensed milk, sweetened condensed milk, sweetened skimmed condensed milk, whole milk powder, skimmed milk powder, cream powder, whey powder, protein concentrate whey powder, buttermilk powder, sweetened milk powder, prepared milk powder, prepared liquid milk, fermented milk such as yogurt, a lactobacillus beverage, or a milk drink. The processed milk product refers to a milk product produced by processing cow's milk as a main raw material or each food comprising the processed milk product as a raw material. Examples of the processed milk product include, but are not limited to, cream, butter, butter oil, cheese, whey, whey concentrate, ice cream, milk concentrate, skimmed milk concentrate, unsweetened condensed milk, unsweetened skimmed condensed milk, sweetened condensed milk, sweetened skimmed condensed milk, whole milk powder, skimmed milk powder, cream powder, whey powder, protein-enriched whey powder, buttermilk powder, sweetened milk powder, prepared milk powder, prepared liquid milk, fermented milk such as yogurt, a lactobacillus beverage, a milk drink, which are produced by processing milk, or a cake, pastry, custard pudding, milk agar, biscuit, cookie, snack, chocolate, bread, white sauce, potage, cream stew, gratin, curry roux, or stew roux, which contain the above material(s) as a raw material.

The type of meat is not particularly limited. In an embodiment, examples include bird meat (e.g., chicken, duck) or mammalian meat (e.g., pork, beef, ram). In an embodiment, the meat is derived from a mammal. In an embodiment, the meat is derived from a cow (Bos taurus). The processed meat product refers to a product produced by processing meat as a main raw material or each food comprising the processed meat product as a raw material. Examples of the processed meat product include, but are not limited to, ham, sausage, bacon, bouillon, consomme, steak, grilled meat, roast beef, tartar steak, hamburg steak, hamburger, meatballs, or nuggets.

The grains generally refer to a foodstuff obtained from a plant and are edible seeds primarily comprising starch. The term “grains”, in a narrow sense, refers only to the seeds of gramineous crops (cereals), but in a broader sense the term also includes the seeds of legumes (beans) and seeds of other crops. Among the grains in a broad sense, the seeds of dicotyledonous plants that are used as grains because they resemble the seeds of grass crops (the cereals of monocotyledons) are collectively called pseudocereals. Examples of the pseudocereals include those from buckwheat (Polygonaceae), amaranth (Amaranthaceae), or quinoa (Chenopodium quinoa, Chenopodiaceae).

In the invention, examples of the gramineous grains include, but are not limited to, bread wheat (Triticum aestivum), barley, rye, oat, timothy, orchardgrass, Anthoxanthum odoratum, millet, foxtail millet, Japanese millet, corn, or adlay. Examples of the grains from the family Polygonaceae include those from Fagopyrum esculentum. In an embodiment, the antigen (allergen) is derived from grains. In an embodiment, the allergen is derived from gramineous grains. In an embodiment, the allergen is derived from wheat.

Examples of the seafood include, but are not limited to, shrimps or crabs belonging to the order Decapoda. Most of the organisms generally recognized as “crustaceans” are included in the order Decapoda. Other examples of the seafood include, but are not limited to, squids or octopuses belonging to the order Teuthoidea and the order Octopoda. Examples further include fish belonging to the family Scombridae or Gadidae. Examples furthermore include shellfish from the family Venus.

Examples of the fruit include, but are not limited to, a fruit belonging to the family Actinidiaceae, Bromeliaceae, Anacardiaceae, Cucurbitaceae, Musaceae, Rutaceae, or Rosaceae. Examples of the vegetable include a fruit belonging to the family Solanaceae, Cucurbitaceae, or Lauraceae. Examples of the nuts (seeds) include nuts (seeds) belonging to the family Anacardiaceae, Rosaceae, or Juglandaceae or nuts (e.g., peanuts) belonging to the family Leguminosae. Examples of the edible grass include edible grass belonging to the family Asteraceae. Examples of the grains include grains belonging to the family Poaceae or Polygonaceae.

In the invention, examples of the egg include, in addition to the above seafood egg, a bird egg. Examples of the bird egg include, but are not limited to, a quail egg or chicken egg. The processed egg product refers to a product produced by processing an egg as a main raw material or each food comprising the processed egg product as a raw material. Examples of the processed egg product include, but are not limited to, a processed fish roe product (e.g., salted cod roe, spicy cod roe, salted salmon roe, salmon roe pickled in soy sauce, a baby sardine, dried mullet roe) or a processed chicken egg product (e.g., mayonnaise, hollandaise sauce, quail egg boiled in water, boiled egg, boiled egg seasoned in soy sauce, omelet, thick omelet, rolled egg, steamed egg, omelet, omelet rice, baked egg, poached egg, quiche, scotch egg, French toast, cake, pastry, custard pudding, biscuit, cookie, snack, bread).

The parasites are not limited, and are, for example, parasites of the family Anisakidae. Examples of the parasites of the family Anisakidae include Anisakis simplex.

As used herein, the allergy refers to a state with an allergic reaction caused by, as an antigen, a protein, etc., contained in a raw material or processed product (e.g., milk or a processed milk product). In the allergy, an allergic reaction can occur in the case of contact with an antigen contained in a raw material or processed product (e.g., milk or a processed milk product) or in the case of ingestion of the antigen. In general, the allergic reaction occurring when a food is ingested refers to, in particular, a food allergy. Milk allergy may be a food allergy.

As used herein, the antigen is a substance that can trigger an allergic reaction. In the case of a protein contained in a raw material for a foodstuff, etc., the antigen is also referred to as an allergen component. The antigen is preferably a protein.

As used herein, the protein is a molecule with a structure in which naturally occurring amino acids are linked via a peptide bond. The number of amino acids contained in the protein is not particularly limited. As used herein, the term “polypeptide” also means a molecule with a structure in which naturally occurring amino acids are linked via a peptide bond. The number of amino acids contained in the polypeptide is not particularly limited. The “polypeptide” is a concept including a “protein”. The polypeptide in which about 2 to 50 amino acids are linked via peptide bonds, in particular, is sometimes called a peptide.

In the case where an amino acid may have an enantiomer, the amino acid is in an L-form unless otherwise indicated. The nomenclature of amino acid sequences of proteins, polypeptides, or peptides used herein is based on standard usage or the nomenclature conventionally used in the art. The amino acid is represented by one-letter code; the leftward direction represents the amino terminal direction, and the rightward direction represents the carboxy terminal direction. Note that in the one-letter amino acid code, X may be any of substances each having an amino group and a carboxyl group and capable of bonding to amino acids at both ends, and may represent any of, in particular, naturally occurring 20 amino acids.

Alanine scanning (or “alanine/glycine scanning”) is a method including: creating variants in which residues in a protein is mutated to alanine (glycine if the original amino acid is alanine) one by one; and site-specifically identifying which residue is important for the structure and the function of the protein. If the binding ability to IgE antibodies from patients remains even after replacement with alanine (glycine if the original amino acid is alanine), the residues are not important for the binding ability to IgE antibodies, and, in this case, the binding ability remains even after replacement of these residues to other amino acids. The binding ability to the IgE antibodies means the detection of the binding and reaction between an IgE antibody and an epitope of interest. In the sequence listing of the present application, a residue denoted by X is an amino acid residue at a site where, even after replacement of the residue with alanine (glycine if the original amino acid is alanine) by the alanine/glycine scanning shown in Example 4, the binding ability to IgE antibodies from patients with allergy remains. It is well-known to those skilled in the art that even when such a site is replaced with any other amino acids, the probability that the binding ability to the IgE antibodies remains is high. Specifically, the residue can be replaced with not only alanine or glycine but also any amino acid residues other than alanine.

The binding of the IgE to an antigen (epitope) and its maintenance are important for a subsequent allergic reaction, and electric charge, hydrophobic bonds, hydrogen bonds, and aromatic interaction of the epitope are responsible for the above binding and maintenance. Even after the loss of them due to alanine/glycine replacement, the binding and its maintenance may remain, which means that the amino acid is dispensable.

Identification of Antigen

Proteins contained in milk were subjected to 2D electrophoresis under the conditions below to identify milk allergy antigens.

For the first dimension, electrophoresis was isoelectric focusing. The gel length was in the range from 5 to 10 cm. The pH range of the gel was from 3 to 10. The pH gradient of the gel in the electrophoresis direction was set while the entire gel length was set to 1, the gel length up to pH 5 was “a”, the gel length between pH 5 and pH 7 was “b”, and the gel length of pH 7 or higher was “c”. In this case, used was a gel with “a” ranging from 0.15 to 0.3, “b” ranging from 0.4 to 0.7, and “c” ranging from 0.15 to 0.3. Specifically, an IPG gel Immobiline Drystrip (pH 3-10 NL), manufactured by GE Healthcare Bioscience, Inc. (hereinafter, abbreviated as GE Company), was used to conduct isoelectric focusing. The electrophoresis apparatus used was an IPGphor, manufactured by GE Company. The upper limit of a current value in the electrophoresis apparatus was set to 75 μA per gel. The voltage program included (1) a constant voltage step of providing a constant voltage of 300 V until 750 Vhr was reached (a change in current for 30 min of electrophoresis before end of the step was 5 μA); (2) gradually increasing the voltage up to 1000 V over 300 Vhr; (3) further gradually increasing the voltage up to 5000 V over 4500 Vhr; and (4) subsequently providing a constant voltage of 5000 V until the total Vhr reached 12000. In this way, 1D isoelectric focusing was performed.

In the second dimension, electrophoresis was a SDS-PAGE, in which the gel concentration at a base end in the electrophoresis direction was set to 3 to 6%, and the gel concentration at a portion on the distal end side in the electrophoresis direction was set to be higher than the gel concentration at a portion on the base end side in the electrophoresis direction. Such a polyacrylamide gel was used. Specifically, a NuPAGE 4-12% Bis-Tris Gels IPG well mini 1 mm, manufactured by Life Technologies, Inc., was used to conduct SDS-PAGE. The electrophoresis apparatus used was a XCell SureLock Mini-Cell, manufactured by Life Technologies, Inc. The running buffer used was 50 mM MOPS, 50 mM Tris Base, 0.1% (w/v) SDS, and 1 mM EDTA. The electrophoresis was performed at a constant voltage of 200 V for about 45 min.

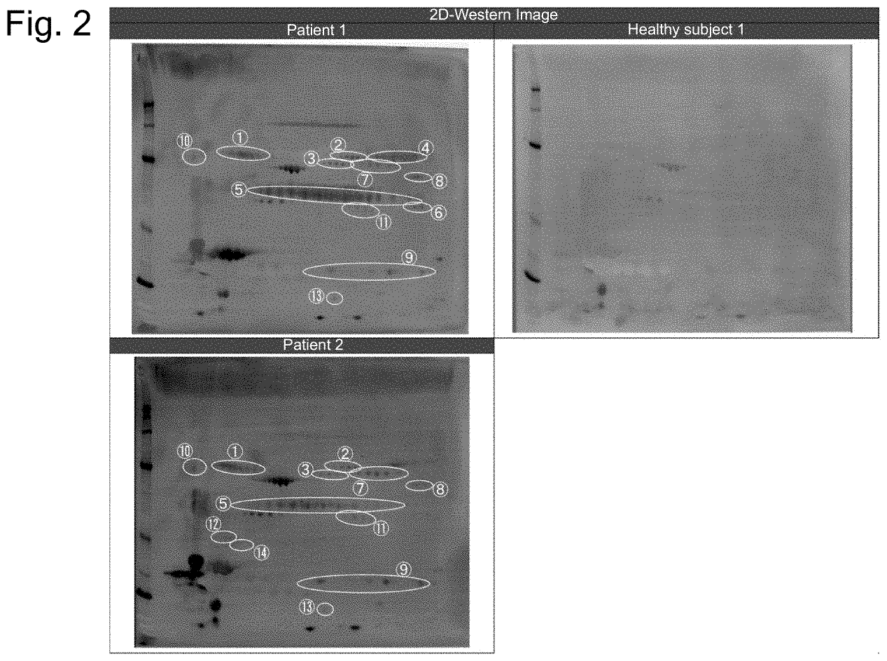

Milk proteins were subjected to 2D electrophoresis under the above conditions. The results have revealed that in the gel, the antigens of the following spots 1 to 14 specifically bind to an IgE antibodies of patients with milk allergy (FIG. 2).

Antigens (Proteins)

The sequences contained in each spot were identified by mass spectrometry. The mass data obtained using a mass spectrometer was analyzed by comparing between the mass data and protein data of Uniprot, NCBI. The results have revealed that each of spots 1 to 14 corresponds to known sequences.

Information about each spot is summarized in Table 1 below.

TABLE 1-1
MW:66kDa

| SPOT No. | | | \textcircled{1} |
|---|---|---|---|
| protein | Isoelectric | Range | 2-8 |
| | point | Preferably | 2.5-7 |
| | | More preferably | 3-6 |
| | Molecular | Range | 40-160 |
| | weight | Preferably | 50-110 |
| | | More preferably | 60-100 |
| | Searched database | | NCBI |
| | Biological species | | Cow (Bos taurus) |
| | accession No. | | AAN60017.1 |
| | Protein name | | immunoglobulin M heavy chain secretory form |
| | Full-length | | SQVQLRESGPSLVKPSQTLSLTCTVSGFSLSDYRVGWVRQAPGKALEWLGVMYNDGTTAYRPALLSRLRITRDNSKNQVALS |
| | sequence | | LSSVTTEDTATYYCTRIIPVCTWPYGDYGCYDILGQGLLVTVSSEGESHPRVFPLVSCVSSPSDESTVALGCLARDFVPNSV |
| | (SEQ ID NO: 2) | | SFSWKFNNSTVSSERFWTFPEVLRDGLWSASSQVVLPSSSAFQGPDDYLVCEVQHPKGGKTVGTVRVVATKAEVLSPVVSVF |
| | | | VPPRNSLSGDGNSKSSLICQATDFSPKQISLSWFRDGKRIVSGISEGQVETVQSSPVTFRAYSMLTITERDWLSQNVYTCQV |
| | | | EHNKETFQKNVSSSCDVAPPSPIGVFTIPPSFADIFLTKSAKLSCLVTNLASYDGLNISWSRQNGKALETHTYFERHLNDIF |
| | | | SARGEASVCSEDWESGEEFTCTVAHSDLPFPEKNTVSKPKDVAMKPPSVYLLPPTREQLSLRESASVTCLVKGFAPADVFVQ |
| | | | WLQRGEPVTKSKYVTSSPAPEPQDPSVYFVHSILTVAEEDWSKGETYTCVVGHEALPHMVTERTVDKSTGKPTLYNVSLVLS |
| | | | DTASTCY |
| DNA | Searched database | | GenBank |
| | accession No. | | AY145128.1 |
| | Full length | | TCCCAGGTGCAGCTGC |
| | sequence | | GGGAGTCGGGCCCCAGCCTGGTGAAGCCCTCACAGACCCTCTCCCTCACCTGCACGGTCTCTGGATTCTC |
| | (SEQ ID NO: 1) | | ATTAAGCGATTATCGTGTAGGCTGGGTCCGCCAGGCTCCAGGAAAGGCGCTGGAGTGGCTCGGTGTAATG |
| | | | TATAATGACGGAACCACTGCGTATAGGCCAGCCCTGCTATCCCGGCTCCGAATCACCAGGGACAACTCCA |
| | | | AGAACCAAGTCGCCCTGTCACTAAGCAGCGTAACAACTGAGGACACGGCCACATACTACTGTACAAGAAT |
| | | | AATTCCTGTGTGTACTTGGCCTTATGGTGACTATGGCTGTTATGACATCTTGGGCCAAGGACTCCTGGTC |
| | | | ACCGTCTCCTCAGAAGGTGAATCACACCCGAGAGTCTTCCCCCTGGTGTCCTGCGTGAGCTCCCCATCCG |
| | | | ATGAGAGCACGGTGGCCCTGGGCTGCCTGGCCCGGGACTTCGTGCCCAATTCAGTCAGCTTCTCCTGGAA |
| | | | GTTCAACAACAGCACAGTCAGCAGCGAGAGATTCTGGACCTTCCCCGAAGTCCTGAGGGACGGCTTGTGG |
| | | | TCGGCCTCCTCTCAGGTGGTCCTGCCCTCCTCAAGCGCCTTTCAAGGGCCGGATGACTACCTGGTGTGCG |
| | | | AAGTCCAGCACCCCAAGGGAGGAAAGACCGTCGGCACCGTGAGGGTGGTCGCTACAAAGGCGGAAGTGCT |
| | | | GTCCCCAGTCGTGAGTGTCTTTGTCCCGCCTCGCAACAGCCTCTCTGGTGACGGCAATAGCAAGTCCAGC |
| | | | CTCATCTGCCAGGCCACGGACTTCAGCCCCAAACAGATCTCCTTGTCCTGGTTTCGTGATGGAAAGCGGA |
| | | | TAGTGTCTGGCATTTCTGAAGGCCAGGTGGAGACTGTGCAGTCCTCACCCGTAACTTTCAGGGCCTACAG |
| | | | CATGCTGACCATCACGGAGAGAGACTGGCTCAGCCAGAACGTGTACACCTGCCAGGTAGAACACAACAAG |
| | | | GAAACCTTCCAGAAGAACGTGTCCTCCTCATGTGATGTTGCACCACCATCTCCCATCGGGGTCTTCACCA |
| | | | TCCCCCCATCCTTCGCCGACATCTTCCTCACGAAGTCAGCCAAGCTGTCCTGTCTGGTCACAAACCTGGC |
| | | | CTCCTATGATGGCCTGAACATCAGCTGGTCCCGTCAGAACGGCAAGGCCCTGGAGACCCACACGTATTTT |
| | | | GAGCGACACCTCAACGACACCTTCAGCGCCCGGGGTGAGGCCTCGGTCTGCTCGGAGGACTGGGAGTCCG |
| | | | GAGAGGAGTTCACGTGCACAGTGGCCCACTCGGACCTGCCCTTCCCAGAAAAGAACACCGTCTCCAAGCC |
| | | | CAAAGATGTCGCCATGAAACCGCCGTCCGTGTACCTGCTGCCTCCAACGCGGGAACAGCTGAGCCTGCGG |
| | | | GAGTCGGCCTCCGTCACCTGCCTGGTGAAGGGCTTCGCGCCCGCGGACGTGTTCGTGCAGTGGCTGCAGA |
| | | | GGGGGGAGCCCGTGACCAAGAGCAAGTACGTGACCAGCAGCCCGGCGCCCGAGCCCCAGGACCCCAGCGT |
| | | | GTACTTTGTGCACAGCATCCTGACGGTGGCCGAGGAGGACTGGAGCAAAGGGGAGACCTACACCTGCGTC |
| | | | GTGGGCCACGAGGCCCTGCCCCACATGGTCACCGAGCGGACCGTGGACAAGTCCACCGGTAAACCCACCC |
| | | | TGTACAACGTGTCCCTGGTCCTGTCTGACACAGCCAGCACCTGCTAC |

TABLE 1-2
MW:78kDa

| SPOT No. | | | \textcircled{2} \textcircled{3} |
|---|---|---|---|
| protein | Isoelectric | Range | 4-11 |
| | point | Preferably | 4.5-10 |
| | | More preferably | 5-9 |
| | Molecular | Range | 40-160 |
| | weight | Preferably | 50-110 |
| | | More preferably | 60-100 |

| | | |
|---|---|---|
| | Searched database | Uniprot |
| | Biological species | Cow (Bos taurus) |
| | accession No. | G3X6N3 |
| | Protein name | Beta-1 metal-binding globulin |
| | Full-length | DPERTVRWCTISTHEANKCASFRENVLRILESGPFVSCVKK |
| | sequence | ISHMDCIKAISNNEADAVILDGGLVYEAGLKPNNLKPVVAEFHGTKDNPQTHYYAVAVVK |
| | (SEQ ID NO: 4) | KDTDFKLNELRGKKSCHTGLGRSAGWNIPMGKLYKELPDPQESIQRAAANFFSASCVPCA |
| | | DQSSFPKLCQLCAGKGIDKCACSNHEPYFGYSGAFKCLMEGAGDVAFVKHSTVEDNLPNP |
| | | EDRKNYELLCGDNTRKSVDNYQECYLAMVPSHAVVARTVGGKEDVIWELLNHAQEHFGKD |
| | | KPDNFQLFQSPHGKDLLEKDSADGELKIPSKMDFELYLGYEYVTALQNLRESKPPDSSKD |
| | | ECMVKWCAIGHQERTKCDRWSGFSGGAIECETAENTEECIAKIMKGEADAMSLDGGYLYI |
| | | AGKCGLVPVLAENYKTEGESCKNTPEKGYLAVAVVKTSDANINWNNLKDKKSCHTAVDRT |
| | | AGWNIPMGLLYSKINNCKFDEFFSAGCAPGSPRNSSLCALCIGSEKGIGKECVPNSNERY |
| | | YGYTGAFRCLVEKGDVAFVKDQTVIQNTDGNNNEAWAKNLKKENFEVLCKDGTRKPVTDA |
| | | ENCHLARGPNHAVVSRKDKATCVEKILNKQQDDFGKSVTDCTSNFCLFQSNSKDLLFRDD |
| | | TKCLASIAKKTYDSYLGDDYVRAMTNLRQCSTSKLLEACTFHKP |
| DNA | Searched database | GenBank |
| | accession No. | NM_177484.3 mutant |
| | Full-length | GACCCTGAGAGAA |
| | sequence | CTGTGAGATGGTGCACCATTTCAACTCATGAGGCCAATAAGTGTGCCAGTTTTCGTGAAAATGTGCTTCG |
| | (SEQ ID NO: 3) | TATCTTGGAGAGTGGTCCTTTTGTCTCCTGTGTGAAGAAAACCTCACACATGGATTGCATCAAGGCTATC |
| | | TCGAATAACGAAGCAGATGCTGTGACGTTGGATGGAGGTTTGGTGTATGAGGCAGGCCTGAAGCCCAACA |
| | | ACCTGAAGCCTGTGGTGGCAGAGTTCCATGGGACAAAAGACAACCCACAAACTCACTATTATGCTGTGGC |
| | | GGTGGTGAAGAAGGACACTGACTTCAAGCTGAATGAGCTCAGAGGCAAGAAGTCCTGCCACACAGGCCTC |
| | | GGCAGGTCCGCTGGGTGGAACATCCCCATGGGCAAACTTTATAAGGAATTGCCTGATCCACAGGAATCAA |
| | | TTCAGAGAGCTGCGGCCAACTTCTTCTCTGCCAGCTGTGTCCCCTGTGCGGATCAATCATCATTTCCCAA |
| | | ACTCTGTCAACTGTGTGCGGGGAAAGGGACAGACAAGTGTGCCTGCTCCAACCACGAACCATACTTCGGC |
| | | TACTCAGGGGCCTTTAAATGTCTGATGGAGGGCGCTGGGGATGTGGCCTTTGTCAAGCACTCAACGGTAT |
| | | TCGACAACCTGCCTAACCCAGAAGACAGGAAAAACTATGAGCTGCTCTGCGGGGACAACACCCGGAAGTC |
| | | TGTAGATAATTACCAAGAATGCTACTTGGCAATGGTTCCTTCCCATGCGGTTGTGGCTCGAACTGTGGGC |
| | | GGCAAGGAGGATGTGATCTGGGAACTTCTCAATCACGCCCAGGAACATTTTGGCAAAGATAAACCAGACA |
| | | ATTTCCAGCTTTTCCAATCCCCTCATGGGAAGGACCTGCTGTTTAAGGACTCTGCTGATGGGTTTTTAAA |
| | | GATTCCTTCTAAGATGGATTTTGAGCTGTACTTGGGATATGAATATGTCACTGCTCTTCAGAATCTAAGA |
| | | GAAAGTAAACCCCCGGACTCCTCAAAGGATGAGTGCATGGTGAAGTGGTGTGCAATTGGTCACCAGGAGA |
| | | GGACAAAGTGTGATCGGTGGAGTGGGTTCAGCGGCGGGGCAATAGAGTGTGAAACAGCAGAGAACACTGA |
| | | AGAGTGCATCGCCAAGATCATGAAAGGAGAAGCTGATGCCATGAGCTTGGACGGAGGCTACCTCTACATA |
| | | GCAGGCAAATGTGGCCTGGTGCCTGTCCTGGCAGAGAACTACAAAACTGAGGGTGAAAGTTGCAAGAACA |
| | | CACCAGAAAAAGGGTATCTTGCTGTAGCTGTGGTTAAAACATCAGATGCTAATATCAACTGGAACAATCT |
| | | GAAAGACAAGAAGTCCTGCCACACTGCAGTAGACAGAACCGCTGGCTGGAACATCCCCATGGGTTTGCTC |
| | | TACAGCAAGATTAATAACTGTAAATTTGATGAATTTTTCAGCGCAGGTTGTGCACCTGGATCTCCGCGAA |
| | | ATTCCAGTCTCTGTGCTCTGTGCATTGGCTCAGAGAAGGGTACAGGAAAGGAGTGTGTTCCCAACAGCAA |
| | | TGAAAGATACTATGGCTATACAGGGGCTTTCAGGTGTCTGGTGGAGAAGGGAGACGTGGCCTTTGTGAAG |
| | | GACCAGACTGTCATACAGAACACTGACGGAAATAATAATGAAGCATGGGCAAAAAATCTGAAGAAGGAAA |
| | | ATTTTGAAGTACTATGCAAAGATGGCACCAGGAAACCTGTGACAGATGCTGAGAACTGCCACCTGGCCCG |
| | | AGGCCCGAATCATGCTGTGGTCTCACGGAAAGATAAGGCAACTTGTGTGGAGAAAATATTAAACAAACAG |
| | | CAGGATGATTTTGGAAAATCTGTAACCGACTGCACGAGCAATTTTTGTTTATTCCAATCAAATTCCAAGG |
| | | ACCTTCTGTTCAGGGATGACACTAAATGTTTGGCTTCAATTGCGAAAAAAACATATGACTCCTACTTAGG |
| | | GGATGACTACGTCAGAGCTATGACCAACCTGAGACAATGCTCAACCTCAAAACTCCTGGAAGCATGCACT |
| | | TTCCACAAACCT |

TABLE 1-3
MW:83kDa

| SPOT No. | | | \textcircled{4} |
|---|---|---|---|
| protein | Isoelectric | Range | 6-12 |
| | point | Preferably | 6.5-11 |
| | | More preferably | 7-10 |
| | Molecular | Range | 40-160 |
| | weight | Preferably | 50-110 |
| | | More preferably | 60-100 |
| | Searched database | | Uniprot |
| | Biological species | | Cow (Bos taurus) |
| | accession No. | | G3MXZ0 |
| | Protein name | | Lactoperoxidase |
| | Full-length | | DTIAQAASTTTISDAVSKV |
| | sequence | | KIQVNKAFLDSRTRLKTTLSSEAPTTQQLSEYFKHAKGRTRTAIRNGQVWEESLKRLRRD |
| | (SEQ ID NO: 6) | | TTLTNVTDPSLDLTALSWEVGCGAPVPLVKCDENSPYRTITGDCNNRRSPALGAANRALA |
| | | | RWLPAEYEDGLALPFGWTQRKTRNGERVPLAREVSNKIVGYLDEEGVLDQNRSLLFMQWG |
| | | | QIVDHDLDFAPETELGSNEHSKTQCEEYCIQGDNCFPIMFPKNDPKLKTQGKCMPFFRAG |
| | | | FVCPTPPYQSLAREQINAVTSFLDASLVYGSEPSLASRLRNLSSPLGLMAVNQEAWDHGL |
| | | | AYLPFNNKKPSPCEFINTTARVPCFLAGDFRASEQILLATAHTLLLREHNRLARELKKLN |
| | | | PHWNGEKLYQEARKILGAFIQIITFRDYLPIVLGSEMQKWIPPYQGYNNSVDPRISNVFT |
| | | | FAFRFGHMEVPSTVSRLDENYQPWGPEAELPLHTLFFNTWRIIKDGGIDPLVRGLLAKKS |
| | | | KLMNQDKMVTSELRNKLFQPTHKIHGEDLAAINLQRCRDHGMPGYNSWRGFCGLSQPKTL |
| | | | KGLQTVLKNKILAKKLMDLYKTPDNIDIWIGGNAEPMVERGRVGPLLACLLGROFQQIRD |
| | | | GDRFWWENPGVFTEKQRDSLQKVSFSRLICDNTHITKVPLHAFQANNYPHDFVDCSTVDK |
| | | | LDLSPWASREN |
| DNA | Searched database | | GenBank |
| | accession No. | | XM_024979549.1 mutant |
| | Full-length | | gacacaattgcacaggccgccagcaccaccaccatctctgatgctgtgagtaaggtcaagatccaggtcaacaaggc |
| | sequence | | cttcctggattcccggaccaggctgaagacgaccttgagctctgaggcacccaccacccaacagctctcagagtact |
| | (SEQ ID NO: 5) | | tcaagcacgcaaagggccggacccgcacggccattcgcaacgggcaggtgtgggaggagtccttaaagaggctgagg |
| | | | cgggacacaaccctgaccaacgtcacagaccctagcctggacttgactgcactctcctgggaggtgggctgcggtgc |
| | | | cccggttcctctggtgaaatgtgatgaaaacagcccttaccgcaccatcacgggagactgtaataacaggaggagcc |
| | | | ccgcactgggcgccgccaacagggcgctggcgcgctggctgccggcggagtacgaggacgggctcgccctgcccttc |
| | | | ggctggacgcagaggaagacgcgcaacggcttccgcgtcccgctggcccgggaggtatccaacaaaattgtaggcta |
| | | | cctggacgaagagggtgttctggaccaaaacaggtccctgctcttcatgcagtggggtcagattgtggaccacgacc |
| | | | tggacttcgccccggaaacggaactggggagcaacgagcactctaaaacccagtgtgaggagtactgtatccaggga |
| | | | gacaactgcttccccatcatgttcccgaaaaatgatcccaagttgaagactcaagggaaatgcatgcctttcttccg |
| | | | agccgggtttgtctgccccactccaccttaccagtcgttggcccgagaacagatcaatgctgtgacctccttcctgg |
| | | | acgccagcttagtgtacggctctgagcccagtctggccagccgtctccggaacctcagcagcccgctgggcctcatg |
| | | | gctgtcaaccaagaagcctgggaccacgggctggcctacctgccctttaacaacaagaagccgagcccctgtgagtt |
| | | | catcaacaccaccgcccgcgtgccctgtttcctggcgggagattttcgagcctcagagcagattctgctggccactg |
| | | | cccacaccctccttctccgggagcacaaccggctggccagagaactaaagaaactcaaccctcactggaatggagag |
| | | | aagctctaccaggaagcccggaaaatcctgggagccttcatacagatcatcacctttagggactacctacccattgt |
| | | | gctaggtagtgagatgcagaagtggatcccgccctaccaaggctataataactctgtggatccccgaatttccaatg |
| | | | tcttcacctttgccttccgctttggccacatggaggttccctccactgtgtcccgcctggatgagaattaccagcca |
| | | | tggggtccggaagcagagctccccctacacaccctcttcttcaacacctggaggataatcaaagacggtggaattga |
| | | | ccctctggtgcggggtctgctggccaagaagtccaaactgatgaatcaggataaaatggtgacgagtgagctgcgca |
| | | | acaagcttttccagcccactcacaagatccacggctttgacctggctgctatcaacttacagcgttgccgagaccat |
| | | | gggatgcctgggtacaactcctggaggggcttctgtggcctctcacagcccaagacactgaaggggctgcagactgt |
| | | | gctgaagaacaagatactggctaagaagttaatggatctctataagacccccgacaacattgacatctggattggag |
| | | | gcaacgctgagcccatggtagaaaggggccgggtggggccactcctggcctgcctcctagggaggcaattccagcag |
| | | | atacgtgatggggacaggttctggtgggagaaccctggggtcttcactgagaagcagcgggactctctacagaaagt |
| | | | gtccttctcacgcctcatctgtgacaacacccacatcacgaaggtcccgctgcatgccttccaggccaacaactacc |
| | | | cacatgactttgtggattgctcaaccgttgataagctggatctctcaccctgggcctccagggagaat |

TABLE 1-4
MW:51kDa

| SPOT No. | | | \textcircled{5} |
|---|---|---|---|
| protein | Isoelectric | Range | 4-12 |
| | point | Preferably | 4.5-11 |
| | | More preferably | 5-10 |
| | Molecular | Range | 40-110 |
| | weight | Preferably | 45-100 |
| | | More preferably | 50-80 |
| | Searched database | | NCBI |
| | Biological species | | Cow (Bos taurus) |
| | accession No. | | S22080 |
| | Protein name | | Ig heavy chain precursor (B/MT.4A.17.H5.A5) |
| | Full-length | | SQVQLRESGPSLVKPSQTLSLTCTVSGFSLSSYALTWVRQAPGKALEWVGGI |
| | sequence | | TSGGTTYYNPALKSRLSITKENSKSQVSLSVSSVTPEDTATYYCARSTYGEVGDGAIADAWGQGLLVTVS |
| | (SEQ ID NO: 8) | | SASTTAPKVYPLSSCCGDKSSSTVTLGCLVSSYMPEPVTVTWNSGALKSGVHTFPALVQSSGLYSLSSMV |
| | | | TVPGSTSGQTFTCNVAHPASSTKVDKAVDPTCKPSPCDCCPPPELPGGPSVFIFPPKPKDTLTISGTPEV |
| | | | TCVVVDVGHDDPEVKFSWFVDDVEVNTATTKPREEQFNSTYRVVSALRIQHQDWIGGKEFKCKVHNEGLP |
| | | | APIVRTISRTKGPAREPQVYVLAPPQEELSKSTVSLTCMVTSFYPDYIAVEWQRNGQPESEDKYGTTPPQ |
| | | | LDADSSYFLYSKLRVDRNSWQEGDTYTCVVMHEALHNHYTQKSTSKSAGK |
| DNA | Searched database | | GenBank |
| | accession No. | | KT761812.1 mutant |
| | Full-length | | TCCCAGGTGCAGCTGC |
| | sequence | | GGGAGTCGGGCCCCAGCCTGGTGAAGCCCTCACAGACCCTCTCCCTCACGTGCACGGTGTCTGGATTCTC |
| | (SEQ ID NO: 7) | | ATTGAGCAGCTATGCTCTAACCTGGGTCCGCCAGGCTCCAGGGAAGGCGCTGGAGTGGGTTGGTGGTATA |
| | | | ACCAGTGGTGGAACCACATACTATAATCCAGCCCTGAAATCCCGGCTCAGCATCACCAAGGAGAACTCCA |
| | | | AGAGCCAAGTCTCTCTGTCAGTGAGCAGCGTGACACCTGAGGACACAGCCACATACTACTGTGCAAGAAG |
| | | | TACTTATGGTGAGGTTGGTGATGGTGCCATCGCCGATGCCTGGGGCCAAGGACTCCTGGTCACCGTCTCC |
| | | | TCAGCCTCCACCACAGCCCCGAAAGTCTACCCTCTGAGTTCTTGCTGCGGGGACAAGTCCAGCTCCACCG |
| | | | TGACCCTGGGCTGCCTGGTCTCCAGCTACATGCCCGAGCCGGTGACCGTGACCTGGAACTCGGGTGCCCT |
| | | | GAAGAGCGGCGTGCACACCTTCCCGGCTGTCCTTCAGTCCTCCGGGCTGTACTCTCTCAGCAGCATGGTG |
| | | | ACCGTGCCCGGCAGCACCTCAGGACAGACCTTCACCTGCAACGTAGCCCACCCGGCCAGCAGCACCAAGG |
| | | | TGGACAAGGCTGTTGATCCCACATGCAAACCATCACCCTGTGACTGTTGCCCACCCCCTGAGCTCCCCGG |
| | | | AGGACCCTCTGTCTTCATCTTCCCACCGAAACCCAAGGACACCCTCACAATCTCGGGAACGCCCGAGGTC |
| | | | ACGTGTGTGGTGGTGGACGTGGGCCACGATGACCCCGAGGTGAAGTTCTCCTGGTTCGTGGACGACGTGG |
| | | | AGGTAAACACAGCCACGACGAAGCCGAGAGAGGAGCAGTTCAACAGCACCTACCGCGTGGTCAGCGCCCT |
| | | | GCGCATCCAGCACCAGGACTGGACTGGAGGAAAGGAGTTCAAGTGCAAGGTCCACAACGAAGGCCTCCCG |
| | | | GCCCCCATCGTGAGGACCATCTCCAGGACCAAAGGGCCGGCCCGGGAGCCGCAGGTGTATGTCCTGGCCC |
| | | | CACCCCAGGAAGAGCTCAGCAAAAGCACGGTCAGCCTCACCTGCATGGTCACCAGCTTCTACCCAGACTA |
| | | | CATCGCCGTGGAGTGGCAGAGAAACGGGCAGCCTGAGTCGGAGGACAAGTACGGCACGACCCCGCCCCAG |
| | | | CTGGACGCCGACAGCTCCTACTTCCTGTACAGCAAGCTCAGGGTGGACAGGAACAGCTGGCAGGAAGGAG |
| | | | ACACCTACACGTGTGTGGTGATGCACGAGGCCCTGCACAATCACTACACGCAGAAGTCCACCTCTAAGTC |
| | | | TGCGGGTAAA |

TABLE 1-5
MW:49kDa

| SPOT No. | | | \textcircled{6} |
|---|---|---|---|
| protein | Isoelectric | Range | 6-12 |
| | point | Preferably | 7-11.5 |
| | | More preferably | 8-11 |
| | Molecular | Range | 20-90 |
| | weight | Preferably | 30-80 |
| | | More preferably | 40-60 |
| | Searched database | | Uniprot |
| | Biological species | | Cow (Bos taurus) |
| | accession No. | | F1N1N6 |
| | Protein name | | Perilipin |
| | Full-length | | MASVAAEPQLSVVTRVANLPLVSSTYDLVSSAYISRKDQYPYLKSLCEMAEKGMKTITSV |
| | sequence | | AVTSALPIIQKLEPQIAVANTYACKGLDRIEEKLPILNQPTNQVVANAKGAMTGAKDAVT |
| | (SEQ ID NO: 10) | | TTVTGAKDSVASTITGVVDRTKGAVTGSVEKTKSVVSGSINTVLRSRVMQLMSSGVENAL |
| | | | TKSELLVDQYLPLTKDELEKEAKKVEGFDMVQKPSYYVRLGSLSTKLRSRAYQQALCRVE |
| | | | EAKRKGQETISQLHSAFNLSELARKNVHNANQKIQDAQDKLYLSWLEWKRSIGYDDTDES |
| | | | HCAEHIESRTLAIARNLTQQLQTMCHTLLSNIQGLPQNIQDRANHLGVMAGDIYSVFRNA |
| | | | ASFKEVSDGLLASSKGQLQKMKESLDDVMDYLVNNTPLNWLVGPFYPQVTESESAQAPGT |
| | | | TRRPGRWSRKHPKPVPVSNAEGSQPDDSSS |
| DNA | Searched database | | GenBank |
| | accession No. | | XM_024995432.1 |
| | Full-length | | ATGGCATCTGTTGCAGCTGAACCACAACTGAGTGTGGTGACCAGGGTGGCCAACCTCCCCTTGGTGAGCT |
| | sequence | | CCACGTATGATCTGGTCTCCTCGGCTTACATCAGTAGAAAGGATCAGTATCCTTACTTGAAGTCTCTGTG |
| | (SEQ ID NO: 9) | | TGAGATGGCAGAGAAGGGCATGAAGACCATCACTTCTGTGGCTGTGACCAGCGCGCTGCCAATCATCCAA |
| | | | AAGTTAGAGCCACAAATTGCTGTTGCCAATACCTATGCGTGTAAGGGGCTAGACAGGATTGAGGAGAAGC |
| | | | TGCCTATTCTGAATCAGCCAACAAACCAGGTTGTGGCCAATGCCAAAGGGGCTATGACTGGGGCAAAAGA |
| | | | TGCTGTGACGACTACTGTGACCGGGGCCAAGGATTCTGTGGCCAGCACAATCACGGGGGTGGTGGACAGG |
| | | | ACCAAGGGAGCTGTGACTGGTAGTGTGGAGAAGACCAAGTCCGTGGTCAGTGGCAGCATTAACACAGTCC |
| | | | TACGAAGTCGGGTGATGCAGCTGATGAGCAGTGGAGTAGAAAATGCACTCACCAAATCAGAGCTGCTGGT |
| | | | AGACCAGTACCTCCCTCTTACCAAGGATGAACTAGAAAAAGAAGCCAAAAAAGTGGAAGGATTCGATATG |
| | | | GTTCAGAAGCCGAGTTACTATGTTAGACTGGGATCTCTGTCCACCAAGCTGCGCTCACGGGCCTACCAGC |
| | | | AGGCCCTTTGTAGGGTTGAAGAAGCTAAGCGAAAAGGCCAGGAGACCATTTCTCAGCTCCATTCCGCCTT |
| | | | CAACCTGAGTGAACTTGCCAGGAAGAATGTGCATAATGCCAACCAGAAAATTCAGGATGCTCAGGATAAG |
| | | | CTCTATCTGTCCTGGCTGGAGTGGAAGAGAAGCATCGGCTACGATGATACAGATGAATCCCACTGTGCTG |
| | | | AGCACATTGAGTCACGTACTCTTGCTATTGCCCGGAACCTGACTCAGCAGCTCCAGACCATGTGCCACAC |
| | | | CCTCCTGTCCAACATCCAGGGGTTACCACAGAACATTCAAGATCGGGCCAACCACTTGGGGGTGATGGCT |
| | | | GGTGACATCTACTCTGTGTTTCGCAATGCTGCCTCCTTTAAAGAAGTGTCTGATGGCCTCCTCGCTTCCA |
| | | | GCAAGGGGCAGCTGCAGAAAATGAAGGAGTCTTTAGATGATGTGATGGATTATCTTGTTAACAACACACC |
| | | | CCTCAACTGGCTGGTAGGTCCCTTTTATCCCCAGGTGACCGAGTCTGAGAGTGCTCAGGCCCCAGGTACA |
| | | | ACCAGGAGGCCTGGCAGGTGGAGTAGAAAACACCCTAAACCCGTCCCTGTCAGCAATGCAGAGGGCAGCC |
| | | | AGCCAGATGACAGCTCCTCT |

TABLE 1-6
MW:82kDa

| SPOT No. | | | \textcircled{7} |
|---|---|---|---|
| protein | Isoelectric | Range | 5-12 |
| | point | Preferably | 5.5-11 |
| | | More preferably | 6-10 |
| | Molecular | Range | 40-160 |
| | weight | Preferably | 50-110 |
| | | More preferably | 60-100 |
| | Searched database | | Uniprot |
| | Biological species | | Cow (Bos taurus) |
| | accession No. | | P81265 |
| | Protein name | | Polymeric immunoglobulin receptor |
| | Full-length | | KSPIFGPEEVTSVEGRSVSIKCYYPPTSVNRHTRKYWCRQGA |
| | sequence | | QGRCTTLISSEGYVSDDYVGRANLINFPESGTFVVDISHLTHKDSGRYKCGLGISSRGLN |
| | (SEQ ID NO: 12) | | FDVSLEVSQDPAQASHAHVYTVDLGRTVTINCPFTRANSEKRKSLCKKTIQDCFQVVDST |
| | | | GYVSNSYKDRAHISILGTNTLVFSVVINRVKLSDAGMYVCQAGDDAKADKINIDLQVLEP |
| | | | EPELVYGDLRSSVTFDCSLGPEVANVPKFLCQKKNGGACNVVINTLGKKAQDFQGRIVSV |
| | | | PKDNGVFSVHITSLRKEDAGRYVCGAQPEGEPQDGWPVQAWQLFVNEETAIPASPSVVKG |
| | | | VRGGSVTVSCPYNPKDANSAKYWCHWEEAQNGRCPRLVESRGLIKEQYEGRLALLTEPGN |
| | | | GTYTVILNQLTDQDTGFYWCVTDGDTRWISTVELKVVQGEPSLKVPKNVTAWLGEPLKLS |
| | | | CHFPCKFYSFEKYWCKWSNRGCSALPTQNDGPSQAFVSCDQNSQVVSLNLDTVTKEDEGW |
| | | | YWCGVKEGPRYGETAAVYVAVESRVKGSQGAKQVKAAPAGAAIQSRAGEIQNKALLDPSF |
| | | | FAKESVKDAAGGPGAPADPGRPTGYSGSSKALVSTLVPLALVLVAGVVAIGVVRARHRKN |
| | | | VDRISIRSYRTDISMSDFENSRDFEGRDNMGASPEAQETSLGGKDEFATTTEDTVESKEP |
| | | | KKAKRSSKEEADEAFTTFLLQAKNLASAATQNGPTEA |
| DNA | Searched database | | GenBank |
| | accession No. | | AAC41620.1 |
| | Full-length | | AAGAGT |
| | sequence | | CCCATCTTCGGTCCCGAGGAGGTGACCAGCGTGGAAGGCCGCTCAGTGTCCATCAAGTGC |
| | (SEQ ID NO: 11) | | TACTACCCGCCCACCTCCGTCAACCGGCACACGCGCAAGTACTGGTGCCGGCAGGGAGCC |
| | | | CAGGGCCGCTGCACGACCCTCATCTCCTCGGAGGGCTACGTCTCCGACGACTACGTGGGC |
| | | | AGAGCCAACCTCACCAACTTCCCGGAGAGCGGCACGTTTGTGGTGGACATCAGCCATCTC |
| | | | ACCCATAAAGACTCAGGGCGCTACAAGTGTGGCCTGGGCATTAGCAGCCGTGGCCTTAAC |
| | | | TTCGATGTGAGCCTGGAGGTCAGCCAAGATCCTGCACAGGCAAGTCATGCCCACGTCTAC |
| | | | ACTGTAGACCTGGGCAGGACTGTGACCATCAACTGCCCTTTCACGCGTGCGAATTCTGAG |
| | | | AAGAGAAAATCCTTGTGCAAGAAGACAATCCAGGACTGTTTCCAAGTTGTCGACTCCACC |
| | | | GGGTATGTGAGCAACAGCTATAAAGACAGAGCACATATCAGTATCCTAGGTACCAACACA |
| | | | TTAGTGTTCAGCGTTGTCATCAACCGAGTCAAGCTCAGTGATGCTGGGATGTATGTCTGC |
| | | | CAGGCTGGGGACGATGCCAAAGCCGATAAAATCAACATTGACCTCCAGGTGCTGGAGCCT |
| | | | GAGCCTGAGCTGGTTTATGGAGACTTGAGGAGCTCGGTGACCTTTGACTGTTCCCTGGGC |
| | | | CCCGAGGTGGCAAATGTGCCCAAATTTCTGTGCCAGAAGAAGAATGGGGGAGCTTGCAAT |
| | | | GTGGTCATCAACACGTTGGGGAAGAAGGCTCAGGACTTCCAGGGCAGGATCGTGTCCGTG |
| | | | CCCAAGGACAATGGTGTCTTCAGTGTGCACATTACCAGCCTGAGGAAAGAGGACGCAGGG |
| | | | CGCTACGTGTGGGGGGCCCAGCCTGAGGGTGAGCCCCAGGACGGCTGGCCTGTGCAGGCC |
| | | | TGGCAACTCTTCGTCAATGAAGAGACGGCAATCCCCGCAAGCCCCTCCGTGGTGAAAGGT |
| | | | GTGAGGGGAGGCTCTGTGACCGTATCTTGCCCCTACAACCCGAAGGATGCCAACAGCGCG |
| | | | AAGTACTGGTGTCACTGGGAAGAGGCTCAAAACGGCCGCTGCCCGCGGCTGGTGGAGAGC |
| | | | CGGGGGCTGATAAAGGAGCAGTACGAGGGCAGGCTGGCGCTGCTTACCGAGCCGGGCAAC |
| | | | GGCACCTACACCGTCATCCTCAACCAGCTCACCGACCAGGACACCGGCTTCTACTGGTGC |
| | | | GTGACCGACGGCGACACGCGCTGGATCTCCACAGTGGAGCTCAAGGTTGTCCAAGGAGAA |
| | | | CCAAGCCTCAAGGTACCCAAGAACGTCACGGCTTGGCTGGGAGAGCCCTTAAAGCTCTCC |
| | | | TGCCACTTCCCCTGCAAATTCTACTCCTTTGAGAAGTACTGGTGTAAGTGGAGCAACAGA |
| | | | GGCTGCAGCGCCCTGCCCACCCAGAACGACGGCCCCAGCCAGGCCTTTGTGAGCTGTGAC |
| | | | CAGAACAGCCAGGTCGTCTCCCTGAACCTGGACACAGTCACCAAGGAGGATGAAGGCTGG |
| | | | TACTGGTGTGGAGTGAAGGAGGGCCCCCGATATGGGGAGACGGCGGCTGTCTACGTGGCA |
| | | | GTGGAGAGCAGGGTGAAGGGGTCCCAAGGCGCCAAGCAAGTGAAAGCTGCCCCTGCGGGG |
| | | | GCGGCAATACAGTCGAGGGCCGGGGAGATCCAGAACAAAGCCCTTCTGGACCCCAGCTTT |
| | | | TTCGCAAAGGAAAGTGTGAAGGACGCTGCTGGTGGACCCGGAGCACCTGCAGATCCTGGC |
| | | | CGCCCTACAGGATACAGCGGGAGCTCCAAAGCACTGGTCTCCACCCTGGTGCCCCTGGCC |
| | | | CTGGTCCTGGTCGCAGGGGTCGTGGCGATCGGGGTGGTCCGAGCCCGGCACAGGAAGAAC |
| | | | GTCGACCGGATTTCAATCAGGAGCTACCGGACAGATATCAGCATGTCAGACTTTGAGAAC |
| | | | TCCAGGGATTTTGAAGGACGTGACAACATGGGAGCCTCTCCAGAGGCCCAAGAGACGTCT |
| | | | CTCGGAGGGAAGGACGAGTTTGCCACCACTACCGAGGACACCGTGGAGAGCAAAGAACCC |
| | | | AAGAAGGCAAAGAGGTCGTCCAAGGAGGAAGCCGACGAGGCCTTCACCACCTTCCTCCTC |
| | | | CAGGCCAAAAACCTGGCCTCCGCCGCAACCCAGAACGGCCCGACAGAAGCC |

TABLE 1-7
MW:74kDa

| SPOT No. | | | \textcircled{8} |
|---|---|---|---|
| protein | Isoelectric | Range | 6-12 |
| | point | Preferably | 7-11.5 |
| | | More preferably | 8-11 |
| | Molecular | Range | 40-110 |
| | weight | Preferably | 50-100 |
| | | More preferably | 60-80 |
| | Searched database | | Uniprot |
| | Biological species | | Cow (Bos taurus) |
| | accession No. | | A0A0F6QNP7 |
| | Protein name | | C3-beta-c |
| | Full-length | | NPMYSMITPNILRVESEETVVLEAHGGQGTIQVSVTVH |
| | sequence | | DFPAKKQVLSNENTQLNSNNGYLSTVTIKIPASKELKSDKGHKFVTVVATFGNVQVEKVV |
| | (SEQ ID NO: 14) | | LISLQSGYLFIQTDKTIYTPGSTVLYRVFTVDHKLLPVGQTVFITIETPDGIPVKRDSKS |
| | | | SQNQFGILTLSWNIPELVNMGVWKIKAYYEDSPQQVFSAEFEVKEYVLPSFEVQLEPEEK |
| | | | FYYIDDPDGLKVNIIARFLYGEQVDGTAFVIFGVQDGDRRISLTHSLTRVPINDGNGEAI |
| | | | LKRQVLLNGVQPSRADALVGKSIYVSATVILQSGSDMVEAERTGIPIVTSPYQIHFTKTP |
| | | | KFFKPAMPFDLMVYVTNPDGSPARHIPVVTQGSNVQSLTQDDGVAKLSINTQNKRDPLTI |
| | | | TVRTKKDNIPEGRQATRTMQALPYNTQGNSNNYLHLSVPRVELKPGETLNVNFHLRTDPG |
| | | | EQAKIRYYTYMIMNKGKLLKVGRQYREPGQDLVVLPLTITSDFIPSFRLVAYYTLINAKG |
| | | | QREVVADSVWVDVKDSCMGTLVVKNGGKEEKHHRPGQQITLKIEADQGARVGLVAVDKGV |
| | | | FVLNKKNKLTQRKIWDVVEKADIGCTPGSGRNYAGVFTDAGLTLKTSQGLETQQRADPQC |
| | | | PQPATRRR |
| DNA | Searched database | | GenBank |
| | accession No. | | AKE14287.1 |
| | Full-length | | AACCCCATGTATTCCATGATCACCCCCAACATCCTGCGTGTGGAGAGCCGAGGAG |
| | sequence | | ACTGTGGTGCTGGAGGCCCACGGAGGACAAGGGACTATTCAGGTCTCGGTCACCGTCCAC |
| | (SEQ ID NO: 13) | | GACTTCCCGGCCAAGAAACAAGTGCTGTCAAACGAAAACACCCAGCTGAACAGCAACAAT |
| | | | GGCTACCTGAGCACCGTCACCATCAAGATCCCGGCCAGCAAAGAGTTAAAATCCGACAAG |
| | | | GGGCACAAGTTCGTGACTGTCGTAGCGACCTTCGGGAATGTCCAGGTGGAGAAGGTGGTG |
| | | | CTGATCAGCCTTCAGAGCGGGTACCTCTTCATCCAGACGGACAAGACCATCTACACCCCA |
| | | | GGCTCCACGGTCCTCTATCGGGTCTTCACTGTTGACCACAAGCTGCTGCCCGTGGGCCAG |
| | | | ACGGTTTTCATCACCATCGAGACCCCTGATGGCATCCCTGTCAAGCGGGACTCCAAGTCT |
| | | | TCTCAGAACCAGTTTGGCATTTTGACCTTGTCCTGGAACATTCCAGAGCTAGTGAACATG |
| | | | GGGGTGTGGAAGATCAAAGCCTACTATGAAGATTCTCCGCAGCAGGTCTTCTCCGCTGAG |
| | | | TTTGAGGTGAAGGAATATGTGCTTCCCAGTTTTGAGGTCCAACTAGAGCCTGAAGAGAAA |
| | | | TTCTACTACATTGATGACCCAGATGGCCTGAAGGTCAACATCATTGCCAGGTTCTTGTAC |
| | | | GGGGAACAGGTGGACGGAACAGCTTTCGTCATCTTTGGGGTCCAGGATGGCGACCGGAGA |
| | | | ATTTCATTGACTCACTCCCTCACCCGTGTTCCGATCAACGATGGTAATGGGGAGGCCATA |
| | | | CTGAAACGTCAGGTTTTGCTGAATGGAGTACAGCCCTCCCGAGCTGATGCCCTGGTGGGC |
| | | | AAGTCCATATATGTGTCCGCCACCGTCATCTTGCAATCAGGCAGTGACATGGTGGAGGCA |
| | | | GAGCGCACCGGGATCCCCATTGTGACTTCCCCCTACCAGATCCATTTCACCAAGACCCCO |
| | | | AAGTTCTTCAAACCTGCCATGCCCTTCGACCTCATGGTGTACGTGACAAACCCCGACGGC |
| | | | TCCCCCGCTCGCCACATCCCGGTGGTGACCCAGGGCAGCAATGTGCAGTCCTTGACCCAG |
| | | | GATGATGGCGTGGCCAAGCTAAGCATCAACACACAGAACAAACGGGATCCCCTGACCATC |
| | | | ACCGTACGCACAAAAAAGGACAATATCCCCGAGGGGCGACAGGCCACCAGGACCATGCAG |
| | | | GCCCTACCCTACAACACCCAGGGGAACTCCAATAACTACCTGCACCTCTCTGTGCCCCGC |
| | | | GTGGAGCTCAAGCCTGGGGAGACTCTCAACGTCAACTTCCACCTGCGCACGGACCCCGGC |
| | | | GAGCAAGCCAAGATCCGCTACTACACCTACATGATCATGAACAAGGGGAAGCTGTTGAAG |
| | | | GTGGGACGCCAGTACCGAGAGCCCGGCCAGGACCTAGTGGTGTTGCCCTTGACCATCACG |
| | | | TCAGACTTCATCCCTTCCTTCCGCCTGGTGGCCTATTACACACTGATTAATGCCAAAGGC |
| | | | CAGAGGGAGGTGGTGGCCGACTCCGTGTGGGTGGATGTCAAGGACTCGTGTATGGGCACG |
| | | | CTGGTGGTGAAAAATGGTGGGAAGGAAGAGAAACACCATCGACCCGGGCAACAGATAACC |
| | | | CTGAAGATCGAGGCTGACCAGGGGGCCCGAGTGGGGCTCGTGGCCGTGGACAAGGGCGTG |
| | | | TTTGTGCTGAATAAGAAGAACAAATTGACCCAGAGAAAGATCTGGGACGTGGTGGAGAAA |
| | | | GCGGACATTGGCTGCACCCCAGGCAGTGGAAGAAACTATGCTGGCGTCTTCACGGATGCA |
| | | | GGACTCACTCTCAAGACCAGCCAGGGCCTGGAGACCCAGCAGAGGGCAGATCCGCAGTGC |
| | | | CCGCAACCAGCCACCCGCCGACGC |

TABLE 1-8
MW:25kDa

| SPOT No. | | | \textcircled{9} |
|---|---|---|---|
| protein | Isoelectric | Range | 4-12 |
| | point | Preferably | 4.5-11 |
| | | More preferably | 5-10 |
| | Molecular | Range | 10-50 |
| | weight | Preferably | 15-40 |
| | | More preferably | 20-30 |
| | Searched database | | Uniprot |
| | Biological species | | Cow (Bos taurus) |
| | accession No. | | Q1RMN8 |
| | Protein name | | Immunoglobulin light chain, lambda gene cluster |
| | Full-length | | QAVLTQPSSVSGSLGQRVSITCSGSSSNIGRDNVGWYQQVP |
| | sequence | | GSGLRTIIYSSSSRPSGVPDRFSGSKSGNTATLTISSLQAEDEADYFCVAYDSSSSTVFG |
| | (SEQ ID NO: 16) | | SGTTLTVLGQPKSPPSVTLFPPSTEELNGNKATLVCLISDFYPGSVTVVWKADGSTITRN |
| | | | VETTRASKQSNSKYAASSYLSLTSSDWKSKGSYSCEVTHEGSTMTVKPSECS |
| DNA | Searched database | | GenBank |
| | accession No. | | AAI14802.1 |
| | Full-length | | CAG |
| | sequence | | GCTGTGCTGACTCAGCCGTCCTCCGTGTCCGGCTCCCTGGGCCAGAGGGTCTCCATCACC |
| | (SEQ ID NO: 15) | | TGCTCTGGAAGCAGCAGCAACATCGGTAGAGATAATGTGGGCTGGTACCAACAGGTCCCA |
| | | | GGATCGGGCCTCAGAACCATCATCTATAGTAGTAGCAGTCGACCCTCGGGGGTCCCCGAC |
| | | | CGATTCTCCGGCTCCAAGTCTGGCAACACAGCCACCCTGACCATCAGCTCCCTCCAGGCT |
| | | | GAGGACGAGGCGGATTATTTCTGTGTAGCTTATGACAGTAGTAGTAGTACTGTTTTCGGC |
| | | | AGCGGGACCACACTGACCGTCCTGGGTCAGCCCAAGTCCCCACCCTCGGTCACCCTGTTC |
| | | | CCGCCCTCCACGGAGGAGCTCAACGGCAACAAGGCCACCCTGGTGTGTCTCATCAGCGAC |
| | | | TTCTACCCGGGTAGCGTGACCGTGGTCTGGAAGGCAGACGGCAGCACCATCACCCGCAAC |
| | | | GTGGAGACCACCCGGGCCTCCAAACAGAGCAACAGCAAGTACGCGGCCAGCAGCTACCTG |
| | | | AGCCTGACGAGCAGCGACTGGAAATCGAAAGGCAGTTACAGCTGCGAGGTCACGCACGAG |
| | | | GGGAGCACCGTGACGAAGACAGTGAAGCCCTCAGAGTGTTCT |

TABLE 1-9
MW:59kDa

| SPOT No. | | | \textcircled{10} |
|---|---|---|---|
| protein | Isoelectric | Range | 1-7 |
| | point | Preferably | 1.5-6 |
| | | More preferably | 2-5 |
| | Molecular | Range | 40-160 |
| | weight | Preferably | 45-110 |
| | | More preferably | 50-100 |
| | Searched database | | Uniprot |
| | Biological species | | Cow (Bos taurus) |
| | accession No. | | F1N726 |
| | Protein name | | Glycoprotein 2 |
| | Full-length | | EQQGYRNSTNTGSYEKDLDCGAPGTPEAQLCFDP |
| | sequence | | CQNYTLLNEPFRSTENTEDIQGCDSDKHGWYFRVGDGGVRMPEDCVPTFRCQTSAPLWLN |
| | (SEQ ID NO: 18) | | GTHPGLGEGIVNRTACAHWSGNCCLWKTEVLVKACPGPYYVYRLEGTPQCSLRYCTDPAT |
| | | | AEDKCDRTCRPEEECRLVSGTWGCFCRQDLNVSDVHSLQPQLDCGDTEIKVSLDKCLLGS |
| | | | LGFGDEVHAYLRDGNWNCSSLRQSEEENWISVTNPTQAGACGNILERNQTHAIYINTLSL |
| | | | VNDFIIRDTILSINFQCAYPLDMKVSLQMALQPIVSSLNITVDGEGEFTVRMALFQDQDY |
| | | | TSPYEGTAVMLSVESMLYVGTILERGDTSRFNLVLKNCYATPTEDKTDPVKYFIIRNSCP |
| | | | NQRDSTISVEENGVSAESRFSVQMFKFAGNYDLVFLHCEVSLCDFIKEECQPSCSRSQLR |
| | | | SEGVAIDPARVLDLGPITRKGAQSLGVMSGTPNTAGFLVAWPLVLLPVLLAGLF |
| DNA | Searched database | | GenBank |
| | accession No. | | NM_001075950.2 |
| | Full-length | | GAACAGCAAGGTTACAGAAACTCCACCAACACTGGTTCCTATGAAAAGGACTTGGATTGCGG |
| | sequence | | AGCCCCTGGCACCCCAGAGGCCCAGCTCTGTTTTGACCCCTGCCAGAATTACACCCTCCTGAATGAACCC |
| | (SEQ ID NO: 17) | | TTTCGAAGCACAGAGAATACAGAAGACATCCAAGGCTGTGACAGTGACAAACACGGCTGGTACCGATTTG |
| | | | TGGGGGACGGAGGAGTGAGGATGCCAGAGGACTGTGTCCCCACGTTCCGATGCCAGACGTCTGCACCCCT |
| | | | GTGGCTGAATGGCACCCATCCTGGCCTTGGGGAGGGCATCGTCAACCGCACTGCCTGTGCCCACTGGAGT |
| | | | GGCAACTGCTGCCTCTGGAAGACAGAGGTGCTGGTGAAAGCCTGCCCGGGCCCATACTACGTGTACCGGT |
| | | | TGGAGGGCACCCCTCAATGTAGTCTGAGATACTGCACAGACCCTGCCACCGCGGAGGACAAGTGTGACAG |
| | | | GACCTGCCGCCCCGAGGAGGAGTGTCGCCTCGTCAGTGGCACCTGGGGCTGCTTCTGCAGACAGGACCTC |
| | | | AACGTCTCCGATGTGCACAGTTTGCAGCCTCAGCTGGACTGTGGGGACACAGAGATCAAAGTGTCACTGG |
| | | | ACAAGTGTCTGCTGGGAAGTCTGGGTTTCGGAGATGAGGTCCATGCCTACCTGCGCGACGGGAACTGGAA |
| | | | CTGCAGCAGCCTCAGGCAGAGTGAAGAGGAGAACTGGATATCTGTGACCAATCCCACCCAGGCTGGTGCC |
| | | | TGCGGAAACATTCTGGAGAGAAACCAGACCCATGCCATCTACATAAACACGCTCTCCTTAGTCAACGATT |
| | | | TCATCATCAGAGACACCATCCTCAGCATCAACTTCCAGTGTGCCTACCCACTGGACATGAAAGTGAGCCT |
| | | | TCAAATGGCCCTGCAGCCCATCGTAAGTTCCCTGAACATCACTGTGGATGGGGAGGGAGAGTTCACTGTC |
| | | | AGGATGGCCCTCTTTCAAGACCAGGACTACACGTCTCCTTATGAAGGGACTGCAGTCATGCTGTCTGTTG |
| | | | AATCCATGCTCTATGTGGGCACCATCTTGGAGAGAGGGGACACGTCCCGATTTAACCTGGTGTTGAAGAA |
| | | | CTGCTACGCCACGCCCACTGAAGACAAGACTGACCCTGTGAAGTACTTCATCATCAGGAACAGCTGCCCA |
| | | | AATCAACGTGATTCCACCATCTCCGTGGAAGAGAATGGGGTGTCAGCAGAAAGCCGGTTCTCAGTTCAGA |
| | | | TGTTCAAGTTTGCTGGAAATTATGACCTAGTTTTCCTCCATTGTGAGGTTTCTCTCTGTGATTTCATTAA |
| | | | AGAAGAGTGCCAACCGTCTTGCTCAAGAAGTCAACTCCGCAGTGAAGGGGTGGCTATTGATCCAGCCCGA |
| | | | GTTCTAGATCTGGGACCCATCACTCGCAAAGGTGCCCAGTCTCTTGGTGTCATGAGTGGAACACCCAACA |
| | | | CTGCAGGGTTCCTGGTGGCCTGGCCCCTGGTTCTCCTGCCTGTCCTCCTGGCTGGGCTGTTC |

TABLE 1-10
MW:47kDa

| SPOT No. | | | \textcircled{11} |
|---|---|---|---|
| protein | Isoelectric | Range | 4-11 |
| | point | Preferably | 4.5-10 |
| | | More preferably | 5-9 |
| | Molecular | Range | 30-80 |
| | weight | Preferably | 35-70 |
| | | More preferably | 40-60 |
| | Searched database | | Uniprot |
| | Biological species | | Cow (Bos taurus) |
| | accession No. | | Q95114 |
| | Protein name | | Lactadherin |
| | Full-length | | ASGDFCDSSLCLHGGTCLLNEDRTPPFYCLCPEGFTGLLCNE |
| | sequence | | TEHGPCFPNPCHNDAECQVTDDSHRGDVFIQYICKCPLGYVGIHCETTCTSPLGMQTGAI |
| | (SEQ ID NO: 20) | | ADSQISASSMHLGFMGLQRWAPELARLHQTGIVNAWTSGNYDKNPWIQVNLMRKMWVTGV |
| | | | VTQGASRAGSAEYLKTFKVAYSTDGRQFQFIQVAGRSGDKIFIGNVNNSGLKINLFDTPL |
| | | | ETQYVRLVPIICHRGCTLRFELLGCELNGCTEPLGLKDNTIPNKQITASSYYKTWGLSAF |
| | | | SWFPYYARLDNQGKFNAWTAQTNSASEWLQIDLGSOKRVTGIITQGARDFGHIQYVAAYR |
| | | | VAYGDDGVTWTEYKDPGASESKIFPGNMDNNSHKKNIFETPFQARFVRIQPVAWHNRITL |
| | | | RVELLGC |
| DNA | Searched database | | GenBank |
| | accession No. | | CAA62997.1 mutant |
| | Full-length | | GCCTCC |
| | sequence | | GGTGACTTCTGTGACTCCAGCTTGTGCCTACATGGTGGGACCTGCTTGTTGAACGAGGAC |
| | (SEQ ID NO: 19) | | AGAACCCCCCCCTTCTACTGCCTCTGCCCCGAAGGCTTCACGGGCCTCCTATGCAACGAG |
| | | | ACAGAGCACGGTCCCTGTTTOCCAAACCCCTGCCACAATGATGCTGAGTGCCAGGTAACT |
| | | | GACGACTCCCACCGAGGGGATGTCTTCATCCAGTACATCTGCAAGTGCCCTCTTGGATAC |
| | | | GTGGGCATCCACTGTGAGACCACCTGCACCTCACCGCTGGGCATGCAGACGGGTGCCATT |
| | | | GCCGACTCCCAGATCTCTGCCTCGTCCATGCATTTGGGCTTCATGGGTTTGCAGCGCTGG |
| | | | GCCCCGGAGCTAGCCCGCCTGCACCAGACGGGCATCGTCAACGCTTGGACATCCGGCAAC |
| | | | TATGACAAAAACCCCTGGATCCAGGTGAACCTGATGCGGAAGATGTGGGTGACGGGTGTG |
| | | | GTGACCCAGGGGGCCAGCCGCGCAGGGAGTGCTGAGTACCTGAAGACTTTTAAAGTGGCC |
| | | | TACAGCACCGATGGGCGCCAATTCCAGTTCATCCAGGTTGCAGGGCGGTCAGGAGATAAG |
| | | | ATATTTATAGGTAATGTGAACAACAGCGGCCTGAAGATTAACCTGTTTGATACCCCCTTG |
| | | | GAGACGCAGTATGTGAGACTGGTACCCATCATCTGCCACCGGGGCTGCACCCTCCGCTTT |
| | | | GAACTCCTTGGCTGTGAGTTGAATGGATGCACTGAACCCCTAGGCCTGAAGGATAATACC |
| | | | ATCCCCAACAAGCAGATCACAGCCTCCAGCTACTACAAAACCTGGGGCCTGAGTGCCTTT |
| | | | AGCTGGTTTCCCTACTACGCACGACTGGATAATCAGGGCAAGTTCAACGCCTGGACCGCC |
| | | | CAGACCAACAGTGCCTCTGAGTGGCTGCAGATTGACCTGGGCTCCCAGAAGCGAGTCACG |
| | | | GGCATCATCACCCAGGGTGCCCGAGACTTTGGCCACATTCAATATGTGGCTGCCTACAGG |
| | | | GTGGCCTATGGTGATGATGGTTTGACCTGGACTGAGTACAAGGACCCGGGGGCCTCAGAA |
| | | | AGCAAGATTTTCCCTGGTAACATGGACAATAATTCCCACAAGAAGAACATATTTGAGACG |
| | | | CCGTTCCAGGCTCGCTTCGTGCGGATCCAGCCCGTGGCCTGGCACAACCGTATCACCCTG |
| | | | CGAGTGGAGCTGCTGGGCTGT |

TABLE 1-11
MW:34kDa

| SPOT No. | | | \textcircled{12} |
|---|---|---|---|
| protein | Isoelectric | Range | 3-8 |
| | point | Preferably | 3.5-7 |
| | | More preferably | 4-6 |
| | Molecular | Range | 20-70 |
| | weight | Preferably | 25-60 |
| | | More preferably | 30-50 |
| | Searched database | | Uniprot |
| | Biological species | | Cow (Bos taurus) |
| | accession No. | | Q3ZCH5 |
| | Protein name | | Zinc-alpha-2-glycoprotein |
| | Full-length | | EETQAGNYSLSFLYTGLSKPREGFPSFOAVAYLNDQPFFHYNS |
| | sequence | | EGRRAEPLAPWSQVEGMEDWEKESALQRAREDIFMETLSDIMDYYKDREGSHTFQGAFGC |
| | (SEQ ID NO: 22) | | ELRNNESSGAFWGYAYDGQDFIKFDKEIPAWVPLDPAAQNTKRKWEAEAVYVQRAKAYLE |
| | | | EECPGMLRRYLPYSRTHLDRQESPSVSVTGHAAPGHKRTLKCLAYDFYPRSIGLHWTRAG |
| | | | DAQEAESGGDVLPSGNGTYQSWVVVGVPPEDQAPYSCHVEHRSLTRPLTVPWDPRQQAE |
| DNA | Searched database | | GenBank |
| | accession No. | | AAI02238.1 |
| | Full-length | | GAGGAGACC |
| | sequence | | CAGGCTGGGAATTACTCTCTAAGCTTCCTCTACACCGGGCTGTCCAAGCCCCGTGAGGGC |
| | (SEQ ID NO: 21) | | TTCCCCAGCTTCCAGGCCGTTGCCTACCTCAATGACCAGCCCTTCTTCCACTACAATAGT |
| | | | GAAGGCAGGAGGGCCGAGCCCCTGGCGCCATGGAGTCAGGTGGAAGGAATGGAGGACTGG |
| | | | GAGAAGGAGAGTGCCCTTCAGAGGGCCAGAGAGGACATCTTCATGGAGACCCTGAGCGAT |
| | | | ATCATGGACTACTACAAGGACAGAGAAGGGTCTCACACCTTTCAAGGAGCATTCGGCTGT |
| | | | GAGCTCCGGAATAACGAAAGTAGTGGAGCATTCTGGGGGTACGCCTACGATGGCCAGGAC |
| | | | TTCATCAAGTTCGACAAAGAAATCCCAGCCTGGGTCCCTCTGGACCCAGCAGCTCAGAAC |
| | | | ACCAAGCGGAAGTGGGAAGCAGAGGCAGTCTACGTGCAGCGGGCCAAGGCCTACCTGGAA |
| | | | GAGGAGTGCCCAGGGATGCTGCGGAGGTACCTGCCCTACAGCAGGACCCACCTGGACCGA |
| | | | CAAGAGTCTCCTTCTGTGTCGGTCACCGGGCACGCGGCTCCAGGGCACAAGAGGACACTC |
| | | | AAGTGCCTGGCCTACGACTTCTACCCACGAAGCATCGGGCTGCACTGGACTCGGGCTGGT |
| | | | GACGCCCAGGAGGCTGAGTCAGGGGGCGACGTGCTCCCCAGCGGCAACGGCACTTACCAG |
| | | | TCCTGGGTGGTGGTGGGGGTCCCCCCTGAGGACCAAGCCCCGTACTCCTGCCACGTGGAG |
| | | | CACAGAAGCCTGACCCGGCCCCTCACCGTCCCGTGGGACCCGCGGCAACAAGCCGAG |

TABLE 1-12
MW:17kDa

| SPOT No. | | | \textcircled{13} |
|---|---|---|---|
| protein | Isoelectric | Range | 4-10 |
| | point | Preferably | 4.5-9 |
| | | More preferably | 5-8 |
| | Molecular | Range | 5-30 |
| | weight | Preferably | 10-25 |
| | | More preferably | 15-20 |
| | Searched database | | Uniprot |
| | Biological species | | Cow (Bos taurus) |
| | accession No. | | P79345 |
| | Protein name | | intracellular cholesterol transporter 2 |
| | Full-length | | EPVKFKDCGSWVGVIKEVNVSPCPTQPCKLHRGQSYSVNVT |
| | sequence | | FTSNTQSQSSKAVVHGIVMGIPVPFPIPESDGCKSGIRCPIEKDKTYNYVNKLPVKNEYP |
| | (SEQ ID NO: 24) | | SIKVVVEWELTDDKNQRFFCWQIPIEVEA |
| DNA | Searched database | | GenBank |
| | accession No. | | CAA59794.1 |
| | Full-length | | GAG |
| | sequence | | CCGGTGAAATTCAAGGACTGCGGTTCTTGGGTCGGAGTTATAAAGGAAGTGAATGTGAGC |
| | (SEQ ID NO: 23) | | CCATGTCCCACCCAGCCTTGCAAACTGCACAGAGGACAGTCTTACAGTGTTAATGTCACG |
| | | | TTCACCAGTAATACTCAGTCTCAAAGTAGCAAGGCTGTGGTACATGGCATTGTGATGGGC |
| | | | ATCCCAGTTCCCTTTCCCATTCCTGAGTCTGATGGTTGTAAGTCTGGAATCAGATGCCCC |
| | | | ATCGAAAAAGATAAGACCTATAACTACGTGAACAAACTACCCGTGAAGAATGAATACCCC |
| | | | TCCATAAAAGTGGTGGTGGAGTGGGAACTCACGGATGACAAAAACCAACGTTTCTTCTGC |
| | | | TGGCAGATCCCAATAGAGGTTGAAGCC |

TABLE 1-13
MW:43kDa

| SPOT No. | | | \textcircled{14} |
|---|---|---|---|
| protein | Isoelectric | Range | 3-8 |
| | point | Preferably | 3.5-7 |
| | | More preferably | 4-6 |
| | Molecular | Range | 20-70 |
| | weight | Preferably | 25-60 |
| | | More preferably | 30-50 |
| | Searched database | | Uniprot |
| | Biological species | | Cow (Bos taurus) |
| | accession No. | | Q32PJ2 |
| | Protein name | | Apolipoprotein A-IV |
| | Full-length | | EVNADQVATVIWDYFSQLGNNAKKAVEHIQKSELTQQLNT |
| | sequence | | LFQDKLGEVSTYTDDLQKKLVPFATELHERLIKDSEKLKEEIRKELEDLRARLLPHATEV |
| | (SEQ ID NO: 26) | | SQKIGDNVRELQQRLGPYAEELRTQVDTQAQQLRRQLTPYVERMEKVMRQNLDQLQASLA |
| | | | PYAEELQATVNQRVEELKGRLTPYADQLQTKIEENVEELRRSLAPYAQDVQGKLNHQLEG |
| | | | LAFQMKKHAEELKAKISAKAEELRQGLVPLVNSVHGSQLGNAEDLQKSLAELSSRLDQQV |
| | | | EDFRRTVGPYGETFNKAMVQQLDTLRQKLGPLAGDVEDHLSFLEKDLRDKVSSFFNTLKE |
| | | | KESQAPALPAQEEMPVPLGG |
| DNA | Searched database | | GenBank |
| | accession No. | | AAI08097.1 |
| | Full-length | | GAGGTCAATGCCGACCAGGTGGCCACCGTGATTTGGGACTACTTCAGCCAGCTGGGCAAC |
| | sequence | | AATGCCAAGAAGGCGGTGGAACATATCCAGAAGTCTGAGCTCACCCAGCAGCTCAACACC |
| | (SEQ ID NO: 25) | | CTCTTCCAAGATAAACTTGGGGAAGTGAGCACCTACACGGACGACCTGCAGAAGAAGCTG |
| | | | GTGCCCTTTGCCACGGAACTGCATGAACGGCTGACCAAGGACTCCGAGAAGCTGAAGGAG |
| | | | GAGATTCGGAAGGAGCTGGAGGACCTGCGGGCCCGGCTGCTGCCCCACGCCACGGAGGTG |
| | | | AGCCAGAAGATCGGGGACAACGTGCGCGAGCTACAGCAGCGCCTGGGACCATACGCAGAG |
| | | | GAGCTGCGCACCCAGGTCGACACCCAGGCGCAGCAGCTGCGGCGCCAGCTGACGCCCTAT |
| | | | GTGGAGCGCATGGAGAAGGTGATGCGGCAGAACCTGGACCAGCTGCAGGCCTCGCTGGCG |
| | | | CCCTACGCCGAGGAGCTCCAGGCCACGGTCAACCAGCGCGTGGAAGAGCTCAAGGGGCGC |
| | | | CTGACGCCCTACGCGGACCAGCTCCAGACCAAGATCGAGGAGAACGTGGAGGAACTGCGC |
| | | | CGCAGCCTGGCCCCTTATGCTCAGGACGTCCAGGGGAAGCTCAACCACCAGCTGGAGGGC |
| | | | CTGGCCTTTCAGATGAAGAAGCATGCCGAGGAGCTAAAGGCCAAGATCTCAGCCAAAGCC |
| | | | GAGGAGCTGCGGCAGGGGCTGGTGCCCCTGGTCAACAGCGTGCATGGAAGTCAGCTGGGC |
| | | | AATGCCGAGGACTTGCAGAAGTCACTGGCCGAGCTGAGCAGCCGCCTGGACCAGCAGGTG |
| | | | GAGGACTTCCGCCGCACCGTGGGGCCCTACGGCGAGACCTTCAACAAAGCCATGGTGCAG |
| | | | CAGCTGGACACGCTCAGGCAGAAGCTGGGCCCCCTGGCGGGGGACGTGGAAGATCACCTG |
| | | | AGTTTCCTGGAGAAGGATCTGAGGGACAAGGTCAGCAGCTTCTTCAACACCCTCAAGGAG |
| | | | AAAGAGAGCCAGGCCCCCGCCCTCCCCGCGCAGGAGGAGATGCCCGTCCCATTGGGGGGC |

TABLE 1-14

| | SPOT | \textcircled{1} | \textcircled{2}\textcircled{3} | \textcircled{4} | \textcircled{5} | \textcircled{6} | \textcircled{7} |
|---|---|---|---|---|---|---|---|
| | [symbol] | NCBI | Uniprot | Uniprot | NCBI | Uniprot | Uniprot |
| | accession | [symbol] | G3XAN3 | G3MX20 | [symbol] | F1N1N6 | [symbol] |
| Grouping | protein name | immunoglobulin M | Beta-1 metal- | Lactoperoxidase | In heavy chain | [symbol] | Polymeric |
| based on | | heavy chain | binding globulin | | precursor | | immunoglobulin |
| functions | | secretory form | | | [symbol] | | receptor |
| Group 1 | Signal transduction | ● | ● | ● | ● | ● | |
| Group 2 | Immune system | ● | | | ● | | ● |
| Group 3 | glycoprotein | | | | | | |
| Group 4 | cholesterol binding | | | | | | |
| | \textcircled{8} | \textcircled{9} | \textcircled{10} | \textcircled{11} | \textcircled{12} | \textcircled{13} | \textcircled{14} |
| | Uniprot | Uniprot | Uniprot | Uniprot | Uniprot | Uniprot | Uniprot |
| | [symbol] | [symbol] | [symbol] | [symbol] | [symbol] | [symbol] | Q32PJ2 |
| Grouping | [symbol] | Immunoglobulin light | Glycoprotein 2 | [symbol] | Zinc-alpha-2- | [symbol] | Apolipoprotein A-IV |
| based on | | chain, lambda [symbol] | | | glycoprotein | cholesterol | |
| functions | | [symbol] | | | | transporter 2 | |
| Group 1 | | | | | | | |
| Group 2 | ● | ● | | | | | |
| Group 3 | | | ● | ● | ● | | |
| Group 4 | | | | | | ● | ● |

[symbol]

indicates data missing or illegible when filed

Spots 2 and 3 are derived from the same protein. The total of 14 different proteins (1) to (13) have been identified as new milk-derived antigens:

(1) immunoglobulin M heavy chain secretory form (spot 1);

(2) beta-1 metal-binding globulin (spots 2 and 3);

(3) lactoperoxidase (spot 4);

(4) Ig heavy chain precursor (B/MT.4A.17. H5. A5) (spot 5);

(5) perilipin (spot 6);

(6) polymeric immunoglobulin receptor (spot 7);

(7) C3-beta-c (spot 8);

(8) immunoglobulin light chain, lambda gene cluster (spot 9);

(9) glycoprotein 2 (spot 10);

(10) lactadherin (spot 11);

(11) zinc-alpha-2-glycoprotein (spot 12);

(12) intracellular cholesterol transporter 2 (spot 13); and

(13) apolipoprotein A-IV (spot 14).

As shown in Table 2, proteins (1) to (13) are classified under 4 groups, each sharing a function:

group 1(signal transduction function): proteins (1), (2), (3), (4), and (5);

group 2(immune system function): proteins (1), (4), (6), (7), and (8);

group 3 (glycoprotein function): proteins (9), (10), and (11); and

group 4 (cholesterol binding function): proteins (12) and (13).

The antigen of spot 1 in the invention is not limited and may be any of the following (1-a) to (1-f).

(1-a) A protein comprising the amino acid sequence set forth in SEQ ID NO: 2;

(1-b) a protein comprising an amino acid sequence with deletion, substitution, insertion or addition of one or several amino acids in SEQ ID NO: 2;

(1-c) a protein comprising an amino acid sequence having 70% or higher identity to the amino acid sequence set forth in SEQ ID NO: 2;

(1-d) a protein comprising an amino acid sequence encoded by a nucleotide sequence with deletion, substitution, insertion or addition of one or several nucleotides in SEQ ID NO: 1;

(1-e) a protein comprising an amino acid sequence encoded by a nucleotide sequence having 70% or higher identity to the nucleotide sequence set forth in SEQ ID NO: 1; and

(1-f) a protein comprising an amino acid sequence encoded by a nucleic acid that hybridizes under stringent conditions with a nucleic acid consisting of a nucleotide sequence complementary to the nucleotide sequence set forth in SEQ ID NO: 1.

The antigen of spot 2 or 3 in the invention is not limited and may be any of the following (2-a) to (2-f).

(2-a) A protein comprising the amino acid sequence set forth in SEQ ID NO: 4;

(2-b) a protein comprising an amino acid sequence with deletion, substitution, insertion or addition of one or several amino acids in SEQ ID NO: 4;

(2-c) a protein comprising an amino acid sequence having 70% or higher identity to the amino acid sequence set forth in SEQ ID NO: 4;

(2-d) a protein comprising an amino acid sequence encoded by a nucleotide sequence with deletion, substitution, insertion or addition of one or several nucleotides in SEQ ID NO: 3;

(2-e) a protein comprising an amino acid sequence encoded by a nucleotide sequence having 70% or higher identity to the nucleotide sequence set forth in SEQ ID NO: 3; and

(2-f) a protein comprising an amino acid sequence encoded by a nucleic acid that hybridizes under stringent conditions with a nucleic acid consisting of a nucleotide sequence complementary to the nucleotide sequence set forth in SEQ ID NO: 3.

The antigen of spot 4 in the invention is not limited and may be any of the following (3-a) to (3-f).

(3-a) A protein comprising the amino acid sequence set forth in SEQ ID NO: 6;

(3-b) a protein comprising an amino acid sequence with deletion, substitution, insertion or addition or one or several amino acids in SEQ ID NO: 6;

(3-c) a protein comprising an amino acid sequence having 70% or higher identity to the amino acid sequence set forth in SEQ ID NO: 6;

(3-d) a protein comprising an amino acid sequence encoded by a nucleotide sequence with deletion, substitution, insertion or addition of one or several nucleotides in SEQ ID NO: 5;

(3-e) a protein comprising an amino acid sequence encoded by a nucleotide sequence having 70% or higher identity to the nucleotide sequence set forth in SEQ ID NO: 5; and

(3-f) a protein comprising an amino acid sequence encoded by a nucleic acid that hybridizes under stringent conditions with a nucleic acid consisting of a nucleotide sequence complementary to the nucleotide sequence set forth in SEQ ID NO: 5.

The antigen of spot 5 in the invention is not limited and may be any of the following (4-a) to (4-f).

(4-a) A protein comprising the amino acid sequence set forth in SEQ ID NO: 8;

(4-b) a protein comprising an amino acid sequence with deletion, substitution, insertion or addition of one or several amino acids in SEQ ID NO: 8;

(4-c) a protein comprising an amino acid sequence having 70% or higher identity to the amino acid sequence set forth in SEQ ID NO: 8;

(4-d) a protein comprising an amino acid sequence encoded by a nucleotide sequence with deletion, substitution, insertion or addition of one or several nucleotides in SEQ ID NO: 7;

(4-e) a protein comprising an amino acid sequence encoded by a nucleotide sequence having 70% or higher identity to the nucleotide sequence set forth in SEQ ID NO: 7; and

(4-f) a protein comprising an amino acid sequence encoded by a nucleic acid that hybridizes under stringent conditions with a nucleic acid consisting of a nucleotide sequence complementary to the nucleotide sequence set forth in SEQ ID NO: 7.

The antigen of spot 6 in the invention is not limited and may be any of the following (5-a) to (5-f).

(5-a) A protein comprising the amino acid sequence set forth in SEQ ID NO: 10;

(5-b) a protein comprising an amino acid sequence with deletion, substitution, insertion or addition of one or several amino acids in SEQ ID NO: 10;

(5-c) a protein comprising an amino acid sequence having 70% or higher identity to the amino acid sequence set forth in SEQ ID NO: 10;

(5-d) a protein comprising an amino acid sequence encoded by the nucleotide sequence with deletion, substitution, insertion or addition of one or several nucleotides in SEQ ID NO: 9;

(5-e) a protein comprising an amino acid sequence encoded by a nucleotide sequence having 70% or higher identity to the nucleotide sequence set forth in SEQ ID NO: 9; and

(5-f) a protein comprising an amino acid sequence encoded by a nucleic acid that hybridizes under stringent conditions with a nucleic acid consisting of a nucleotide sequence complementary to the nucleotide sequence set forth in SEQ ID NO: 9.

The antigen of spot 7 in the invention is not limited and may be any of the following (6-a) to (6-f).

(6-a) A protein comprising the amino acid sequence set forth in SEQ ID NO: 12;

(6-b) a protein comprising an amino acid sequence with deletion, substitution, insertion or addition of one or several amino acids in SEQ ID NO: 12;

(6-c) a protein comprising an amino acid sequence having 70% or higher identity to the amino acid sequence set forth in SEQ ID NO: 12;

(6-d) a protein comprising an amino acid sequence encoded by a nucleotide sequence with deletion, substitution, insertion or addition of one or several nucleotides in SEQ ID NO: 11;

(6-e) a protein comprising an amino acid sequence encoded by a nucleotide sequence having 70% or higher identity to the nucleotide sequence set forth in SEQ ID NO: 11; and

(6-f) a protein comprising an amino acid sequence encoded by a nucleic acid that hybridizes under stringent conditions with a nucleic acid consisting of a nucleotide sequence complementary to the nucleotide sequence set forth in SEQ ID NO: 11.

The antigen of spot 8 in the invention is not limited and may be any of the following (7-a) to (7-f).

(7-a) A protein comprising the amino acid sequence set forth in SEQ ID NO: 14;

(7-b) a protein comprising the amino acid sequence with deletion, substitution, insertion or addition of one or several amino acids in SEQ ID NO: 14;

(7-c) a protein comprising an amino acid sequence having 70% or higher identity to the amino acid sequence set forth in SEQ ID NO: 14;

(7-d) a protein comprising an amino acid sequence encoded by the nucleotide sequence with deletion, substitution, insertion or addition of one or several nucleotides in SEQ ID NO: 13;

(7-e) a protein comprising an amino acid sequence encoded by a nucleotide sequence having 70% or higher identity to the nucleotide sequence set forth in SEQ ID NO: 13; and

(7-f) a protein comprising an amino acid sequence encoded by a nucleic acid that hybridizes under stringent conditions with a nucleic acid consisting of a nucleotide sequence complementary to the nucleotide sequence set forth in SEQ ID NO: 13.

The antigen of spot 9 in the invention is not limited and may be any of the following (8-a) to (8-f).

(8-a) A protein comprising the amino acid sequence set forth in SEQ ID NO: 16;

(8-b) a protein comprising an amino acid sequence with deletion, substitution, insertion or addition of one or several amino acids in SEQ ID NO: 16;

(8-c) a protein comprising an amino acid sequence having 70% or higher identity to the amino acid sequence set forth in SEQ ID NO: 16;

(8-d) a protein comprising an amino acid sequence encoded by a nucleotide sequence with deletion, substitution, insertion or addition of one or several nucleotides in SEQ ID NO: 15;

(8-e) a protein comprising an amino acid sequence encoded by a nucleotide sequence having 70% or higher identity to the nucleotide sequence set forth in SEQ ID NO: 15; and

(8-f) a protein comprising an amino acid sequence encoded by a nucleic acid that hybridizes under stringent conditions with a nucleic acid consisting of a nucleotide sequence complementary to the nucleotide sequence set forth in SEQ ID NO: 15.

The antigen of spot 10 in the invention is not limited and may be any of the following (9-a) to (9-f).

(9-a) A protein comprising the amino acid sequence set forth in SEQ ID NO: 18;

(9-b) a protein comprising an amino acid sequence with deletion, substitution, insertion or addition of one or several amino acids in SEQ ID NO: 18;

(9-c) a protein comprising an amino acid sequence having 70% or higher identity to the amino acid sequence set forth in SEQ ID NO: 18;

(9-d) a protein comprising an amino acid sequence encoded by the nucleotide sequence with deletion, substitution, insertion or addition of one or several nucleotides in SEQ ID NO: 17;

(9-e) a protein comprising an amino acid sequence encoded by a nucleotide sequence having 70% or higher identity to the nucleotide sequence set forth in SEQ ID NO: 17; and

(9-f) a protein comprising an amino acid sequence encoded by a nucleic acid that hybridizes under stringent conditions with a nucleic acid consisting of a nucleotide sequence complementary to the nucleotide sequence set forth in SEQ ID NO: 17.

The antigen of spot 11 in the invention is not limited and may be any of the following (10-a) to (10-f).

(10-a) A protein comprising the amino acid sequence set forth in SEQ ID NO: 20;

(10-b) a protein comprising an amino acid sequence with deletion, substitution, insertion or addition of one or several amino acids in SEQ ID NO: 20;

(10-c) a protein comprising an amino acid sequence having 70% or higher identity to the amino acid sequence set forth in SEQ ID NO: 20;

(10-d) a protein comprising an amino acid sequence encoded by the nucleotide sequence with deletion, substitution, insertion or addition of one or several nucleotides in SEQ ID NO: 19;

(10-e) a protein comprising an amino acid sequence encoded by a nucleotide sequence having 70% or higher identity to the nucleotide sequence set forth in SEQ ID NO: 19; and

(10-f) a protein comprising an amino acid sequence encoded by a nucleic acid that hybridizes under stringent conditions with a nucleic acid consisting of a nucleotide sequence complementary to the nucleotide sequence set forth in SEQ ID NO: 19.

The antigen of spot 12 in the invention is not limited and may be any of the following (11-a) to (11-f).

(11-a) A protein comprising the amino acid sequence set forth in SEQ ID NO: 22;

(11-b) a protein comprising an amino acid sequence with deletion, substitution, insertion or addition of one or several amino acids in SEQ ID NO: 22;

(11-c) a protein comprising an amino acid sequence having 70% or higher identity to the amino acid sequence set forth in SEQ ID NO: 22;

(11-d) a protein comprising an amino acid sequence encoded by the nucleotide sequence with deletion, substitution, insertion or addition of one or several nucleotides in SEQ ID NO: 21;

(11-e) a protein comprising an amino acid sequence encoded by a nucleotide sequence having 70% or higher identity to the nucleotide sequence set forth in SEQ ID NO: 21; and

(11-f) a protein comprising an amino acid sequence encoded by a nucleic acid that hybridizes under stringent conditions with a nucleic acid consisting of a nucleotide sequence complementary to the nucleotide sequence set forth in SEQ ID NO: 21.

The antigen of spot 13 in the invention is not limited and may be any of the following (12-a) to (12-f).

(12-a) A protein comprising the amino acid sequence set forth in SEQ ID NO: 24;

(12-b) a protein comprising an amino acid sequence with deletion, substitution, insertion or addition of one or several amino acids in SEQ ID NO: 24;

(12-c) a protein comprising an amino acid sequence having 70% or higher identity to the amino acid sequence set forth in SEQ ID NO: 24;

(12-d) a protein comprising an amino acid sequence encoded by the nucleotide sequence with deletion, substitution, insertion or addition of one or several nucleotides in SEQ ID NO: 23;

(12-e) a protein comprising an amino acid sequence encoded by a nucleotide sequence having 70% or higher identity to the nucleotide sequence set forth in SEQ ID NO: 23; and

(12-f) a protein comprising an amino acid sequence encoded by a nucleic acid that hybridizes under stringent conditions with a nucleic acid consisting of a nucleotide sequence complementary to the nucleotide sequence set forth in SEQ ID NO: 23.

The antigen of spot 14 in the invention is not limited and may be any of the following (13-a) to (13-f).

(13-a) A protein comprising the amino acid sequence set forth in SEQ ID NO: 26;

(13-b) a protein comprising an amino acid sequence with deletion, substitution, insertion or addition of one or several amino acids in SEQ ID NO: 26;

(13-c) a protein comprising an amino acid sequence having 70% or higher identity to the amino acid sequence set forth in SEQ ID NO: 26;

(13-d) a protein comprising an amino acid sequence encoded by the nucleotide sequence with deletion, substitution, insertion or addition of one or several nucleotides in SEQ ID NO: 25;

(13-e) a protein comprising an amino acid sequence encoded by a nucleotide sequence having 70% or higher identity to the nucleotide sequence set forth in SEQ ID NO: 25; and

(13-f) a protein comprising an amino acid sequence encoded by a nucleic acid that hybridizes under stringent conditions with a nucleic acid consisting of a nucleotide sequence complementary to the nucleotide sequence set forth in SEQ ID NO: 25.

In an embodiment, the above antigen proteins (1) to (13) and the below-described polypeptides (E1) to (E25) include the corresponding proteins or polypeptides, the amino acid residue(s) of which is modified by, for instance, phosphorylation, glycosylation, amino acylation, ring-opening, or deamidation.

Preferably, the above antigen proteins (1) to (13) and the below-described polypeptides (E1) to (E25) are allergy antigens.

As used herein, an amino acid sequence “with deletion, substitution, insertion or addition of one or several amino acids” means that the amino acid sequence of interest refers to an amino acid sequence in which one to several amino acids are deleted and/or substituted with other amino acid(s), other amino acid(s) are inserted, and/or other amino acid(s) are added. The number of “several amino acids” is not limited and means 200 or less, 100 or less, 50 or less, 30 or less, 20 or less, 15 or less, 12 or less, 10 or less, 8 or less, 6 or less, 4 or less, or 3 or less amino acids. Alternatively, the several amino acids mean 30% and preferably 25%, 20%, 15%, 10%, 5%, 3%, 2%, or 1% amino acids based on the full-length amino acid sequence.

The above substitution is preferably conservative substitution. In the conservative substitution, a certain amino acid residue(s) is replaced by a residue(s) having similar physicochemical characteristics. Any substitution is allowed unless the features of the original sequence structure are not substantially changed. For example, any substitution is allowed unless the substituted amino acid(s) destroys the helix present in the original sequence or destroys another type of the secondary structure characteristic of the original sequence. Hereinbelow, examples of a residue where the amino acid residue can be substituted by conservative substitution are illustrated while classified. However, the replaceable amino acid residue is not limited to those listed below.

Group A: leucine, isoleucine, valine, alanine, methionine, glycine, cysteine, or proline.

Group B: aspartic acid or glutamic acid.

Group C: asparagine or glutamine.

Group D: lysine or arginine.

Group E: serine or threonine.

Group F: phenylalanine, tyrosine, tryptophan, or histidine.

In the case of non-conservative substitution, a member of any one of the above groups may be replaced by a member of another group. For example, to exclude unwanted glycosylation, an amino acid(s) of the above group B, D, or E may be substituted by an amino acid(s) of any of the other groups. In addition, to prevent folding into a protein conformation, a cysteine may be deleted or substituted by another amino acid. In addition, to keep a hydrophilicity/hydrophobicity balance or to increase hydrophilicity to make synthesis easy, an amino acid(s) may be substituted in consideration of amino acid hydropathy index (J. Kyte and R. Doolittle, J. Mol. Biol., Vol. 157, p. 105-132, 1982) as an indicator for amino acid hydrophilicity/hydrophobicity.

In another embodiment, an amino acid of interest may be substituted by an amino acid with less steric hindrance than the original amino acid. For example, an amino acid of group F may be substituted by an amino acid of any of group A, B, C, D, or E; or an amino acid with a charge may be substituted by an amino acid without a charge. For instance, a group B amino acid may be substituted by a group C amino acid. This may improve binding ability to IgE antibodies.

As used herein, the identity (%) between two amino acid sequences may be determined by a visual test or mathematical calculation. In addition, a computer program may be used to determine the identity (%). Examples of such a computer program include BLAST or ClustalW. Various conditions (parameters) of identity search by, in particular, BLAST program have been described by Altschul and colleagues (Nucl. Acids. Res., 25, p. 3389-3402, 1997). The information (BLAST manual, Altschul, et al., NCB/NLM/NIH Bethesda, Md. 20894; Altschul et al.) is publicly available from the website of NCBI or DNA Data Bank of Japan (DDBJ). In addition, a program such as genetic information processing software GENETYX Ver.7 (GENETYX), DNASIS Pro (Hitachi Software), or Vector NTI (Infomax) may be used for determination.

As used herein, a nucleotide sequence with “deletion, substitution, insertion or addition of one or several nucleotides” means that the nucleotide sequence of interest refers to a nucleotide sequence in which one or several nucleotides are deleted or substituted by other nucleotide(s); or other nucleotide(s) is inserted and/or added to give a nucleotide sequence. The number of “several nucleotides” is not limited and means 600 or less, 300 or less, 150 or less, 100 or less, 50 or less, 30 or less, 20 or less, 15 or less, 12 or less, 10 or less, 8 or less, 6 or less, 4 or less, or 3 or less nucleotides. Alternatively, the several nucleotides mean 30% or preferably 25%, 20%, 15%, 10%, 5%, 3%, 2%, or 1% of nucleotides based on the full-length nucleotide sequence. It is preferable that the above nucleotide deletion, substitution, insertion, or addition causes no frame shift in the amino acid-encoding sequence.

As used herein, the identity (%) between two nucleotide sequences may be determined by a visual test or mathematical calculation. In addition, a computer program may be used to determine the identity (%). Examples of such a sequence comparison computer program include BLASTN program, which is available from the website (https://blast.ncbi.nlm.nih.gov/Blast.cgi) of United States National Library of Medicine (Altschul et al. (1990) J. Mol. Biol. 215: 403-10): Ver. 2.2.7 or WU-BLAST 2.0 algorithm. Standard default parameters of WU-BLAST 2.0 may be set using those described in the following internet site: http://blast.wustl.edu.

As used herein, the wording “under stringent conditions” means to hybridize under moderately or highly stringent conditions. Specifically, the moderately stringent conditions may be easily determined, based on, for example, the DNA length, by those having ordinary skill. The essential conditions are disclosed by Sambrook et al., Molecular Cloning: A Laboratory Manual, 3rd ed., Chapters 6 to 7, Cold Spring Harbor Laboratory Press, 2001. The moderately stringent hybridization conditions are preferably conditions of 1×SSC to 6×SSC at 42° C. to 55° C., more preferably conditions of 1×SSC to 3×SSC at 45° C. to 50° C., and most preferably conditions of 2×SSC at 50° C. When the hybridization solution contains, for instance, about 50% formamide, the temperature 5 to 15° C. lower than the above temperature should be adopted. Examples of the washing conditions include 0.5×SSC to 6×SSC at 40° C. to 60° C. During hybridization or washing, 0.05% to 0.2% SDS is routinely added, and about 0.1% SDS is preferably added. The highly stringent conditions may also be easily determined, based on, for example, the DNA length, by those skilled in the art. In general, the highly stringent conditions include hybridization and/or washing at a temperature higher than and/or a salt concentration lower than those under the moderately stringent conditions. The hybridization conditions are, for example, conditions of 0.1×SSC to 2×SSC at 55° C. to 65° C., more preferably conditions of 0.1×SSC to 1×SSC at 60° C. to 65° C., and most preferably conditions of 0.2×SSC at 63° C. Washing conditions are, for example, 0.2×SSC to 2×SSC at 50° C. to 68° C. and more preferably 0.2×SSC at 60 to 65° C.

The variants corresponding to items (b) to (f) of the above (1) to (13) preferably include, but are not limited to, the below-described epitope (variant) amino acid sequences. The epitope amino acid sequences included are not limited to one sequence, and preferably include all of the sequences of epitopes derived from each of the respective proteins. For example, (E1) and (E2) are epitopes derived from protein (1). (1-b) to (1-f) preferably include at least one of an (E1) or (E2) epitope amino acid sequence (including a variant).

Each antigen may be separated/purified from milk by combining protein purification procedures well-known to those skilled in the art. Alternatively, each antigen may be separated/purified by expressing the antigen as a recombinant protein by a gene recombinant technology well-known to those skilled in the art and then performing a protein purification procedure well-known to those skilled in the art.

Examples of the protein purification procedure include: solubility-based methods (e.g., salting out, solvent precipitation); methods using differences in molecular weight (e.g., dialysis, ultrafiltration, gel filtration, SDS-PAGE); charge-based methods (e.g., ion exchange chromatography, hydroxyapatite chromatography); specific affinity-based methods (e.g., affinity chromatography); methods using differences in hydrophobicity (e.g., reversed-phase high-performance liquid chromatography); or methods using differences in isoelectric point (e.g., isoelectric focusing).

Each protein may be prepared using a gene recombinant technology by constructing an expression vector comprising the antigen-coding nucleic acid, transfecting or transforming the expression vector in a suitable host cell, culturing the host cell under conditions fit for expressing a recombinant protein, and collecting the recombinant protein expressed in the host cell.

The “vector” is a nucleic acid capable of being used for introducing, into a host cell, nucleic acid linked to the vector. The “expression vector” is a vector capable of inducing expression of a protein encoded by the nucleic acid introduced in the vector. Examples of the vector include a plasmid vector or a viral vector. Those skilled in the art can select, depending on the type of host cell used, an expression vector suitable for expressing a recombinant protein.

The “host cell” is a cell transfected or transformed with a vector. The host cell can be selected, if appropriate, depending on a vector to be used, by those skilled in the art. The host cell may be derived from a prokaryote such as E. coli. When a prokaryotic cell such as E. coli is used as a host, an antigen of the invention may include an N-terminal methionine residue so as to easily express a recombinant protein in the prokaryotic cell. This N-terminal methionine may be cleaved from the recombinant protein after expression. Alternatively, the host cell may be a eukaryote-derived cell such as a unicellular eukaryote (e.g., yeast), a plant cell, or an animal cell (e.g., a human cell, a monkey cell, a hamster cell, a rat cell, a mouse cell, an insect cell), or a Bombyx mori.

Gene transfer or genetic transformation of an expression vector into a host cell may be carried out, if appropriate, using a known technique by those skilled in the art. In addition, those skilled in the art can select, if appropriate, conditions suitable for expressing a recombinant protein in accordance with the type of host cell and then culturing the host cell to express the recombinant protein. Then, the host cell expressing the recombinant protein may be homogenized. The resulting homogenate may be subjected to a suitable combination of the above-mentioned protein purification procedures. Finally, the antigen as expressed as the recombinant protein can be separated/purified. The above expression vector or synthesized double-stranded DNA, or mRNA transcribed from the DNA may be introduced into a cell-free protein synthesis system for expression. The expressed protein may be separated/purified to prepare the antigen.

Each antigen in the invention can specifically bind to an IgE antibody from an allergic patient.

Diagnosis Kit/Diagnosis Method (1)

The invention provides a method for providing an indicator for diagnosing an allergy in a subject, comprising the steps of:

(i) contacting a sample obtained from the subject with an antigen, wherein the sample is a solution comprising an IgE antibody;

(ii) detecting binding between the IgE antibody present in the sample obtained from the subject and the antigen; and

(iii) when the binding between the IgE antibody in the subject and the antigen is detected, an indicator of the fact that the subject is allergic is provided, wherein the antigen is at least one protein specified as any of the antigens (1) to (13).

The “allergy” is not limited, and is, in one embodiment, an allergy to milk and preferably an allergy to cow's milk.

As used herein, the term “diagnosis” generally includes not only a (definitive) diagnosis by a physician, but also mere “detection,” including the possibility. As used herein, the term “diagnosis” or “detection” refers to, in one embodiment, in vivo, in vitro, or ex vivo “diagnosis” or “detection. Preferred is in vitro “diagnosis” or “detection”.

A sample obtained from a subject is a solution comprising an IgE antibody collected from the subject. Examples of such a solution include blood, saliva, sputum, snot, urine, sweat, or tears. Each sample obtained from the subject may be pretreated to increase the concentration of IgE antibody in the sample prior to contact with an antigen. The sample pretreatment may include, for example, obtaining serum or plasma from blood. In addition, a Fab portion, which is the portion of binding to an antigen, may be purified. In a particularly preferred embodiment, step (i) is carried out by bringing the antigen into contact with an IgE antibody in serum obtained from the subject.

The IgE antibody may be an IgE antibody itself or a mast cell to which the IgE antibody is bound.

The contact or binding between the sample obtained from the subject and the antigen may be detected by a known procedure. Examples of such a procedure include ELISA (Enzyme-Linked Immunosorbent Assay), sandwich immunoassay, immunoblotting, immunoprecipitation, or immunochromatography-mediated detection. In any of them, the technique includes: bringing an IgE antibody from a subject into contact with an antigen for binding; applying an enzyme-labeled secondary antibody to the IgE antibody specifically bound to the antigen; adding an enzyme substrate (usually a chromogenic or luminescent reagent) to detecting an enzymatic reaction product; and then detecting binding of the antigen to the IgE antibody from the subject. Alternatively, a fluorescently labeled secondary antibody may be used for detection in the procedure. Otherwise, detection by surface plasmon resonance (SPR) or other measuring procedures that can be used to evaluate the binding of an antigen to an IgE antibody may be used. Multiple antigen-specific IgE antibodies may be mixed.

Each antigen may be in the form of an isolated antigen immobilized on or inside a carrier. In this case, ELISA, sandwich immunoassay, immunochromatography, surface plasmon resonance, etc. can be used in steps (i) and (ii), and in step (i), the sample obtained from the subject is brought into contact with a surface on which the antigen is immobilized. The isolated antigen may be obtained from an object of interest (e.g., a raw material, a processed product) by isolation/purification using a combination of protein purification procedures well-known to those skilled in the art, or by preparation using gene recombinant technology. The antibody may also be attached onto a surface.

The antigen is not necessarily immobilized on a carrier. In this case, flow cytometry, etc., can be used in steps (i) and (ii), and the presence of the antigen bound to an antibody can be confirmed by laser light. Examples include the basophil activation test (BAT). Other examples include the histamine release test (HRT), in which an antigen is further brought into contact with blood cells in a sample to determine whether or not histamine is released.

Each antigen may also be separated by 2D electrophoresis, transferred, and detected by immunoblotting. Here, 2D electrophoresis is a technique for separating protein samples by performing isoelectric focusing in the first dimension and SDS-PAGE in the second dimension. In this case, the conditions of 2D electrophoresis are not particularly limited as long as each antigen in the invention can be separated. For example, the conditions for 2D electrophoresis described in the above section “To Identify Antigen” can be used. Alternatively, the conditions for electrophoresis may be determined by referring to the descriptions in the above-mentioned PTLs 1 to 4. The 2D electrophoresis can be performed using conditions satisfying the following at least one condition selected from the group consisting of:

(A) when the length of a gel as a first-dimensional isoelectric focusing gel is in the range of 5 to 10 cm, the pH range of the gel is from 3 to 10, and the pH gradient of the gel in the electrophoresis direction is “a” for the gel length up to pH 5, “b” for the gel length between pH 5 and 7, and “c” for the gel length above pH 7, the relationships “a<b” and “b>c” are satisfied;

(B) in the case of (A) where the total length of the gel is set to 1, “a” is in the range 0.15-0.3, “b” is in the range 0.4-0.7, and “c” is in the range 0.15-0.3;

(C) in the first dimensional isoelectric focusing, a constant voltage step is performed by applying a constant voltage with a value in the range of 100 V to 600 V to each gel comprising the sample, and a voltage increase step is started to increase the voltage from the above constant voltage after an electrophoresis change per 30 min of running becomes within the range of 5 μA;

(D) in the case of (C), the final voltage of the voltage increase step is in the range of 3000 V to 6000 V;

(E) the longitudinal gel length of the first-dimensional isoelectric focusing gel is from 5 to 10 cm, and the gel concentration at the base end of the second-dimensional electrophoresis gel in the electrophoresis direction is from 3 to 6%; and

(F) in the case of (E), the gel concentration at the tip of the second dimensional electrophoresis gel in the electrophoresis direction is set to be higher than that at the base end in the electrophoresis direction.

The above antigens (1) to (13) are an antigen that can specifically bind to an IgE antibody of an allergic patient (e.g., a patient with milk allergy). This makes it possible to provide an indicator indicating that the subject has the allergy when the binding of the antigen to the IgE antibody from the subject is detected.

The invention also provides a kit for diagnosing an allergy comprising at least one of the above antigens (1) to (13). The diagnosis kit of the invention may be used in a method for providing an indicator for diagnosing an allergy as described above, or in a diagnosis method described below. The diagnosis kit of the invention may comprise at least one of the above antigens (1) to (13) as well as an enzyme-labeled anti-IgE antibody and a chromogenic or luminescent substrate that serves as a substrate for the enzyme. It is also possible to use a fluorescently labeled anti-IgE antibody. In the diagnosis kit of the invention, the antigen may be provided in a state where the antigen is immobilized on or inside a carrier. The diagnosis kit of the invention may also be provided with instructions on the procedure for diagnosis and a package comprising the instructions.

In another embodiment, the above diagnosis kit contains a companion diagnostic agent for allergy. The companion diagnostic agent may be used to identify a patient who is expected to benefit from a drug or who is at a risk of serious adverse effects from a drug, or to study the response of a drug to optimize treatment with the drug. Here, the treatment optimization includes, for example, determining a dosage regimen and a dose, deciding discontinuation of administration, and identifying which allergen component is used to produce immunological tolerance.

The invention also provides a composition for diagnosing an allergy comprising at least one of the above antigens (1) to (13). The diagnostic composition of the invention may be used in the following diagnosis method. The diagnostic composition of the invention may optionally contain a pharmaceutically acceptable carrier and/or an additive(s) commonly used with an antigen of the invention.

An embodiment of the invention provides a method for diagnosing an allergy in a subject, comprising the steps of:

(i) contacting a sample obtained from the subject with an antigen;

(ii) detecting binding of the antigen to an IgE antibody in the sample obtained from the subject; and

(iii) determining that the subject has the allergy when the binding of the antigen to the IgE antibody from the subject is detected,

wherein the antigen is at least one protein specified as any of the antigens (1) to (13). Here, steps (i) and (ii) are performed as described for each step of the method of providing an indicator for diagnosing an allergy.

In another embodiment, the invention provides a method for diagnosing an allergy in a subject, comprising administering to the subject at least one of the above antigens (1) to (13). The method may be in the form of a skin test characterized by applying the antigen to the skin. Examples of the form of the skin test include: a prick test, in which a diagnostic composition is applied onto the skin, a small cut is given to a degree to which no bleeding occurs, and an antigen is permeated into the skin; a scratch test, in which a diagnostic composition is applied, and the skin is slightly scratched to observe the resulting reaction; a patch test, in which a diagnostic composition in the form of a cream or ointment is applied to the skin and the reaction is observed; or an intracutaneous test, in which an antigen is administered intracutaneously and the reaction is observed. In the case of occurrence of a skin reaction, such as skin swelling, in an antigen-applied site, the subject is diagnosed as having an allergy. Here, the amount of antigen applied to the skin may be, for example, a dose of 100 μg or less per application.

In the allergy diagnosis, a loading test is often performed to identify the antigen. At least one of the above antigens (1) to (13) may be used as an active ingredient for the loading test to diagnose an allergy. Here, each antigen protein used in the loading test may be an expressed and purified protein. Examples include pollen rice, in which the cedar pollen antigen gene is transformed into rice and the antigen protein is expressed in the rice. In this way, the antigen protein may be expressed in a raw material/processed product.

In one embodiment, the above diagnostic composition or diagnosis kit may be used for a prick test, scratch test, patch test, intracutaneous test, etc.

In addition, another embodiment of the invention provides at least one of the above antigens (1) to (13) for use in allergy diagnosis. Here included is providing at least one of the above antigens (1) to (13) in a mixture with a known antigen(s).

Still another embodiment of the invention provides use of at least one of the above antigens (1) to (13) in the manufacture of a composition for diagnosing an allergy.

Composition/Treatment Method (1)

The invention provides a composition comprising at least one of the above antigens (1) to (13).

In one embodiment, the composition of the invention is a pharmaceutical composition. In one embodiment, the composition of the invention is a quasi-drug composition or a non-medicinal composition (e.g., a cosmetic composition, a food composition).

In one embodiment, the above composition is used to treat an allergy (e.g., an allergy to milk). As used herein, the “allergy treatment” means increasing the limit of the amount of antigen that does not cause any symptom when taken into the body, with the ultimate goal of achieving a state (remission) in which the disease does not develop with normal intake of the antigen.

The invention also provides a method for treating an allergy, comprising administering to a patient in need of allergy treatment at least one of the above antigens (1) to (13).

Another embodiment of the invention provides at least one of the above antigens (1) to (13) for use in allergy treatment. Still another embodiment of the invention provides use of at least one of the above antigens (1) to (13) in the manufacture of a drug for an allergy.

In the allergy treatment, desensitization therapy, which aims to induce immune tolerance by administering an antigen to a patient, is often used. At least one of the above antigens (1) to (13) may be used as an active ingredient for the allergy desensitization therapy. The antigenic protein used for the desensitization therapy may be an expressed and purified protein, or may be an antigen protein expressed in a raw material/processed product, such as pollen rice, in which the cedar pollen antigen gene is transformed into rice and the resulting antigen protein is expressed in the rice.

The composition of the invention may be administered by a regular administration route. Examples of the regular administration route include oral, sublingual, transdermal, intracutaneous, subcutaneous, intravascular, nasal, intramuscular, intraperitoneal, or intrarectal administration.

The composition of the invention may be used as a composition comprising a pharmaceutically acceptable adjuvant, an excipient, or various types of additive(s) (e.g., a stabilizer, a dissolution aid, an emulsifier, a buffer, a preservative, a colorant) generally used together with an antigen of the invention, if necessary. The dosage form of the composition can be selected, if appropriate, depending on the administration route by those skilled in the art. Examples of the form include tablets, capsules, pastilles, sublingual tablets, injections, nasal sprays, cataplasms, liquids, creams, lotions, or suppositories. The dosage, frequency of administration, and/or period of administration of the composition of the invention may be selected, if appropriate, by a physician in view of, for instance, the route of administration, symptoms, and patient characteristics such as the age and body weight. For example, in the case of adults, a dose of 100 μg or less per administration may be given. The dosing interval may be, for example, daily, once weekly, twice monthly, or once every three months. The administration period may be, for example, several weeks to several years. The dosage in the administration method may be increased in a stepwise manner over the course of the administration period.

Tester Composition (1)

The invention provides a tester composition comprising an antibody against at least one of the above antigens (1) to (13).

The antibody can be produced by a conventional procedure. For example, the antibody may be produced by immunizing a mammal such as a rabbit with any of the antigen antigens (1) to (13). The antibody may be an Ig antibody, a polyclonal antibody, a monoclonal antibody, or an antigen-binding fragment thereof (e.g., a Fab, F(ab′)2, Fab′).

In addition, in the tester composition described above, the antibody may be provided in a form immobilized or bound onto or inside a carrier. The carrier is not particularly limited as long as the carrier can be used for detection of antibody-antigen binding. Any carrier known to those skilled in the art can be utilized.

Examples of the method for determining the presence or absence of an antigen include:
a method comprising bringing a produced Ig antibody-comprising tester composition into contact with a sample prepared from a raw material/processed product, detecting biding of the antibody to an antigen in the sample by using, for instance, ELISA, and determining, when the binding of the Ig antibody to the antigen is detected, that the antigen is contained in the raw material/processed product of interest; or
a method comprising soaking a raw material/processed product into a filter paper and allowing a reaction with an antibody solution so as to detect an antigen contained therein.

Another embodiment of the invention includes a tester composition for determining the presence or absence of an allergic antigen in an object of interest, comprising a primer having a nucleotide sequence complementary to a portion of at least one of the nucleotide sequence set forth in SEQ ID NO: 1, 3, 5, 7, 9, 11, 13, 15, 17, 19, 21, 23, or 25. The above primer is not limited and, for example, has a nucleotide sequence complementary to, preferably, 12, 15, 20, or 25 nucleotides in a 3′-terminal portion or a center portion of at least one sequence of the nucleotide sequence set forth in SEQ ID NO: 1, 3, 5, 7, 9, 11, 13, 15, 17, 19, 21, 23, or 25. Particularly when the object of interest is mRNA, a primer complementary to the poly A tail is included. In a preferred embodiment, the above primer-comprising tester composition may further comprise a primer comprising a nucleotide sequence of a 5′-terminal portion, preferably a nucleotide sequence consisting of 12, 15, 20, or 25 nucleotides, of at least one sequence of the nucleotide sequence set forth in SEQ ID NO: 1, 3, 5, 7, 9, 11, 13, 15, 17, 19, 21, 23, or 25.

For example, using the above complementary primer and, as a template, DNA or mRNA obtained from milk, cDNA may be amplified by PCR (Polymerase Chain Reaction) including RT-PCR (Reverse Transcription-Polymerase Chain Reaction), and the sequence of the cDNA amplified may be compared to that of SEQ ID NO: 1, 3, 5, 7, 9, 11, 13, 15, 17, 19, 21, 23, or 25 to determine the presence or absence of the antigen. Examples of the PCR amplification method include RACE (Rapid Amplification of cDNA End) protocol. In this case, even if a point mutation encoding the same amino acid exists in comparison of the amplified cDNA with SEQ ID NO: 1, 3, 5, 7, 9, 11, 13, 15, 17, 19, 21, 23, or 25, or even if the nucleotide sequence of the amplified cDNA has insertion, deletion, substitution, or addition of nucleotides in the nucleotide sequence set forth in SEQ ID NO: 1, 3, 5, 7, 9, 11, 13, 15, 17, 19, 21, 23, or 25, the antigen is determined to be present when the amino acid sequence encoded by the amplified cDNA has 70% or higher, preferably 80, 90, 95, 98, or 99% or higher identity to the amino acid sequence set forth in SEQ ID NO: 2, 4, 6, 8, 10, 12, 14, 16, 18, 20, 22, 24, or 26.

In one embodiment, the tester composition described above is used to test whether or not an antigen is included in an object of interest, for example, in a foodstuff (milk) or in a food production line. The above-mentioned tester composition may be used for quality inspection of production lines and pre-shipment products by manufacturers, or for checking whether or not an antigen is included in raw materials and processed products of interest by consumers themselves. In addition, the tester composition can also be used for checking inclusion of the antigen by measuring a change in the protein peak(s) by mass spectroscopy.

Method of Determining Presence or Absence of Antigen (1)

The invention encompasses a method for determining the presence or absence of any of the above antigens (1) to (13) in an object of interest, the method comprising bringing an antibody against at least one of the above antigens (1) to (13) into contact with a raw material/processed product (including a liquid).

The raw material may be a foodstuff or may be a cosmetic raw material, a pharmaceutical raw material, or the like. The processed product may be a processed food product or may be a cosmetic product, a pharmaceutical product, or the like.

The antibody, the method of producing the antibody, the method of bringing the antibody into contact with a raw material/processed product, and the binding of the antibody to an antigen are as described above in the “Tester Composition (1)”.

Antigen-Removed Food, Etc. (1)

The invention provides a raw material or processed product characterized in that at least one of the above antigens (1) to (13) is eliminated or reduced. In one embodiment, the “raw material or processed product” may be a “milk or processed milk product” and may be a “beef or processed beef product”.

The method of removing or reducing an antigen of the invention from a raw material or processed product is not limited. Any method may be carried out to remove or reduce the antigen as long as the antigen of the invention can be eliminated or reduced.

For example, a raw material (e.g., milk or beef) from which an antigen of the invention has been eliminated or reduced may be obtained from cows in which expression of the antigen of the invention has been altered using a genetic modification technology.

Any of techniques known to those skilled in the art may be used for the genetic modification technology. For instance, Oishi, et al. (Scientific Reports, Vol. 6, Article number: 23980, 2016, doi: 10.1038/srep23980) discloses that a genome editing technology CRISPER/Cas9 is applied to a chicken primordial germ cell to produce an ovomucoid gene-deleted individual. A similar technique may be used to obtain a cow in which expression of an antigen of the invention has been altered, and from such a cow, beef or milk from which an antigen of the invention has been eliminated or reduced may be obtained.

In addition, cross-breeding with a cow, in which the antigen is not expressed or the expression level is low, by using artificial insemination may allow generation of a cow in which the expression of the antigen of the invention has been eliminated or reduced, and from such a cow, beef or milk from which the antigen of the invention has been eliminated or reduced may be obtained. The cow may be artificially bred by a common procedure.

The processed product, from which an antigen of the invention has been eliminated or reduced, may be a processed product using, as a material, the raw material from which an antigen of the invention has been eliminated or reduced. In the case of using ordinary raw materials as materials, the antigen-removing or reducing treatment in the invention may be carried out before or after preparation of the processed product. Examples of the method of removing or reducing an antigen of the invention from a processed product using ordinary raw materials as materials include: a method for removing a protein component from a raw material/processed product by, for instance, high-pressure treatment, neutral salt solution-using elution, or high-temperature steaming; or a method for using thermal treatment- or acid treatment-mediated hydrolysis/denaturation/amino acid modification (e.g., chemical modification, elimination or the like of a side chain).

Method of Producing Raw Material or Processed Product with Antigen Eliminated or Reduced (1)

The invention provides a method for producing a raw material or processed product in which an antigen is eliminated or reduced, the method comprising the step of confirming that the antigen has been eliminated or reduced in a production process of the raw material or the processed product, wherein the antigen is at least one of the above antigens (1) to (13).

The step of confirming that the antigen has been eliminated or reduced in a production process of the raw material or processed product in which the antigen is eliminated or reduced may be carried out by confirming the presence or absence of the antigen by a procedure described in the above section “Tester Composition (1)”.

The milk or processed milk product or beef or processed beef product in which the antigen is eliminated or reduced may be produced by the procedure described in the above section “Antigen-Removed Food, etc. (1)”.

Epitopes

For the antigens identified as shown in Examples 1 to 3, the epitopes and the amino acids within the epitopes that are each important for binding to an IgE antibody from an allergic patient were identified as demonstrated in Example 4.

The results are summarized in Table 2. In Table 2, P1 to P25 are the patient numbers assigned to the respective 25 allergic patients.

TABLE 2-1
E1 Spot No. 1

| | | | | Cross-reactivity- | | Synthesized | |
|---|---|---|---|---|---|---|---|
| Protein name | immunoglobulin M heavy chain secretory form | | SEQ ID NO: | confirmed food | Graph No. | sequence | SEQ ID NO: |
| 15-residue sequence | QNVYTCQVEHNKETF | | 27 | | | | |
| P1 | Key | QNVYTCQVEHNKETF | 28 | | | | |
| | Key | SQNVYTC | 29 | | | | |
| | Key | TCQVEHN | 30 | | | | |
| | Key | HNKETFQ | 31 | | | | |
| P2 | Key | QNVYTCQVEHNKETF | 32 | | | | |
| | Key | LSQNVYTC | 33 | | | | |
| | Key | HNKE | 34 | | | | |
| P3 | Key | QNVYTCQVEHNKETF | 35 | | | | |
| | Key | HNKE | 36 | | | | |
| P4 | Preferable | QNVYTCQVEHNKETF | 37 | | | | |
| | Key | XXXYXCQXXXNKXXX | 38 | | | | |
| | Key | HNKE | 39 | | | | |
| P5 | Key | QNVYTCQVEHNKETF | 40 | | | | |
| | Key | DWLSQNVYTC | 41 | | | | |
| P6 | Key | QNVYTCQVEHNKETF | 42 | | | | |
| | Key | LSQNVYTC | 43 | | | | |
| | Key | NVYTCQVEHN | 44 | | | | |
| | Key | YTNVEHNKE | 45 | | | | |
| | Key | HNKETF | 46 | | | | |
| P8 | Key | QNVYTCQVEHNKETF | 47 | | | | |
| P9 | Key | QNVYTCQVEHNKETF | 48 | | | | |
| P20 | Preferable | XXXYXXQXXXNKETF | 49 | | | | |
| | Key | XXXXXXXXXXNKEXF | 50 | | | | |
| | Preferable | YTCQVEHNKE | 51 | Sheep | 6 | YTCQVEHNKE | 51 |
| | Key | YXXQXXXNKE | 52 | Chicken | 6 | YLSQTEENKE | 1115 |
| | More preferable | HNKETF | 53 | | | | |
| | Preferable | XNKETF | 54 | | | | |
| | Key | XNKEXF | 55 | | | | |
| P21 | Key | HNKETF | 56 | | | | |
| P22 | Key | YTCQVEHNKE | 57 | | | | |
| | Key | HNKETF | 58 | | | | |

TABLE 2-2
E2 Spot No. 1

| | | | SEQ |
|---|---|---|---|
| Protein | immunoglobulin M | | ID |
| name | heavy chain secretory form | | NO: |
| 15- | TKSKYVTSSPAPEPQ | | 59 |
| residue | | | |
| sequence | | | |
| P1 | More preferable | TKSKYVTSSPAPEPQ | 60 |
| | Preferable | XKSKXXXSSPAPEPQ | 61 |
| | Key | XKXKXXXXXXXXXPQ | 62 |
| | Preferable | KSKYVTSSP | 63 |
| | Key | KSKXXXSSP | 64 |
| | Preferable | KSKYVTSS | 65 |
| | Key | KSKXXXSS | 66 |
| | Key | YVTS | 67 |
| P2 | Preferable | TKSKYVTSSPAPEPQ | 68 |
| | Key | XKXKXXXXSPAXEPQ | 69 |
| | Key | SKYVTS | 70 |
| | Key | KYVTS | 71 |
| | Preferable | PAPEPQDPSV | 72 |
| | Key | PAXEPQDPSV | 73 |
| P8 | Key | TKSKYVTSSPAPEPQ | 74 |
| | Key | SKYVTSS | 75 |
| P9 | Key | TKSKYVTSSPAPEPQ | 76 |
| | More preferable | KSKYVTSSP | 77 |
| | Preferable | KSKYVTSS | 78 |
| | Key | SKYVTS | 79 |
| | Key | YVTSSPAPEP | 80 |
| P12 | Preferable | TKSKYVTSSPAPEPQ | 81 |
| | Key | XKSKXXTSSPAXEPQ | 82 |
| | More preferable | KSKYVTSS | 83 |
| | Preferable | SKYVTS | 84 |
| | Key | SKXXTS | 85 |
| P16 | Key | KYVTS | 86 |
| P18 | Preferable | KYVTSS | 87 |
| | Key | YVTS | 88 |
| P20 | Key | SKYVTS | 89 |
| P21 | Preferable | KYVTSS | 90 |
| | Key | YVTS | 91 |
| P23 | Preferable | XKXKYXTSXPAPEPQ | 92 |
| | Key | XXXKYXTXXPAXXXX | 93 |
| | Still more preferable | KSKYVTSSP | 94 |
| | More preferable | KYVTSSPAPE | 95 |
| | Preferable | KYVTS | 96 |
| | Key | KYXTS | 97 |

TABLE 2-3
E3 Spot No. 2, 3

| | | | SEQ |
|---|---|---|---|
| Protein | | | ID |
| name | Beta-1 metal-binding globulin | | NO: |
| 15- | CGDNTRKSVDNYQEC | | 98 |
| residue | | | |
| sequence | | | |
| P2 | More preferable | CGDNTRKSVDNYQEC | 99 |
| | Preferable | CGDNTRKSVDNYXXX | 100 |
| | Key | XGDNTRKSXDNXXXX | 101 |
| | More preferable | KSVDNYQECY | 102 |
| | Preferable | KSVDNYXXXY | 103 |
| | Key | KSXDNXXXXY | 104 |
| | More preferable | DNYQECYLA | 105 |
| | Preferable | DNYXXXYLA | 106 |
| | Key | DNXXXXYLA | 107 |
| P3 | More preferable | CGDNTRKSVDNYQEC | 108 |
| | Preferable | XGDNXRKSXDNYXXX | 109 |
| | Key | XGXXXXKSXDNYXXX | 110 |
| | Preferable | KSVDNYQECY | 111 |
| | Key | KSXDNYXXXY | 112 |
| | Key | DNYQECYLA | 113 |
| P4 | Preferable | CGDNTRKSVDNYQEC | 114 |
| | Key | XXDXXRKSXDNXXXX | 115 |
| | Key | DNTR | 116 |
| | Preferable | TRKSVDNYQE | 117 |
| | Key | XRKSXDNXXX | 118 |
| | Preferable | KSVDNYQECY | 119 |
| | Key | KSXDNXXXXY | 120 |
| P8 | Key | CGDNTRKSVDNYQEC | 121 |
| P12 | More preferable | CGDNTRKSVDNYQEC | 122 |
| | Preferable | XGDNTRKXXDNYXXX | 123 |
| | Key | XGDNTRXXXXNXXXX | 124 |
| | Key | DNTR | 125 |
| | Preferable | KSVDNYQECY | 126 |
| | Key | KXXDNYXXXY | 127 |
| P17 | Key | XXDXXRKSXDNYXXX | 128 |
| P18 | Preferable | XGXXXRKSVDNYXXX | 129 |
| | Key | XXXXXXKXXDNYXXX | 130 |
| P21 | Preferable | XGDNTRKSVDNYQXX | 131 |
| | Key | XXXXXRKXXDNYXXX | 132 |
| P22 | Preferable | XXXXXRKSVDNYQXX | 133 |
| | Key | XXXXXXKXVDNYXXX | 134 |
| | Preferable | DNTRKSV | 135 |
| | Key | XXXRKSV | 136 |
| | More preferable | TRKSVDNYQE | 137 |
| | Preferable | XRKSVDNYQX | 138 |
| | Key | XXKXVDNYXX | 139 |
| P24 | Preferable | XGDNTRKXXDNXXXX | 140 |
| | Key | XXDNXRXXXXNXXXX | 141 |
| P25 | Preferable | XGXNTRKSXDNXQXX | 142 |
| | Key | XXXXXRKSXDNXXXX | 143 |
| | Key | DNTR | 144 |

TABLE 2-4
E4 Spot No. 2, 3

| | | | SEQ |
|---|---|---|---|
| Protein | | | ID |
| name | Beta-1 metal-binding globulin | | NO: |
| 15- | SPHGKDLLFKDSADG | | 145 |
| residue | | | |
| sequence | | | |
| P1 | Key | SPHGKDLLFKDSADG | 146 |
| | Key | SPHGKD | 147 |
| | Key | KDLLFKDS | 148 |
| P2 | Key | SPHGKDLLFKDSADG | 149 |
| | Key | QLFQSPHGKD | 150 |
| P3 | Preferable | SPHGKDLLFKDSADG | 151 |
| | Key | XXHGKDXXXKXXXXX | 152 |
| | Key | KDSADGFLK | 153 |
| P4 | Preferable | SPHGKDLLFKDSADG | 154 |
| | Key | XXHGKXXXXKDXAXX | 155 |
| | Preferable | QSPHGKDLL | 156 |
| | Key | QXXHGKXXX | 157 |
| | Key | KDSADG | 158 |
| P5 | Preferable | SPHGKDLLFKDSADG | 159 |
| | Key | XPHGKXXXXXXXXXX | 160 |
| | Preferable | QLFQSPHGKD | 161 |
| | Key | QLFQXPHGKX | 162 |
| | Key | GKDLL | 163 |
| | Key | KDSADGFL | 164 |
| P6 | Key | SPHGKDLLFKDSADG | 165 |
| | Key | KDSADGFL | 166 |
| P7 | Key | SPHGKDLLFKDSADG | 167 |
| | Key | QLFQSPHGKD | 168 |
| P8 | Key | SPHGKDLLFKDSADG | 169 |
| P9 | Preferable | SPHGKDLLFKDSADG | 170 |
| | Key | SXHXKDXXXXDXXDG | 171 |
| | Preferable | KDLLFK | 172 |
| | Key | DLLFK | 173 |
| | Preferable | FKDSADGFLK | 174 |
| | Key | XXDXXDGFLK | 175 |
| P12 | Key | SPHGKDLLFKDSADG | 176 |
| | Key | PHGKDLLFK | 177 |
| | Key | GKDLLFKDSA | 178 |
| | Key | KDSADG | 179 |
| P16 | Key | XXHXKXXXXKDSXXX | 180 |
| | Key | DLLFKDS | 181 |
| P17 | Key | GKDLL | 182 |
| P20 | Key | XXHXKXXLFKXXXXX | 183 |
| | Preferable | KDLLFK | 184 |
| | Key | DLLFK | 185 |
| P21 | Key | KDSADG | 186 |
| P22 | Preferable | XXHXKDXLFKDSXXX | 187 |
| | Key | XXHXKXXXXKDXXXX | 188 |
| | Preferable | KDLLFK | 189 |
| | Key | KDXLFK | 190 |
| P23 | Key | XXXXKXLLFKXXXXX | 191 |
| | Key | LLFK | 192 |
| P24 | Key | SPHGKD | 193 |
| | Preferable | FKDSADG | 194 |
| | Key | KDSA | 195 |

TABLE 2-5
E5 Spot No. 2, 3

| | | | | Cross-reactivity- | | Synthesized | |
|---|---|---|---|---|---|---|---|
| Protein name | Beta-1 metal-binding globulin | | SEQ ID NO: | confirmed food | Graph No. | sequence | SEQ ID NO: |
| 15-residue sequence | HSTVFDNLPNPEDRK | | 196 | | | | |
| P1 | Preferable | HSTVFDNLPNPEDRK | 197 | | | | |
| | Key | XXXXXXXXXNPEDRK | 198 | | | | |
| | Key | FVKHSTVFDN | 199 | | | | |
| | Key | VKHSTVFD | 200 | | | | |
| | Key | KHSTVFD | 201 | | | | |
| | Preferable | NPEDRK | 202 | | | | |
| | Key | NPEDR | 203 | Sheep | 3 | NPEDR | 203 |
| P2 | Key | HSTVFDNLPNPEDRK | 204 | | | | |
| | Preferable | KHSTVFD | 205 | | | | |
| | Key | HSTVFD | 206 | | | | |
| | Preferable | NPEDRK | 207 | | | | |
| | Key | NPEDR | 208 | | | | |
| P3 | Key | HSTVFDNLPNPEDRK | 209 | | | | |
| | More preferable | FVKHSTVFD | 210 | | | | |
| | Preferable | KHSTVFD | 211 | | | | |
| | Key | HSTVFD | 212 | | | | |
| | Key | NPEDR | 213 | | | | |
| P4 | Key | HSTVFDNLPNPEDRK | 214 | | | | |
| | Preferable | KHSTVFD | 215 | | | | |
| | Key | HSTVFD | 216 | | | | |
| | Key | NPEDR | 217 | | | | |
| P5 | Key | HSTVFDNLPNPEDRK | 218 | | | | |
| | Key | HSTVFD | 219 | | | | |
| | Key | NPEDR | 220 | Sheep | 4 | NPEDR | 220 |
| P6 | Key | HSTVFDNLPNPEDRK | 221 | | | | |
| P8 | Preferable | HSTVFDNLPNPEDRK | 222 | | | | |
| | Key | XXXXXXXXXXPEDRK | 223 | | | | |
| | Key | KHSTVFD | 224 | | | | |
| | Preferable | NPEDRK | 225 | | 5 | NPEDRK | 225 |
| | Key | XPEDRK | 226 | Sheep | 5 | HPEDRK | 1116 |
| P9 | Key | HSTVFDNLPNPEDRK | 227 | | | | |
| P11 | Key | HSTVFDNLPNPEDRK | 228 | | | | |
| P15 | Key | TVFD | 229 | | | | |
| | Key | NPEDR | 230 | | | | |
| P16 | Key | STVFD | 231 | | | | |
| | Preferable | NPEDRK | 232 | | | | |
| | Key | NPEDR | 233 | | | | |
| P17 | Key | STVFD | 234 | | | | |
| | Key | NPEDR | 235 | | | | |
| P18 | Preferable | HSTVFD | 236 | | | | |
| | Key | TVFD | 237 | | | | |
| | Key | NPEDR | 238 | | | | |
| P20 | Key | XSXXXXXXXXPEDRX | 239 | | | | |
| | More preferable | DNLPNPEDRK | 240 | | | | |
| | Preferable | NPEDR | 241 | Sheep | 6 | NPEDR | 241 |
| | Key | XPEDR | 242 | Chicken | 6 | KPEDR | 1117 |
| | | | | Sheep | 6 | VPEDR | 1118 |
| P21 | Key | NPEDRK | 243 | | | | |
| P22 | Key | HSTVFD | 244 | | | | |
| | Key | NPEDRK | 245 | | | | |
| P23 | Preferable | STVFD | 246 | | | | |
| | Key | TVFD | 247 | | | | |
| | Key | NPEDRK | 248 | | | | |
| P24 | More preferable | XXXXXXXXXNPEDRK | 249 | | | | |
| | Preferable | XXXXXXXXXXPEDRK | 250 | | | | |
| | Key | XXXXXXXXXXPEDRX | 251 | | | | |
| | More preferable | NPEDRK | 252 | | | | |
| | Preferable | NPEDR | 253 | | | | |
| | Key | XPEDR | 254 | | | | |

TABLE 2-6
E6 Spot No. 2, 3

| | | | SEQ |
|---|---|---|---|
| Protein | | | ID |
| name | Beta-1 metal-binding globulin | | NO: |
| 15- | DCTSNFCLFQSNSKD | | 255 |
| residue | | | |
| sequence | | | |
| P2 | Key | DCTSNFCLFQSNSKD | 256 |
| | Key | QSNSK | 257 |
| P3 | Preferable | DCTSNFCLFQSNSKD | 258 |
| | Key | XXXXXXXXFQSNSKD | 259 |
| | Preferable | CLFQSNSK | 260 |
| | Key | QSNSK | 261 |
| P4 | Preferable | DCTSNFCLFQSNSKD | 262 |
| | Key | XXXXXXXXFQSNSKD | 263 |
| | Key | QSNSK | 264 |
| P8 | Preferable | DCTSNFCLFQSNSKD | 265 |
| | Key | XXXXXFCLFQSXSKD | 266 |
| | More preferable | LFQSNSKD | 267 |
| | Preferable | FQSNSK | 268 |
| | Key | FQSXSK | 269 |
| | Key | QSNS | 270 |
| P10 | Preferable | DCTSNFCLFQSNSKD | 271 |
| | Key | XXXXXXCLFXXXXKX | 272 |
| | More preferable | FQSNSK | 273 |
| | Preferable | QSNSK | 274 |
| | Key | QSNS | 275 |
| P16 | Key | QSNSK | 276 |
| P17 | Key | XXXXXXXXFXXNSKD | 277 |
| | Key | QSNSK | 278 |
| P20 | Preferable | CLFQSNSK | 279 |
| | Key | QSNSK | 280 |
| P21 | Key | QSNSK | 281 |
| P22 | Key | XXXXXXCLFXXXSKD | 282 |
| | Preferable | CLFQSNSK | 283 |
| | Key | QSNSK | 284 |
| P23 | Preferable | QSNSK | 285 |
| | Key | QSNS | 286 |
| P25 | Key | XXXXXXXXFXXNSKD | 287 |
| | Key | QSNSK | 288 |

TABLE 2-7
E7 Spot No. 2, 3

| | | | | Cross-reactivity- | | Synthesized | |
|---|---|---|---|---|---|---|---|
| Protein name | Beta-1 metal-binding globulin | | SEQ ID NO: | confirmed food | Graph No. | sequence | SEQ ID NO: |
| 15-residue sequence | LAENYKTEGESCKNT | | 289 | | | | |
| P2 | Preferable | LAENYKTEGESCKNT | 290 | | | | |
| | Key | XXXXXKXXXESXKXT | 291 | | | | |
| | Key | AENYKTEG | 292 | | | | |
| | Preferable | YKTEGESCK | 293 | | | | |
| | Key | XKXXXESXK | 294 | | | | |
| | More preferable | ESCKNTPEK | 295 | | | | |
| | Preferable | ESCKNTPE | 296 | | | | |
| | Key | ESXKXTPE | 297 | | | | |
| P3 | Preferable | LAENYKTEGESCKNT | 298 | | | | |
| | Key | XXXXYKXXGESCKNT | 299 | | | | |
| | Preferable | YKTEGESCK | 300 | | | | |
| | Key | KTEGE | 301 | | | | |
| | Key | YKXXGESCK | 302 | | | | |
| | Key | ESCKNTPE | 303 | | | | |
| P4 | Preferable | LAENYKTEGESCKNT | 304 | | | | |
| | Key | XXEXXKXEGEXCKXX | 305 | | | | |
| | Preferable | GESCKNTPE | 306 | | | | |
| | Key | GEXCKXXPE | 307 | | | | |
| P8 | Preferable | LAENYKTEGESCKNT | 308 | | | | |
| | Key | XXXXXXXXXESXKNX | 309 | | | | |
| | Key | KTEGESCK | 310 | | | | |
| | Preferable | ESCKNTPEK | 311 | | | | |
| | Key | ESXKNXPEK | 312 | | | | |
| P9 | Preferable | LAENYKTEGESCKNT | 313 | | | | |
| | Key | XXXXXXXEGESXKXX | 314 | | | | |
| | Preferable | NYKTEGESCK | 315 | | | | |
| | Key | XXXXEGESXK | 316 | | | | |
| | Preferable | ESCKNTPEK | 317 | | | | |
| | Key | ESXKXXPEK | 318 | | | | |
| P16 | Preferable | XXXXYKXEGEXCKNT | 319 | | | | |
| | Key | XXXXXXXXGXXXKNT | 320 | | | | |
| | Preferable | YKTEGESCK | 321 | | | | |
| | Key | YKXEGEXCK | 322 | | | | |
| P20 | Key | XXEXYKXXGEXCKNT | 323 | | | | |
| | More preferable | YKTEGESCK | 324 | | | | |
| | Preferable | KTEGESC | 325 | | 6 | KTEGESC | 325 |
| | Key | KXXGEXC | 326 | Chicken | 6 | KRRGESC | 1119 |
| | | | | Sheep | 6 | KADGESC | 1120 |
| P21 | Key | XXENYKXXXXXCKNT | 327 | | | | |
| | Preferable | YKTEGESCK | 328 | | | | |
| | Key | KTEGES | 329 | | | | |
| P23 | Preferable | XXXXYXXXXESCKNT | 330 | | | | |
| | Key | XXXXXXXXXXSCKNT | 331 | | | | |
| | Key | KTEGES | 332 | | | | |
| P24 | Key | XXXXXKXXGESXKXX | 333 | | | | |

TABLE 2-8
E8 Spot No. 2, 3

| | | | SEQ |
|---|---|---|---|
| Protein | | | ID |
| name | Beta-1 metal-binding globulin | | NO: |
| 15- | NHAVVSRKDKATCVE | | 334 |
| residue | | | |
| sequence | | | |
| P2 | Key | NHAVVSRKDKATCVE | 335 |
| P3 | Key | NHAVVSRKDKATCVE | 336 |
| P7 | Key | NHAVVSRKDKATCVE | 337 |
| | Key | RGPNHAVVS | 338 |
| P8 | Preferable | NHAVVSRKDKATCVE | 339 |
| | Key | XXXXVXXKDXAXXXX | 340 |
| | Key | RKDKAT | 341 |
| P9 | Key | NHAVVSRKDKATCVE | 342 |
| P10 | Key | NHAVVSRKDKATCVE | 343 |
| | Key | RKDKAT | 344 |
| P16 | Key | XXXVVSRKDKAXXXX | 345 |
| | Key | RKDK | 346 |
| P21 | Key | XXXXVXRKXKATXXX | 347 |
| | Preferable | RKDKAT | 348 |
| | Key | RKXKAI | 349 |
| P22 | Key | XXXXVSXKXKAXXXX | 350 |
| P23 | Preferable | XXXVVSRKDKATXXX | 351 |
| | Key | XXXXVSRKDKXXXXX | 352 |

TABLE 2-9
E9 Spot No. 4

| | | | | Cross-reactivity- | | Synthesized | |
|---|---|---|---|---|---|---|---|
| Protein name | Lactoperoxidase | | SEQ ID NO: | confirmed food | Graph No. | sequence | SEQ ID NO: |
| 15-residue sequence | ETELGSNEHSKTQCE | | 353 | | | | |
| P2 | Key | ETELGSNEHSKTQCE | 354 | | | | |
| | Preferable | SKTQC | 355 | | | | |
| | Key | SKTQ | 356 | | | | |
| P3 | Key | ETELGSNEHSKTQCE | 357 | | | | |
| | Key | SKTQC | 358 | | | | |
| P4 | Key | ETELGSNEHSKTQCE | 359 | | | | |
| | Preferable | HSKTQC | 360 | | | | |
| | Key | SKTQC | 361 | | | | |
| P5 | Key | ETELGSNEHSKTQCE | 362 | | 4 | ETELGSNEEISKTQCE | 362 |
| | Key | FAPETELGS | 363 | | | | |
| | Preferable | SKTQC | 364 | | | | |
| | Key | SKTQ | 365 | | | | |
| P8 | Key | ETELGSNEHSKTQCE | 366 | | 5 | ETELGSNEHSKTQCE | 366 |
| P9 | Key | ETELGSNEHSKTQCE | 367 | | | | |
| P12 | Key | ETELGSNEHSKTQCE | 368 | | | | |
| | Preferable | ENSKTQC | 369 | | | | |
| | Key | SKTQ | 370 | | | | |
| P17 | Key | SKTQ | 371 | | | | |
| P20 | Key | XXXXXXXEXXKTXXE | 372 | Chicken, Sheep | 6 | ETELGSNEHSKTQCE | 362 |
| | | | | Chicken | 6 | ENQNRLEEIKKTMFE | 1121 |
| | | | | Sheep | 6 | EKTFECGECGKTFWE | 1122 |
| | Key | SKTQ | 373 | | | | |
| P24 | Preferable | SKTQC | 374 | | | | |
| | Key | SKTQ | 375 | | | | |
| P25 | Key | SKR) | 376 | | | | |

TABLE 2-10
E10 Spot No. 4

| | | | SEQ |
|---|---|---|---|
| Protein | | | ID |
| name | Lactoperoxidase | | NO: |
| 15- | LHTLFFNTWRIIKDG | | 377 |
| residue | | | |
| sequence | | | |
| P1 | Preferable | LHTLFFNTWRIIKDG | 378 |
| | Key | XXXXXXXXXXXIKDG | 379 |
| | Key | LPLHTLFFNT | 380 |
| P2 | Key | LHTLFFNTWRIIKDG | 381 |
| P3 | Key | LHTLFFNTWRIIKDG | 382 |
| P8 | Key | LHTLFFNTWRIIKDG | 383 |
| P23 | Key | RIIK | 384 |

TABLE 2-11
E11 Spot No. 7

| | | | SEQ |
|---|---|---|---|
| Protein | | | ID |
| name | Polymeric immunoglobulin receptor | | NO: |
| 15- | SFEKYWCKWSNRGCS | | 385 |
| residue | | | |
| sequence | | | |
| P2 | More preferable | SFEKYWCKWSNRGCS | 386 |
| | Preferable | XFEKYWCKWSNRGCS | 387 |
| | Key | XXEKYWCKWSNXXXX | 388 |
| | Preferable | FEKYW | 389 |
| | Key | XEKYW | 390 |
| | Preferable | YWCKWSNRGC | 391 |
| | Key | YWCKWSNXXX | 392 |
| P3 | More preferable | SFEKYWCKWSNRGCS | 393 |
| | Preferable | XFEKYWCKWSNRGCS | 394 |
| | Key | XXEKYWCKWSNRGCX | 395 |
| P4 | Still more preferable | SFEKYWCKWSNRGCS | 396 |
| | More preferable | XFEKYXCKWSNXXXX | 397 |
| | Preferable | XXEKYXCKWSNXXXX | 398 |
| | Key | XXEXXXXXWSNXXXX | 399 |
| P6 | Key | SFEKYWCKWSNRGCS | 400 |
| P8 | More preferable | SFEKYWCKWSNRGCS | 401 |
| | Preferable | XFEKYWCKWSNRGCS | 402 |
| | Key | XXEKYXCKWSNRXXX | 403 |
| P9 | Preferable | SFEKYWCKWSNRGCS | 404 |
| | Key | XXXKYWCXXSXRGXX | 405 |
| P10 | Preferable | SFEKYWCKWSNRGCS | 406 |
| | Key | XXXKYWCKWSNRXXX | 407 |
| P17 | Preferable | XFXKYWCKWSNRXXX | 408 |
| | Key | XXXKYXXKWSNXXXX | 409 |
| P19 | Key | XFXKYWCKWSNRGXX | 410 |
| P23 | Key | XXXXXXXXXXNRGCS | 411 |

TABLE 2-12
E12 Spot No. 7

| | | | SEQ |
|---|---|---|---|
| Protein | | | ID |
| name | Polymeric immunoglobulin receptor | | NO: |
| 15- | NLDTVTKEDEGWYWC | | 412 |
| residue | | | |
| sequence | | | |
| P2 | Key | NLDTVTKEDEGWYWC | 413 |
| | More preferable | SLNLDTVT | 414 |
| | Preferable | LNLDTVT | 415 |
| | Key | LDTVT | 416 |
| | Preferable | TKEDEGWYWC | 417 |
| | Key | EGWY | 418 |
| P3 | Key | NLDTVTKEDEGWYWC | 419 |
| | Preferable | VVSLNLDTVT | 420 |
| | Key | SLNLDTVT | 421 |
| | Preferable | VTKEDEGWYW | 422 |
| | Key | KEDEGWY | 423 |
| | Key | EGWYWCGVKE | 424 |
| P4 | Key | NLDTVTKEDEGWYWC | 425 |
| | Preferable | VVSLNLDTVT | 426 |
| | Key | VSLNLDTVT | 427 |
| | Preferable | TKEDEGWYWC | 428 |
| | Key | KEDEGWYWC | 429 |
| P8 | Preferable | NLDTVTKEDEGWYWC | 430 |
| | Key | XXXTVXKEXEXXXXX | 431 |
| | Preferable | VSLNLDTVT | 432 |
| | Key | VSLXXXTVX | 433 |
| | More preferable | TKEDEGWYWC | 434 |
| | Preferable | TKEDEGWYW | 435 |
| | Key | KEDEGWYW | 436 |
| P20 | Key | LDTVTKEDEG | 437 |
| | Key | KEDEGWY | 438 |
| P22 | Preferable | XXXXVXKEDEXXXXX | 439 |
| | Key | XXXXXXKEDEXXXXX | 440 |
| | More preferable | VTKEDEGWYW | 441 |
| | Preferable | TKEDEGWYW | 442 |
| | Key | XKEDEXXXX | 443 |
| P23 | Key | NLDTVT | 444 |
| | Preferable | TKEDEGWYW | 445 |
| | Key | KEDEGWY | 446 |
| P25 | Key | XXXTVTKEDEXXXXX | 447 |
| | More preferable | KEDEGWYWC | 448 |
| | Preferable | KEDEG | 449 |
| | Key | KEDEX | 450 |

TABLE 2-13
E13 Spot No. 8

| | | | | Cross-reactivity- | | Synthesized | |
|---|---|---|---|---|---|---|---|
| Protein name | C3-beta-c | | SEQ ID NO: | confirmed food | Graph No. | sequence | SEQ ID NO: |
| 15-residue sequence | VKNGGKEEKHHRPGQ | | 451 | | | | |
| P1 | Key | VKNGGKEEKHHRPGQ | 452 | | | | |
| | Key | KNGGKEE | 453 | | | | |
| | Key | GGKE | 454 | | | | |
| P2 | Preferable | VKNQGKEEKHHRPGQ | 455 | | | | |
| | Key | XXNXXXEEXXXXPGQ | 456 | | | | |
| | Preferable | KNGGKEEK | 457 | | | | |
| | Key | KNGGKE | 458 | | | | |
| | Preferable | EKHHRPGQQI | 459 | | | | |
| | Key | EXXXXPGQQI | 460 | | | | |
| P3 | Key | VKNGGKEEKHHRPGQ | 461 | | | | |
| | Key | TLVVKNGGKE | 462 | | | | |
| | Preferable | NGGKEEK | 463 | | | | |
| | Key | GGKEEK | 464 | | | | |
| | Key | EKHHRPGQQI | 465 | | | | |
| P5 | Preferable | VKNGGKEEKHHRPGQ | 466 | | | | |
| | Key | XXXGXKEEXXXXXGQ | 467 | | | | |
| | Most preferable | KNGGKEEKHH | 468 | | | | |
| | Still more preferable | KNGGKEEKH | 469 | | | | |
| | More preferable | KNGGKEEKH | 470 | | | | |
| | Preferable | NGGKEEK | 471 | Sheep | 4 | NGGKEEK | 471 |
| | Key | XGXKEEX | 472 | Chicken | 4 | TGGKEEK | 1123 |
| | Preferable | KEEKHHRPGQ | 473 | Sheep | 4 | KEEKHHRPGQ | 473 |
| | Key | KEEXXXXXGQ | 474 | Chicken | 4 | KEESVPLVGQ | 1124 |
| | Preferable | EKHHRPGQQI | 475 | | | | |
| | Key | EXXXXXGQQI | 476 | | | | |
| P8 | Key | VKNGGKEEKHHRPGQ | 477 | | | | |
| | Preferable | VKNGGKEEKH | 478 | | | | |
| | Key | KNGGKEEK | 479 | | | | |
| | Key | KEEKHHRP | 480 | | | | |
| | Key | EKHHRPGQQI | 481 | | | | |
| P9 | Key | VKNGGKEEKHHRPGQ | 482 | | | | |
| | Key | TLVVKNGGK | 483 | | | | |
| | Key | KEEK | 484 | | | | |
| | Key | KPHRPGQQI | 485 | | | | |
| P10 | Key | VKNGGKEEKHHRPGQ | 486 | | | | |
| | Preferable | TIVVQIGGKF | 487 | | | | |
| | Key | TLVVKNGGK | 488 | | | | |
| P16 | Key | XXXXXXEEXHHXPGG | 489 | | | | |
| | More preferable | KNGGKEEK | 490 | | | | |
| | Preferable | GKEEKHHRP | 491 | | | | |
| | Key | KEEK | 492 | | | | |
| P17 | More preferable | KNGGKEEK | 493 | | | | |
| | Preferable | GGKEEK | 494 | | | | |
| | Key | GKEEK | 495 | | | | |
| P20 | More preferable | KNGGKEEK | 496 | | | | |
| | Preferable | GGKEEKHHRP | 497 | | | | |
| | Key | KEEK | 498 | | | | |
| P21 | Key | GGKEEK | 499 | | | | |
| P22 | Key | XXXGXXEEKXXXXXQ | 500 | | | | |

TABLE 2-14
E14 Spot No. 5

| | | | | Cross-reactivity- | | Synthesized | |
|---|---|---|---|---|---|---|---|
| Protein name | Ig heavy chain precursor (B/MT. 4A. 17. H5. A5) | | SEQ ID NO | confirmed food | Graph No | sequence | SEQ ID NO |
| 15-residue sequence | ESEDKYGTTPPQLDA | | 501 | | | | |
| P1 | More preferable | ESEDKYGTTPPQLDA | 501 | | | | |
| | Preferable | XXEDKYXXXPXXXXX | 503 | | | | |
| | Key | XXEDKYXXXXXXXXX | 504 | | | | |
| | Preferable | DKYGTTP | 505 | | 3 | DKYGTTP | 505 |
| | Key | DKYXXXP | 506 | Sheep | 3 | DKYELPP | 1125 |
| | Key | YGTTPPQLDA | 507 | | | | |
| P2 | Preferable | ESEDKYGTKPPQLDA | 508 | | | | |
| | Key | XSXDKYXXXXXXXXX | 509 | | | | |
| | Preferable | EDKYGTT | 510 | | | | |
| | Key | EDKYGT | 511 | | | | |
| | Preferable | YGTTPPQLDA | 512 | | | | |
| | Key | YGTTPPQLD | 513 | | | | |
| P3 | Preferable | ESEDKYGTTPPQLDA | 514 | | | | |
| | Key | XSEDKYXXXXXXXXX | 515 | | | | |
| | Key | EDKY | 516 | | | | |
| | Key | YGTTPPQLDA | 517 | | | | |
| P5 | Still more preferable | ESEDKYGTTPPQLDA | 518 | | | | |
| | More preferable | XSEDKYGXTPPQLXX | 519 | | | | |
| | Preferable | XXEDKYGXXXPQXXX | 520 | | | | |
| | Key | XXXDKYGXXXPQXXX | 521 | | | | |
| | Preferable | DKYGT | 522 | | | | |
| | Key | DKYGX | 523 | | | | |
| | More preferable | YGTTPPQLDA | 524 | | | | |
| | Preferable | GTTPPQLDA | 525 | | | | |
| | Key | GXTPPQLXX | 526 | | | | |
| P7 | Preferable | ESEDKYGTTPPQLDA | 527 | | | | |
| | Key | XSXDKYXXTXPXXXX | 228 | | | | |
| | More preferable | SEDKYGTTPP | 529 | | | | |
| | Preferable | EDKYGTT | 530 | | | | |
| | Key | XDKYXXT | 531 | | | | |
| | Preferable | YGTTPPQLDA | 532 | | | | |
| | Key | YGTTPPQLD | 533 | | | | |
| P8 | Preferable | ESEQKYGTTPPQLDA | 534 | | | | |
| | Key | XSEDKYGXTXXXXXX | 535 | | | | |
| | Still more preferable | SEDKYGTTP | 536 | | | | |
| | More Preferable | EDRYGTTP | 537 | | | | |
| | Preferable | EDKYGTT | 538 | | | | |
| | Key | EDKYGXT | 539 | | | | |
| P12 | Key | ESEDKYGTTPPQLDA | 540 | | | | |
| | Preferable | EDKYGT | 541 | | | | |
| | Key | EDKY | 542 | | | | |
| | Key | YGTTPPQLDA | 543 | | | | |
| P14 | More Preferable | XSEDKYXXTXPXXXX | 544 | | | | |
| | Preferable | XSXDKYXXTXPXXXX | 545 | | | | |
| | Key | XSXDKYXXXXXXXXX | 546 | | | | |
| | Preferable | EDKYG | 547 | | | | |
| | Key | EDKY | 548 | | | | |
| | Preferable | YGTTPPQLDA | 549 | | | | |
| | Key | YGTTPPQLD | 550 | | | | |
| P16 | Preferable | XXXDKYXXTPPXLDA | 551 | | | | |
| | Key | XXXDKYXXTXXXXXX | 552 | | | | |
| | Still more preferable | SEDKYGTTPP | 553 | | | | |
| | More preferable | EDKYGTTPP | 554 | | | | |
| | Preferable | XDKYXXTP | 555 | | | | |
| | Key | XDKYXXTX | 556 | | | | |
| | More preferable | YGTTPPQLDA | 557 | | | | |
| | Preferable | GTTPPQLD | 558 | | | | |
| | Key | XXTPPXLD | 559 | | | | |
| P17 | Preferable | XSXDKYXXTPPQXXX | 560 | | | | |
| | Key | XXXDKYXXXXXQXXX | 561 | | | | |
| | Preferable | SEDKYGTTP | 562 | | | | |
| | Key | EDKY | 563 | | | | |
| | More preferable | YGTTPPQLDA | 564 | | | | |
| | Preferable | TTPPQLD | 565 | | | | |
| | Key | XTPPDXX | 566 | | | | |
| P16 | Preferable | XSXDRYXXTPPDXXX | 567 | | | | |
| | Key | XXXDKYXXXXPXXXX | 568 | | | | |
| | More preferable | EDKYGTTP | 569 | | | | |
| | Preferable | YGTTPPQLDA | 570 | | | | |
| | Key | YGTIP | 571 | | | | |
| P20 | Preferable | XXEDKYXXTPPDXDA | 572 | | | | |
| | Key | XXEDKYXXXXXXXXA | 573 | | | | |
| | More preferable | EDKYGTTPP | 574 | | 6 | EDKYGTTP | 574 |
| | Preferable | EDKYXXTPP | 575 | | | | |
| | Key | EDKYXXXXX | 576 | Chicken | 6 | EDKYRKIN | 1126 |
| | | | | Sheep | 6 | EDKYGTTT | 1127 |
| | More Preferable | YGTTPPQLDA | 577 | | | | |
| | Preferable | YGTTPPQLD | 578 | | | | |
| | Key | YXXTPPQXD | 579 | | | | |
| P21 | More Preferable | XSXDKYXXTPPQLDA | 580 | | | | |
| | Preferable | XXXQKYXXTXPQLXA | 581 | | | | |
| | Key | XXXQKYXXTXPDXXX | 582 | | | | |
| | More Preferable | SEDKYGTTP | 583 | | | | |
| | Preferable | SEDKY | 584 | | | | |
| | Key | SXDKY | 585 | | | | |
| | Most preferable | YGTTPPQLD | 586 | | | | |
| | Still more preferable | YGTTPPQL | 587 | | | | |
| | More preferable | YXXTPPQL | 588 | | | | |
| | Preferable | YXXTXPQL | 589 | | | | |
| | Key | YXXTXPQX | 590 | | | | |
| P23 | More preferable | XSXXKYXTTXPQLDX | 591 | | | | |
| | Preferable | XXXXKYXTTXPQLXX | 592 | | | | |
| | Key | XXXXKYXXTXPDXXX | 593 | | | | |
| | Still more preferable | YGTTPPQLD | 594 | | | | |
| | More preferable | YGTTPPQL | 595 | | | | |
| | Preferable | YXTTXPQL | 596 | | | | |
| | Key | YXXTXPDX | 597 | | | | |
| P24 | Preferable | XXEDKYXXXXPXXXX | 598 | | | | |
| | Key | XXEDKYXXXXXXXXX | 509 | | | | |
| | More preferable | EDKYGTTP | 600 | | | | |
| | Preferable | EDKYGTT | 601 | | | | |
| | Key | EDKYXXX | 602 | | | | |
| | Preferable | YGTTPPQLDA | 603 | | | | |
| | Key | GTTPPQLD | 604 | | | | |

TABLE 2-15
E15 Spot No. 5

| | | | | Cross-reactivity- | | Synthesized | |
|---|---|---|---|---|---|---|---|
| Protein name | Ig heavy chain precursor (B/MT. 4A. 17. H5. A5) | | SEQ ID NO | confirmed food | Graph No | sequence | SEQ ID NO |
| 15-residue sequence | TKENSKSQVSLSVSS | | 605 | | | | |
| P1 | Still more preferable | TKENSKSQVSLSVSS | 606 | | | | |
| | More preferable | XKXNSKSQVSLSVSX | 607 | | | | |
| | Preferable | XKXXSKSQXSLSXSX | 608 | | | | |
| | Key | XKXXSKSQXSXSXXX | 609 | | | | |
| | Preferable | KENSK | 610 | | 3 | KENSK | 610 |
| | Key | KXNSK | 611 | Sheep | 3 | KINSK | 1126 |
| | Key | ENSK | 612 | | | | |
| P2 | Key | TKENSKSQVSLSVSS | 613 | | | | |
| P3 | Key | TKENSKSQVSLSVSS | 614 | | | | |
| | Key | VSLSVSSVTP | 615 | | | | |
| P4 | More preferable | TKENSKSQVSLSVSS | 616 | | | | |
| | Preferable | XXENSKSQXXXXXXX | 617 | | | | |
| | Key | XXXNSKSXXXXXXXX | 618 | | | | |
| P5 | Preferable | TKENSKSQVSLSVSS | 619 | | | | |
| | Key | ANNInbUAAAAAAA | 620 | | | | |
| P8 | Key | TKENSKSQVSLSVSS | 621 | | | | |
| | Key | SLSVSSVTP | 622 | | | | |
| P9 | Key | TKENSKSQVSLSVSS | 623 | | | | |
| P10 | Key | TKENSKSQVSLSVSS | 624 | | | | |
| P16 | Key | XKXXSKXXXXXXXSS | 625 | | | | |
| | Key | NSKS | 626 | | | | |
| P17 | Key | XXENSKXXXXXXXXX | 627 | | | | |
| P21 | Key | XXXNSKSXXXXXXXX | 628 | | | | |
| P23 | Preferable | XXEXSKXQXXLSVSS | 629 | | | | |
| | Key | XXEXSKXXXXLXVSS | 630 | | | | |
| P25 | Key | TXENSKXXXXXXXXX | 631 | | | | |
| | Key | ENSK | 632 | | | | |

TABLE 2-16
E16 Spot No. 6

| | | | SEQ |
|---|---|---|---|
| Protein | | | ID |
| name | Perilipin | | NO: |
| 15- | TTVTGAKDSVASTIT | | 633 |
| residue | | | |
| sequence | | | |
| P2 | Preferable | TTVTGAKDSVASTIT | 634 |
| | Key | XXXXXXKDSXXSXXX | 635 |
| | Preferable | AVTTTVTGAK | 636 |
| | Key | AVTTTVTGA | 637 |
| | Key | KDSV | 638 |
| P5 | More preferable | TTVTGAKDSVASTIT | 639 |
| | Preferable | TTVTGXKDXVAXXXX | 640 |
| | Key | TXVXGXKXXVXXXXX | 641 |
| | Most preferable | DAVTTTVTGA | 642 |
| | Still more preferable | AVTTTVTGA | 643 |
| | More preferable | TTTVTGA | 644 |
| | Preferable | TTTVTGX | 645 |
| | Key | TTXVXGX | 646 |
| | More preferable | TGAKDSVAS | 647 |
| | Preferable | DSVAS | 648 |
| | Key | DSVA | 649 |
| P8 | Key | TTVTGAKDSVASTIT | 650 |
| | Key | AVTTTVTGA | 651 |
| | Key | DSVAST | 652 |
| P9 | Key | TTVTGAKDSVASTIT | 653 |
| P10 | Key | TTVTGAKDSVASTIT | 654 |
| | Preferable | AVTTTVTGAK | 655 |
| | Key | TTTVTGA | 656 |
| | Key | TVTGAKDSVA | 657 |
| | Preferable | KDSVASTI | 658 |
| | Key | DSVAST | 659 |
| P13 | Preferable | TTVTGAKDSVASTIT | 660 |
| | Key | XXXXXXKXXVASXIX | 661 |
| | Key | AVTTTVTGA | 662 |
| | Preferable | KDSVASTI | 663 |
| | Key | KXXVASXI | 664 |
| P16 | More preferable | XXXXGXKDSVAXXXX | 665 |
| | Preferable | XXXXXXKDSVAXXXX | 666 |
| | Key | XXXXXXKDSXAXXXX | 667 |
| | Preferable | KDSVA | 668 |
| | Key | KDSV | 669 |
| P17 | Preferable | TXXTGAKXSXXXXXX | 670 |
| | Key | TXXTGXKXSXXXXXX | 671 |
| | Preferable | GAKDSVA | 672 |
| | Key | GAKXSXX | 673 |

TABLE 2-17
E17 Spot No. 6

| | | | SEQ |
|---|---|---|---|
| Protein | | | ID |
| name | Perilipin | | NO: |
| 15- | NQKIQDAQDKLYLSW | | 674 |
| residue | | | |
| sequence | | | |
| P2 | Key | NQKIQDAQDKLYLSW | 675 |
| | Key | AQDKLYLSWL | 676 |
| P3 | Key | NQKIQDAQDKLYLSW | 677 |
| P5 | More preferable | NQKIQDAQDKLYLSW | 678 |
| | Preferable | XQKXXXXXXKLYLSW | 679 |
| | Key | XQKXXXXXXXLYLSW | 680 |
| | More preferable | AQDKLYLSWL | 681 |
| | Preferable | XXXKLYLSWL | 682 |
| | Key | XXXXLYLSWL | 683 |
| P8 | Key | NQKIQDAQDKLYLSW | 684 |
| P9 | Key | NQKIQDAQDKLYLSW | 685 |
| | Key | DKLYLSWLEW | 686 |
| P16 | Key | NQKIQXXXXXXXXXX | 687 |
| P21 | More preferable | NQKIQXXQXKLYLSX | 688 |
| | Preferable | XQKIQXXXXKLYLSX | 689 |
| | Key | XQKIQXXXXXXYLXX | 690 |
| | More preferable | QDKLYLSW | 691 |
| | Preferable | QXKLYLSX | 692 |
| | Key | XXKLYLSX | 693 |

TABLE 2-18
E18 Spot No. 12

| | | | | Cross-reactivity- | | Synthesized | |
|---|---|---|---|---|---|---|---|
| Protein name | Zinc-alpha-2-glycoprotein | | SEQ ID NO | confirmed food | Graph No | sequence | SEQ ID NO |
| 15-residue sequence | EDWEKESALQRARED | | 694 | | | | |
| P1 | More preferable | EDWEKESALQPARED | 695 | | | | |
| | Preferable | XXWEKEXXLQXXXED | 696 | | | | |
| | Key | XXWEKEXXXXXXXXX | 697 | | | | |
| | More preferable | WEKESAL | 698 | | 3 | WEKESAL | 698 |
| | Preferable | WEKEXXL | 699 | Sheep | 3 | WEKEMKL | 1129 |
| | Key | WEKEXXX | 700 | | | | |
| | Preferable | EKESAL | 701 | | | | |
| | Key | EKEXXL | 702 | | | | |
| P2 | Preferable | EDWEKESALQRARED | 703 | | | | |
| | Key | XXWEKEXXXXXXXXX | 704 | | | | |
| | Most preferable | MEDWEKESAL | 705 | | | | |
| | Still more preferable | DWEKESALQR | 706 | | | | |
| | More preferable | DWEKESAL | 707 | | | | |
| | Preferable | DWEKESA | 708 | | | | |
| | Key | XWEKEXX | 709 | | | | |
| P3 | Key | EDWEKESALQRARED | 710 | | | | |
| | Key | WEKE | 711 | | | | |
| P4 | Key | EDWEKESALQRARED | 712 | | | | |
| | Still more preferable | DWEKESAL | 713 | | | | |
| | More preferable | DWEKESAL | 714 | | | | |
| | Preferable | WEKES | 715 | | | | |
| | Key | WEKE | 716 | | | | |
| P5 | Preferable | EDWEKESALQRARED | 717 | | | | |
| | Key | XXXXKEXALXRXRXD | 718 | | | | |
| | Preferable | WEKESAL | 719 | | | | |
| | Key | WEKES | 720 | | | | |
| | Preferable | KESALQRARE | 721 | | | | |
| | Key | KEXALXRXRX | 722 | | | | |
| P8 | Key | EDWEKESALQRARED | 723 | | | | |
| | Preferable | WEKESA | 724 | | | | |
| | Key | WEKE | 725 | | | | |
| | Key | KESALQRARE | 726 | | | | |
| P10 | Key | EDWEKESALQRARED | 727 | | | | |
| | Key | MEDWEKE | 728 | | | | |
| | Key | KESALQR | 729 | | | | |
| | Key | LQRARED\|FM | 730 | | | | |
| P12 | Key | EDWEKESALQRARED | 731 | | | | |
| | Key | WEKE | 732 | | | | |
| P16 | Key | XXXXKEXAXXRXRXD | 733 | | | | |
| | More preferable | DWEKESALQR | 734 | | | | |
| | Preferable | WEKESALQR | 735 | | | | |
| | Key | WEKESAL | 736 | | | | |
| | Preferable | KESALQRARE | 737 | | | | |
| | Key | KEXAXXRXRX | 738 | | | | |
| P17 | Preferable | WEKESAL | 739 | | | | |
| | Key | EKESAL | 740 | | | | |
| P20 | Preferable | XXWEKEXXXXXXRED | 741 | | | | |
| | Key | XXXXKXXXXXXXRED | 742 | | | | |
| | Preferable | WEKESALQR | 743 | | | | |
| | Key | KESAL | 744 | | | | |
| P22 | Key | XXWEKEXXXXXXRXX | 745 | | | | |
| | Preferable | DWEKESALQR | 746 | | | | |
| | Key | XWEKEXXXXX | 747 | | | | |
| P23 | Key | DWEKESALQR | 748 | | | | |
| | Key | ESALQRAR | 749 | | | | |
| P24 | Preferable | WEKES | 750 | | | | |
| | Key | WEKE | 751 | | | | |
| P25 | Preferable | XXWEKEXAXXXXRXX | 752 | | | | |
| | Key | XXWEKEXXXXXXXXX | 753 | | | | |
| | Still more preferable | WEKESALQR | 754 | | | | |
| | More preferable | WEKESAL | 755 | | | | |
| | Preferable | WEKEXAX | 756 | | | | |
| | Key | WEKEXXX | 757 | | | | |

TABLE 2-19
E19 Spot No. 12

| | | | SEQ |
|---|---|---|---|
| Protein | | | ID |
| name | Zinc-alpha-2-glycoprotein | | NO: |
| 15- | LSDIMDYYKDREGSH | | 758 |
| residue | | | |
| sequence | | | |
| P1 | Key | LSDIMDYYKDREGSH | 759 |
| | Key | KDREGS | 760 |
| P2 | More preferable | LSDIMDYYKDREGSH | 761 |
| | Preferable | XXXXXXXXKXREGSH | 762 |
| | Key | XXXXXXXXKXXEGSH | 763 |
| | More preferable | KDREGS | 764 |
| | Preferable | KDREG | 765 |
| | Key | KXREG | 766 |
| | Key | DREG | 767 |
| P3 | Preferable | LSDIMDYYKDREGSH | 768 |
| | Key | XXXXXXXXKXREGXX | 769 |
| | Preferable | KDREG | 770 |
| | Key | KXREG | 771 |
| | Key | DREG | 772 |
| P4 | Key | LSDIMDYYKDREGSH | 773 |
| | Key | KDREGS | 774 |
| P8 | Key | LSDIMDYYKDREGSH | 775 |
| | Key | DREG | 776 |
| P16 | More preferable | KDREGS | 777 |
| | Preferable | KDREG | 778 |
| | Key | DREG | 779 |
| P17 | Preferable | KDREGS | 780 |
| | Key | KDRE | 781 |
| P20 | Key | KDREG | 782 |
| P21 | Key | XXXXXXYYKDXXXXX | 783 |
| P22 | Key | KDREG | 784 |
| P23 | Preferable | XXXXXXXXKDREGSH | 785 |
| | Key | XXXXXXXXKXXEGSH | 786 |
| | More preferable | KDREGS | 787 |
| | Preferable | KDREG | 788 |
| | Key | DREG | 789 |
| P24 | Key | XXXXXXXXKDXEGXX | 790 |
| | Key | DREG | 791 |

TABLE 2-20
E20 Spot No. 10

| | | | | Cross-reactivity- | | Synthesized | |
|---|---|---|---|---|---|---|---|
| Protein name | Glycoprotein 2 | | SEQ ID NO | confirmed food | Graph No | sequence | SEQ ID NO |
| 15-resdue sequence | DPATAEDKCDRTCRP | | 792 | | | | |
| P1 | Preferable | DPATAEDKCDRTCRP | 793 | | | | |
| | Key | XXATAEDKCDRICRP | 794 | | | | |
| | Key | ATAEDKCDRT | 795 | | | | |
| | Key | AEDKCDRTCR | 796 | | | | |
| P2 | Still more preferable | DPATAEDKCDRTCRP | 797 | | | | |
| | More preferable | DPATAEDKCDRTXRP | 798 | | | | |
| | Preferable | XXATAEDKCDRXXXP | 799 | | | | |
| | Key | XXATAEDKCDRXXXX | 800 | | | | |
| | Key | EDKC | 801 | | | | |
| P3 | More preferable | DPATAEDKCDRTCRP | 802 | | | | |
| | Preferable | XXATXEDKCDRTXXX | 803 | | | | |
| | Key | XXXXXXDKXDRXXXX | 804 | | | | |
| P4 | More preferable | DPATAEDKCDRTCRP | 805 | | | | |
| | Preferable | DPATXXDKCDRTCRP | 806 | | | | |
| | Key | XPATXXDKCDRTCRP | 807 | | | | |
| | More preferable | AEDKCDRT | 808 | | | | |
| | Preferable | AEDKCDRT | 809 | | | | |
| | Key | EDKCDR | 810 | | | | |
| P5 | Still more preferable | DPATAEDKCDRTCRP | 811 | | | | |
| | More preferable | DPATAEDKCDRTXRP | 812 | | | | |
| | Preferable | XPATAEDKCDRTXXX | 813 | | | | |
| | Key | XPATXXDKCDRXXXX | 814 | | | | |
| | More preferable | RYCTDPATAE | 815 | | | | |
| | Preferable | RYCTXPATAE | 816 | | | | |
| | Key | RYCTXPATXX | 817 | | | | |
| | Preferable | AEDKCDRTCR | 818 | | 4 | AEDKCDRTCR | 818 |
| | Key | XXDKCDRXXX | 819 | Sheep | 4 | LNDKCDRLRK | 1130 |
| P8 | More preferable | DPATAEDKCDRTCRP | 820 | | | | |
| | Preferable | XXXTXEDKXDRTXXX | 821 | | | | |
| | Key | XXXXXXDKXDRXXXX | 822 | | | | |
| | Key | EDKCD | 823 | | | | |
| P10 | Key | DPATAEDKCDRTCRP | 824 | | | | |
| | Key | YCTDPATAE | 825 | | | | |
| | Key | TDPATAEDKC | 826 | | | | |
| | Key | ATAEDKCDR | 827 | | | | |
| P12 | Preferable | DPATAEDKCDRTCRP | 828 | | | | |
| | Key | XXXXXXDKXDXTCXX | 829 | | | | |
| | Key | EDKCD | 830 | | | | |
| P16 | Preferable | XXXTAEDKCDRTXXX | 831 | | | | |
| | Key | XXXXXXDKCDRXXXX | 832 | | | | |
| | Key | AEDKC | 833 | | | | |
| P17 | Preferable | DXXTAXDKCDRXXXX | 834 | | | | |
| | Key | XXXTXXDKXXRXXXX | 835 | | | | |
| | Preferable | DPATAEDKCD | 836 | | | | |
| | Key | DXXTAXDKCD | 837 | | | | |
| | More preferable | ARDKCDRIC | 838 | | | | |
| | Preferable | AEDKCDRT | 839 | | | | |
| | Key | AXDKCDRX | 840 | | | | |
| P18 | Preferable | XXXXXXDKCDRTXRP | 841 | | | | |
| | Key | XXXXXXDKCDRTXXX | 842 | | | | |
| | Still more preferable | ATAEDKCDR | 843 | | | | |
| | More preferable | EDKCDRTCR | 844 | | | | |
| | Preferable | EDKCDR | 845 | | | | |
| | Key | XDKCDR | 846 | | | | |
| P20 | Preferable | XPATAEDKXXXXXXX | 847 | | | | |
| | Key | XPAXXXDKXXXXXXX | 848 | | | | |
| P21 | Preferable | XXXTAXXXCDRXXXX | 849 | | | | |
| | Key | XXXTAXXXXDRXXXX | 850 | | | | |
| | Preferable | AEDKCDRTC | 851 | | | | |
| | Key | AEDKCD | 852 | | | | |
| P23 | More preferable | XXXXAXDKCDRXXXX | 853 | | | | |
| | Preferable | YAXXXXDKCDRXXXX | 854 | | | | |
| | Key | XXXXXXOKXDRXXXX | 855 | | | | |
| | Still more preferable | AEDKCDR | 856 | | | | |
| | More preferable | AEDKCD | 857 | | | | |
| | Preferable | AXDKCD | 858 | | | | |
| | Key | XXDKCD | 859 | | | | |
| P25 | More preferable | DXXTAEDKCDRTCRP | 860 | | | | |
| | Preferable | XXXTXEDKCDRTXRP | 861 | | | | |
| | Key | XXXTXXDKCDRTXRP | 862 | | | | |
| | Most preferable | PATAEDKCDR | 863 | | | | |
| | Even more preferable | AEDKCDRTC | 864 | | | | |
| | Still more preferable | AEDKCDR | 865 | | | | |
| | More preferable | AEDKCD | 866 | | | | |
| | Preferable | XEDKCD | 867 | | | | |
| | Key | XXDKCD | 868 | | | | |

TABLE 2-21
E21 Spot No. 10

| | | | SEQ |
|---|---|---|---|
| Protein | | | ID |
| name | Glycoprotein 2 | | NO: |
| 15- | TEDIQGCDSDKHGWY | | 869 |
| residue | | | |
| sequence | | | |
| P1 | More preferable | TEDIQGCDSDKHGWY | 870 |
| | Preferable | TXDIQXXDXDKHXXY | 871 |
| | Key | TXDIQXXDXDKXXXY | 872 |
| | More preferable | IQGCDSDKHG | 873 |
| | Preferable | IQXXDXDKHX | 874 |
| | Key | IQXXDXDKXX | 875 |
| | More preferable | DSDKHGWYR | 876 |
| | Preferable | DXDKHXXYR | 877 |
| | Key | DXDKXXXYR | 878 |
| | More preferable | DKHGWYRFVG | 879 |
| | Preferable | DKHXXYRFVG | 880 |
| | Key | DKXXXYRFVG | 881 |
| P2 | Preferable | TEDIQGCDSDKHGWY | 882 |
| | Key | TXDIQXXXXXXXXXX | 883 |
| | Preferable | ENTEDIQG | 884 |
| | Key | ENTXDIQX | 885 |
| | Key | IQGCDSDKHG | 886 |
| | Key | DKHGWY | 887 |
| P3 | Key | TEDIQGCDSDKHGWY | 888 |
| | Key | ENTEDIQGCD | 889 |
| P4 | Key | TEDIQGCDSDKHGWY | 890 |
| | Key | DSDK | 891 |
| | Key | DKHGWYRFVG | 892 |
| P8 | Key | TEDIQGCDSDKHGWY | 893 |
| | Key | NTEDIQG | 894 |
| P9 | Key | TEDIQGCDSDKHGWY | 895 |
| | Key | STENTEDIQG | 896 |
| | More preferable | EDIQGCDSDK | 897 |
| | Preferable | IQGCDSDKHG | 898 |
| | Key | IQGCDSDK | 899 |
| | Key | DKHGWYR | 900 |
| | Key | DKHGWY | 901 |
| P10 | Key | TEDIQGCDSDKHGWY | 902 |
| | Key | IQGCDSDKHG | 903 |
| | Key | DKHGWY | 904 |
| P12 | Key | TEDIQGCDSDKHGWY | 905 |
| P13 | More preferable | TEDIQGCDSDKHGWY | 906 |
| | Preferable | XXDXQXXXXXKXGWY | 907 |
| | Key | XXXXQXXXXXXXGWY | 908 |
| | Preferable | DKHGWY | 909 |
| | Key | XKXGWY | 910 |
| P16 | Key | XXDIQXXXXXKXXXX | 911 |
| | Preferable | IQGCDSDKHG | 912 |
| | Key | IQGCDSDKH | 913 |
| | Key | DKHGWY | 914 |
| P17 | Preferable | DKHGWY | 915 |
| | Key | DKHG | 916 |
| P20 | More preferable | EDIQGCDSDK | 917 |
| | Preferable | IQGCDSDKHG | 918 |
| | Key | IQGCDSDK | 919 |
| | Key | DKHGWY | 920 |
| P23 | More preferable | IQGCDSDKHG | 921 |
| | Preferable | SDKHGWY | 922 |
| | Key | SDKH | 923 |
| P25 | Preferable | XXDIQXCDXDKHGWY | 924 |
| | Key | XXDXQXXXXDKXXXX | 925 |
| | Preferable | IQGCDSDKHG | 926 |
| | Key | DKHGWY | 927 |

TABLE 2-22
E22 Spot No. 11

| | | | SEQ |
|---|---|---|---|
| Protein | | | ID |
| name | Lactadherin | | NO: |
| 15- | SESKIFPGNMDNNSH | | 928 |
| residue | | | |
| sequence | | | |
| P1 | Key | SESKIFPGNMDNNSH | 929 |
| | Key | ESKIF | 930 |
| | Preferable | NMDNNSHKKN | 931 |
| | Key | MDNNSHKK | 932 |
| P2 | Key | SESKIFPGNMDNNSH | 933 |
| | Preferable | ESKIFPGNM | 934 |
| | Key | ESKIFPGN | 935 |
| | More preferable | NMDNNSHKK | 936 |
| | Preferable | MDNNSHKK | 937 |
| | Key | MDNNS | 938 |
| P3 | Key | SESKIFPGNMDNNSH | 939 |
| | Key | SKIF | 940 |
| | Key | NMDNNSHKKN | 941 |
| P4 | Preferable | SESKIFPGNMDNNSH | 942 |
| | Key | SESKXXXXXXXXNXX | 943 |
| | Key | SESKIF | 944 |
| | Key | SKIFPGNMDN | 945 |
| | More preferable | MDNNSHK | 946 |
| | Preferable | MDNNSH | 947 |
| | Key | MDNNS | 948 |
| P5 | Preferable | SESKIFPGNMDNNSH | 949 |
| | Key | XXXXXFPGNMDNNSH | 950 |
| | Preferable | ESKIFPGNM | 951 |
| | Key | XXXXFPGNM | 952 |
| | Key | FPGNMDNNSH | 953 |
| | Preferable | NMDNNSHKK | 954 |
| | Key | MDNNSH | 955 |
| P8 | Key | SESKIFPGNMDNNSH | 956 |
| | Preferable | ESKIF | 957 |
| | Key | SKIF | 958 |
| P10 | Key | SESKIFPGNMDNNSH | 959 |
| P11 | Key | SESKIFPGNMDNNSH | 960 |
| | Key | ASESKIF | 961 |
| | Key | MDNNS | 962 |
| P12 | Key | SESKIFPGNMDNNSH | 963 |
| | Key | ESKIF | 964 |
| P14 | Key | SESKIFPXXXXXXXX | 965 |
| | Key | ESKIF | 966 |
| P16 | Preferable | ESKIFPGN | 967 |
| | Key | ESKIF | 968 |
| P17 | Key | XEXKXXXXXXXXXSH | 969 |
| | Preferable | ESKIFPG | 970 |
| | Key | ESKIF | 971 |
| | Preferable | MDNNSH | 972 |
| | Key | MDNNS | 973 |
| P19 | Key | XXXXXFXGNMDNNSH | 974 |
| P20 | Key | ESKIF | 975 |
| | Key | MDNNSH | 976 |
| P21 | Preferable | XXXXXXXXNMDNNSH | 977 |
| | Key | XXXXXXXXNMDXNSH | 978 |
| | Key | SESKIFPGNM | 979 |
| | Preferable | SKIFPGNMDN | 980 |
| | Key | XXXXXXNMDN | 981 |
| | Preferable | MDNNS | 982 |
| | Key | MDXNS | 983 |
| P23 | More preferable | ESKIFPGNM | 984 |
| | Preferable | SKIFPGNM | 985 |
| | Key | SKIF | 986 |
| | Key | MDNNS | 987 |
| P24 | Preferable | ESKIFPG | 988 |
| | Key | ESKIF | 989 |
| P25 | Preferable | SESKIFPXXXXXXSX | 990 |
| | Key | SXXKIFXXXXXXXXX | 991 |
| | More preferable | SESKIFPGNM | 992 |
| | Preferable | SESKIFPXXX | 993 |
| | Key | SXXKIFXXXX | 994 |
| | Preferable | SKIFPGNMDN | 995 |
| | Key | SKIFPXXXXX | 996 |

TABLE 2-23
E23 Spot No. 9

| | | | SEQ |
|---|---|---|---|
| Protein | Immunoglobulin light chain, | | ID |
| name | lambda gene cluster | | NO: |
| 15- | QPKSPPSVTLFPPST | | 997 |
| residue | | | |
| sequence | | | |
| P2 | Still more preferable | QPKSPPSVTLFPPST | 998 |
| | More preferable | XXKSXXSXTLFXPST | 999 |
| | Preferable | XXKXXXSXTLFXPSX | 1000 |
| | Key | XXKXXXXXXLFXPSX | 1001 |
| | Preferable | KSPPSV | 1002 |
| | Key | SPPSV | 1003 |
| | Still more preferable | SVTLFPPSTE | 1004 |
| | More preferable | SXTLFXPSTE | 1005 |
| | Preferable | SXTLFXPSXE | 1006 |
| | Key | XXXLFXPSXE | 1007 |
| P3 | Key | QPKSPPSVTLFPPST | 1008 |
| | Key | VLGQPKSPPS | 1009 |
| | Key | GQPKSPPSVT | 1010 |
| | Preferable | KSPPSVTLF | 1011 |
| | Key | SPPS | 1012 |
| | Key | LFPPSTE | 1013 |
| P4 | Preferable | QPKSPPSVTLFPPST | 1014 |
| | Key | QXKSXPSXXXXXXXX | 1015 |
| | Preferable | KSPPS | 1016 |
| | Key | KSXPS | 1017 |
| P10 | More preferable | QPKSPPSVTLFPPST | 1018 |
| | Preferable | XXKSXXSVXLFXXXX | 1019 |
| | Key | XXKSXXXVXLFXXXX | 1020 |
| | Key | TVLGQPKSPP | 1021 |
| | Key | SPPSV | 1022 |
| | Key | SVTLFPPSTE | 1023 |
| | Key | LFPPSTEELN | 1024 |
| P12 | Key | QPKSPPSVTLFPPST | 1025 |
| | Key | VLGQPKSPP | 1026 |
| | More preferable | KSPPSVTL | 1027 |
| | Preferable | KSPPSVT | 1028 |
| | Key | KSPP | 1029 |
| | Preferable | LFPPSTEELN | 1030 |
| | Key | LFPPSTEE | 1031 |
| P21 | Preferable | QPKSXXXXXXFPPST | 1032 |
| | Key | XXKSXXXXXXXXPST | 1033 |
| | Key | SPPS | 1034 |
| P23 | Key | XXKSXXSXXXFXXXX | 1035 |
| | Key | SPPS | 1036 |
| | Key | SVTLFPPST | 1037 |

TABLE 2-24
E24 Spot No. 13

| | | | SEQ |
|---|---|---|---|
| Protein | | | ID |
| name | intracellular cholesterol transporter 2 | | NO: |
| 15- | GIPVPFPIPESDGCK | | 1038 |
| residue | | | |
| sequence | | | |
| P2 | More preferable | GIPVPFPIPESDGCK | 1039 |
| | Preferable | XXPVPFPIXESDGXK | 1040 |
| | Key | XXXVPFPXXESDXXX | 1041 |
| | Preferable | ESDGCK | 1042 |
| | Key | ESDGXK | 1043 |
| P4 | Key | GIPVPFPIPESDGCK | 1044 |
| | Key | ESDGCK | 1045 |
| P10 | Key | GIPVPFPIPESDGCK | 1046 |
| | Preferable | IVMGIPVPF | 1047 |
| | Key | IPVPF | 1048 |
| | Preferable | ESDGCK | 1049 |
| | Key | ESDG | 1050 |
| P12 | Key | GIPVPFPIPESDGCK | 1051 |
| | Key | ESDGCK | 1052 |
| P13 | Preferable | GIPVPFPIPESDGCK | 1053 |
| | Key | XXXXXXXXXESDGXK | 1054 |
| | Preferable | ESDGCK | 1055 |
| | Key | ESDGXK | 1056 |
| P21 | Preferable | GXXXXFPIPESXXXK | 1057 |
| | Key | XXXXXFXXPESXXXK | 1058 |
| | More preferable | IPVPFPIPES | 1059 |
| | Preferable | XXXXFPIPES | 1060 |
| | Key | XXXXFXXPES | 1061 |
| | Preferable | ESDGCK | 1062 |
| | Key | ESDG | 1063 |
| P23 | More preferable | GIPVPFPIP | 1064 |
| | Preferable | IPVPFPIP | 1065 |
| | Key | IPVPFPI | 1066 |
| | Key | FPIPESDGCK | 1067 |
| P24 | Preferable | XXXXXXXXXXSDGCK | 1068 |
| | Key | XXXXXXXXXXSDGXK | 1069 |
| | More preferable | ESDGCK | 1070 |
| | Preferable | XSDGCK | 1071 |
| | Key | XSDGXK | 1072 |
| P25 | Key | XXXXXXXXXESDXCK | 1073 |
| | Preferable | ESDGCK | 1074 |
| | Key | ESDXCK | 1075 |

TABLE 2-25
E25 Spot No. 14

| | | | | Cross-reactivity- | | Synthesized | |
|---|---|---|---|---|---|---|---|
| Protein name | Apolipoprotein A-IV | | SEQ ID NO | confirmed food | Graph No | sequence | SEQ ID NO |
| 15-residue sequence | LTKDSEKLKEEIRKE | | 1076 | | | | |
| | Preferable | LTKDSEKLKEEIRKE | 1077 | | | | |
| P1 | Key | XXXXXXKLKEEXRXE | 1078 | | | | |
| | Key | KEEI | 1079 | | | | |
| P2 | Key | LTKDSEKLKEEIRKE | 1080 | | | | |
| | Key | KDSE | 1081 | | | | |
| | Key | KLKEEIRKE | 1082 | | | | |
| | Key | KEEIRKELED | 1083 | | | | |
| P3 | Preferable | LTKDSEKLKEEIRKE | 1084 | | | | |
| | Key | XXXDXEXXKXXXRXE | 1085 | | | | |
| P8 | Preferable | LTKDSEKLKEEIRKE | 1086 | | | | |
| | Key | XXXXXEKLKXXXRKE | 1087 | | | | |
| | Key | ERLTKDSE | 1088 | | | | |
| | Preferable | LTKDSEKLKE | 1089 | | | | |
| | Key | XXXXXEKLKX | 1090 | | | | |
| | Preferable | LKEEIRK | 1091 | | | | |
| | Key | LKXXXRK | 1092 | | | | |
| P9 | Key | LTKDSEKLKEEIRKE | 1093 | | | | |
| | Key | RLTKDSEKLK | 1094 | | | | |
| | Key | KLKEEI | 1095 | | | | |
| P16 | Preferable | XXKDSEKLKEEXXXX | 1096 | | | | |
| | Key | XXKDXXXLKEEXXXX | 1097 | | | | |
| | Key | KDSE | 1098 | | | | |
| | More preferable | KLKEEIRKE | 1099 | | | | |
| | Preferable | KLKEEXXXX | 1100 | | | | |
| | Key | XLKEEXXXX | 1101 | | | | |
| P17 | Key | LTKDSE | 1102 | | | | |
| | More preferable | KDSEKLKEEI | 1103 | | | | |
| | Preferable | KLKEEIRKE | 1104 | | | | |
| | Key | KLKEEI | 1105 | | | | |
| P20 | Preferable | XTXDXEKLKEEXRKE | 1106 | | | | |
| | Key | XXXDXXKLKEXXRKX | 1107 | | | | |
| | Still more preferable | EKLKEEIR | 1108 | | | | |
| | More preferable | EKLKEE | 1109 | | | | |
| | Preferable | KLKEE | 1110 | Sheep | 6 | KLKEE | 1110 |
| | Key | KLKEX | 1111 | Chicken | 6 | KLKER | 1131 |
| P22 | Key | LTXXSXXXKEEIXXE | 1112 | | | | |
| | Preferable | KEEIR | 1113 | | | | |
| | Key | KEEI | 1114 | | | | |

TABLE 2-26

| | Food with cross-reactivity (BLAST target food) | | | | | |
|---|---|---|---|---|---|---|
| Patient | Chicken | | | | | Sheep |
| No. | egg | Wheat | Peanut | Shrimp | Buckwheat | milk |
| 1 | | | | | | ● |
| 2 | ● | ● | ● | ● | ● | ● |
| 3 | ● | ● | ● | | ● | ● |
| 4 | ● | ● | | | | ● |
| 5 | ● | | | | | ● |
| 6 | ● | | | | | ● |
| 7 | ● | | | | | ● |
| 8 | | | | | | ● |
| 9 | | | | | | ● |
| 10 | ● | | | | | ● |
| 11 | ● | | | | | ● |
| 12 | | | | | | ● |
| 13 | ● | | | | | ● |
| 14 | ● | | ● | ● | ● | ● |
| 15 | | | ● | | | ● |
| 16 | ● | | | | | ● |
| 17 | ● | | | | | ● |
| 18 | | ● | | | | ● |
| 19 | ● | ● | | | | ● |
| 20 | ● | | | | | ● |
| 21 | ● | ● | | ● | | ● |
| 22 | | | | | | ● |
| 23 | | ● | | | | ● |
| 24 | | | | | | ● |
| 25 | ● | | | | | ● |

●: Allergic food

Table 2-1 to Table 2-25 show the specific sequences identified as epitopes that each bind to an IgE antibody in Example 4, and the sequences for which epitope cross-reactivity was confirmed in Example 5. Table 2-26 shows an example of foodstuffs to which 25 patients are allergic, and is a table summarizing the cases analyzed for epitope cross-reactivity in Example 5.

The invention provides a polypeptide comprising each amino acid sequence of (E1) to (E25) which comprises or consists of each of amino acid sequences of SEQ ID NOs: 27 to 1131 listed in Table 2 as a polypeptide comprising an amino acid sequence that specifically binds to an IgE antibody from an allergic patient. (E1) to (E25) are each an amino acid sequence that binds to an IgE antibody and is derived from one of the proteins listed in “Protein name” in Table 2 (hereinafter, sometimes referred to as an “epitope”).

(1) (E1), (E2): derived from an immunoglobulin M heavy chain secretory form protein;

(2) (E3), (E4), (E5), (E6), (E7), (E8) derived from a beta-1 metal-binding globulin protein;

(3) (E9), (E10): derived from a lactoperoxidase protein;

(4) (E14), (E15): derived from a an Ig heavy chain precursor (B/MT.4A.17. H5. A5) protein;

(5) (E16), (E17): derived from a perilipin protein;

(6) (E11), (E12): derived from a polymeric immunoglobulin receptor protein;

(7) (E13): derived from a protein: (7) C3-beta-c;

(8) (E23): derived from an immunoglobulin light chain, lambda gene cluster protein;

(9) (E20), (E21): derived from a glycoprotein 2 protein;

(10) (E22): derived from a lactadherin protein;

(11) (E18), (E19): derived from a zinc-alpha-2-glycoprotein protein;

(12) (E24): derived from an intracellular cholesterol transporter 2 protein; and

(13) (E25): derived from an apolipoprotein A-IV protein.

The epitope antigen of the invention is not limited, and preferably includes at least one of the following polypeptides.

(E1) A polypeptide comprising at least one amino acid sequence selected from amino acid sequences set forth in SEQ ID NOs: 27 to 58 and 1115;

(E2) a polypeptide comprising at least one amino acid sequence selected from amino acid sequences set forth in SEQ ID NOs: 59 to 97;

(E3) a polypeptide comprising at least one amino acid sequence selected from amino acid sequences set forth in SEQ ID NOs: 98 to 144;

(E4) a polypeptide comprising at least one amino acid sequence selected from amino acid sequences set forth in SEQ ID NOs: 145 to 195;

(E5) a polypeptide comprising at least one amino acid sequence selected from amino acid sequences set forth in SEQ ID NOs: 196 to 254 and 1116 to 1118;

(E6) a polypeptide comprising at least one amino acid sequence selected from amino acid sequences set forth in SEQ ID NOs: 255 to 288;

(E7) a polypeptide comprising at least one amino acid sequence selected from amino acid sequences set forth in SEQ ID NOs: 289 to 333 and 1119 to 1120;

(E8) a polypeptide comprising at least one amino acid sequence selected from amino acid sequences set forth in SEQ ID NOs: 334 to 352;

(E9) a polypeptide comprising at least one amino acid sequence selected from amino acid sequences set forth in SEQ ID NOs: 353 to 376 and 1121 to 1122;

(E10) a polypeptide comprising at least one amino acid sequence selected from amino acid sequences set forth in SEQ ID NOs: 377 to 384;

(E11) a polypeptide comprising at least one amino acid sequence selected from amino acid sequences set forth in SEQ ID NOs:385 to 411;

(E12) a polypeptide comprising at least one amino acid sequence selected from amino acid sequences set forth in SEQ ID NOs: 412 to 450;

(E13) a polypeptide comprising at least one amino acid sequence selected from amino acid sequences set forth in SEQ ID NOs: 451 to 500 and 1123 to 1124;

(E14) a polypeptide comprising at least one amino acid sequence selected from amino acid sequences set forth in SEQ ID NOs: 501 to 604 and 1125 to 1127;

(E15) a polypeptide comprising at least one amino acid sequence selected from amino acid sequences set forth in SEQ ID NOs: 605 to 632 and 1128;

(E16) a polypeptide comprising at least one amino acid sequence selected from amino acid sequences set forth in SEQ ID NOs: 633 to 673;

(E17) a polypeptide comprising at least one amino acid sequence selected from amino acid sequences set forth in SEQ ID NOs: 674 to 693;

(E18) a polypeptide comprising at least one amino acid sequence selected from amino acid sequences set forth in SEQ ID NOs: 694 to 757 and 1129;

(E19) a polypeptide comprising at least one amino acid sequence selected from amino acid sequences set forth in SEQ ID NOs: 758 to 791;

(E20) a polypeptide comprising at least one amino acid sequence selected from amino acid sequences set forth in SEQ ID NOs: 792 to 868 and 1130;

(E21) a polypeptide comprising at least one amino acid sequence selected from amino acid sequences set forth in SEQ ID NOs: 869 to 927;

(E22) a polypeptide comprising at least one amino acid sequence selected from amino acid sequences set forth in SEQ ID NOs: 928 to 996;

(E23) a polypeptide comprising at least one amino acid sequence selected from amino acid sequences set forth in SEQ ID NOs: 997 to 1037;

(E24) a polypeptide comprising at least one amino acid sequence selected from amino acid sequences set forth in SEQ ID NOs: 1038 to 1075; or

(E25) a polypeptide comprising at least one amino acid sequence selected from amino acid sequences set forth in SEQ ID NOs:1076 to 1114 and 1131.

Polypeptides (E1) to (E25), which have been described as a preferred embodiment, have each a specific sequence identified as an epitope that binds to an IgE in Examples herein as listed in Table 2. The epitope antigens of the invention may include not only the polypeptides (E1) to (E25) of the above-described preferred embodiment, but also variants, etc., described below. Hereinbelow, forms (variants) that can be included in the epitope antigens of the invention will be described.

Here, 25 different sequences including SEQ ID NO:27, 59, 98, 145, 196, 255, 289, 334, 353, 377, 385, 412, 451, 501, 605, 633, 674, 694, 758, 792, 869, 928, 997, 1038, or 1076, which have been described in “15-residue sequence” of Table 2, are 15-common-amino-acid-residue sequences each identified, as an epitope that binds to an IgE antibody, in epitopes (E1) to (E-25), by epitope mapping based on overlapping. One embodiment of the invention provides polypeptides comprising or consisting of these amino acid sequences. The epitope sequence included in the polypeptide of the invention may be the entire or portion of the 15 common amino acid residues in the epitope. Each epitope sequence has 4 amino acid residues or more, 5 amino acid residues or more, 6 amino acid residues or more, 7 amino acid residues or more, 8 amino acid residues or more, 9 amino acid residues or more, 10 amino acid residues or more, 11 amino acid residues or more, 12 amino acid residues or more, 13 amino acid residues or more, or 14 amino acid residues or more.

In the Examples herein, for example, a large number of polypeptides consisting of four amino acid residues (e.g., SEQ ID NO: 34, 67, 116, 195, 229, 275) have been identified as an epitope that binds to an IgE antibody. In addition, the binding to an IgE antibody was confirmed in the case of five or more amino acid residues. Thus, the presence of at least four amino acid residues is useful as an epitope sequence.

According to an embodiment of the invention, examples of a polypeptide variant include a polypeptide comprising four or more amino acid residues in the amino acid sequence specifically described in any of the above (E1) to (E25). For instance, the (E1) variant may include a “polypeptide comprising four or more amino acid residues in at least one amino acid sequence among the amino acid sequences set forth in SEQ ID NOs: 27 to 58 and 1115”. Preferred are 4 amino acid residues or more, 5 amino acid residues or more, 6 amino acid residues or more, 7 amino acid residues or more, 8 amino acid residues or more, 9 amino acid residues or more, 10 amino acid residues or more, 11 amino acid residues or more, 12 amino acid residues or more, 13 amino acid residues or more, or 14 amino acid residues or more in at least one amino acid sequence among the amino acid sequences set forth in SEQ ID NOs: 27 to 58 and 1115. The same applies to (E2) to (E25).

The “Preferable” sequence in Table 2 refers to a shorter partial sequence that can function as an epitope in the “15-residue sequence”. The “More preferable” sequence refers to a sequence more preferable than the above shorter partial sequence in view of improving the binding to an IgE antibody. The “Key” sequence refers to a sequence that seems to be critical in the “15-residue sequence”. In the “Key” sequence, the amino acid sequence denoted by “X” is an amino acid residue confirmed to have a binding ability to IgE antibodies which remains even after the amino acid residue is replaced with any alanine (glycine if the original amino acid residue is alanine) by alanine/glycine scanning. Thus, X can be any amino acid residue, preferably alanine (or glycine). Note that each sequence including no “X” denoted in the “Key” sequence is a case where no amino acid residues, the change of which still preserves the binding to an IgE antibody, have been found by alanine/glycine scanning.

For each of the epitopes listed in Table 2, each amino acid residue denoted by X is an amino acid residue, the change of which has been found to maintain the binding ability to an IgE antibody. The invention preferably allows one or more of the amino acid residues denoted as X in the “key” sequence corresponding to each “Preferable” sequence to be replaced by a given amino acid residue(s). For example, in one embodiment, in the preferable sequence SEQ ID NO: 27, there are multiple corresponding key sequences such as SEQ ID NOs: 28, 34, etc. Hereinafter, the same applies to the other “Preferable” sequences in (E1), the “key” sequences, or (E2) to (E25).

The number of amino acid residues that may be substituted is not limited, and is preferably 6 or less, 5 or less, 4 or less, 3 or less, 2 or less, or 1 or less. Hereinafter, the same applies to (E2) to (E25).

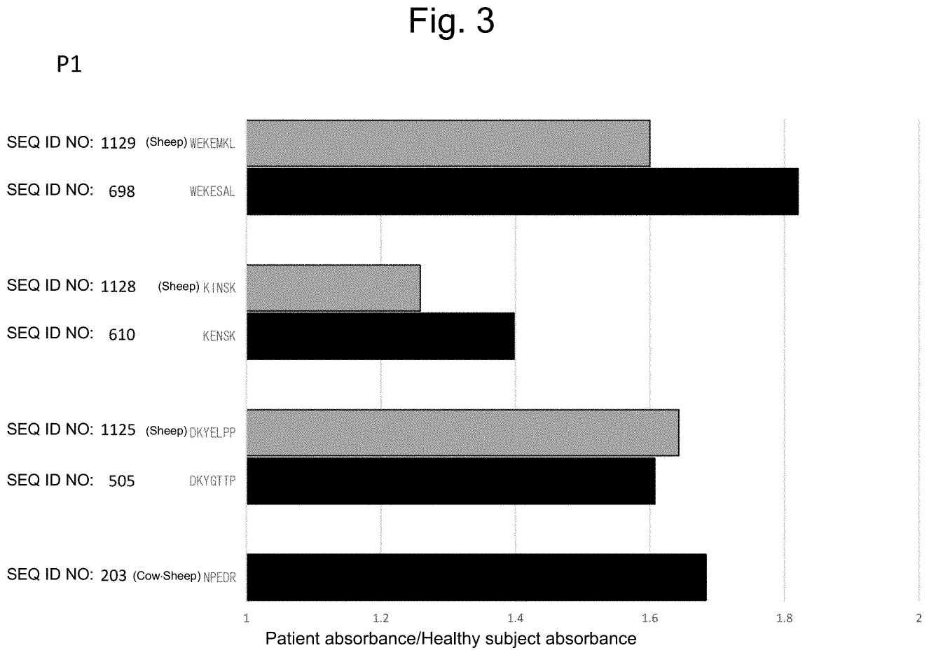

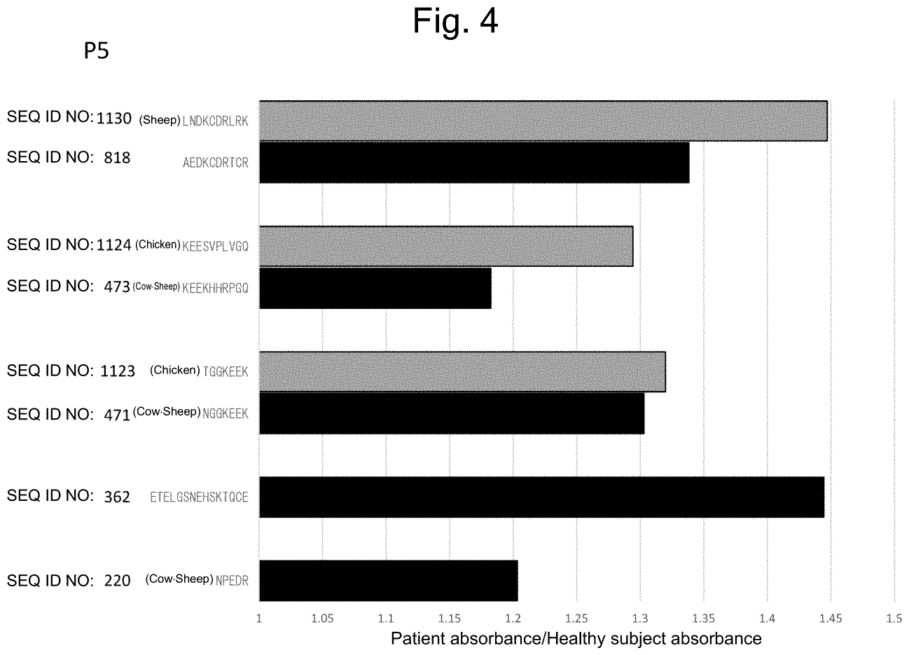

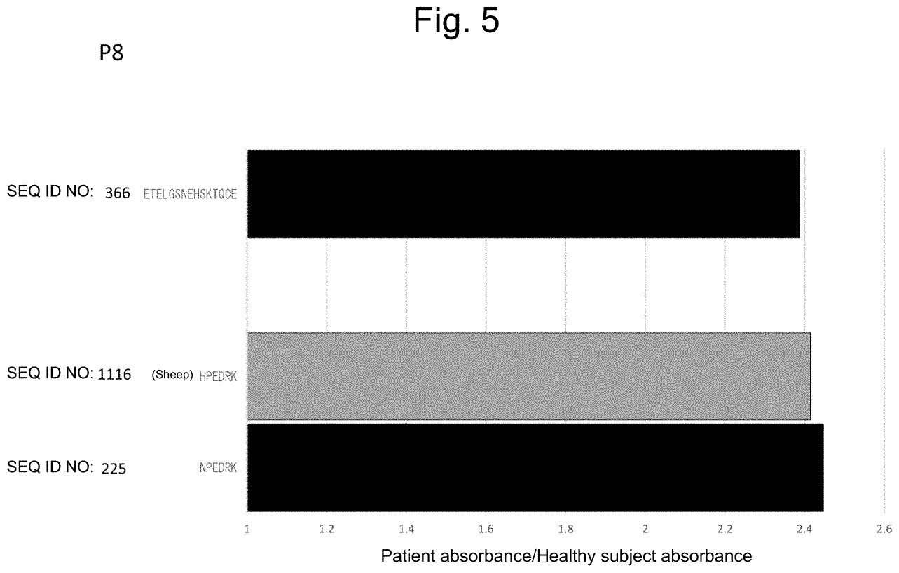

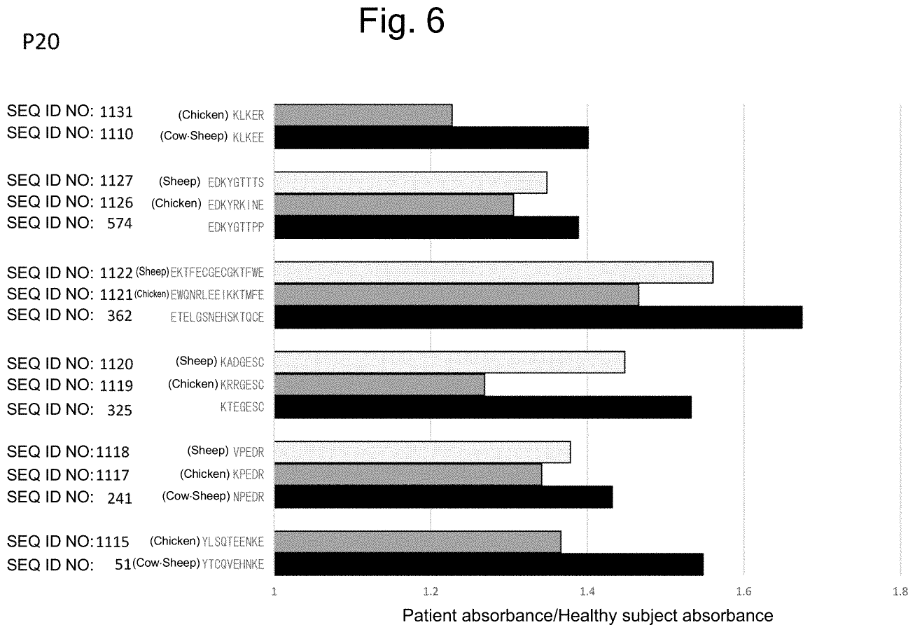

Each sequence listed in the “Synthesized sequence” and/or “SEQ ID NO:” column to the right of the Graph No. in Table 2 is the sequence for which the epitope cross-reactivity was found in Example 5. The wording “epitope cross-reactivity was found” means that an IgE antibody in serum of each allergic patient showed higher binding ability than IgE antibodies in serum of a non-allergic subject (specifically, larger than “1” in FIGS. 3 to 6). Thus, in one embodiment, the polypeptides of the invention include these amino acid sequences, or polypeptides comprising these amino acid sequences. In one embodiment, the IgE antibody in serum of each allergic patient binds to the polypeptide in the invention by 1.05-fold, 1.10-fold, 1.15-fold, 1.20-fold, or 1.25-fold more than the IgE antibodies in serum of a non-allergic subject.

As used herein, the “polypeptide comprising any of the amino acid sequences of (E1) to (E25)” includes a polypeptide comprising or consisting of any of the amino acid sequences of the polypeptides (E1) to (E25) described above as a preferred embodiment, as well as each form (variant) in which an amino acid residue(s) is substituted as described above. The wording “comprising any of the amino acid sequences set forth in SEQ ID NOs: . . . ” means that any other amino acid sequence may be included as long as it does not affect the binding ability (i.e., the function as each epitope) of any of the amino acid sequences set forth in SEQ ID NOs: . . . (including the above substituted forms) to an IgE antibody. A “polypeptide comprising any of the amino acid sequences set forth in (E1) to (E25)” may be a polypeptide in which two or more polypeptides comprising any of the amino acid sequences set forth in (E1) to (E25) are linked together with or without a spacer. The type of spacer is not particularly limited, and those routinely used by those skilled in the art for linking multiple peptides can be used. The spacer may be a polypeptide such as a hydrocarbon chain (e.g., Acp(6)-OH) or an amino acid chain.

Amino acid residues in the polypeptide other than those specifically identified can be optionally selected as long as they do not affect the binding to an IgE antibody (i.e., the function as an epitope). The amino acid residue(s) is not limited, and is preferably selected, if appropriate, from the sequence of the respective corresponding underlying epitope or the sequence of the underlying protein. For example, in SEQ ID NO: 34, only “HNKE” is specified, but if other amino acid residue(s) is to be added, they should be selected, if appropriate, from the base sequence SEQ ID NO: 27. Then, for the sequence listed in Table 2-1 as (E1), the amino acid residues of the sequence derived from the base protein (1) (corresponding to spot (1)) should be added.

The polypeptide comprising any of the amino acid sequences (E1)-(E25) above may be prepared by a chemical synthesis procedure such as solid phase peptide synthesis. Alternatively, each epitope-comprising polypeptide may be separated/produced by expressing the polypeptide as a recombinant polypeptide by a gene recombinant technology well-known to those skilled in the art and then performing a protein production procedure well-known to those skilled in the art. A polypeptide may be obtained by combining and linking two or more polypeptides, or may be obtained by repeatedly linking a single epitope. In this case, the binding ability to an Ig antibody is generally improved.

The length of the polypeptide comprising any of the amino acid sequences (E1) to (E25) is not limited. In a preferred embodiment, the length of the polypeptide comprising any of the amino acid sequences (E1) to (E25) may be 500 amino acids or less, 300 amino acids or less, 200 amino acids or less, 100 amino acids or less, 50 amino acids or less, 30 amino acids or less, 20 amino acids or less, 15 amino acids or less, 10 amino acids or less, or 5 amino acids or less. If the polypeptide is obtained by repeatedly linking one or more of the above amino acid sequences (E1) to (E25) one or two or more times, the length of the amino acid sequence portion may preferably be 1000 amino acids or less, 750 amino acids or less, 500 amino acids or less, 250 amino acids or less, 100 amino acids or less, 75 amino acids or less, 50 amino acids or less, 30 amino acids or less, 15 amino acids or less, 10 amino acids or less, or 5 amino acids or less. The number of amino acid residues listed as the length of the above polypeptide in a preferred embodiment is the sum of the lengths of the sequences (excluding the spacer) before and after the spacer.

Each antigen in the invention can specifically bind to an IgE antibody from an allergic patient.

Diagnosis Kit/Diagnosis Method (2)

The invention provides a method for providing an indicator for diagnosing an allergy in a subject, comprising the steps of:

(i) contacting a sample obtained from the subject with an antigen, wherein the sample is a solution comprising an IgE antibody;

(ii) detecting binding between the IgE antibody present in the sample obtained from the subject and the antigen; and

(iii) when the binding between the IgE antibody in the subject and the antigen is detected, an indicator of the fact that the subject is allergic is provided,

wherein the antigen is a polypeptide which is at least one polypeptide comprising any of the amino acid sequences set forth in (E1) to (E25) or a polypeptide in which two or more polypeptides comprising any of the amino acid sequences set forth in (E1) to (E25) are linked together with or without a spacer.

The “at least one polypeptide comprising any of the amino acid sequences set forth in (E1) to (E25)” or “the polypeptide in which two or more polypeptides comprising any of the amino acid sequences set forth in (E1) to (E25) are linked together with or without a spacer” may be herein referred to as the “antigen comprising any of the above (E1) to (E25).” The type of spacer is not particularly limited, and those routinely used by those skilled in the art for linking multiple peptides can be used. The spacer may be a hydrocarbon chain such as Acp(6)-OH or a polypeptide such as an amino acid chain.

In the case where the polypeptides comprising any of the above amino acid sequences (E1) to (E25) are linked together with or without a spacer, the number of polypeptides linked is not particularly limited. In one embodiment, the number is 2 or more, 3 or more, 4 or more, 5 or more, 6 or more, 8 or more, 10 or more, or 15 or more. In one embodiment, the number is 30 or less, 20 or less, 15 or less, 10 or less, 8 or less, 6 or less, 5 or less, 3 or less, or 2 or less.

In the polypeptide comprising any of the above amino acid sequences (E1) to (E25), the same unit may be repeated, or different units may be linked. In this way, when multiple polypeptides comprising any of the above amino acid sequences (E1) to (E25) are linked, the resulting polypeptide of the invention is applicable to the method, the kit, or the composition of the invention.

The sample obtained from a subject is as described in the above section “Diagnosis Kit and Diagnosis Method (1)”.

Detection of contact and the binding between a sample obtained from a subject and the polypeptide may be performed by the known procedure described in the above section “Diagnosis Kit and Diagnosis Method (1)”. Examples of the procedure that can be performed include ELISA (Enzyme-Linked Immunosorbent Assay), sandwich immunoassay, immunoblotting, immunoprecipitation, or immunochromatography.

The polypeptide comprising any of the above amino acid sequences (E1) to (E25) may be immobilized on or inside a carrier. In this case, steps (i) and (ii), can be performed using ELISA, sandwich immunoassay, immunochromatography, surface plasmon resonance, etc., and step (i) mentioned above can be performed by bringing the sample obtained from the subject into contact with a surface on which the polypeptide comprising any of the above amino acid sequences (E1) to (E25) is immobilized. In addition, the IgE antibody from the subject may be immobilized on or inside a carrier and the resulting material may then be used. The binding to the polypeptide comprising any of the amino acid sequences (E1)-(E25) may be detected by this technique. To establish binding onto a carrier or space from the carrier, or to facilitate contact of a polypeptide with the antibody, a spacer or a tag such as biotin may be added to the N-terminus or C-terminus of the polypeptide. In the case of binding to biotin, the carrier preferably has avidin.

The polypeptide comprising any of the above amino acid sequences (E1) to (E25) may not be necessarily immobilized on or inside a carrier. In this case, flow cytometry, etc., can be used in steps (i) and (ii), and the presence of the IgE-bound polypeptide comprising the amino acid sequences (E1) to (E25) can be confirmed with laser light. Examples of this procedure include the basophil activation test (BAT), which detects a surface antigen CD203c that appears when basophils are activated by contact with the polypeptide comprising any of the amino acid sequences (E1) to (E25). Other examples include the histamine release test (HRT), in which the polypeptide comprising any of the above amino acid sequences (E1) to (E25) is further brought into contact with blood cells in a sample to examine whether or not histamine is released.

The polypeptide comprising any of the amino acid sequences (E1)-(E25) is an antigen that specifically binds to an IgE antibody in an allergic patient. Therefore, when binding between an IgE antibody from a subject and the antigen is detected, an indicator indicating that the subject is allergic (including an indicator for cross-reactivity) is provided. To facilitate the synthesis of the polypeptide comprising any of the above amino acid sequences (E1) to (E25) using, for example, E. coli, a sequence may be added before or after the epitope so that the sequence length is made longer. In such a case, when the binding of the IgE antibody from a subject to any of the amino acid sequences (E1) to (E25) is detected, an indicator indicating that the subject is allergic (including an indicator for cross-reactivity) is provided. Thus, any sequence may be added before or after the epitope, namely any of the amino acid sequences (E1) to (E25).

The invention also provides a kit for diagnosing an allergy comprising at least one polypeptide comprising any of the above amino acid sequences (E1) to (E25). The diagnosis kit of the invention may be used in a method for providing an indicator for diagnosing an allergy as described above, or in a diagnosis method described below. The diagnosis kit of the invention may comprise at least one polypeptide comprising any of the above amino acid sequences (E1) to (E25), as well as an enzyme-labeled anti-IgE antibody and a chromogenic or luminescent substrate that serves as a substrate for the enzyme. It is also possible to use a fluorescently labeled anti-IgE antibody. In the diagnosis kit of the invention, the polypeptide comprising any of the above amino acid sequences (E1) to (E25) may be immobilized on or inside a carrier. The diagnosis kit of the invention may also be provided with instructions on the procedure for diagnosis and a package comprising the instructions.

In another embodiment, the above diagnosis kit contains a companion diagnostic agent for allergy. The companion diagnostic agent may be used to identify a patient who is expected to benefit from a drug or who is at a risk of severe adverse effects from a drug, or to study the reactivity of a drug to optimize treatment with the drug. Here, the treatment optimization includes, for example, determining a dosage regimen and a dose, deciding discontinuation of administration, and confirming an allergen component to be use for immunological tolerance.

The invention also provides a composition for diagnosing an allergy comprising at least one polypeptide comprising any of the above amino acid sequences (E1) to (E25). The diagnostic composition of the invention may be used in the following diagnosis method. The diagnostic composition of the invention may optionally contain a pharmaceutically acceptable carrier and/or an additive(s) commonly used with the polypeptide in the invention.

An embodiment of the invention provides a method for diagnosing an allergy in a subject, comprising the steps of:

(i) contacting a sample obtained from the subject with an antigen;

(ii) detecting binding of the antigen to an IgE antibody in the sample obtained from the subject; and

(iii) determining that the subject has the allergy when the binding of the antigen to the IgE antibody from the subject is detected, wherein the antigen is at least one polypeptide specified as a polypeptide comprising any of the above amino acid sequences (E1) to (E25). Here, steps (i) and (ii) are performed as described for each step of the method of providing an indicator for diagnosing an allergy.

In another embodiment, the invention provides a method for diagnosing an allergy in a subject, comprising administering to the subject at least one polypeptide comprising any of the above amino acid sequences (E1) to (E25). The method may be in the form of a skin test characterized by applying to the skin the polypeptide comprising any of the above amino acid sequences (E1) to (E25). Examples of the form of the skin test include: a prick test, in which a diagnostic composition is applied onto the skin, a small cut is given to a degree to which no bleeding occurs, to allow the polypeptide comprising any of the above amino acid sequences (E1) to (E25) to penetrate into the skin, thereby observing a skin reaction; a scratch test, in which a diagnostic composition is applied, and the skin is slightly scratched to observe the resulting reaction; a patch test, in which a diagnostic composition in the form of a cream or ointment is applied to the skin and the reaction is observed; or an intracutaneous test, in which the polypeptide comprising any of the above amino acid sequences (E1) to (E25) is administered intracutaneously and the reaction is observed. In the case of occurrence of a skin reaction, such as skin swelling, at the site where the polypeptide comprising any of the above amino acid sequences (E1) to (E25) has been applied, the subject is diagnosed as having an allergy. Here, the amount of polypeptide to be applied to the skin may be, for example, a dose of 100 μg or less per application.

In the allergy diagnosis, an oral loading test is often performed to identify the antigen and to examine the degree of antigen intake and symptoms. At least one polypeptide comprising any of the above amino acid sequences (E1) to (E25) may be used as an active ingredient for the oral loading test to diagnose an allergy. Here, the polypeptide to be used for the oral loading test may be an expressed and purified polypeptide, or may be a polypeptide expressed in a raw material/processed product, such as pollen rice expressing pollen allergens which are obtained by transforming rice with a gene of a cedar pollen antigen gene and expressing the polypeptide in the rice.

In one embodiment, the above diagnostic composition or diagnosis kit may be used for a prick test, scratch test, patch test, intracutaneous test, etc.

In addition, another embodiment of the invention also provides at least one polypeptide comprising any of the above amino acid sequences (E1) to (E25) for use in allergy diagnosis.

Still another embodiment of the invention provides use of at least one polypeptide comprising any of the above amino acid sequences (E1) to (E25) in the manufacture of an allergy diagnostic agent.

In this section, the allergy to be diagnosed may be an allergy to the polypeptide comprising any of the above amino acid sequences (E1) to (E25). That is, allergy diagnosis including detecting an allergy and providing a diagnostic indicator is a diagnosis of not only an allergy to a single polypeptide comprising any of the above amino acid sequences (E1) to (E25), but also an allergy including cross-reactivity.

Composition/Treatment Method (2)

The invention also provides a composition comprising at least one polypeptide comprising any of the above amino acid sequences (E1) to (E25).

In one embodiment, the composition of the invention is a pharmaceutical composition. In one embodiment, the composition of the invention is a quasi-drug composition or a non-medicinal composition (e.g., a cosmetic composition, a food composition).

In one embodiment, the above composition is used to treat an allergy. The allergy treatment means increasing the limit amount of a polypeptide in which the polypeptide does not cause any symptom even when taken into the body, and the treatment is ultimately aimed at achieving a state (remission) in which the disease does not develop with normal intake of the polypeptide.

The invention also provides a method for treating an allergy, comprising administering to a patient in need of allergy treatment at least one polypeptide comprising any of the above amino acid sequences (E1) to (E25).

Another embodiment of the invention also provides at least one polypeptide comprising any of the above amino acid sequences (E1) to (E25) for use in allergy treatment. Still another embodiment of the invention provides use of at least one polypeptide comprising any of the above amino acid sequences (E1) to (E25) in the manufacture of an allergy therapeutic agent.

In the allergy treatment, hyposensitization therapy, which aims to induce immunological tolerance by administering an antigen to a patient, is often used. At least one polypeptide comprising any of the above amino acid sequences (E1) to (E25) may be used as an active ingredient for hyposensitization therapy for an allergy.

Here, each antigen to be used in the hyposensitization therapy may be an expressed and purified polypeptide, or may be a polypeptide expressed in a raw material/processed product, such as pollen rice.

The administration route, the dosage, the administration frequency, and/or the administration period of the composition of the invention as well as other component(s) contained in the composition and the dosage form may be as described in the above section “Composition/Treatment Method (1)”. The dose in the case of using the polypeptide comprising any of the above amino acid sequences (E1) to (E25) may be, for instance, a dose of 100 μg or less per dosing in the case of adults.

In this section, the allergy of treatment interest may be an allergy to the polypeptide comprising any of the above amino acid sequences (E1) to (E25). Specifically, in the allergy treatment, not only an allergy to a single polypeptide comprising any of the above amino acid sequences (E1) to (E25), but also an allergy including cross-reactivity should be treated.

Tester Composition (2)

The invention provides a tester composition comprising an antibody against at least one polypeptide comprising any of the above amino acid sequences (E1) to (E25).

The antibody can be produced by a conventional procedure. For example, the antibody may be produced by immunizing a mammal such as a rabbit with the polypeptide comprising any of the above amino acid sequences (E1) to (E25). The antibody may be an Ig antibody, a polyclonal antibody, a monoclonal antibody, or an antigen-binding fragment thereof (e.g., a Fab, F(ab′)2, Fab′).

In addition, in the tester composition described above, the antibody may be provided in a form immobilized or bound onto or inside a carrier. The carrier is not particularly limited as long as the carrier can be used for detecting the binding between the antibody and the polypeptide comprising any of the above amino acid sequences (E1) to (E25). Any carrier known to those skilled in the art can be utilized. It is also preferable that the antibody against the polypeptide comprising any of the above amino acid sequences (E1) to (E25) be an antibody against any of the epitopes described in the above section “Epitopes” or polypeptides having an amino acid sequence in which the critical amino acid is identical thereto. This makes it possible to provide a tester composition also capable of detecting cross-reactivity.

Examples of the method for determining the presence or absence of the polypeptide comprising any of the above amino acid sequences (E1) to (E25) include the following method.
A method comprising bringing a produced antibody-comprising tester composition into contact with a sample prepared from a raw material/processed product, detecting biding of the antibody to a polypeptide comprising any of the above amino acid sequences (E1) to (E25) in the sample by using, for instance, ELISA, and determining, when the binding of the antibody to the polypeptide is detected, that the polypeptide is contained in the raw material/processed product of interest (the “method for determining the presence or absence of the polypeptide” comprises determining that, when the binding of the antibody to the polypeptide is decreased, the polypeptide has been eliminated or reduced).
A method comprising soaking a raw material/processed product into a filter paper and allowing a reaction with an antibody solution so as to detect, therein, a polypeptide comprising any of the above amino acid sequences (E1) to (E25).

Another embodiment of the invention encompasses a tester composition for determining the presence or absence of a polypeptide comprising any of the above amino acid sequences (E1) to (E25) in an object of interest, the tester composition comprising a primer corresponding to an epitope or a polypeptide having an amino acid sequence in which the critical amino acid is identical thereto. The above primer is not limited, and may be designed so as to include a portion of the nucleotide sequence, or a complementary strand thereof, of a nucleic acid encoding, for instance, the amino acid sequence specified in any of the above (E1) to (E25). Alternatively, the primer may be designed so that the primer is a nucleotide sequence of a region upstream of a portion encoding an epitope having an amino acid sequence specified in any of the above (E1) to (E25), or a polypeptide having an amino acid sequence in which the critical amino acid is identical thereto, or a nucleotide sequence of a complementary strand of a region downstream of the portion encoding the epitope or a polypeptide having an amino acid sequences in which the critical amino acid is identical thereto, in nucleic acids encoding proteins comprising the epitope and the polypeptide having the amino acid sequence in which the critical amino acid is identical thereto. Examples of such a primer include a primer which is a portion of at least one nucleotide sequence selected from the group consisting of SEQ ID NOs: 1, 3, 5, 7, 9, 11, 13, 15, 17, 19, 21, 23, and 25 and/or a primer which is a portion of a sequence complementary to at least one nucleotide sequence selected from the group consisting of SEQ ID NOs: 1, 3, 5, 7, 9, 11, 13, 15, 17, 19, 21, 23, and 25. Here, the position of the epitope in the full-length sequence of an antigen is as specified in Table 2 based on the results of the Examples. In addition, particularly when mRNA is of interest, a primer complementary to the poly A tail may be contained.

For example, DNA or mRNA obtained from a sample is used as a template, and the DNA is amplified by PCR (Polymerase Chain Reaction) including RT-PCR using the primer described above. The presence or absence of an antigen comprising any of the above (E1) to (E25) is then determined by determining the presence or absence of a nucleic acid encoding the amino acid sequence specified in any of the above (E1) to (E25) in the sequence of the amplified DNA sequence. Examples of the methods of PCR amplification for mRNA of interest include RACE method. When one of the amino acid sequences encoded by three possible open reading frames in the amplified DNA contains the amino acid sequence specified in any of the above (E1) to (E25), it is determined that the antigen is present. When no DNA is amplification, it is determined that no antigen is included.

In one embodiment, the tester composition described above is used to determine the presence or absence of a polypeptide comprising any of the above amino acid sequences (E1) to (E25) in an object of interest, for example, in a raw material or in a processed product production line. The raw material may be a foodstuff or may be a cosmetic raw material, a pharmaceutical raw material, or the like. The processed product may be a processed food product or may be a cosmetic, a pharmaceutical product, or the like. The above-mentioned tester composition may be used for searching for a biological species contained as a raw material, may be used for quality inspection of production lines and pre-shipment products by manufacturers, or for checking whether or not an antigen in raw materials and processed products of interest by consumers or users themselves. In addition, the tester composition may be used for checking inclusion of the polypeptide by measuring a change in the polypeptide peak(s) by mass spectroscopy.

Method of Determining Presence or Absence of Polypeptide (2)

The invention also encompasses a method for determining the presence or absence of a polypeptide comprising any of the above amino acid sequences (E1) to (E25) in a raw material/processed product. This method comprises detecting a polypeptide having the whole or a portion of the amino acid sequence of a polypeptide comprising any of the above amino acid sequences (E1) to (E25) in a raw material/processed product.

In one embodiment, the method of the invention comprises a step of bringing an antibody against at least one polypeptide comprising any of the above amino acid sequences (E1) to (E25) into contact with a raw material/processed product (including a liquid) to determine the presence or absence of the polypeptide in an object of interest.

The antibody, the definition of the raw material/processed product, the method of producing the antibody, the method of bringing the antibody into contact with a raw material/processed product, and the binding of the antibody to the antigen, for example, are as described above in the “Tester Composition (2)”.

Alternatively, in an embodiment, the method of determining the presence or absence of an antigen comprises detecting a portion of an epitope in the polypeptide comprising any of the above amino acid sequences (E1) to (E25) contained in an antigen. The “portion of an epitope” preferably has 4 amino acid residues or more, 6 amino acid residues or more, or 8 amino acid residues or more. The portion of epitope may be detected by a known procedure for detecting a specific amino acid sequence that is a portion of the polypeptide. For instance, it is possible to consider a method comprising cleaving proteins in a raw material/processed product (e.g., foodstuff) of interest with a digestive enzyme for removing an antigen, separating the digested raw material/processed product by HPLC or the like, and measuring whether or not the peak(s) assigned to a given epitope peptide is reduced by the antigen-removing treatment. Alternatively, an antibody recognizing a portion of a polypeptide comprising any of the above amino acid sequences (E1) to (E25) may be used to determine the presence or absence of the polypeptide-comprising antigen in an object of interest.

Antigen-Removed Raw Material, etc. (2)

The invention provides a raw material or processed product characterized in that at least one polypeptide comprising any of the above amino acid sequences (E1) to (E25) is eliminated or reduced.

The method of removing or reducing the antigen of the invention from a raw material or processed product is not limited. Any method of removing or reducing an antigen may be implemented as long as the polypeptide comprising any of the above amino acid sequences (E1) to (E25) can be eliminated or reduced. For instance, it is possible to use technique described in the above section “Antigen-Removed Food, etc. (1)”.

Removal or reduction of at least one polypeptide comprising any of the above amino acid sequences (E1) to (E25) may be achieved by removing or reducing the whole amino acid sequence or by cleaving or eliminating a portion having an amino acid sequence of the above (E1) to (E25) from an antigen protein. The term “eliminating” includes deleting or modifying the whole or a portion of the portion having the sequence specified by any of the above (E1) to (E25).

For example, the raw material, from which the polypeptide comprising any of the above amino acid sequences (E1) to (E25) has been eliminated or reduced, may be prepared using a raw material in which the polypeptide comprising any of the above amino acid sequences (E1) to (E25) is not any more expressed by using a gene modification technology. Any of techniques known to those skilled in the art may be used for the gene modification knockout technology.

The processed product, from which the polypeptide comprising any of the above amino acid sequences (E1) to (E25) has been eliminated or reduced, may be a processed product using a raw material, from which the polypeptide comprising any of the above amino acid sequences (E1) to (E25) has been eliminated or reduced, such as powdered milk using digested proteins as a raw material. In the case of using a regular raw material, treatment for removing or reducing the polypeptide comprising any of the above amino acid sequences (E1) to (E25) should be carried out before, during, or after preparation of the processed product. The “preparation of the processed product” means, for instance, preparing a processed food product from a food raw material (e.g., milk or beef). For instance, the wording “preparation of a processed milk product” means preparing a milk product obtained by processing a main raw material milk or preparing a food comprising the milk product as a raw material. Examples include, but are not limited to, preparation of a milk product such as cream, butter, butter oil, cheese, whey, whey concentrate, ice cream, milk concentrate, skimmed milk concentrate, unsweetened condensed milk, unsweetened skimmed condensed milk, sweetened condensed milk, sweetened skimmed condensed milk, whole milk powder, skimmed milk powder, cream powder, whey powder, protein-enriched whey powder, buttermilk powder, sweetened milk powder, prepared milk powder, prepared liquid milk, fermented milk such as yogurt, a lactobacillus beverage, a milk drink, which are produced by processing milk or a cake, pastry, custard pudding, milk agar, biscuit, cookie, snack, chocolate, bread, white sauce, potage, cream stew, gratin, curry roux, or stew roux, which contain the above material(s) as a raw material. In addition, the wording “preparation of a processed beef product” means preparing a product obtained by processing a main raw material beef or preparing a food comprising the product as a raw material. Examples of the processed meat product include, but are not limited to, preparation of ham, sausage, bacon, bouillon, consomme, steak, grilled meat, roast beef, tartar steak, hamburger steak, hamburger, meatballs, or nuggets.

It is possible to use techniques described in the above section “Antigen-Removed Food, etc. (1)” as methods of removing or reducing a polypeptide comprising any of the above amino acid sequences (E1) to (E25) in a processed product using ordinary raw materials. Examples of the method for cleaving a polypeptide comprising any of the above amino acid sequences (E1) to (E25) include a method for treating the peptide by cleavage with a specific digestive enzyme.

Method of Producing Raw Material or Processed Product with Antigen Eliminated or Reduced (2)

The invention provides a method for producing a raw material or processed product with at least one polypeptide comprising any of the amino acid sequences (E1) to (E25) eliminated or reduced, the method comprising the step of confirming that the antigen has been eliminated or reduced in a production process of the processed product.

In the production method, removal or reduction of the polypeptide comprising any of the amino acid sequences (E1) to (E25) means that at least one polypeptide comprising any of the above amino acid sequences (E1) to (E25) has been eliminated or reduced, or a portion of a sequence specified in any of the above (E1) to (E25) has been cleaved or eliminated from the antigen.

The technique for confirming that the polypeptide has been eliminated or reduced in a production process of a raw material or processed product is not particularly limited. Any technique may be used that can detect at least one polypeptide comprising any of the above amino acid sequences (E1) to (E25). For instance, the presence or absence of the polypeptides in a raw material or processed product may be confirmed based on the binding ability of an antibody against at least one polypeptide comprising any of the above amino acid sequences (E1) to (E25) to a sample comprising the raw material or a material occurring in the production process of the processed product. The details of such a method are as described in the above section “Diagnosis Kit and Diagnosis Method (2)”. That is, in the above production method, the “IgE antibody from a subject” in the above section “Diagnosis Kit and Diagnosis Method (2)” may be replaced with the “antibody against at least one polypeptide comprising any of the above amino acid sequences (E1) to (E25)”, and the “antigen” or “polypeptide” in the above section “Diagnosis Kit and Diagnosis Method (2)” may be replaced with the “sample comprising a material occurring in the production process of a processed product”. Then, techniques described in the above section “Diagnosis Kit and Diagnosis Method (2)” can be used to confirm that the antigen has been eliminated or reduced in the production process of a processed product. In addition, the tester composition described in the above section “Tester Composition (2)” can be used.

The present invention also pertains to: use of a kit in a method for diagnosing an allergy; use of a kit for diagnosing an allergy and/or providing an indicator for diagnosis; a composition for use in a method for diagnosing an allergy; use of a composition for diagnosing an allergy and/or for providing an indicator for diagnosis; a method for diagnosing an allergy and/or providing an indicator for diagnosis; use of an antigen (protein antigen or epitope antigen) in a method for detecting the presence or absence of an IgE antibody in a sample obtained from a subject (a living body such as a human); an antigen (protein antigen or epitope antigen) for use in treatment of an allergy; a kit or composition for detecting binding between an antigen (protein antigen or epitope antigen) and an IgE antibody in a sample obtained from a subject (a living body such as a human), the kit or composition comprising the antigen (protein antigen or epitope antigen); or use of a kit or composition for detecting binding between an antigen (protein antigen or epitope antigen) and an IgE antibody in a sample obtained from a subject (a living body such as a human), the kit or composition comprising the antigen (protein antigen or epitope antigen). Each term such as the “antigen” is as described above.

### EXAMPLES

Examples of the invention will be described below. The technical scope of the invention, however, is not limited to these Examples.

### Example 1: Confirmation of Protein Pattern

The below-described 2D electrophoresis was used to examine proteins contained in cow milk (Bos taurus-derived milk).

Extraction of Proteins

Proteins contained in milk were extracted and purified as follows. A urea buffer was added to milk to extract proteins. The composition of the urea buffer was the following:

30 mM Tris

2 M thiourea

7 M urea

4% (w/v) CHAPS:

3-[(3-cholamidopropyl)dimethylammonio]propanesulfonate

Suitable amount of dilute hydrochloric acid

Distilled water was added to adjust the total volume to 100 mL. The pH was 8.5. Thereafter, the precipitation operation was repeated twice using the 2D-CleanUP kit (manufactured by GE Company). The first precipitation operation was performed by adding TCA (trichloroacetic acid) to the above collected protein extract liquid to collect the resulting precipitate (TCA precipitate) occurring in this operation. The second precipitation operation was performed by adding acetone to the above collected TCA precipitate to collect the resulting precipitate (sample) obtained by this operation.

To Prepare Sample Solution

Part of the resulting sample (100 μg as a protein weight basis) was solubilized in 150 μl of DeStreak Rehydration Solution (manufactured by GE Company), which is a swelling buffer for a first-dimensional isoelectric focusing gel, to prepare a first-dimensional isoelectric focusing sample solution (sample swelling solution). The composition of DeStreak Rehydration Solution was the following:

7 M thiourea

2 M urea

4% (w/v) CHAPS

0.5% (v/v) IPG buffer; manufactured by GE Company

Appropriate amount of BPB (bromophenol blue)

Permeation of Sample into First-Dimensional Isoelectric Focusing Gel

A first-dimensional isoelectric focusing gel (IPG gel Immobiline Drystrip (pH 3-10 NL); manufactured by GE Company) was immersed in 140 μl of 1D isoelectric focusing sample solution (sample swelling solution), and allowed to permeate the solution overnight at room temperature.

In this Example, an IPGphor, manufactured by GE Company, was used as an electrophoresis apparatus.

An electrophoresis tray was filled with silicon oil. Filter paper moistened with water was positioned at both ends of the gel impregnated with the sample, and the gel was set in the electrophoresis tray so as to be covered with silicon oil. Each electrode was set such that the filter paper was interposed between the gel and the electrode.

The upper limit of current value in the isoelectric focusing apparatus was set to 75 μA per gel. First-dimensional isoelectric focusing was performed using the voltage program set as follows: (1) a constant voltage step was performed at a constant voltage of 300 V until 750 Vhr (a current change range during 30-min electrophoresis before the end of this step was 5 μA); (2) the voltage was gradually increased up to 1000 V over 300 Vhr; (3) the voltage was further increased gradually up to 5000 V over 4500 Vhr; and then (4) the voltage was kept at a constant voltage of 5000 V until the total Vhr reached 12000.

SDS Equilibration in Isoelectric Focusing Gel

After the above first-dimensional isoelectric focusing was performed, the gel was removed from the isoelectric focusing apparatus. Then, the gel was immersed in a reducing agent-comprising equilibration buffer, and was shaken at room temperature for 15 min. The composition of the reducing agent-comprising equilibration buffer was as follows:

100 mM Tris-HCl (pH 8.0)

6 M urea

30% (v/v) glycerol

2% (w/v) SDS

1% (w/v) DTT

Next, the reducing agent-comprising equilibration buffer was removed. The gel was then immersed in an alkylating agent-comprising equilibration buffer, and shaken at room temperature for 15 min to obtain an SDS-equilibrated gel. The composition of the alkylating agent-comprising equilibration buffer was as follows:

100 mM Tris-HCl (pH 8.0)

6 M urea

30% (v/v) glycerol

2% (w/v) SDS

2.5% (w/v) iodoacetamide

### Second-Dimensional SDS-PAGE

In this Example, the electrophoresis apparatus used was a XCell SureLock Mini-Cell, manufactured by Life Technologies, Inc. The gel used for second-dimensional electrophoresis was NuPAGE 4-12% Bis-Tris Gels, manufactured by Life Technologies. In addition, an electrophoresis buffer having the following composition was prepared and used.

50 mM MOPS

50 mM Tris base

0.1% (w/v) SDS

1 mM EDTA

Also, in this Example, an agarose solution for adhesion was used in which 0.5% (w/v) agarose S (manufactured by Nippon Gene) and an appropriate amount of BPB (bromophenol blue) were dissolved in the electrophoresis buffer.

A SDS-PAGE well was washed adequately with the electrophoresis buffer, and the buffer used for the washing was removed. Next, a thoroughly dissolved agarose solution for adhesion was added to the well. Then, the SDS-equilibrated gel was immersed in the agarose. Thereafter, the SDS-equilibrated gel was adhered closely to the second-dimensional electrophoresis gel with forceps. Subsequently, it was confirmed that the agarose was sufficiently solidified in a state where the two gels were closely adhered. Finally, electrophoresis was conducted at a constant voltage of 200 V for about 45 min.

Fluorescence Staining of Gel

Fluorescence staining of the gel was performed using SYPRO Ruby (Life Technologies, Inc.).

First, a sealed container to be used was thoroughly washed with 98% (v/v) ethanol beforehand. The electrophoresed second dimensional electrophoresis gel was removed from the SDS-PAGE apparatus and was placed in the washed sealed container. The gel was twice treated by immersion in an aqueous solution comprising 50% (v/v) methanol and 7% (v/v) acetic acid for 30 min. Next, the aqueous solution was replaced by water, followed by immersion for 10 min. Then, the second-dimensional electrophoresis gel was immersed in 40 ml of SYPRO Ruby, and shaken overnight at room temperature. Subsequently, the SYPRO Ruby was removed. After washed with water, the second-dimensional electrophoresis gel was shaken in an aqueous solution comprising 10% (v/v) methanol and 7% (v/v) acetic acid for 30 min. Further, the aqueous solution was replaced by water, followed by shaking for 30 min or longer.

Analysis

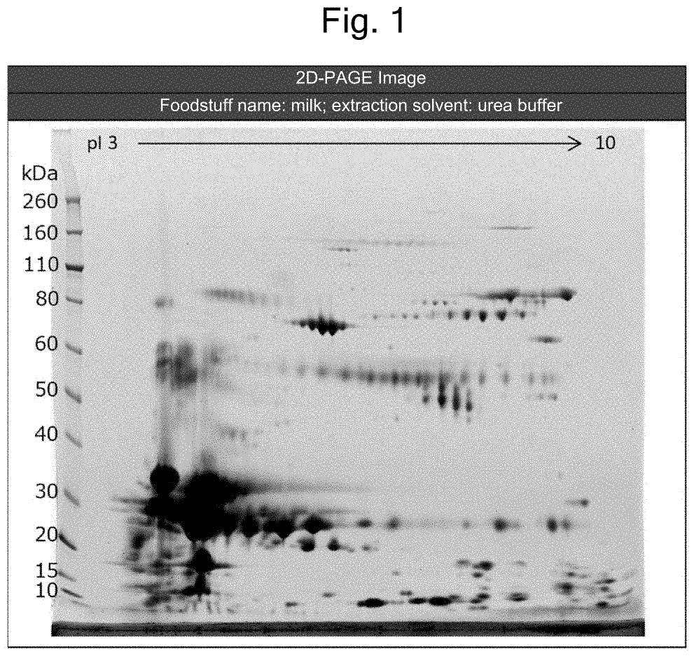

The second-dimensional electrophoresis gel, which underwent the series of the foregoing treatments, was subjected to fluorescence image scanning using a Typhoon 9500 (manufactured by GE Company). FIG. 1 shows the results of second-dimensional electrophoresis of proteins contained in milk. On the left side of the gel photograph in each figure, molecular weight marker bands can be seen, and the position of each band indicates a specific molecular weight (KDa).

### Example 2: Identification of Antigen by Immunoblotting

Each antigen was identified by immunoblotting as follows. The procedure described in Example 1 was performed until the “2D SDS-PAGE”, and then, the following operations “Membrane Transfer”, “Immunoblotting”, and “Analysis” were carried out.

Transfer to Membrane

Transfer to membrane was conducted using the following transfer apparatus and transfer buffer.

Transfer apparatus: XCell SureLock Mini-Cell and XCell II Blotting Module (manufactured by Life Technologies)

Transfer buffer: NuPAGE transfer buffer (×20) (manufactured by Life Technologies) diluted 20-fold with milliQ water was used.

Specifically, proteins in the second-dimensional electrophoresis gel were transferred to a membrane (PVDF membrane) according to the following procedure.

(1) A PVDF membrane was immersed in 100% methanol and then milliQ water, and then placed in a transfer buffer for hydrophilic treatment of the PVDF membrane.

(2) A sponge, a filter paper, the gel after the second dimensional SDS-PAGE, the hydrophilized PVDF membrane, a filter paper, and a sponge were set in this order, and a constant voltage was applied thereto in the transfer apparatus for 1 h.

Immunoblotting

Immunoblotting of the membrane was conducted using, as a primary antibody, serum from a patient with milk allergy or serum from a subject without milk allergy.

The immunoblotting of the membrane was performed according to the following procedure.

(1) The transferred membrane was shaken in Pierce (registered trademark) Fast Blocking Buffer (manufactured by Thermo, Inc.) (hereinafter referred to as “Fast Block”) for 1 h at room temperature.

(2) 10% serum/Fast Block solution was added as a primary antibody, and the mixture was allowed to stand for 1 h at room temperature.

(3) The membrane was washed with PBST solution (PBS buffer comprising 0.1% nonionic surfactant Tween (registered trademark) 20) (5 min x 3 times).

(4) Anti-human IgE-HRP (horseradish peroxidase) was diluted 5000-fold with Fast Block solution to give a secondary antibody, and the membrane was shaken in the resulting solution for 1 h at room temperature.

(5) The membrane was washed with PBST solution (5 min x 3 times).

(6) In Pierce Western Blotting Substrate Plus (manufactured by Thermo, Inc.), the membrane was allowed to stand for 5 min.

Analysis

The membrane, which underwent the foregoing series of treatments, was subjected to fluorescence image scanning using a Typhoon 9500 (manufactured by GE Company)

The immunoblot obtained by using serum from each patient with milk allergy was compared with the immunoblot obtained by using serum from a control, a subject without milk allergy. The immunoblot obtained by using serum from a patient with milk allergy was different, in terms of proteins contained in milk, from that in the case of using serum from a subject without milk allergy. Here, 14 spots, which differ from those of known milk allergen proteins, were detected (FIG. 2). As a result of analyzing the amino acid sequences in Example 3, spots 2 and 3 were found to be derived from the same protein. The isoelectric point of each spot was listed in Table 1.

### Example 3: Mass Spectrometry and Antigen Identification

The amino acid sequences of the antigens which generate the spots described above were identified by mass spectroscopy.

Specifically, the following procedure was used for protein extraction and mass spectrometry.

(1) Milk was subjected to protein extraction, second-dimensional electrophoresis, and transfer to membrane according to the procedures of Examples 1 and 2, and the membrane was stained with 0.008% Direct blue/40% ethanol and 10% acetic acid under shaking.

(2) The membrane was destained by treatment with 40% ethanol and 10% acetic acid for 5 min three times, washed with water for 5 min, and air-dried.

(3) Each spot of interest was cut out with a clean cutter blade and placed in a centrifuge tube. After hydrophilic treatment of the membrane with 50 μL of methanol, the membrane was washed twice with 100 μL of water, and centrifuged to remove water. Then, 20 μL of 20 mM NH₄HCO₃ and 50% acetonitrile was added.

(4) 1 μL of 1 μmol/μL lysyl endopeptidase (WAKO) was added, and the mixture was allowed to stand at 37° C. for 60 min. The resulting solution was collected in a new centrifuge tube. Subsequently, 20 μL of 20 mM NH₄HCO₃/70% acetonitrile was added to the membrane, which was immersed for 10 min at room temperature and the resulting solution was further collected. The material was dissolved in 10 μL of 0.1% formic acid and 4% acetonitrile, and then transferred into the tube.

(5) The collected solution was dried under reduced pressure, dissolved in 15 μl of solution A (0.1% formic acid and 4% acetonitrile solution), and subjected to mass spectrometry (ESI-TOF6600, manufactured by AB Sciex, Inc.).

(6) Protein identification based on the mass data obtained from the mass spectrometer was performed by searching Uniprot and NCBI.

Results

The amino acid sequence of each spot was detected. The mass data of each spot as obtained using a mass spectrometer was further analyzed using Uniprot, NCBI, and each spot was identified as each protein listed in Table 1.

Spots 2 and 3 are derived from the same protein. These spots were detected as different spots in the immunoblot of Example 2. This is because although the amino acid sequence is identical, the isoelectric point and/or the molecular weight were changed due to post-translational modifications such as glycosylation and phosphorylation. Although the post-translational modifications were different, the binding to an IgE antibody was still detected. This indicates that there was no influence of the post-translational modifications.

### Example 4: Identification of Epitopes

Epitopes of allergenic components in milk Epitopes of allergen components in milk were identified using the following procedures.

(A) Milk Epitope Mapping (1)

Epitope mapping was performed using a library of overlapping peptides (length: 15 amino acids) corresponding to amino acid sequences identified as allergic components in milk. Specifically, a library of overlapping peptides was prepared based on the amino acid sequences of SEQ ID NOs: 2, 4, 6, 8, 10, 12, 14, 16, 18, 20, 22, 24, and 26.

Each peptides to be synthesized was shifted by 10 amino acids. That is, each peptide has a 5-amino-acid overlap with the previous and subsequent peptides.

For the peptide array preparation, the Intavis CelluSpots (trademark) technology was used. Specifically, the procedure was as follows: (1) peptides of interest were synthesized on amino-modified cellulose discs using an automated synthesizer (Intavis MultiPep RS); (2) each amino-modified cellulose disc was dissolved to obtain a cellulose-bound peptide solution; and (3) each cellulose-bound peptide was spotted on a coated glass slide. The details of each step were as follows.

(1) Synthesis of Peptides

Each peptide was synthesized stepwise on an amino-modified cellulose disc in a 384-well synthesis plate by using 9-fluorenylmethoxycarbonyl (Fmoc) chemical reaction. Specifically, the amino acid with an amino group bound to the Fmoc group was activated in a solution comprising N,N′-diisopropylcarbodiimide (DIC) and 1-hydroxybenzotriazole (HOBt) in dimethylformamide (DMF). The mixture was added dropwise onto the cellulose disc so that the Fmoc-bound amino acid was bound to an amino group on the cellulose disc (coupling). Unreacted amino groups were capped with acetic anhydride, and the disc was washed with DMF. The Fmoc groups were eliminated from the amino groups of the amino acids bound to the amino groups on the cellulose discs by treating with piperidine and washing with DMF. The amino acids bound to the amino group on the cellulose disc was repeatedly subjected to the above coupling, capping, and Fmoc group, thereby extending the amino terminus for peptide synthesis.

(2) Dissolution of Amino-Modified Cellulose Disc

The cellulose disc, on which a peptide of interest as obtained in the above “(1) To Synthesize Peptide” was bound, was transferred to a 96-well plate, and treated with a side chain deprotection mixture comprising trifluoroacetic acid (TFA), dichloromethane, triisopropylsilane (TIPS), and water for the amino acid side chain deprotection. Next, the deprotected cellulose-bound peptide was dissolved in a mixed solution of TFA, perfluoromethanesulfonic acid (TFMSA), TIPS, and water. The peptide was precipitated in tetrabutyl methyl ether (TBME), resuspend in dimethylsulfoxide (DMSO), and mixed with a mixed solution of NaCl, Na citrate, and water to prepare a peptide solution for slide spotting.

(3) Spotting of Cellulose-bound Peptide Solution

The peptide solution for slide spotting as obtained in the above “(2) To Dissolve Amino-Modified Cellulose Disc” was spotted on an Intavis CelluSpots (trademark) slide by using an Intavis slide spotting robot, and the slide was dried to prepare a peptide array.

The peptide array was used to measure whether or not each peptide fragment was bound to an IgE antibody in serum from each patient with milk allergy through an antigen/antibody reaction. The measurement was conducted according to the following procedure.

(1) Peptides in Pierce Protein-Free (PBS) Blocking Buffer (manufactured by Thermo, Inc.) were shaken for 1 h at room temperature.

(2) The array was shaken overnight at 4° C. in 2% serum/Pierce Protein-Free (PBS) Blocking Buffer (manufactured by Thermo, Inc.).

(3) The array was washed with PBST (PBS buffer comprising 3% nonionic surfactant Tween (registered trademark) 20) for 5 min (×3 times).

(4) Anti-human IgE antibody-HRP (1:20,000, Pierce Protein-Free (PBS) Blocking Buffer (manufactured by Thermo, Inc.)) was added, and the mixture was shaken for 1 h at room temperature.

(5) The array was washed with PBST for 5 min (×3 times).

(6) A Pierce ECL Plus Western Blotting Substrate (manufactured by Thermo, Inc.) was added, and the array was shaken for 5 min at room temperature.

(7) The chemiluminescence of each peptide treated in (1) to (6) above was measured using Amersham Imager 600.

ImageQuant TL (GE Healthcare, Inc.) was used to quantify and the results were expressed numerically, the level of chemiluminescence in an image obtained during the above measurement (7). The second highest value among the numeric values for the respective images obtained from the results using serum from each of 6 subjects without milk allergy was defined as The N2nd value. The N2nd value of each peptide was subtracted from numeric value obtained from images obtained from the results using serum from each of 25 patients. It was determined that a peptide having the difference of 800,000 or higher was a peptide bound to an IgE antibody in a patient-specific manner.

The peptides (SEQ ID NOs: 27, 59, 98, 145, 196, 255, 289, 334, 353, 377, 385, 412, 451, 501, 605, 633, 674, 694, 758, 792, 869, 928, 997, 1038, and 1076) derived from spots 1 to 14 (spots 2 and 3 are derived from the same protein), in which known epitopes were not included, were each found to be bound to an IgE antibody in a patient-specific manner.

(B) Milk Epitope Mapping (2): Overlapping

As described in (A), on the basis of the sequences (SEQ ID NOs: 27, 59, 98, 145, 196, 255, 289, 334, 353, 377, 385, 412, 451, 501, 605, 633, 674, 694, 758, 792, 869, 928, 997, 1038, and 1076) of the peptides bound to an IgE antibody in serum in a patient-specific manner, a library of overlapping peptide fragments (the length: 10 amino acids) was constructed using in each peptide sequence or an allergic component amino acid sequence comprising the peptide sequence with sequences before and after the peptide being added. Then, epitope mapping was performed.

Each peptide to be synthesized was shifted by one amino acid. That is, each peptide has a 9-amino-acid overlap with the previous and subsequent peptides.

The library was constructed by substantially the same procedure as in (A). The same technique as above was used to evaluate each peptide fragment for binding to an IgE antibody in a patient's serum. The image obtained as the results of using the patient's serum was quantified to be represented numerically. This value was compared with a value obtained using each overlapping-based peptide. The peptide having lost or markedly decreased binding ability to an IgE antibody from a patient as compared with the peptide shifted one amino acid was determined as a peptide having no binding ability to the IgE antibody.

The level of chemiluminescence in an image obtained by measurement in the same manner as in (A) was converted into a numeric value. Among the values determined from the images obtained from the results of using a patient's serum, the value obtained using an overlapping base sequence (SEQ ID NO: 27, 59, 98, 145, 196, 255, 289, 334, 353, 377, 385, 412, 451, 501, 605, 633, 674, 694, 758, 792, 869, 928, 997, 1038, or 1076) was defined as 100%. In comparison with these peptides, the binding ability of other peptides are judged as follows: the value of less than 30% indicated no binding ability to the IgE antibody; the value of 30% or more and less than 50% indicated poor but some binding ability to the IgE antibody; the value of 50% or more and less than 70% indicated binding ability, though somewhat poor, to the IgE antibody; and the value of 70% or more indicated no difference in binding ability to the IgE antibody or indicated sufficient binding ability to the IgE antibody.

In this way, the peptide was determined to be a peptide having remaining binding ability to the IgE antibody.

This analysis has revealed a critical region for binding to IgE antibody from patients, in each overlapping base sequence.

(C) Milk Epitope Mapping (3): Alanine/Glycine Scanning

The amino acid sequences identified in (A) were used to construct, using substantially the same technique as above, a library of peptide fragments in which the amino acids were substituted from the amino-terminal side with alanine (glycine if the original amino acid was alanine) one by one by using a technique called alanine/glycine scanning (NPL 1). The same technique as above was used to evaluate each peptide fragment for binding to an IgE antibody in a patient's serum. The amino acid at a position where the binding ability to a patient's IgE antibody was lost or markedly decreased by alanine/glycine substitution was determined to be an amino acid critical for exertion of original antigenicity or an amino acid affecting exertion of original antigenicity. The amino acid, the substitution of which did not result in a loss or a marked decrease in the binding ability to the patient's IgE antibody, was determined as a replaceable amino acid that is not critical for exertion of original antigenicity.

The level of chemiluminescence in an image obtained by measurement in the same manner as in (A) was converted into a numeric value. Among the values determined from the images obtained from the results of using sera from 25 patients, the value obtained using each alanine/glycine scanning base sequence (SEQ ID NO: 27, 59, 98, 145, 196, 255, 289, 334, 353, 377, 385, 412, 451, 501, 605, 633, 674, 694, 758, 792, 869, 928, 997, 1038, or 1076) was defined as 100%. In comparison with these peptides, the binding ability of other peptides are judged as follows: the value of less than 30% indicated no binding ability to the IgE antibody; the value of 30% or more and less than 50% indicated poor but some binding ability to the IgE antibody; the value of 50% or more and less than 70% indicated binding ability, though somewhat poor, to the IgE antibody but indicated binding to the IgE antibody; and the value of 70% or more indicated no difference in binding ability to the IgE antibody or indicated sufficient binding ability to the IgE antibody. In this way, the peptide was determined to be a peptide having remaining binding ability to the IgE antibody. The results of (A) to (C) have been analyzed to find a consensus sequence important for exertion of original antigenicity in regions important for binding to IgE antibody from patients, in the alanine/glycine scanning base sequence. The sequences for all 25 different epitopes were identified, and the results were summarized in Table 2.

### Example 5: Confirmation of Epitope Cross-Reactivity

Using a key sequence in which amino acids other than amino acids important for maintaining binding to IgE antibodies, in each of the epitope sequences found in the milk proteins in Table 2 were defined as any amino acid (X), proteins having the key sequence in allergen foodstuff in common among patients listed in Table 2-26 were searched by BLAST. Then, BLAST was used to search for proteins having the same key sequence in foodstuffs allergic to each patient at the same time as listed in Table 2-26. As a result, each amino acid sequence was identified which was included in sequences of each food listed in, for example, the column “Cross-reactivity-checked food” in Table 2.

Each peptide comprising an amino acid sequence listed in the column “Synthesized sequence” of Table 2 was evaluated, by ELISA, for binding to an IgE antibody from a patient allergic to each food listed in the column “Cross-reactivity-confirmed food” of Table 2. The peptide was synthesized by Fmoc method so that the peptide was N-terminally biotinylated.

The ELISA was performed according to the following procedure.

(1) The concentrations of the biotinylated peptides were adjusted to 10 μg/mL with PBST (0.1% Tween (registered trademark) 20).

(2) 20 μL of each peptide solution was added to each well of a streptavidin-coated 384-well plate and shaken for 1 h at room temperature. The solution was collected, and the well was then washed five times with PBS.

(3) 80 μL of Pierce Protein-Free (PBS) Blocking Buffer (manufactured by Thermo, Inc.) was added, and the mixture was shaken for 1 h at room temperature. The solution was removed, and the plate was then washed three times with PBST.

(4) 40 μL of 2% serum/Canget Signal Solution I (manufactured by TOYOBO, Inc.) was added, and the mixture was shaken for 1 h at room temperature. The solution was removed, and the plate was then washed five times with PBST.

(5) 40 μL of diluted secondary antibody solution (1:10000, Canget Signal Solution II (manufactured by TOYOBO, Inc.)) was added, and the mixture was shaken for 1 h at room temperature. The solution was removed, and the plate was then washed three times with PBST.

(6) 40 μL of 1-Step Ultra TMB-ELISA (manufactured by Thermo, Inc.) was added, and the mixture was shaken for 15 min at room temperature.

(7) 40 μL of 2 M H2504 was added. The absorbance at 450 nm was then measured.

Peptides having these amino acid sequences were prepared according to substantially the same procedure as in (A) of Example 4, and whether an IgE antibody in serum from an allergic patient or a non-allergic subject could bind to the peptides was measured. Serum from each of two non-allergic subjects was used for measurement. The results were averaged. Then, values divided by the average was used.

FIGS. 3 to 6 show the results. P1, P5, P8, and P20 each represent the patient number. Bar graphs in each figure show absorbance of allergic patients/absorbance of non-allergic patients (healthy subjects). The origin of foodstuff as designated in each graph is the origin of foodstuff comprising a polypeptide including a base amino acid sequence of each synthetic sequence used. If not indicated, the origin of foodstuff is “Cow”.

As clearly demonstrated in FIGS. 3 to 6, all the polypeptides with each amino acid sequence designated in each figure exhibited higher (more than “1”) binding ability to an IgE antibody from each allergic patient than an IgE antibody in serum from each non-allergic subject. Thus, the cross-reactivity of each polypeptide was demonstrated. This indicates that these epitopes can be utilized to detect the cross-reactivity to an antigen other than that from cow milk. Further, it has been supported that the site “X” can be any amino acid residue.

1. A kit for diagnosing an allergy, comprising at least one of the following polypeptides (E1) to (E25):
(E1) a polypeptide comprising at least one amino acid sequence selected from amino acid sequences set forth in SEQ ID NOs: 27 to 58 and 1115;
(E2) a polypeptide comprising at least one amino acid sequence selected from amino acid sequences set forth in SEQ ID NOs: 59 to 97;
(E3) a polypeptide comprising at least one amino acid sequence selected from amino acid sequences set forth in SEQ ID NOs: 98 to 144;
(E4) a polypeptide comprising at least one amino acid sequence selected from amino acid sequences set forth in SEQ ID NOs: 145 to 195;
(E5) a polypeptide comprising at least one amino acid sequence selected from amino acid sequences set forth in SEQ ID NOs: 196 to 254 and 1116 to 1118;
(E6) a polypeptide comprising at least one amino acid sequence selected from amino acid sequences set forth in SEQ ID NOs: 255 to 288;
(E7) a polypeptide comprising at least one amino acid sequence selected from amino acid sequences set forth in SEQ ID NOs: 289 to 333 and 1119 to 1120;
(E8) a polypeptide comprising at least one amino acid sequence selected from amino acid sequences set forth in SEQ ID NOs: 334 to 352;
(E9) a polypeptide comprising at least one amino acid sequence selected from amino acid sequences set forth in SEQ ID NOs: 353 to 376 and 1121 to 1122;
(E10) a polypeptide comprising at least one amino acid sequence selected from amino acid sequences set forth in SEQ ID NOs: 377 to 384;
(E11) a polypeptide comprising at least one amino acid sequence selected from amino acid sequences set forth in SEQ ID NOs:385 to 411;
(E12) a polypeptide comprising at least one amino acid sequence selected from amino acid sequences set forth in SEQ ID NOs: 412 to 450;
(E13) a polypeptide comprising at least one amino acid sequence selected from amino acid sequences set forth in SEQ ID NOs: 451 to 500 and 1123 to 1124;
(E14) a polypeptide comprising at least one amino acid sequence selected from amino acid sequences set forth in SEQ ID NOs: 501 to 604 and 1125 to 1127;
(E15) a polypeptide comprising at least one amino acid sequence selected from amino acid sequences set forth in SEQ ID NOs: 605 to 632 and 1128;
(E16) a polypeptide comprising at least one amino acid sequence selected from amino acid sequences set forth in SEQ ID NOs: 633 to 673;
(E17) a polypeptide comprising at least one amino acid sequence selected from amino acid sequences set forth in SEQ ID NOs: 674 to 693;
(E18) a polypeptide comprising at least one amino acid sequence selected from amino acid sequences set forth in SEQ ID NOs: 694 to 757 and 1129;
(E19) a polypeptide comprising at least one amino acid sequence selected from amino acid sequences set forth in SEQ ID NOs: 758 to 791;
(E20) a polypeptide comprising at least one amino acid sequence selected from amino acid sequences set forth in SEQ ID NOs: 792 to 868 and 1130;
(E21) a polypeptide comprising at least one amino acid sequence selected from amino acid sequences set forth in SEQ ID NOs: 869 to 927;
(E22) a polypeptide comprising at least one amino acid sequence selected from amino acid sequences set forth in SEQ ID NOs: 928 to 996;
(E23) a polypeptide comprising at least one amino acid sequence selected from amino acid sequences set forth in SEQ ID NOs: 997 to 1037;
(E24) a polypeptide comprising at least one amino acid sequence selected from amino acid sequences set forth in SEQ ID NOs: 1038 to 1075; or
(E25) a polypeptide comprising at least one amino acid sequence selected from amino acid sequences set forth in SEQ ID NOs:1076 to 1114 and 1131.

2. A composition for diagnosing an allergy, the composition comprising at least one polypeptide as defined in any of (E1) to (E25) of claim 1.

3. A method for providing an indicator for diagnosing an allergy in a subject, the method comprising the steps of:
(i) contacting a sample obtained from the subject with an antigen, wherein the sample is a solution comprising an IgE antibody;
(ii) detecting binding between the IgE antibody present in the sample obtained from the subject and the antigen; and
(iii) when the binding between the IgE antibody in the subject and the antigen is detected, an indicator of the fact that the subject is allergic is provided,
wherein the antigen is at least one polypeptide as defined in any of (E1) to (E25) of claim 1.

4. An antigen, which is at least one polypeptide as defined in any of (E1) to (E25) of claim 1 and is causative of an allergy.

5. A composition comprising at least one antigen according to claim 4.

6. The composition according to claim 5 wherein the composition is intended for the treatment of an allergy.

7. A tester composition for determining the presence or absence of an antigen in an object of interest, the tester composition comprising an antibody that binds to at least one polypeptide as defined in any of (E1) to (E25) of claim 1.

8. A tester composition for determining the presence or absence of an antigen in an object of interest, the tester composition comprising at least one primer comprising a portion of a nucleotide sequence, and/or a portion of a complementary strand thereof, of a nucleic acid encoding a polypeptide as defined in any of (E1) to (E25) of claim 1.

9. A tester composition for determining the presence or absence of an IgE antibody in a subject, the tester composition comprising a polypeptide as defined in any of (E1) to (E25) of claim 1.

10. A method for determining the presence or absence of a polypeptide as defined in any of (E1) to (E25) of claim 1 in a raw material or processed product, the method comprising detecting the polypeptide as defined in any of (E1) to (E25) of claim 1 in the raw material or processed product.

11. A raw material or processed product in which an antigen is eliminated or reduced, wherein the antigen is at least one polypeptide as defined in any of (E1) to (E25) of claim 1.

12. A method for producing a raw material or processed product in which an antigen is eliminated or reduced, the method comprising the step of confirming that the antigen has been eliminated or reduced in a production process of the raw material or the processed product, wherein the antigen is at least one polypeptide as defined in any of (E1) to (E25) of claim 1.
